(19) United States
(12) Patent Application Publication
(10) Pub. No.: US 2026/0124256 A1
(43) Pub. Date: May 7, 2026

(54) LIPID ENCAPSULATED PROBIOTIC COMPOSITIONS

(71) Applicant: VitaKey Inc., Washington, DC (US)

(72) Inventors: Aaron C. Anselmo, Chapel Hill, NC (US); Shannon M. Toffton, Raleigh, NC (US); James D. Sievert, Pittsboro, NC (US); Kevin M. Luo, Raleigh, NC (US); Anant Shankar Balijepalli, Durham, NC (US); Tiffany B. Hendley, Whitsett, NC (US); Cheyenne N. Roth, Durham, NC (US); Andrew D. Christenson, Raleigh, NC (US); Joseph Collins, Auckland (NZ); Andrea Stamp, Brookline, MA (US); Ana Jaklenec, Lexington, MA (US); Catherine B. Reynolds, Mountain Brook, AL (US); Robert S. Langer, Newton, MA (US); Megan V. Frisbee, Raleigh, NC (US)

(21) Appl. No.: 19/117,725

(22) PCT Filed: Oct. 3, 2023
(86) PCT No.: PCT/US2023/034395

§ 371 Date: Apr. 2, 2025

Related U.S. Application Data
(60) Provisional application No. 63/412,848, filed on Oct. 3, 2022.
(60) Provisional application No. 63/477,016, filed on Dec. 23, 2022.
(60) Provisional application No. 63/440,903, filed on Jan. 24, 2023.
(60) Provisional application No. 63/468,505, filed on May 23, 2023.
(60) Provisional application No. 63/505,882, filed on Jun. 2, 2023.
(60) Provisional application No. 63/515,063, filed on Jul. 21, 2023.
(60) Provisional application No. 63/520,344, filed on Aug. 17, 2023.

(51) Int. Cl. A61K 35/744; A23C 9/123; A23G 3/36; A23L 2/52; A23L 29/00; A61K 9/00; A61K 9/48; A61K 9/50; A61K 31/047; A61K 35/00; A61K 35/745; A61K 35/747

(52) U.S. Cl. CPC A61K 35/744; A23C 9/1234; A23C 9/1238; A23G 3/366; A23L 2/52; A23L 29/065; A61K 9/0053; A61K 9/0056; A61K 9/4833; A61K 9/485; A61K 9/4858; A61K 9/5015; A61K 9/5089; A61K 31/047; A61K 35/745; A61K 35/747; A23C 2220/204; A23V 2400/175; A23V 2400/531; A61K 2035/115

(57) ABSTRACT

A probiotic composition includes microbes and one or more barrier materials including, on a dry weight basis: about 40% to about 99% (w/w) lipid, and about 0% to about 59% (w/w) of: a carbohydrate, a protein, a polymer, and/or combinations thereof.

### CROSS-REFERENCE TO RELATED APPLICATIONS

This application claims priority to U.S. Provisional Application No. 63/520,344, filed on Aug. 17, 2023: U.S. Provisional Application No. 63/515,063, filed on Jul. 21, 2023; U.S. Provisional Application No. 63/505,882, filed on Jun. 2, 2023; U.S. Provisional Application No. 63/468,505, filed on May 23, 2023; U.S. Provisional Application No. 63/440,903, filed on Jan. 24, 2023; U.S. Provisional Application No. 63/477,016, filed on Dec. 23, 2022; and U.S. Provisional Application No. 63/412,848, filed on Oct. 3, 2022. The text of each of the above-referenced applications is incorporated herein by reference.

### TECHNICAL FIELD

The present disclosure is generally related to probiotic compositions (e.g., probiotic particle preparations) and technologies (e.g., methods of preparation, use, etc.) relating thereto.

### BACKGROUND

The health benefits of probiotics to mammals such as humans have been studied for several decades. However, in many cases, the challenges associated with the delivery and viability of probiotic microbes (among other challenges) remain unsolved. As such, probiotics have yet to realize their full potential.

### SUMMARY

A probiotic preparation (e.g., a probiotic composition, e.g., a probiotic particle preparation) can be included in a supplement, a food, a supplemented (i.e., fortified) food product, a beverage, a supplemented (i.e., fortified) beverage product, a powder, a gummy product (e.g., gummy bear, gummy worm, gelatin-based gummy product, pectin-based gummy product, etc.), or a supplemented (i.e., fortified) powder product intended to confer health benefits.

Popular nutraceuticals include probiotics, prebiotics, microbes, microbial metabolites, bacteria, yeast, phage for example, Lactobacillus, Bifidobacterium, Bacillus, Saccharomyces, Streptococcus. Enterococcus, Escherichia, oligo polysaccharides, polysaccharides, lactic acid, prebiotics, short-chain fatty acids, etc.

In some embodiments, the present disclosure provides technologies that involve utilizing microbes (e.g., probiotics) to confer health benefits.

In some embodiments, the present disclosure provides technologies in which probiotics are provided as a preparation that is compatible, for example, with an ingestible source such as a supplement, and/or a food or nutrient source, and/or a drinking source, consumed by a human or an animal (e.g., a chicken, a cow, a dog, etc.).

In some embodiments, provided particle preparations (e.g., probiotic compositions) achieve one or more advantages such as stability, extended shelf-life, controlled release, improved survival after ingestion, and compatibility with other materials.

Disclosed herein, among other things, are compositions and methods for manufacture, maintenance (e.g., storage) and/or use (e.g., administration or delivery) of probiotic compositions (e.g., particle preparations comprising microbes comprising at least one of Lactobacillus rhamnosus, Lacticaseibacillus rhamnosus, Lactobacillus acidophilus, Bifidobacterium lactis, Bifidobacterium animalis. Bifidobacterium animalis subsp. Lactis, and/or mineral such as calcium carbonate).

In some embodiments, the nutraceutical is or comprises, for example, one or more antioxidants, macronutrients, micronutrients, minerals, prebiotics, probiotics, vitamins, or combinations thereof. In some embodiments, a nutraceutical is or comprises a probiotic such as Lactobacillus rhamnosus, Lacticaseiobacillus rhamnosus, Lactobacillus acidophilus, Bifidobacterium lactis, Bifidobacterium animalis. Bifidobacterium animalis subsp. Lactis. In some embodiments, a nutraceutical is or comprises a mineral such as calcium carbonate.

In some embodiments, provided probiotic preparations (e.g., probiotic compositions) are characterized by one or more of the following advantages: (i) encapsulation and protection of probiotics within a hydrophobic or lipid material; (ii) improved shelf-life and resistance to degradation and resistance to viability or colony forming unit (CFU) losses at decreased temperatures (e.g., −80° C., −20° C., and/or 4° C.), elevated temperatures (e.g., 22° C., 25° C., 30° C., 35° C., and/or 40° C.), in food and/or food products, in beverages and/or beverage products, in supplements, in dry powders, in the presence of high relative humidity (e.g., up to 100%) or moisture, or a combination thereof; (iii) stability enhancement for payload component (e.g., microbes, probiotics) in supplements, foods, beverages, physiological fluids, the host's body; (iv) encapsulation and protection of probiotics within a hydrophobic or lipid material; (v) improved shelf-life in food or beverage matrices (e.g., protein bars, dry powders, milk powders, whey powders, yogurt, drinkable yogurt, water, etc.); (vi) improved compatibility with other components of nutraceutical products and/or compositions that include them (e.g., supplements, foods, drinks, or other edible materials), specifically compatibility with: high water activity powders such as milk powders), liquids containing nutrients (e.g., sugars, carbohydrates, proteins, amino acids, lipids, etc.: e.g., such as, yogurt and/or milk); (vii) compatibility attributes that permit stable combination of payload components (e.g., microbes [e.g., probiotics]) when with (e.g., by mixture with and/or integration into) supplements and/or complex foods and/or beverages (e.g., milk, milk powder, yogurt) and/or ingredients (e.g., non-encapsulated probiotics, high water activity powders) in various harsh environments, such as: decreased temperatures (e.g., −80° C.-20° C. and/or 4° C.), elevated temperatures (e.g., 22° C., 25° C. 30° C., 35° C., and/or 40° C.), high humidity and/or moisture, stomach acids, acids, proton-rich liquid environments, or a combination thereof; (viii) stability of particles and payload in an aqueous liquid against heat, acid, protons, salt, light, water, oxidation, antimicrobial peptides, and/or elevated temperatures; (ix) improved survival and resistance to viability or colony forming unit (CFU) losses during manufacturing processes such as pasteurization, shear mixing, elevated pressurized processes, elevated temperature processes, etc.; (x) stability of particles and/or payload in, or as, a dry powder against heat, acid, protons, salt, light, water, moisture, humidity, oxidation, antimicrobial peptides, and/or elevated temperatures; (xi) resistance and mitigation of water uptake as determined by moisture content; (xii) tunable properties including size, coating thickness, morphology, geometry, loading, dose, interactions with the surrounding environment, and release conditions, etc.; (xiii) improved anti-caking, anti-clumping, anti-agglomerating, and/or anti-aggregating functionality at elevated temperatures; (xiv) maintenance and preservation of probiotic composition morphology (e.g., particle geometry) when exposed to typically degrading conditions, such as: decreased temperatures (e.g., −80° C., −20° C., and/or 4° C.), elevated temperatures (e.g., 22° C., 25° C., 30° C., 35° C., and/or 40° C.), in foods and/or food products, in beverages and/or beverage products, in supplements, in dry powders, in the presence of high relative humidity (e.g., up to 100%) or moisture, or a combination thereof; (xv) stability of particles and payload in high water activity environments such as packaged with or incorporated within a gummy product (e.g., gummy bear, gummy worm, gelatin-based gummy product, pectin-based gummy product, etc.).

As disclosed herein, microbes (e.g., probiotics) may be used to confer health benefits in a human or animal. For example, microbes may be administered to a human or animal to contribute to modifying microbiome diversity, modulating the microbiome, influencing colonization of other microbes, modulating mucus barrier strength, modulating epithelial barrier strength in the gastrointestinal tract, secreting beneficial metabolites (e.g., lactic acid), conferring immune-boosting benefits (e.g., via stimulating the immune system), and/or supporting bowel movements, etc.

Without being bound by any theory, microbes (e.g., probiotics) which confer health benefits, may interact with microbes that already exist in the host (e.g., commensal microbes), by preventing the colonization or attachment of pathogenic or pathobiont microbes, interacting with the host's own cells, secreting metabolites, colonizing the host, stimulating the host to produce metabolites or compounds, and/or modulating the microbiome, etc. Accordingly, these approaches may be useful in conferring health benefits (e.g., increased immunity, improved bowel movements, reduction of pathogenic or pathobiont species in the host's microbiome, etc.).

Microbes (e.g., probiotics) of the present disclosure may be used to produce compounds. Microbe-produced compounds may be beneficial to organisms (e.g., humans, animals, etc.). In some instances, microbes may produce compounds with value and/or benefit, e.g., including but not limited to vitamins, amino acids, lipids, carbohydrates, lactic acid, and immune system stimulating agents).

Further, microbes (e.g., probiotics) of the present disclosure may be used to modulate the microbiome. Microbiome modulation may be beneficial to organisms (e.g., human, animals, etc.) through various mechanisms (e.g., prevention of colonization of pathogens and/or pathobionts, modulation of the existing microbiome).

Microbes (e.g., probiotics) of the present disclosure may also be used to modulate biological barriers in the gastrointestinal tract (e.g., mucus barrier, epithelial barrier, etc.). Biological barrier modulation may be beneficial to organisms (e.g., humans, animals, etc.) through various mechanisms (e.g., mitigate diffusion and/or transport of toxic and/or host-damaging compounds).

Microbes (e.g., probiotics) of the present disclosure may also interact with a host's immune system (e.g., immune cells, etc.). Interaction with the host's immune system may be beneficial to organisms (e.g., humans, animals, etc.) through various mechanisms (e.g., increased antibody production, increased immune cell proliferation, increased stimulation of immune cells, inhibition of immune cells, induction of immune tolerance, etc.).

A persistent challenge in the use of microbes (e.g., probiotics) is achieving sufficient microbial survival after host ingestion due to microbe sensitivity to various physiological conditions (e.g., the low pH environment of the stomach. Microbe (e.g., probiotic) sensitivity to various physiological conditions may reduce microbial viability (i.e., impair the ability of the microbe to form colony forming units) and of and/or reduce any one or more of the benefits conferred by microbes (e.g., probiotics) as described above (e.g., production of compounds with value and/or benefit, reduced microbiome modulation, reduced modulation of biological barriers, etc.).

An additional challenge in using microbes (e.g., probiotics) is achieving sufficient microbe interactions with the host organism or with the microbes already present within the host's microbiome (e.g., commensal organisms). Insufficient interaction may result in a reduced amount of time that probiotics spend in the host, reduce the amount of time that probiotics spend secreting metabolites in the host, reduce the beneficial interactions between the probiotic and the host, and/or prevent localization of probiotics with physiological regions within the host (e.g., regions that probiotics must interact with to provide health benefits).

Another challenge in using microbes (e.g., probiotics) is achieving a sufficient microbe dose in various supplement, food, and/or beverage formats to confer health benefits in a host. Microbes (e.g., probiotics) may be sensitive to the properties of the various supplement, food, and/or beverage products, and/or the conditions in which these products are typically stored (e.g., high water activity, high humidity, high moisture, high temperatures). Microbe (e.g., probiotic) sensitivity may result in reduced ability to form colony forming units, and/or reduced ability to confer one or more benefits as described above (e.g., production of compounds with value and/or benefit, reduced microbiome modulation, reduced modulation of biological barriers, etc.).

In view of the foregoing challenges, technologies that can preserve and maintain probiotic viability and/or ability to form CFUs throughout the probiotic's lifetime (e.g., fermentation, processing, manufacturing, storage, ingestion, digestion, etc.) are disclosed herein.

Preserving probiotic viability and/or ability to form CFUs is important because many of the beneficial functions provided by probiotics require their viability (e.g., metabolite secretion, colonization, etc.).

When considering methods for withstanding challenges encountered throughout a probiotic's lifetime (e.g., fermentation, processing, manufacturing, incorporation into consumer products, storage, ingestion, digestion, etc.), encapsulation of the probiotic in barriers that prevent, limit, and/or control interactions with the surrounding environment may help in solving these issues.

In some embodiments, probiotic preparations of the present disclosure comprise promoters that increase metabolite secretion, improve probiotic colonization, and/or improve probiotic survival. In some embodiments, probiotic preparations of the present disclosure may comprise promoters that respond to the environment that the probiotic is currently in (e.g., dry powders, beverages, stomach acids) to modulate or change that environment such that it is more favorable for probiotic survival, CFU maintenance, preservation, stability, and/or viability.

In some embodiments, probiotic preparations of the present disclosure may comprise promoters that increase probiotic growth.

In some embodiments, probiotic preparations of the present disclosure may comprise barriers that prevent, limit, and/or control interactions with the surrounding environment (which changes throughout the probiotic's lifetime).

In some embodiments, probiotic preparations of the present disclosure comprise a sufficient amount of one or more microbes (e.g., probiotics) such that when ingested by a host, the one or more microbes (e.g., probiotics) confer one or more health benefits. In some embodiments, probiotic preparations of the present disclosure comprise a sufficient amount of one or more microbes (e.g. probiotics) such that when added to a consumer product (e.g., a supplement, a food product, a beverage, a powder, or a gummy) the one or more microbes (e.g., probiotics) confer one or more health benefits when ingested by a host.

In some embodiments, the present disclosure provides compositions that are or comprise a particle preparation, wherein the particles of the particle preparation comprise (i) a lipid component; and (ii) a payload component, wherein the lipid component comprises a lipid that is stable to and/or resists moisture uptake and/or resists high water activity in the chosen supplement, food, beverage, and/or physiological fluid/environment (e.g., stomach acids, stomach, intestines, etc.); and the payload component comprises a microbe (e.g., a probiotic), and wherein the particle preparation enables protection (maintenance/preservation of CFUs and viability) of the probiotic in supplements, foods, beverages, and/or physiological fluids/environment into the particle (e.g., stomach acids, stomach, intestines, etc.), and/or processing/manufacturing environments (e.g., high pressure pasteurization, high temperature pasteurization, etc.). In some such embodiments, the composition and/or the particle preparation is characterized in that the payload component shows increased stability (e.g., is protected against one or more of degradation, oxidation, pressure, other physical and/or chemical changes) when exposed to one or more environmental conditions such as, for example, heat, acid, protons, pasteurization, shear, high pressure, salt, light, water, oxidation, antimicrobial peptides, elevated temperatures, and/or in the context of a complex material. Alternatively or additionally, in some such embodiments, the compositions and/or the particle preparation is characterized in that it releases the payload component in the host's gastrointestinal tract (e.g., esophagus, stomach, small intestine, large intestine, etc.).

In certain embodiments, the present disclosure provides human and/or animal consumable compositions (e.g., supplement products, food products, powder products, beverage products, liquid products, gummy products, etc.) comprising disclosed probiotic preparations, at least one probiotic, or a combination thereof. In some instances, particle preparations (e.g., probiotic preparations) further comprise at least one nutraceutical. In some embodiments, particle preparations (e.g., probiotic preparations) further comprise at least one compound (e.g., glucose) to improve probiotic growth rate. In some embodiments, particle preparations (e.g., probiotic preparations) further comprise at least one compound (e.g., amino acids) to improve probiotic metabolism.

In some embodiments, humans may be a prenatal human, infant, toddler, child, teenager, adolescent, young adult, adult, geriatric, medical patient, athlete, student, etc.

In some embodiments, animals may be an agricultural animal (e.g., a horse, a cow, a camel, a goat, a sheep, a fish, a crab, etc.), a pet (e.g., a dog, a cat, a fish, a duck, etc.), and/or a wild animal (e.g., a raccoon, a deer, a moose, a bear, a whale, an ant, a bee, a wasp, etc.).

In some aspects, consumable compositions comprising probiotic compositions (e.g., probiotic preparations) may be edible. In some aspects, an edible composition may be a gummy product or a powder or slurry that is mixed with food (e.g., a freshly prepared meal, a pre-prepared meal, a prepared food product, etc.) prior to consumption.

In some aspects, consumable compositions comprising probiotic compositions (e.g., probiotic preparations) may be drinkable. In some aspects, a drinkable composition may be a powder or slurry that is mixed with a beverage (e.g., water, soda, electrolyte beverage, a protein shake, etc.) prior to consumption.

In one aspect, the present embodiments are directed to a method for preparing a nutraceutical payload component (e.g., a probiotic), the method comprising one or more steps of (i) formulation (e.g., encapsulation, association, and/or complexation with materials); (ii) post-formulation processing (e.g., drying, characterization, additions of excipients, etc.); (iii) storing (e.g., bagging in aluminum sachets, addition of nitrogen or vacuum environments, etc.); (iv) combining with foods and/or beverages (e.g., as supplements); (v) ingesting (e.g., swallowing as a capsule, addition to other existing food and/or beverages); or (vi) a combination of (i), (ii), (iii), (iv), and (v).

In another aspect, the present embodiments are directed to a microbe and/or probiotic prepared by any of the methods described herein.

In another aspect, the present embodiments are directed to a microbe and/or probiotic preparation comprising a carrier component and a payload component, wherein the payload component is associated with (e.g., encapsulated in, adhered to, dispersed in) the carrier component; and wherein the payload component comprises: (i) a microbe and/or probiotic component; (ii) a nutrient that the payload utilizes for metabolism (e.g., sugar, glucose, protein); (ii) a component that modulates the environment (e.g., food matrix, liquid environment, physiological fluid, tissue/organ such as stomach, etc.) to preserve or maintain CFUs or viability of probiotics or microbes; (iv) one or more other payload component(s), or (v) a combination of (i), (ii), (iii) or (iv).

In some embodiments, provided probiotic compositions (e.g., probiotic preparations) are or comprise particles (e.g., microparticles) that include a matrix component (e.g., a polymer component) and a payload component (e.g., microbe payload component). In some instances, one or more layers of matrix components are present.

In some embodiments, a matrix component is or comprises a hydrophobic component. In some embodiments, a hydrophobic component is or comprises a sugar, a polysaccharide, a carbohydrate, an oil, a fat, a wax, a protein, or a combination thereof. In some instances, a matrix component comprises a salt (e.g., calcium carbonate). In some embodiments, a matrix component comprises a surfactant (e.g., sodium dodecyl sulfate). In some embodiments, one or more layers of payload components are present.

In some embodiments, a matrix component comprises a biocompatible material. In some embodiments a biocompatible material is or comprises a sugar, a polysaccharide, a carbohydrate, an oil, a fat, a wax, a lipid, a protein, an amino acid, a peptide, or a combination thereof. In some embodiments, a matrix component comprises a salt (e.g., calcium carbonate). In some embodiments, a matrix component comprises a surfactant (e.g., sodium dodecyl sulfate).

In some embodiments, a matrix component further comprises one or more microbial (e.g., bacterial, yeast, etc.) species.

The present disclosure provides technologies for making and/or characterizing matrix components comprising a lipid component described herein, and/or compositions that include them. In some embodiments, the disclosed processes and methodologies to generate lipid matrices include extrusion, granulation, extrusion-based methods, melt processing, shear-based granulation methods, atomization, prilling, spray chilling, and/or spray congealing methods.

In some embodiments, the carrier component comprises at least one carbohydrate, at least one polymer, and/or at least one lipid.

In some embodiments, a microbe component comprises a microbe selected from the group consisting of: a naturally-occurring microbe, a commercially-available microbe or probiotic, a probiotic preparation (e.g., freeze-dried or already-formulated probiotics), and a microbe prepared by any method described herein. As such, in some embodiments of the present disclosure, a microbe component comprises a commercially-available probiotic powder that includes a carrier or matrix component that is further encapsulated in a lipid carrier, as described herein. In some embodiments, an inner carrier contain the probiotic is itself encapsulated in one or more outer lipid layers or carrier.

In some embodiments, a probiotic composition includes at least 10\hat{ }8 probiotics.

In some embodiments, a probiotic composition comprises particles.

In some embodiments, a probiotic composition includes at least 10\hat{ }8 CFU probiotics per gram of probiotic particles (e.g., probiotic preparation).

In some embodiments, a probiotic composition, when combined with a food, beverage, and/or product (e.g., milk powder, yogurt, gummy bear, etc.) is characterized by prevention of viability loss and/or CFU loss after storage at various conditions (e.g., temperatures, humidity, oxygen content, etc.).

In another aspect, some embodiments of the present disclosure are directed to a method of promoting health or longevity in a human, comprising: providing an effective amount of a probiotic composition as described herein.

Regardless of the shape of particles, a particle “diameter” (i.e., a particle size) is the longest distance from one end of the particle to another end of the particle. In some embodiments, probiotic compositions (e.g., particle preparations) are or comprise particles (e.g., polymer microparticles) with a distribution of particle diameters (e.g., D[3,2], D[4,3], etc.). In some embodiments, probiotic compositions (e.g., probiotic preparations) are or comprise particles with a distribution of particle diameters (e.g., D[3,2], D[4,3], etc.) of up to about 3000 μm, up to about 2000 μm, up to about 1000 μm, of up to about 500 μm, up to about 400 μm, up to about 300 μm, up to about 200 μm, up to about 100 μm, up to about 50 μm, up to about 40 μm, up to about 30 μm, up to about 20 μm, up to about 10 μm, or up to about 5 μm.

In some embodiments, probiotic compositions (e.g., probiotic preparations) are or comprise particles with an average diameter (e.g., D[3,2], D[4,3], i.e., Sauter mean diameter, De Brouckere mean diameter, etc.) of particles in a range of about 5-3000 μm, about 5-2000 μm, about 5-1000 μm, about 5-500 μm, about 5-250 μm, about 5-175 μm, about 5-100 μm, about 5-50 μm, about 5-10 μm, or about 4-6 μm.

In some instances, particle preparations (e.g., probiotic compositions) comprise particles (e.g., lipid microparticles comprising a nutraceutical payload component) characterized by an average particle diameter (e.g., D[3,2], D[4,3], etc.) within a range of about 60 μm to about 300 m.

In some instances, particle preparations (e.g., probiotic compositions) comprise particles (e.g., lipid microparticles comprising a nutraceutical payload component) characterized by an average particle diameter (e.g., D[3,2], D[4,3], etc.) within arrange of about 100 μm to about 1000 μm.

In some instances, particle preparations (e.g., probiotic compositions) comprise particles (e.g., lipid microparticles comprising a nutraceutical payload component) characterized by an average particle diameter (e.g., D[3,2], D[4,3], etc.) within arrange of about 5 μm to about 60 μm.

In some instances, particle preparations (e.g., probiotic compositions) comprise particles (e.g., lipid microparticles comprising a nutraceutical payload component) characterized by an average particle diameter (e.g., D[3,2], D[4,3], etc.) within arrange of about 5 μm to about 300 μm.

In some embodiments, particle preparations (e.g., probiotic compositions) may have any shape or form, for example, particle preparations may have a cross-section shape of a circle, an oval, a triangle, a square, a hexagon, or an irregular shape. In some embodiments, probiotic compositions comprise particles (e.g., microparticles), wherein a majority of particles have a common shape. In some embodiments, probiotic compositions are or comprise particles of various shapes in combination.

In some embodiments, provided particle preparations (e.g., probiotic compositions) are characterized by having a layered structure wherein adjacent components in the particle preparations have different chemical structures.

In some embodiments, provided probiotic compositions are characterized by having multiple polymer components, wherein the particle preparations (e.g., probiotic compositions) may be additionally encapsulated with a separate polymer component.

In some embodiments of a particle preparation (e.g., probiotic composition) provided by the present disclosure, a first layer is or comprises a hydrophilic material and/or a water-soluble material and a second layer is or comprises a hydrophobic material and/or a fat-soluble material. For example, in some embodiments, a water-soluble payload material may be or comprise probiotics and/or microbes: in some embodiments, such water-soluble payload material may be encapsulated or otherwise dispersed within a hydrophilic material (e.g., maltodextrin). In some embodiments, a hydrophilic material forms a layer and a hydrophilic payload material (e.g., probiotics) may be encapsulated or otherwise dispersed with a hydrophobic polymer (e.g., paraffin wax, stearic acid, palmitic acid, etc.) in a different layer or in a core (or vice versa) of relevant particles. In some embodiments, the layers are reversed.

In some embodiments, provided particle preparations (e.g., probiotic compositions) are characterized by low water uptake in high humidity or high moisture conditions. In some embodiments, the present disclosure provides technologies for preparing and/or characterizing probiotic compositions comprising low water/moisture uptake when exposed to conditions that typically increase water/moisture uptake.

Particle preparations (e.g., probiotic compositions) comprising low water uptake provides benefits over existing products, among other things because water uptake in probiotics leads to viability and/or CFU loss during manufacturing and/or during shelf-storage.

In some embodiments, the present disclosure provides technologies for manufacturing provided probiotic compositions (e.g., probiotic preparations) that reduce uptake of water during and/or following manufacturing (thereby minimizing or avoiding risk of water uptake in probiotic supplement/food/beverage/gummy products).

In some embodiments, the present disclosure provides technologies for manufacturing provided probiotic compositions (e.g., probiotic preparations) that comprise a residual solvent content lower than a predetermined amount. In some embodiments, the residual solvent is an organic solvent, for example, hexane, ethanol, ethyl acetate, acetone, methylene chloride, methanol, dichloromethane, isopropyl alcohol (i.e., 2-propanol), or any combination thereof.

In some embodiments, the present disclosure provides probiotic compositions (e.g., probiotic compositions) with resistance (e.g., mitigation of viability and/or CFU losses) to stomach acids, simulated gastric fluids, proton-rich liquids, or low-pH (e.g., less than a pH of 3) fluids and/or liquids and/or beverages. The present disclosed probiotic compositions, therefore, may provide benefits over existing products, among other things because acidic solutions lead to viability and/or CFU loss of probiotics after ingestion, and/or upon contact with the stomach and/or stomach fluids and/or upon contact with beverages. Thus, the present disclosure provides technologies with a variety of advantages.

In some embodiments, the present disclosure provides probiotic preparations (e.g., probiotic compositions) with enhanced survival (e.g., maintenance and preservation of viability and/or CFU) in supplements, gummy products, foods and/or beverages at elevated temperatures, water activities, humidity and/or moisture. This provides benefits over existing products, among other things because these conditions lead to rapid viability and/or CFU loss of probiotics after incorporation with products and during shelf-storage. Thus, the present disclosure provides technologies with a variety of advantages.

In some embodiments, the present disclosure provides probiotic preparations (e.g., probiotic compositions) with enhanced survival (e.g., maintenance and preservation of viability and/or CFU) during processing and/or manufacturing.

In some embodiments, particle preparations (e.g., probiotic compositions) disclosed herein are: 1) less susceptible to stomach acids or other physiological fluids which lead to viability and/or CFU losses; 2) less susceptible to loss of viability due to shelf-storage in supplements, gummy products, foods, and/or beverages; 3) less susceptible to loss of viability due to elevated temperatures, high humidity, high moisture, or high water activity; and/or 4) less susceptible to loss of viability due to processing or manufacturing approaches for incorporating into supplements, foods, and/or beverages.

In some embodiments, probiotic compositions (e.g., particle preparations) of the present disclosure maintain stability and viability when distributed and/or incorporated into high water activity products (e.g., supplements, foods, and/or beverages).

In some embodiments, probiotic compositions (e.g., particle preparations) of the present disclosure maintain probiotic stability and viability when contacted with and transiting through the stomach and stomach fluids following ingestion.

In some embodiments, provided particle preparations (e.g., probiotic compositions) may comprise both low residual solvent content and have low water activity and provide benefits to stomach and/or stomach acid survival.

In some embodiments, provided particle preparations (e.g., probiotic compositions) may comprise low residual solvent content and/or have low water activity and/or have low moisture content and provide benefits to stomach and/or stomach acid survival.

In some embodiments, a bile-responsive, pH-responsive, and/or microbiome-responsive polymer component can facilitate location-specific release of the probiotics into the small and/or large intestines by leveraging the physiological or environmental differences in stomach and intestinal fluids, tissues and/or organs.

In some embodiments, a temperature-responsive polymer is more readily processed at lower temperatures (e.g., glass transition temperature) through addition of payloads or plasticizers. In some embodiments, payloads alone can lower the glass transition temperature of temperature-responsive polymers. Collectively, this facilitates manufacturing and processing approaches at lower temperatures, since polymer component and payload component can more easily transition from flowable homogenous liquid states to solid states (e.g., particles). This specifically addresses challenges related to processing and formulation of probiotics, since they are highly sensitive to elevated temperatures.

In some instances, a provided probiotic composition (e.g., that is or comprises a probiotic preparation) provides increased shelf-life in milk powders at 18° C., 25° C., 30° C., 35° C.

In some instances, a provided probiotic composition (e.g., that is or comprises a probiotic preparation) provides increased shelf-life in yogurt at 4° C., 18° C., 25° C., 30° C., 35° C.

In some instances, a provided probiotic composition (e.g., that is or comprises a probiotic preparation) provides increased shelf-life in a gummy product (e.g., gummy bear, gummy worm, gelatin-based gummy product, pectin-based gummy product, etc.) at 18° C., 25° C., 30° C., 35° C.

In some instances, a provided probiotic composition (e.g., that is or comprises a probiotic preparation) provides increased shelf-life in capsules at 4° C., 18° C., 25° C., 30° C., 35° C.

In some embodiments, a provided microbe preparation (i.e., probiotic composition) (e.g., that is or comprises a particle preparation) may be or is effective at protecting payload components (e.g., nutraceutical payload component) against a physical change, a chemical change, or both (e.g., degradation, oxidation, hydrolysis, isomerization, fragmentation, or a combination thereof).

For example, in some embodiments a physical or chemical change may be induced by one or more of heat, light, water, other microbes, other ingredients in a product, etc. In some instances, degradation of a payload component (e.g., a probiotic component) is characterized by utilizing CFU enumeration on agar to compare CFUs of a payload component after being incorporated in particle preparations.

In some embodiments, a provided probiotic composition (e.g., that is or comprises a probiotic preparation) may be or remain stable. e.g., to store for a particular period of time under particular conditions.

For example, in some embodiments, 99% of a payload component present in a provided composition at a particular point in time remains present, and/or one or more size characteristics (e.g., average diameter and/or one or more features of size distribution of a particle composition) remains stable throughout a period of time during which the composition is maintained under particular conditions. For example, a payload component present in a provided composition may remain stable in a dry powder form (e.g., a high water activity powder) for a period of time. For example, a payload component present in a provided composition may remain stable in a gummy product (e.g., gummy bear, gummy worm, gelatin-based gummy product, pectin-based gummy product, etc.) for a period of time. In some embodiments, a payload component present in a provided composition may remain stable for a period of time when dispersed within solid food (at chilled, room temperature, and/or elevated temperatures). In some embodiments, a payload component present in a provided composition may remain stable for a period of time when dispersed within a beverage (at chilled, room temperature, and/or elevated temperatures). In some embodiments, a payload component present in a provided composition may remain stable for a period of time when dispersed within an acidic solution (for example, at a pH<3).

In some embodiments, the period of time is at least 2, 3, 4, 5, 6, 7, 8, 9, 10, 11, 12, 13, 14, 15, 16, 17, 18, 19, 20, 21, 22, 23, 24 weeks or more, and/or at least about 1, 2, 3, 4, 5, 6, 7, 8, 9, 10, 11, 12 months or more, and/or at least about 1, 2, 3, 4, 5 years or more.

In some such embodiments, the particular conditions comprise ambient temperature. In some such embodiments, the particular conditions comprise elevated (above ambient) temperature. Alternatively or additionally, in some embodiments, the particular conditions comprise aqueous conditions (e.g., aqueous liquid conditions). In some embodiments, the period of time is at least two months and the particular conditions comprise ambient temperature.

In some embodiments, disclosed particle preparations (e.g., probiotic compositions) may be or are effective to protect against permeation of water.

As used herein, protection against permeation of water should not be construed to be limited as to occurring only in water.

In some embodiments, protection against permeation of water may occur in an environment in which water may be present and/or introduced. For example, protection against permeation of water may occur in water, aqueous-based liquid, consumable liquid (e.g., milk, juice, etc.) non-aqueous-based liquid, oils, and/or dry environments. For example, protection against permeation of water and/or moisture may occur in dry powders or solid foods.

In some embodiments, disclosed particle preparations (e.g., probiotic compositions) may be or are effective to protect against permeation of water, for example, in water, aqueous-based liquid, consumable liquid (e.g., milk, juice, etc.), non-aqueous-based liquid, oils, or dry environments, or combinations thereof. In some instances, the particle preparations are stable (>65% chemical stability) up to about 200 days, about 1 year, about 2 years, about 3 years, up to 4 years, about 5 years in water, aqueous-based liquid, consumable liquid (e.g., milk, juice, soda, etc.), non-aqueous-based liquid, oils, dry environments, and/or a combination thereof. Accordingly, the present embodiments provide liquid stability probiotics, both in general and in particle preparations.

In some embodiments, particle preparations (e.g., probiotic compositions) are stable (>10% probiotic viability) when combined with milk powders. In some instances, the formulation does not induce viability loss of probiotics when combined with milk powders.

In some embodiments, particle preparations (e.g., probiotic compositions) are stable (>10% probiotic viability) when combined in a gummy product (e.g., gummy bear, gummy worm, gelatin-based gummy product, pectin-based gummy product, etc.). In some instances, the formulation does not induce viability loss of probiotics when combined with a gummy product.

In some embodiments, particle preparations (e.g., probiotic compositions) exhibit improved anti-caking, anti-clumping, anti-agglomerating, and/or anti-aggregating performance over a polymer component with or without the probiotic compositions.

In some embodiments, a particle preparation may further comprise an excipient component (e.g., an anti-caking component, an anti-clumping component, a plasticizer, an anti-agglomerating component, and/or an anti-aggregating component [e.g., any of an excipient comprising microcrystalline cellulose, starches, calcium carbonate, etc.], wherein an excipient component is at least about 99 wt %, at least about 90 wt %, at least about 85 wt %, at least about 80 wt %, at least about 75 wt %, at least about 70 wt %, at least about 65 wt %, at least about 60 wt %, at least about 55 wt %, at least about 50 wt %, at least about 45 wt %, at least about 40 wt %, at least about 35 wt %, at least about 30 wt %, at least about 25 wt %, at least about 20 wt %, at least about 15 wt %, at least about 10 wt %, at least about 5 wt %, at least about 1 wt %, at least about 0.8 wt %, at least about 0.5 wt %, at least about 0.1 wt % of a particle preparation (i.e., a probiotic composition).

The disclosed probiotic compositions (e.g., particle preparations comprising a probiotic payload) may be particularly useful for stabilizing and extending shelf-life (preserving CFUs) of payload components in consumable compositions (e.g., a food product, a beverage product, an animal-consumable product, dry powders, supplements, a gummy product, etc.), where probiotic components typically lose viability when exposed to high water activity entities.

In certain embodiments, the present disclosure provides consumable compositions (e.g., a food product, a beverage product, an animal-consumable product, dry powders, a supplement, etc.) comprising disclosed probiotic compositions, at least one probiotic, or a combination thereof. In some instances, particle preparations (e.g., probiotic compositions) further comprise at least one probiotic.

In some embodiments, particle preparations (e.g., probiotic compositions) comprising low water uptake may be used to stabilize payload components in consumable compositions (e.g., a food product, a beverage product, an animal-consumable item, dry powders, etc.). In some aspects, provided particle preparations (e.g., probiotic compositions) are or may be useful for improving health or longevity in humans and/or animals. In some aspects, provided consumable compositions are or may be useful for improving health or longevity in humans and/or animals.

In some aspects, consumable compositions comprising particle preparations (e.g., probiotic compositions) may be edible. In some aspects, an edible composition may be a protein bar, a cereal, a protein powder, a milk powder, a salad dressing, a nutritional supplement, a baby formula, a smoothie, a yogurt, an ice cream, a sachet, a spice, a food additive, a candy, a sprinkle packet, a pet food, an agricultural seed, a dry powder, and/or a fertilizer.

In some aspects, consumable compositions comprising particle preparations (e.g., probiotic compositions) are drinkable. In some aspects, a drinkable composition may be a sports drink, beer, wine, tea, coffee, milk, juice, water, yogurt, soda, carbonated water, or a liquid pharmaceutical formulation.

In some embodiments, the present disclosure provides for preparations of formulations comprising water-resistant polymers associated with (e.g., encapsulating and/or otherwise complexed with) one or more probiotics or payloads, thereby providing compositions and methods for storage in food and/or beverage products.

In some embodiments, protection against heat, acid, protons, salt, light, water, oxidation, antimicrobial peptides, and/or elevated temperatures of payload component is maintained after storage (e.g., with or within a consumable composition) in a freezer (−85° C. to 0° C.), a refrigerator (1-10° C.), or atmospheric temperature (−10° C.-40° C.) for time periods between 0-1 week. 0-1 month. 0-1 year or 1-5 years.

In another aspect, the present embodiments are directed to a food product coated with a probiotic composition as described herein, the food product including a gelatin-based matrix and/or a pectin-based matrix.

In some embodiments, the gelatin-based matrix and/or pectin-based matrix includes a water activity in a range from 0.50 plus or minus 0.01 to 0.70 plus or minus 0.01.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

Aspects and embodiments of the present embodiments are set forth with particularity in the appended claims. A better understanding of certain features and advantages of various aspects of the present disclosed embodiments may be obtained by reference to the following detailed description that sets forth illustrative embodiments, e.g., in which the principles of the embodiments are utilized, and the accompanying figures of the drawing, of which:

FIG. 1A shows, in a non-limiting example, scanning electron and brightfield micrographs of exemplary probiotic compositions comprising a payload component (e.g., 5% (w/w) Lacticaseibacillus rhamnosus HN001, 95% (w/w) paraffin wax).

FIG. 1B shows, in a non-limiting example, scanning electron and brightfield micrographs of exemplary probiotic compositions comprising a payload component (e.g., 5% (w/w) Lacticaseibacillus rhamnosus HN001, 95% (w/w) hydrogenated palm oil).

FIG. 1C shows, in a non-limiting example, scanning electron and brightfield micrographs of exemplary probiotic compositions comprising a payload component (e.g., 5% (w/w) Lacticaseibacillus rhamnosus HN001, 95% (w/w) palmitic acid).

FIG. 1D shows, in a non-limiting example, brightfield micrograph of exemplary probiotic compositions comprising a payload component (e.g., 5% (w/w) Bifidobacterium lactis HN019, 95% (w/w) paraffin wax).

FIG. 1E shows, in a non-limiting example, brightfield micrographs of exemplary probiotic compositions comprising a payload component (e.g., 5% (w/w) Bifidobacterium lactis HN019, 95% (w/w) hydrogenated palm oil).

FIG. 1F shows, in a non-limiting example, brightfield micrographs of exemplary probiotic compositions comprising a payload component (e.g., 35% (w/w) Lacticaseibacillus rhamnosus HN001, 65% (w/w) paraffin wax).

FIG. 1G shows, in a non-limiting example, brightfield micrographs of exemplary probiotic compositions (e.g., extrudate) comprising a payload component (e.g. 5% (w/w) Bifidobacterium lactis HN019, 10% (w/w) calcium carbonate, 85% (w/w) hydrogenated palm oil).

FIG. 1H shows, in a non-limiting example, brightfield micrographs of exemplary probiotic compositions obtained from size reduction (e.g., burr milling) of extrudate presented in FIG. 1G.

FIG. 1I shows, in a non-limiting example, a schematic of exemplary particle preparations which may comprise carrier components, payload components, excipient components, and combinations thereof. Additionally or alternatively, exemplary particle preparations may comprise a particle comprising at least one carrier component, at least one payload component, at least one excipient component, at least one matrix component, or a combination thereof.

FIG. 2A presents exemplary particle morphologies (e.g., particle diameter distributions) of nutraceutical payload components (e.g., probiotic) of Lacticaseibacillus rhamnosus HN001.

FIG. 2B presents exemplary particle morphologies (e.g., particle diameter distributions) of nutraceutical payload components (e.g., probiotic) of Bifidobacterium lactis HN019 after milling.

FIG. 2C illustrates exemplary morphologies (e.g., particle diameter distributions) of probiotic compositions comprising a nutraceutical payload component (e.g. 5% (w/w) Lacticaseibacillus rhamnosus HN001, 95% (w/w) paraffin wax.

FIG. 2D illustrates exemplary particle morphologies (e.g., particle diameter distributions) of nutraceutical payload components (e.g., probiotic) of 5% (w/w) Lacticaseibacillus rhamnosus HN001, 95% (w/w) hydrogenated palm oil.

FIG. 2E illustrates exemplary particle morphologies (e.g., particle diameter distributions) of nutraceutical payload components (e.g., probiotic) of 35% (w/w) Lacticaseibacillus rhamnosus HN001, 65% (w/w) paraffin wax.

FIG. 2F illustrates exemplary particle morphologies (e.g., particle diameter distributions) of nutraceutical payload components (e.g., probiotic) of 5% (w/w) Bifidobacterium lactis HN019, 95% (w/w) hydrogenated palm oil.

FIG. 2G illustrates exemplary particle morphologies (e.g., particle diameter distributions) of nutraceutical payload components (e.g., probiotic) of 5% (w/w) Bifidobacterium lactis HN019, 95% (w/w) paraffin wax.

FIG. 2H illustrates exemplary particle morphologies (e.g., particle diameter distributions) of nutraceutical payload components (e.g., probiotic) of 5% (w/w) Bifidobacterium lactis BI-07, 95% (w/w) paraffin wax.

FIG. 2I illustrates exemplary particle morphologies (e.g., particle diameter distributions) of nutraceutical payload components (e.g., probiotic) of and 5% (w/w) Lactobacillus acidophilus, 95% (w/w) paraffin wax (FIG. 2I).

FIG. 3A shows, in a non-limiting example, a schematic of a method used to apply an additional barrier to a probiotic composition, referred to herein as “pan-coating”.

FIG. 3B presents, in a non-limiting example, brightfield micrographs of exemplary probiotic compositions comprising a payload component (e.g., 5% (w/w) Lacticaseibacillus rhamnosus HN001, 95% (w/w) hydrogenated palm oil), following coating with an additional barrier (e.g., shellac) via pan-coating without any washing.

FIG. 3C presents, in a non-limiting example, brightfield micrographs of exemplary probiotic compositions comprising a payload component (e.g., 5% (w/w) Lacticaseibacillus rhamnosus HN001, 95% (w/w) hydrogenated palm oil), following coating with an additional barrier (e.g., shellac) via pan-coating and after being washed in peptone salt solution for 30 minutes.

FIG. 3D presents, in a non-limiting example, brightfield micrographs of exemplary probiotic compositions comprising a payload component (e.g., 5% (w/w) Lacticaseibacillus rhamnosus H4N001, 95% (w/w) hydrogenated palm oil), following coating with an additional barrier (e.g., shellac) via pan-coating and after being washed in peptone salt solution for 60 minutes.

FIG. 4A presents, in a non-limiting example, a table enumerating viable probiotic (e.g. Lacticaseibacillus rhamnosus HN001 and Bifidobacterium lactis HN019, Bifidobacterium lactis BI-07 and Lactobacillus acidophilus) loading into exemplary probiotic compositions, demonstrating that payload concentration (e.g. CFU/g) can be controlled, for example, by adjusting the ratio of payload component to lipid carrier component (e.g., 5% (w/w) probiotic: 95% lipid carrier component. 35% (w/w) probiotic: 65% lipid carrier component, or 50% (w/w) probiotic: 50% lipid carrier component) during the manufacturing process.

FIG. 4B shows, in a non-limiting example, brightfield micrographs of a probiotic composition comprising two different probiotic payload components (e.g., 2.5% (w/w) Lacticaseibacillus rhamnosus HN001, 2.5% (w/w) Bifidobacterium lactis HN019, and 95% (w/w) hydrogenated palm oil).

FIG. 4C shows, in a non-limiting example, brightfield micrographs of a probiotic composition comprising two different types of payload components (e.g., a micronutrient (5% (w/w) Lutein), a probiotic (5% (w/w) Bifidobacterium lactis HN019, and 90% (w/w) hydrogenated palm oil).

FIG. 5A presents a plot enumerating cell loading in exemplary probiotic compositions (e.g. 5% (w/w) Lacticaseibacillus rhamnosus HN001 in separate formulations of 95% (w/w) paraffin wax, 95% (w/w) hydrogenated palm oil, and 95% (w/w) palmitic acid) relative to enumeration of an equivalent amount (in CFU) of unformulated payload component (e.g. probiotic powder), demonstrating that formulation process(es) do not result in loss of viability (e.g. reduction in CFU/g), as further described herein.

FIG. 5B presents, in a non-limiting example, a plot of log(CFU/g) over time, indicating retention of probiotic viability (e.g., zero log(CFU/g) loss) with exemplary probiotic compositions (e.g., 5% (w/w) Lacticaseibacillus rhamnosus HN001, 95% (w/w) paraffin wax; 5% (w/w) Bifidobacterium lactis HN019, 95% (w/w) paraffin wax) at −20° C. for roughly 5 months. In another non-limiting example, a plot of log(CFU/g) over time, indicating retention of probiotic viability (e.g., 0 log(CFU/g) loss) with exemplary probiotic compositions (5% (w/w) DANISCO floraFIT B. lactis Bi-07, 95% (w/w) paraffin wax; 5% (w/w) Lacticaseibacillus rhamnosus HN001, 95% (w/w) hydrogenated palm oil; 35% (w/w) Lacticaseibacillus rhamnosus HN001, 65% (w/w) paraffin wax) at −20° C. for 1 month.

FIG. 5C presents, in a non-limiting example, a plot of log(CFU/g) over time, indicating retention of probiotic viability (e.g., zero log(CFU/g) loss) with exemplary probiotic compositions (5% (w/w) DANISCO floraFIT B. lactis Bi-07, 95% (w/w) paraffin wax; 5% (w/w) Lacticaseibacillus rhamnosus HN001, 95% (w/w) hydrogenated palm oil; 35% (w/w) Lacticaseibacillus rhamnosus HN001, 65% (w/w) paraffin wax) at 4° C. for 1 month.

FIG. 5D presents, in a non-limiting example, a plot of log(CFU/g) over time, indicating retention of probiotic viability (e.g., 0 log(CFU/g) loss) with exemplary probiotic composition (35% (w/w) Lacticaseibacillus rhamnosus HN001, 65% (w/w) paraffin wax) at 25° C. for 1 month.

FIG. 6A shows, in a non-limiting example, that unformulated probiotics 601 (Lacticaseibacillus rhamnosus HN0001), 602 (Bifidobacterium lactis HN019), 603 (Culturelle Lacticaseibacillus rhamnosus GG), 604 (Nature's Bounty Lactobacillus acidophilus LA14), 605 (DANISCO floraFIT Bifidobacterium lactic Bi-07), and 606 (KP-HOWARU Dophilus 200BL Lactobacillus acidophilus) experience complete viability loss (>7 log (CFU/ml) when subjected to simulated gastric fluid at 37° C. for 1 hour.

FIG. 6B illustrates viability data for unformulated probiotics (602, Bifidobacterium lactis HN019) and a non-limiting exemplary probiotic composition 607 comprising 5% (w/w) Bifidobacterium lactis HN019 and 95% (w/w) paraffin wax, which demonstrate <1 log (CFU/ml) viability loss when subjected to simulated gastric fluid at 37° C. for 1 hour.

FIG. 6C illustrates viability data for unformulated probiotics (601, Lacticaseibacillus rhamnosus HN001) and a non-limiting exemplary probiotic composition 608 comprising 5% (w/w) Lacticaseibacillus rhamnosus HN001 and 95% (w/w) paraffin wax which demonstrate <1.2 log (CFU/ml) viability loss when subjected to simulated gastric fluid at 37° C. for 24 hours.

FIG. 6D illustrates viability data for unformulated probiotics (601, Lacticaseibacillus rhamnosus HN001), for unformulated, peptone treated probiotics (611), and for non-limiting exemplary probiotic compositions comprising: 5% (w/w) Lacticaseibacillus rhamnosus HN001 and 95% (w/w) paraffin wax (608); 5% (w/w) Lacticaseibacillus rhamnosus HN001 and 95% (w/w) hydrogenated palm oil (609); and 5% (w/w) Lacticaseibacillus rhamnosus HN001 and 95% (w/w) palmitic acid (610), which demonstrate <0.6 log (CFU/ml) viability loss, <1.0 log (CFU/ml) viability loss, and <2.0 log (CFU/ml) viability loss, respectively, when subjected to simulated gastric fluid at 37° C. for 1 hour.

FIG. 6E illustrates viability data for unformulated probiotics (601, Lacticaseibacillus rhamnosus HN001) and for a non-limiting exemplary probiotic composition comprising 5% (w/w) Lacticaseibacillus rhamnosus HN001 and 95% (w/w) paraffin wax (608), which demonstrate <0.6 log (CFU/ml) viability loss, and <1.0 log (CFU/ml) viability loss, respectively, when subjected to simulated gastric fluid at 37° C. for 3 hours.

FIG. 6F illustrates viability data for unformulated probiotics (Bifidobacterium lactis BI-07 605; Lactobacillus acidophilus 613) and non-limiting exemplary probiotic compositions comprising: 5% Bifidobacterium lactis BI-07 with 95% (w/w) paraffin wax (612); and 5% Lactobacillus acidophilus with 95% (w/w) paraffin wax (614), which demonstrate <1.1 log (CFU/ml) viability loss and <1.0 log (CFU/ml) viability loss, respectively, when subjected to simulated gastric fluid at 37° C. for 3 hours.

FIG. 6G illustrates viability data for unformulated probiotics (Lacticaseibacillus rhamnosus HN0001 601), and for a non-limiting exemplary probiotic composition comprising 35% Lacticaseibacillus rhamnosus HN001 with 65% (w/w) paraffin wax (615), which demonstrate <0.7 log (CFU/ml) viability loss when subjected to simulated gastric fluid at 37° C. for 3 hours.

FIG. 7 presents, in a non-limiting example, that probiotics (e.g., Lacticaseibacillus rhamnosus HN001) in probiotic compositions (5% (w/w) Lacticaseibacillus rhamnosus HN001, 95% (w/w) paraffin wax) remain encapsulated, viable and recoverable after incubation in tryptic soy broth for 24 hours.

FIG. 8A presents, in a non-limiting example, a schematic of a novel oil extraction method to enumerate viability of probiotics (e.g., CFU, log(CFU), CFU/g, log(CFU/g)) encapsulated in probiotic composition(s). The oil extraction methodology provided herein improves upon the recovery achieved with traditional aqueous-only methods by achieving complete extraction and enumeration of all cells in the probiotic composition, even from lipid matrices.

FIG. 8B illustrates exemplary probiotic compositions of 5% (w/w) Lacticaseibacillus rhamnosus HN001 with 95% (w/w) paraffin wax, as well as 5% (w/w) Bifidobacterium lactis HN019 with 95% (w/w) paraffin wax, both demonstrated accurate and consistent CFU enumeration using the extraction methodology shown in FIG. 8A.

FIG. 8C shows, in a non-limiting example, that an oil extraction, as described herein, can be performed on a sample of probiotic composition(s) dispersed in food products (e.g. in milk powder or yogurt) to demonstrate maintenance of probiotic payload component (e.g Lacticaseibacillus rhamnosus HN001 and Bifidobacterium lactis HN019) viability and accurate determination of CFUs.

FIG. 8D demonstrates, in a non-limiting example, that traditional methods are not sufficient to extract and enumerate all bacteria from a lipid based probiotic particle (e.g., 5% (w/w) Lacticaseibacillus rhamnosus HN001 with 95% (w/w) paraffin wax); the oil extraction method provided herein helps to promote cell viability in the probiotic compositions provided herein.

FIG. 9A shows, in a non-limiting example, that when probiotic compositions (e.g. 5% (w/w) Lacticaseibacillus rhamnosus HN001 with 95% (w/w) paraffin wax; and 5% (w/w) Lacticaseibacillus rhamnosus HN001 with 95% (w/w) hydrogenated palm oil are incubated in MRS broth, the growth of surface-accessible probiotics is not inhibited by the presence of lipid carrier material(s), as seen with optical density curves reaching the same final levels of optical density as un-encapsulated Lacticaseibacillus rhamnosus within about 15 hours, and with comparable half-maximal time (about 8 hours).

FIG. 9B shows, in a non-limiting example, that when probiotic compositions 5% (w/w) Bifidobacterium lactis H1N019 with 95% (w/w) paraffin wax) are incubated in MRS broth, the growth of surface-accessible probiotics is not inhibited by the presence of lipid carrier material(s), as seen with optical density curves reaching the same final levels of optical density as un-encapsulated Bifidobacterium lactis HN019 within about 25 hours, and with comparable quarter-maximal time (about 6 hours).

FIG. 10 shows, in a non-limiting example, that when probiotic compositions (e.g. 5% (w/w) Lacticaseibacillus rhamnosus HN001 with 95% (w/w) paraffin wax; 5% (w/w) Lacticaseibacillus rhamnosus HN001 with 95% (w/w) hydrogenated palm oil; 5% (w/w) Lacticaseibacillus rhamnosus HN001 with 95% (w/w) palmitic acid) are incubated in MRS broth, the production of a lactic acid metabolite by probiotics is not inhibited by the presence of lipid carrier material(s) when compared to un-encapsulated Lacticaseibacillus rhamnosus HN001.

FIG. 11A shows, in a non-limiting example, improved retention of viability (i.e., log(CFU)/g) for probiotics encapsulated in probiotic compositions (e.g. 5% (w/w) Lacticaseibacillus rhamnosus HN001 with 95% (w/w) paraffin wax; 5% (w/w) Lacticaseibacillus rhamnosus HN001 with 95% (w/w) hydrogenated palm oil) when dispersed and incubated in high water activity (>0.25) milk powder for a period of time (e.g. incubation period) at 25° C., as compared to that of un-encapsulated probiotic payload component(s) dispersed in milk powder.

FIG. 11B shows, in a non-limiting example, improved retention of viability (i.e., log(CFU)/g) for probiotics encapsulated in probiotic compositions (e.g. 5% (w/w) Lacticaseibacillus rhamnosus HN001 with 95% (w/w) paraffin wax; 5% (w/w) Lacticaseibacillus rhamnosus HN001 with 95% (w/w) hydrogenated palm oil) when dispersed and incubated in high water activity (>0.25) milk powder for a period of time (e.g. incubation period) at 37° C., as compared to that of un-encapsulated probiotic payload component(s) dispersed in milk powder.

FIG. 11C presents, in anon-limiting example, brightfield micrographs of compositions (5% (w/w) Lacticaseibacillus rhamnosus HN001 with 95% (w/w) hydrogenated palm oil) that after being incubated with milk powder for 3 weeks at both 37° C. and 50° C., rinsed, and filtered, demonstrate retention of physical appearance (e.g., size, shape, surface texture) compared to that of freshly prepared compositions seen in FIG. 1A-1E.

FIG. 11D presents, in a non-limiting example, brightfield micrographs of compositions (5% (w/w) Lacticaseibacillus rhamnosus HN001 with 95% (w/w) hydrogenated palm oil) that after being incubated with milk powder for 6 weeks at both 25° C. and 37° C., rinsed, and filtered, demonstrate retention of physical appearance (e.g., size, shape, surface texture) compared to that of freshly prepared compositions seen in FIG. 1A-1E.

FIG. 12A shows, in a non-limiting example, improved retention of viability (i.e., log(CFU)/g) for probiotics encapsulated in probiotic compositions (e.g. 5% (w/w) Lacticaseibacillus rhamnosus HN001 with 95% (w/w) hydrogenated palm oil) when dispersed and incubated in yogurt for 9 weeks at 30° C., as compared to that of un-encapsulated probiotic payload component(s) dispersed in yogurt.

FIG. 12B illustrates, in a non-limiting example, a brightfield microscopy image of a compositions (e.g., 5% (w/w) Lacticaseibacillus rhamnosus HN001 with 95% (w/w) paraffin wax) that after being incubated with yogurt at 30° C. for 8 weeks, rinsed, and filtered, demonstrating retention of physical appearance (e.g., size, shape, surface texture) compared to that of freshly prepared compositions seen in FIG. 1A-1E.

FIG. 12C illustrates, in a non-limiting example, a brightfield microscopy image of a compositions (e.g., 5% (w/w) Lacticaseibacillus rhamnosus HN001 with 95% (w/w) hydrogenated palm oil) that after being incubated with yogurt at 30° C. for 8 weeks, rinsed, and filtered, demonstrating retention of physical appearance (e.g., size, shape, surface texture) compared to that of freshly prepared compositions seen in FIG. 1A-1E.

FIG. 12D illustrates, in a non-limiting example, a brightfield microscopy image of a compositions (e.g., 5% (w/w) Lacticaseibacillus rhamnosus HN001 with 95% (w/w) palmitic acid) that after being incubated with yogurt at 30° C. for 8 weeks, rinsed, and filtered, demonstrating retention of physical appearance (e.g., size, shape, surface texture) compared to that of freshly prepared compositions seen in FIG. 1A-1E.

FIG. 13 shows, in a non-limiting example, that probiotic compositions (e.g., 5% (w/w) Lacticaseibacillus rhamnosus HN001 with 95% (w/w) paraffin wax; 5% (w/w) Lacticaseibacillus rhamnosus HN001 with 95% (w/w) hydrogenated palm oil) mitigate moisture uptake, relative to un-encapsulated probiotics (e.g., Lacticaseibacillus rhamnosus HN001) when exposed to conditions of 33%, 53% and 75% relative humidity for 4 days at 25° C.

FIG. 14 shows, in a non-limiting example, that probiotic compositions (e.g., 5% (w/w) Lacticaseibacillus rhamnosus HN001 with 95% (w/w) paraffin wax; 5% (w/w) Lacticaseibacillus rhamnosus HN001 with 95% (w/w) hydrogenated palm oil) mitigate increase in water activity as compared to un-encapsulated probiotic payload component(s) when exposed to conditions of 33%, 53% and 75% relative humidity for 4 days at 25° C.

FIG. 15A shows, in a non-limiting example, reduced log(CFU/g) viability loss of probiotics in probiotic compositions (e.g. 5% (w/w) Lacticaseibacillus rhamnosus HN001 with 95% (w/w) paraffin wax; 5% (w/w) Lacticaseibacillus rhamnosus HN001 with 95% (w/w) hydrogenated palm oil; 5% (w/w) Lacticaseibacillus rhamnosus HN001 with 95% (w/w) palmitic acid) vs. un-encapsulated probiotic payload component(s) when dispersed and incubated in high water activity (>0.25) milk powder at 35% relative humidity at 25° C. for up to 12 weeks.

FIG. 15B shows, in a non-limiting example, reduced log(CFU/g) viability loss of probiotics in probiotic compositions (e.g. 5% (w/w) Lacticaseibacillus rhamnosus HN001 with 95% (w/w) paraffin wax; 5% (w/w) Lacticaseibacillus rhamnosus HN001 with 95% (w/w) hydrogenated palm oil; 5% (w/w) Lacticaseibacillus rhamnosus HN001 with 95% (w/w) palmitic acid) vs. un-encapsulated probiotic payload component(s) when dispersed and incubated in high water activity (>0.25) milk powder at 50% relative humidity at 25° C. for up to 12 weeks.

FIG. 15C summarizes the log(CFU/g) losses from the examples of FIG. 15A and FIG. 15B.

FIG. 16 shows, in a non-limiting example, that probiotic compositions (e.g. 5% (w/w) Bifidobacterium lactis HN019 with 95% (w/w) hydrogenated palm oil; 5% (w/w) Lacticaseibacillus rhamnosus HN001 with 95% (w/w) hydrogenated palm oil; 5% (w/w) Bifidobacterium lactis HN019 with 95% (w/w) paraffin wax; and 35% (w/w) Lacticaseibacillus rhamnosus HN001 with 95% (w/w) paraffin wax) can be dried at any time (for example, just prior to bagging for long term storage or packaging), using a moisture absorber such as drierite. Using this method, water activity was reduced by up to around 50%.

FIG. 17A shows, in a non-limiting example, that probiotic compositions (e.g. 5% (w/w) Lacticaseibacillus rhamnosus HN001 with 95% (w/w) paraffin wax: 5% (w/w) Lacticaseibacillus rhamnosus HN001 with 95% (w/w) hydrogenated palm oil; 5% (w/w) Lacticaseibacillus rhamnosus HN001 with 95% (w/w) palmitic acid) exhibit reduced caking, agglomeration, aggregation, or clumping as compared to un-encapsulated probiotic payload component(s), when exposed to 33%, 53%, and 75% relative humidity at 25° C.

FIG. 17B shows, in a non-limiting example, that probiotic compositions (e.g., 5% (w/w) Lacticaseibacillus rhamnosus HN001 with 95% (w/w) hydrogenated palm oil: 5% (w/w) Bifidobacterium lactis HN019 with 95% (w/w) hydrogenated palm oil) exhibit reduced caking and better flowability compared to un-encapsulated probiotic payload component(s).

FIG. 18A illustrates, in a non-limiting example, brightfield micrographs of probiotic compositions (e.g., 5% (w/w) Bifidobacterium lactis HN019 with 95% (w/w) hydrogenated palm oil) blended homogeneously with commercially available food product (i.e., milk powder), imparting minimal change to visible appearance (e.g., color and texture).

FIG. 18B illustrates, in a non-limiting example, brightfield micrographs of probiotic compositions (e.g., 5% (w/w) Bifidobacterium lactis HN019 with 95% (w/w) hydrogenated palm oil) blended homogeneously with commercially available food product (i.e., peanut butter), imparting minimal change to visible appearance (e.g., color and texture).

FIG. 18C illustrates, in a non-limiting example, brightfield micrographs of probiotic compositions (e.g., 5% (w/w) Bifidobacterium lactis HN019 with 95% (w/w) hydrogenated palm oil) blended homogeneously with commercially available food products (i.e., taco meat), imparting minimal change to visible appearance (e.g., color and texture).

FIG. 19 shows, in a non-limiting example, images demonstrating the brightening/whitening capabilities of probiotic compositions (5% (w/w) Bifidobacterium lactis HN019 with 95% (w/w) hydrogenated palm oil) when blended with un-encapsulated probiotics (e.g., Bifidobacterium lactis HN019).

FIG. 20 illustrates viability data for unformulated probiotics (IFF Ingredient KP Howaru Dophilus) and for non-limiting exemplary probiotic compositions comprising 5% (w/w) IFF KP Howaru Dophilus and 95% (w/w) paraffin wax. Camera images show formulated VK-probiotics coated onto gummy bears and VK-gummies and non-formulated probiotics alone coated onto gummy bears and VK-gummies.

FIG. 21 shows, in a non-limiting example, improved retention of viability (i.e., log(CFU)/g) for probiotics encapsulated in probiotic compositions (e.g. 5% (w/w) Lacticaseibacillus rhamnosus HN001 with 95% (w/w) hydrogenated palm oil) when dispersed and incubated in high water activity (0.22) milk powder for a period of time (e.g., incubation period) at 25° C., 30° C., and 35° C. as compared to that of un-encapsulated probiotic payload component(s) dispersed in milk powder.

FIG. 22 shows, in a non-limiting example, improved retention of viability (i.e., log(CFU)/g) for probiotics encapsulated in probiotic compositions (e.g. 35% (w/w) Lacticaseibacillus rhamnosus HN001 with 65% (w/w) hydrogenated palm oil) when dispersed and incubated in high water activity (0.22) milk powder for a period of time (e.g., incubation period) at 25° C., 30° C., and 35° C. as compared to that of un-encapsulated probiotic payload component(s) dispersed in milk powder.

FIG. 23 shows, in a non-limiting example, improved retention of viability (i.e., log(CFU)/g) for probiotics encapsulated in probiotic compositions (e.g. 5% (w/w) Lacticaseibacillus rhamnosus HN001 with 95% (w/w) hydrogenated palm oil) when dispersed and incubated in high water activity (0.27) milk powder for a period of time (e.g., incubation period) at 25° C., 30° C., and 35° C. as compared to that of un-encapsulated probiotic payload component(s) dispersed in milk powder.

FIG. 24 shows, in a non-limiting example, improved retention of viability (i.e., log(CFU)/g) for probiotics encapsulated in probiotic compositions (e.g. 35% (w/w) Lacticaseibacillus rhamnosus HN001 with 65% (w/w) hydrogenated palm oil) when dispersed and incubated in high water activity (0.27) milk powder for a period of time (e.g., incubation period) at 25° C., 30° C., and 35° C. as compared to that of un-encapsulated probiotic payload component(s) dispersed in milk powder.

FIG. 25 shows, in a non-limiting example, decreased retention of viability (i.e., log(CFU)/g) for probiotics encapsulated in probiotic compositions (e.g., 5% (w/w) Bifidobacterium lactis HN019 with 95% (w/w) hydrogenated palm oil) when dispersed and incubated in high water activity (0.22) milk powder for a period of time (e.g., incubation period) at 25° C., 30° C., and 35° C. as compared to that of un-encapsulated probiotic payload component(s) dispersed in milk powder.

FIG. 26 shows, in a non-limiting example, decreased retention of viability (i.e., log(CFU)/g) for probiotics encapsulated in probiotic compositions (e.g., 35% (w/w) Bifidobacterium lactis HN019 with 65% (w/w) hydrogenated palm oil) when dispersed and incubated in high water activity (0.22) milk powder for a period of time (e.g., incubation period) at 25° C., 30° C., and 35° C. as compared to that of un-encapsulated probiotic payload component(s) dispersed in milk powder.

FIG. 27 shows, in a non-limiting example, decreased retention of viability (i.e., log(CFU)/g) for probiotics encapsulated in probiotic compositions (e.g., 5% (w/w) Bifidobacterium lactis HN019 with 95% (w/w) hydrogenated palm oil) when dispersed and incubated in high water activity (0.27) milk powder for a period of time (e.g. incubation period) at 25° C. and 30° C. as compared to that of un-encapsulated probiotic payload component(s) dispersed in milk powder. At 35° C., improved retention of viability (i.e., log(CFU)/g) for probiotics encapsulated in probiotic compositions (e.g., 5% (w/w) Bifidobacterium lactis HN019 with 95% (w/w) hydrogenated palm oil) when dispersed and incubated in high water activity (0.27) milk powder for a period of time (e.g. incubation period) was observed as compared to that of un-encapsulated probiotic payload component(s) dispersed in milk powder.

FIG. 28 shows, in a non-limiting example, decreased retention of viability (i.e., log(CFU)/g) for probiotics encapsulated in probiotic compositions (e.g., 35% (w/w) Bifidobacterium lactis HN019 with 65% (w/w) hydrogenated palm oil) when dispersed and incubated in high water activity (0.27) milk powder for a period of time (e.g., incubation period) at 25° C. and 30° C. as compared to that of un-encapsulated probiotic payload component(s) dispersed in milk powder. At 35° C., improved retention of viability (i.e., log(CFU)/g) for probiotics encapsulated in probiotic compositions (e.g., 35% (w/w) Bifidobacterium lactis HN019 with 65% (w/w) hydrogenated palm oil) when dispersed and incubated in high water activity (0.27) milk powder for a period of time (e.g., incubation period) was observed as compared to that of un-encapsulated probiotic payload component(s) dispersed in milk powder.

FIG. 29 shows, in a non-limiting example, water activity of commercial milk and/or dairy protein powders (i.e., aW) for a period of time (e.g., incubation period of 4 weeks) at room temperature. 5 days out of a 7 day week, containers storing the milk and/or dairy protein powders are opened to expose the powders to ambient moisture to evaluate how water activity changes in the powders during routine use. Of particular note, during the 4 week study, commercial powder products are typically less than 0.23 aW, indicating the target aW performance criteria.

FIG. 30 shows, in a non-limiting example, (A) improved retention of viability (i.e., log(CFU)/ml) for probiotics encapsulated in probiotic compositions (e.g., 10% (w/w) Bifidobacterium lactis HN019 with 90% (w/w) hydrogenated palm oil (sourced as either Dritex or ADM)) when dispersed and incubated in yogurt for a period of time (e.g. incubation period) at 4° C. as compared to that of un-encapsulated probiotic payload component(s) dispersed in yogurt: (B) corresponding log loss (comparing the endpoint at 59 days to day 0) for each group; (C) a harvested and intact encapsulated probiotic formulation from yogurt after 28 days.

FIG. 31 shows, in a non-limiting example, improved retention of viability (i.e., log(CFU)/ml) for probiotics encapsulated in probiotic compositions (e.g. 10% (w/w) Bifidobacterium lactis HN019 with 90% (w/w) hydrogenated palm oil (GV60 from ADM)) when dispersed and incubated in yogurt for a period of time (e.g., incubation period) at 25° C., 30° C., 35° C. as compared to that of un-encapsulated probiotic payload component(s) dispersed in yogurt; (B) camera image of the 10% (w/w) Bifidobacterium lactis HN019 with 90% (w/w) hydrogenated palm oil (GV60 from ADM) formulation; (C) microscope image of the 10% (w/w) Bifidobacterium lactis HN019 with 90% (w/w) hydrogenated palm oil (GV60 from ADM)); (D) a microscope image of the 10% (w/w) Bifidobacterium lactis HN019 with 95% (w/w) hydrogenated palm oil (GV60 from ADM) demonstrating maintenance of size, shape, and physical characteristics after storage in yogurt; (E) is a graph showing that pH was maintained at 25° C., 30° C., and 35° C. for the duration of at least four weeks.

FIG. 32 shows, in a non-limiting example, improved retention of viability (i.e., log(CFU)/ml) for probiotics encapsulated in probiotic compositions (e.g., 5% (w/w) Bifidobacterium lactis HN019 with 47.5% (w/w) beeswax and 47.5% stearic acid) when dispersed and incubated in yogurt for a period of time (e.g., incubation period) at 25° C., 30° C., 35° C. as compared to that of un-encapsulated probiotic payload component(s) dispersed in yogurt; (B) camera image of 5% (w/w) Bifidobacterium lactis HN019 with 47.5% (w/w) beeswax and 47.5% stearic acid formulation; (C) microscope image of the 5% (w/w) Bifidobacterium lactis HN019 with 47.5% (w/w) beeswax and 47.5% stearic acid.

FIG. 33 shows, in a non-limiting example, a camera image of 10% (w/w) Bifidobacterium lactis HN019 with 90% (w/w) hydrogenated palm oil (GV60 from ADM) after 2 weeks of storage in yogurt. Controls of HN019 in yogurt (without encapsulation) and yogurt alone are also shown.

FIG. 34 shows, in a non-limiting example, improved retention of viability (i.e., log(CFU)/ml) for probiotics encapsulated in probiotic compositions (e.g., 10% (w/w) Lacticaseibacillus rhamnosus HN001 with 90% (w/w) hydrogenated palm oil (GV60 from ADM)) when dispersed and incubated in yogurt for a period of time (e.g. incubation period) at 25° C., 30° C., 35° C. as compared to that of un-encapsulated probiotic payload component(s) dispersed in yogurt; (B) camera image of 10% HN001 with 90% hydrogenated palm oil (GV60 from ADM); (C) microscope image of 10% HN001 with 90% hydrogenated palm oil (GV60 from ADM).

FIG. 35 shows, in a non-limiting example, retention of viability (i.e., log(CFU)/ml) for probiotics encapsulated in probiotic compositions (e.g., 10% (w/w) Lacticaseibacillus rhamnosus HN001 with 90% (w/w) hydrogenated palm oil (GV60 from ADM)) when dispersed and incubated in yogurt for a period of time (e.g., incubation period) at 25° C. 30° C., 35° C. as compared to that of un-encapsulated probiotic payload component(s) dispersed in yogurt at loadings (for the encapsulated probiotics) of between (A) 10⁷-10⁸ CFU/ml and (B) 10⁶-10⁷ CFU/ml.

FIG. 36 shows, in a non-limiting example, retention of viability (i.e., log(CFU)/ml) for probiotics encapsulated in probiotic compositions (e.g., 5% (w/w) Lacticaseibacillus rhamnosus with 47.5% (w/w) beeswax with 47.5% stearic acid) when dispersed and incubated in yogurt for a period of time (e.g., incubation period) at 25° C., 30° C., 35° C. as compared to that of un-encapsulated probiotic payload component(s) dispersed in yogurt.

FIG. 37 shows, in a non-limiting example, retention of viability (i.e., log(CFU)/ml) for probiotics encapsulated in probiotic compositions (e.g., 5% (w/w) Lacticaseibacillus rhamnosus with 10% (w/w) polyethylene glycol (PEG) with 42.5% (w/w) hydrogenated palm oil (GV60 from ADM) with 42.5% stearic acid) when dispersed and incubated in yogurt for a period of time (e.g., incubation period) at 25° C., 30° C., 35° C. as compared to that of un-encapsulated probiotic payload component(s) dispersed in yogurt.

FIG. 38 shows, in a non-limiting example, (A) retention of viability (i.e., log(CFU)/ml) for probiotics encapsulated in probiotic compositions (e.g., 5% (w/w)-10% (w/w) Lacticaseibacillus rhamnosus with various encapsulants/excipients including beef gelatin, isomalt, calcium lactate gluconate, polydectrose, calcium carbonate, GV60 (hydrogenated palm oil), amidated Lm pectin, calcium chloride, sodium alginate, sunflower lecithin) when dispersed and incubated in yogurt for 1 month at 30° C. as compared to that of un-encapsulated probiotic payload component(s) dispersed in yogurt for 1 month at 30° C.; and (B) corresponding log loss (comparing the CFU/ml at the endpoint at 1 month to initial CFU/ml added to yogurt at day 0) for each group.

FIG. 39 shows, in a non-limiting example, improved retention of viability (i.e., log(CFU)/g) for probiotics encapsulated in probiotic compositions (e.g., 5% (w/w) Lacticaseibacillus rhamnosus HN001 with 95% (w/w) hydrogenated palm oil) when dispersed and incubated in high water activity (0.22) milk powder for a period of time (e.g., incubation period) up to 9 months at 25° C., 30° C., and 35° C. as compared to that of un-encapsulated probiotic payload component(s) dispersed in milk powder.

FIG. 40 shows, in a non-limiting example, improved retention of viability (i.e., log(CFU)/g) for probiotics encapsulated in probiotic compositions (e.g., 5% (w/w) Lacticaseibacillus rhamnosus HN001 with 95% (w/w) hydrogenated palm oil) when dispersed and incubated in high water activity (0.27) milk powder for a period of time (e.g., incubation period) up to 9 months at 25° C., 30° C., and 35° C. as compared to that of un-encapsulated probiotic payload component(s) dispersed in milk powder.

FIG. 41 shows, in a non-limiting example, decreased retention of viability (i.e., log(CFU)/g) for probiotics encapsulated in probiotic compositions (e.g., 5% (w/w) Bifidobacterium animalis subsp. lactis HN019 with 95% (w/w) hydrogenated palm oil) when dispersed and incubated in high water activity (0.22) milk powder for a period of time (e.g., incubation period) up to 9 months at 25° C., 30° C., and 35° C. as compared to that of un-encapsulated probiotic payload component(s) dispersed in milk powder.

FIG. 42 shows, in a non-limiting example, decreased retention of viability (i.e., log(CFU)/g) for probiotics encapsulated in probiotic compositions (e.g., 5% (w/w) Bifidobacterium animalis subsp. Lactis HN019 with 95% (w/w) hydrogenated palm oil) when dispersed and incubated in high water activity (0.27) milk powder for a period of time (e.g., incubation period) up to 9 months at 25° C. and 30° C. as compared to that of un-encapsulated probiotic payload component(s) dispersed in milk powder. At 35° C., improved retention of viability (i.e., log(CFU)/g) for probiotics encapsulated in probiotic compositions (e.g., 5% (w/w) Bifidobacterium lactis HN019 with 95% (w/w) hydrogenated palm oil) was observed as compared to that of un-encapsulated probiotic payload component(s) dispersed in milk powder.

FIG. 43 shows, in a non-limiting example, improved retention of viability (i.e., log(CFU)/ml) for probiotics encapsulated in probiotic compositions (e.g. 10% (w/w) Bifidobacterium animalis subsp. lactis HN019 with 90% (w/w) hydrogenated palm oil (GV60 from ADM) when dispersed and incubated in yogurt for a period of time (e.g. incubation period) up to 12 weeks at 25° C., 30° C. 35° C. as compared to that of un-encapsulated probiotic payload component(s) dispersed in yogurt.

FIG. 44 shows, in a non-limiting example, improved retention of viability (i.e., log(CFU)/ml) for probiotics encapsulated in probiotic compositions (e.g., 5% (w/w) Bifidobacterium animalis subsp. lactis HN019 with 47.5% (w/w) beeswax and 47.5% stearic acid) when dispersed and incubated in yogurt for a period of time (e.g., incubation period) at 25° C., 30° C., 35° C. as compared to that of un-encapsulated probiotic payload component(s) dispersed in yogurt.

FIG. 45 shows, in a non-limiting example, improved retention of viability (i.e., log(CFU)/ml) for probiotics encapsulated in probiotic compositions (e.g., 10% (w/w) Lacticaseibacillus rhamnosus HN001 with 90% (w/w) hydrogenated palm oil (GV60 from ADM)) when dispersed and incubated in yogurt for a period of time (e.g. incubation period) at 25° C., 30° C., 35° C. as compared to that of un-encapsulated probiotic payload component(s) dispersed in yogurt.

FIG. 46 shows, in a non-limiting example, improved retention of viability (i.e., log(CFU)/serving) as compared to control (unencapsulated) (black circles), when dispersed and incubated in Gatorade at 25° C. for probiotics encapsulated in probiotic compositions: FIG. 46A shows 10% (w/w) Lacticaseibacillus rhamnosus HN001 with 90% (w/w) hydrogenated palm oil/GV60 (grey squares) or core-shell probiotic particles [CORE (2% HN001; 67.40% GV60)+SHELL (19.10% Shellac; 9.55% Ethyl Cellulose; 1.91% Stearic Acid)](grey triangles) at 24 hours, and FIG. 46B shows 10% (w/w) Lacticaseibacillus rhamnosus HN001 with 90% (w/w) hydrogenated palm oil/GV60 (grey squares) at 168 hours.

FIGS. 47-50B show images of exemplary formulations disclosed herein.

FIG. 51 shows a plot of particle size distribution of exemplary formulations disclosed herein.

FIGS. 52A-53B show images of exemplary formulations disclosed herein.

FIG. 54 shows a plot of particle size distribution of exemplary formulations disclosed herein.

FIGS. 55A-56B show images of exemplary formulations disclosed herein.

FIG. 57 shows a plot of particle size distribution of exemplary formulations disclosed herein.

FIGS. 58A-58B show images of exemplary formulations disclosed herein.

FIG. 58C shows a plot of particle size distribution of exemplary formulations disclosed herein.

FIGS. 59A-60 show images of an exemplary formulation (FIGS. 59A-59B) and a corresponding plot of particle size distribution (FIG. 60).

FIGS. 61A-62 show images of an exemplary formulation (FIGS. 61A-61B) and a corresponding plot of particle size distribution (FIG. 62).

FIGS. 63-64 show a plot of particle size distribution for an exemplary formulation disclosed herein and a photograph of its inclusion in water.

FIG. 65 shows a photograph of an exemplary formulation included in water.

FIGS. 66A-66C show data plots of an unencapsulated exemplary probiotic included in an enteral feed product (e.g., Compleat).

FIGS. 67A-67G show data plots of exemplary probiotic formulations included in an enteral feed product (e.g., Compleat).

FIGS. 68A-68C show data plots of an unencapsulated exemplary probiotic included in an enteral feed product (e.g., Compleat).

FIGS. 69A-69G show data plots of exemplary probiotic formulations included in an enteral feed product (e.g., Compleat).

FIG. 70 shows a data plot comparing performance in commercial bottled water of exemplary probiotic formulations v. unencapsulated exemplary probiotics.

FIGS. 71A-71B show data plots comparing performance in milk of exemplary probiotic formulation v. unencapsulated exemplary probiotics (FIG. 71A) and the corresponding pH during trial (FIG. 71B).

FIGS. 72A-72B show data plots comparing performance in an electrolyte beverage (e.g., Gatorade) of exemplary probiotic formulations v. unencapsulated exemplary probiotics.

FIG. 73 shows a data plot comparing performance in boiling water of exemplary probiotic formulations v. unencapsulated exemplary probiotics.

FIG. 74 shows a data plot comparing performance in yogurt of exemplary probiotic formulations v. unencapsulated exemplary probiotics.

FIG. 75 shows a data plot of performance of exemplary formulations in simulated gastric fluid (“SGF”).

FIG. 76 shows a comparison of performance in a carbonated beverage (e.g., a soda) of non-limiting exemplary probiotic formulations v. unencapsulated exemplary probiotics.

### DETAILED DESCRIPTION

### A. Certain Terminology

Section headings used herein are for organizational purposes only and are not to be construed as limiting the subject matter described.

Unless defined otherwise, technical and scientific terms used herein have the same meaning as is commonly understood to which the claimed subject matter belongs. In the event that there are a plurality of definitions for terms herein, those in this section prevail.

It is to be understood that the general description and the detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Definition of standard chemistry terms may be found in reference works, including but not limited to, Carey and Sundberg “Advanced Organic Chemistry 4th Ed.” Vols. A (2000) and B (2001), Plenum Press, New York.

As used herein, the symbol “<” means less than or fewer than. As used herein, the symbol “>” means more than.

As used herein, the term “about” or “approximately” means within 10%, preferably within 10%, and more preferably within 5% of a given value or range.

Ambient: The term “ambient”, as used herein, refers to a typical indoor (e.g., climate-controlled) temperature, usually within a range of about 18° C. to about 32° C., and/or typical indoor (e.g., climate-controlled) humidity, usually within a range of about 30% to 50%. In some embodiments, ambient temperature is within a range of about 20° C. to about 30° C. In some embodiments, ambient temperature is 25±5° C. In some embodiments, ambient temperature is approximately 21° C. In some embodiments, ambient temperature is 18° C. In some embodiments, ambient temperature is 19° C. In some embodiments, ambient temperature is 20° C. In some embodiments, ambient temperature is 21° C. In some embodiments, ambient temperature is 22° C. In some embodiments, ambient temperature is 23° C. In some embodiments, ambient temperature is 24° C. In some embodiments, ambient temperature is 25° C. In some embodiments, ambient temperature is 26° C. In some embodiments, ambient temperature is 27° C. In some embodiments, ambient temperature is 28° C. In some embodiments, ambient temperature is 29° C. In some embodiments, ambient temperature is 30° C. In some embodiments, ambient may be used to describe outdoor conditions, and may include temperatures ranging from about 15° C. to about 40° C., or from about 25° C. to about 40° C. In some embodiments, ambient humidity is within a range of about 35% to about 45%. In some embodiments, ambient temperature is 35%. In some embodiments, ambient temperature is 36%. In some embodiments, ambient temperature is 37%. In some embodiments, ambient temperature is 38%. In some embodiments, ambient temperature is 39%. In some embodiments, ambient temperature is 40%. In some embodiments, ambient temperature is 410%. In some embodiments, ambient temperature is 42%. In some embodiments, ambient temperature is 43%. In some embodiments, ambient temperature is 44%. In some embodiments, ambient temperature is 45%.

Beverage: As used herein, the term “beverage” is used to refer to a potable liquid (e.g., that can be ingested, swallowed, drunk, or consumed by a person or animal without material risk to the person or animal). For example, beverage can be or comprise beer, juice, milk, a sports drink, tea, water, soda, yogurt, etc. In some embodiments, a “beverage” may be or comprise a pharmaceutical formulation in liquid form.

Biocompatible: As used herein, the term “biocompatible” is used to describe a characteristic of not causing significant detectable harm to living tissue when placed in contact therewith e.g., in vivo. In certain embodiments, materials are “biocompatible” if they are not significantly toxic to cells, e.g., when contacted therewith in a relevant amount and/or under relevant conditions such as over a relevant period of time. In certain embodiments, materials are “biocompatible” if their addition to cells in vitro results in less than or equal to 20% cell death, and/or their administration in vivo does not induce significant inflammation or other adverse effects.

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Cryoprotectant: As used herein the term “cryoprotectant” refers to a chemical or compound that is used to prevent the formation of ice crystals during the supercooling of a water containing sample.

Degradation: As used herein, the term “degradation” refers to a change in chemical structure and often involves breakage of at least one chemical bond. To say that a chemical compound is degraded typically means that the chemical structure of the chemical compound has changed (e.g., a chemical bond is broken). Common mechanisms of degradation include, for example, oxidation, hydrolysis, isomerization, fragmentation, or a combination thereof.

Diameter: As used herein, the term “diameter” is used to refer to the longest distance from one end of a particle to another end of the particle. Those skilled in the art will appreciate that a variety of techniques are available for use in characterizing particle diameters (i.e., particle sizes). In some instances, for example, size of particles (e.g., diameter of particles) can be measured by a Coulter Counter. In some instances, for example, size of particles (e.g., diameter of particles) can be measured by a Malvern Mastersizer. In some embodiments, a population of particles is characterized by an average size (e.g., D[3,2], D[4,3], etc.) and/or by particular characteristics of size distribution (e.g., absence of particles above or below particular sizes [e.g., Dv10, Dv20, Dv30, Dv40, Dv50, Dv60, Dv70, Dv80, Dv90, Dv99, etc.], a unimodal, bimodal, or multimodal distribution, etc.).

Dispersity: As used herein, the term “dispersity” is used to refer to the breadth of particle size distribution relative to the average particle size. In some instances, for example, size of particles (e.g., diameter of particles) can be measured by a Coulter Counter. In some instances, for example, size of particles (e.g., diameter of particles) can be measured by a Malvern Mastersizer. In some embodiments, the population of particles is characterized by, for example, an average size (e.g., Dv50) and, for example, a corresponding standard deviation. In some instances, the dispersity of a population of particles refers to double (e.g., 2-fold) the ratio of standard deviation (e.g., a) to average particle diameter (e.g., Dv50).

Encapsulated: As used herein, the term “encapsulated” is used to refer to a characteristic of being physically associated with, and in some embodiments partly or wholly covered or coated. For example, in many embodiments of the present disclosure, a payload component (e.g., a microbe component and/or a nutrient component) is described as being encapsulated by a polymer component.

Food: As used herein, the term “food” is used to refer to an edible solid (e.g., that can be ingested, swallowed, chewed, or consumed by a person or animal without material risk to the person or animal). For example, food can be or comprise agricultural seed, baby formula, bread, candy, capsule, cake, cereal, chip, cookie, dry powder, fertilizer, food additive, ice cream, kefir, nutrition supplement, packaged food, pet feed, pet food, protein bar, protein powder, sachet, salad dressing, smoothie, spice, sprinkle packet, tablet, yogurt, a gummy product (e.g., gummy bear, gummy worm, gelatin-based gummy product, pectin-based gummy product, etc.) etc. In some embodiments, a “food” may be or comprise a pharmaceutical formulation in solid form.

Gummy Product: As used herein, the term “gummy product” is used to refer to an edible solid of water activity between 0.50 and 0.70, that can be ingested, swallowed, chewed, or consumed by a person or animal without material risk to the person or animal. For example, a gummy product can be a gelatin-based gummy product, a pectin-based gummy product, a gummy bear, a gummy worm, and/or combinations thereof, etc.

HLB: As used herein, the term “HLB” is used to refer to the hydrophilic lipophilic balance that is an inherent property of, for example, a nonionic surfactant. In some instances, the HLB value of a given non-ionic surfactant is obtained from a commonly accessible tabular source. In some embodiments, non-ionic surfactants characterized as having a low HLB value (e.g., <8) are compatible emulsifiers for lipid systems. In some embodiments, nonionic surfactants characterized as having a high HLB value (e.g., >15) are compatible emulsifiers for aqueous systems. In some embodiments, non-ionic surfactants characterized as having an intermediate HLB value (e.g., >8 and <15) are compatible emulsifiers with both lipid and aqueous systems.

Homogenous: As used herein, the term “homogenous” means of substantially uniform structure and/or composition throughout.

Hydrophobic: As used herein, the term “hydrophobic” is used to refer to the propensity of a material to reject association, chemically and/or physically, with water. In some instances, a material characterized as being hydrophobic is biologically derived and/or synthetically derived. In some instances, a material characterized as being hydrophobic is a lipid, protein, and/or carbohydrate. In some instances, a material characterized as being hydrophobic is a polymer and/or small molecule. Alternatively, or additionally, in some embodiments, composites, mixtures, blends, or super-structures of several materials are collectively referred to as hydrophobic based on their observed propensity to reject association, chemically and/or physically, with water.

Incorporation: As used herein, the term “incorporation” is used to refer to a characteristic of being physically associated with, and in some embodiments, dispersed within, embedded within, or mixed in a bulk material (e.g., a lipid matrix component).

Layer: As used herein, the term “layer” typically refers to a material disposed above or below a distinguishable material. In some embodiments, a particular entity or preparation (e.g., particle preparation) is described as “layered” if it is prepared via a process in which a first material is laid down and then a second material is applied atop or underneath the first material (e.g., as by dipping or spraying, etc); in some such embodiments, physical or chemical distinctness of layers may be maintained over time, whereas in some such embodiments, physical or chemical distinctness of layers may decay over time, at least at layer interface(s). Alternatively or additionally, in some embodiments, a particular sample or preparation may be described as layered, independent of its mode of preparation, so long as at a particular point in time and/or using a particular mode of assessment, distinct materials can be identified in a layered structure. In some embodiments, a “layered” particle may include one or more layers that wholly encapsulate a material below. In some embodiments, a “layered” particle may include one or more layers that does not wholly encapsulate a material below. In some embodiments, at least one layer of a layered preparation is or comprises a polymer, e.g., a hydrophobic polymer or hydrophilic polymer. In some embodiments, each layer of a layered preparation is or comprises a polymer, e.g., a pH responsive polymer or a temperature-responsive polymer.

Lipid: As used herein, the term “lipid” is used to refer to a class of chemical structures characterized as hydrophobic materials. In some instances, a lipid material is derived from a biological source. In other instances, a lipid material is derived from a synthetic source. In some instances, a lipid comprises one or more aliphatic alcohols and/or acids linked by glycerol and/or glycol moieties. In other instances, a lipid comprises aliphatic chains, linear conjugated, aromatic, and/or cyclic aliphatic moieties. In some embodiments, a lipid refers to a pure chemical entity. In other embodiments, a lipid refers to a mixture of several pure chemical entities. For example, lipids include, but are not limited to: paraffin wax, montan wax, microcrystalline wax, polyethylene wax, petrolatum wax, ozokerite wax, ceresin wax, beeswax, lanolin wax, spermaceti wax, tallow wax, lac wax, chinese insect wax, ambergris wax, soy wax, camauba wax, candelilla wax, coconut wax, palm kemel wax, rice bran wax, butyric acid, n-butanol, pentanoic acid, n-pentanol, hexanoic acid, n-hexanol, heptanoic acid, n-heptanol, caprylic acid, n-octanol, nonanoic acid, n-nonanol, capric acid, n-decanol, lauric acid, n-dodecanol, myristic acid, n-tetradecanol, palmitic acid, n-hexadecanol, stearic acid, n-octadecanol, arachidonic acid, n-icosanol, fatty alcohol monoglyceride ethers, fatty acid monoglyceride esters, fatty alcohol diglyceride ethers, fatty acid diglyceride esters, fatty alcohol triglyceride ethers, fatty acid triglyceride esters, fatty alcohol glycol monoether, fatty acid glycol monoesters, fatty alcohol glycol diethers, fatty acid glycol diesters, fatty alcohol poly(glycerol) ethers, fatty acid poly(glycerol) esters, fatty alcohol poly(glycol) ethers, fatty acid poly(glycol) esters, coconut oil, corn oil, cottonseed oil, olive oil, palm oil, peanut oil, rapeseed oil, safflower oil, sesame oil, soybean oil, sunflower oil, almond oil, pine nut oil, cashew oil, fully hydrogenated palm oil, partially hydrogenated palm oil, fully hydrogenated sunflower oil, partially hydrogenated sunflower oil, fully hydrogenated soybean oil, partially hydrogenated soybean oil, fully hydrogenated vegetable oil, partially hydrogenated vegetable oil, fully hydrogenated cottonseed oil, partially hydrogenated cottonseed oil, cholesterol, cholenic acid, ursolic acid, or betulinic acid.

Lyophilized: As used herein, the term “lyophilized” is used to refer to the end product of a process by which water is removed from a material via sublimation. In some instances, prior to sublimation of water, the material is cooled to <−10° C., <−20° C., <−30° C., <−40° C., <−50° C., <−60° C. and/or <−70° C. In some instances, prior to the sublimation of water, the pressure is lowered to <200 torr, <150 torr, <100 torr, <50 torr, <10 torr, <5 torr, and/or <1 torr. Those skilled in the art recognize that the cooling temperature and pressure influence the physicochemical properties of the end product; it is understood that “lyophilized” ecompasses all suitable manners of cooling and vacuum protocol.

Nutraceutical composition: As used herein, the term “nutraceutical composition” refers to a substance or material that is or comprises a nutraceutical agent (e.g., a nutraceutical). Those skilled in the art will be aware of a variety of agents understood in the art to be nutraceutical agents such as, for example, agents that are or comprise one or more antioxidants, macronutrients, micronutrients, minerals, prebiotics, probiotics, probiotic powders, probiotic ingredients, probiotic food ingredients, probiotic supplement ingredients, prebiotics, vitamins, or combinations thereof. In some embodiments, a nutraceutical is or comprises a carotenoid compound such as alpha-lipoic acid, astaxanthin, adonixanthin, adonirubin, beta-carotene, coenzyme Q10, lutein, lycopene, or zeaxanthin. In some embodiments, a nutraceutical is or comprises a vitamin such as vitamin D. In many embodiments, a nutraceutical agent is a natural product, and in certain such embodiments it is a product produced by plants. Many nutraceutical agents are compounds that have been reported or demonstrated to confer a benefit or provide protection against a disease in an animal or a plant. In some embodiments, nutraceuticals may be used to improve health, delay the aging process, protect against chronic diseases, increase life expectancy, or support the structure or function of the body of an animal, such as a human, a pet animal, an agricultural animal, or another domesticated animal. As used in the present disclosure, which focuses on probiotics, the term “nutraceutical composition” will generally be understood to mean a composition comprising at least one probiotic component, among other potential components (including one or more of the nutraceutical agents disclosed above). As such, as used in the present disclosure, the terms “nutraceutical composition,” “probiotic preparation,” “probiotic composition.” “particle preparation,” “microbe composition,” etc. may all be generally understood to describe compositions, preparations, and/or particles that include one or more probiotics (for example, encapsulated probiotics).

Particle: As used herein, the term “particle” is used to refer to a discrete physical entity, typically having a size (e.g., a longest cross-section, such as a diameter) within a range. For example, a particle can have a size of about 5-3000 μm, about 5-2000 μm, about 5-1000 μm, about 5-500 μm, about 5-50 μm, about 5-300 μm, about 5-200 μm, about 5-100 μm, about 5-50 μm, about 5-25 μm, or about 5-10 μm. In some embodiments, a particle may describe or include animal pellets ranging in size up to 1 mm, 5 mm, 10 mm, 25 mm, and even about 50 mm (about 2 inches) in diameter. A “particle” is not limited to a particular shape or form, for example, having a cross-section shape of a sphere, an oval, a triangle, a square, a hexagon, or an irregular shape. In some embodiments, particles can be solid particles. In some embodiments, particles can be liquid particles. In some embodiments, particles can be gel or gel-like particles. In some embodiments, particles may have a particle-in-particle structure wherein a layer of one material (e.g., one type of polymer component) encapsulates another material (e.g., another type of polymer component, which may itself encapsulate yet another, or rather may be or comprise a “core”—e.g., a polymer matrix core—of the particle).

Parts per million (ppm): As used herein, 1 ppm (“parts per million”) is equivalent to 1 milligram per liter (mg/L) or 1 milligram per kilogram (mg/kg).

pH Responsive: The term “pH-responsive” is used to refer to certain polymer component(s) as described herein, and in particular means that the relevant polymer component is characterized in that one or more aspects of its structure or arrangement is altered when exposed to a change in pH condition (e.g., to a particular pH and/or to a pH change of particular magnitude). In some embodiments, a polymer component is considered to be “pH-responsive” if, when the relevant polymer component is associated with a payload component in a particle preparation as described herein, the particle preparation releases the payload component under specific pH condition(s). In some embodiments, >90% of payload component is released from a particle preparation that includes a pH-responsive polymer component within 15 minutes when the particle preparation is exposed to a particular defined pH condition (e.g., within a range of defined pH values and/or at a specific pH value); in some embodiments, such release results when such contacting occurs at temperatures between 33-40° C., and in aqueous-based buffers of ionic strength ranging from 0.001-0.151 M (e.g., water, simulated gastric fluid, gastric fluid, simulated intestinal fluid, intestinal fluid) with osmolality between 1-615 mOsm/kg. In some embodiments, a pH-responsive polymer component is one that degrades when exposed to a particular pH or pH change. Alternatively or additionally, in some embodiments, a pH-responsive polymer component is one that becomes soluble, or significantly (e.g., (e.g., by at least about 5%) increases its solubility when exposed to a particular pH level, or pH change. In some embodiments, a pH-responsive polymer component includes one or more moieties whose protonation state changes at the relevant pH or in response to the relevant pH change. For example, in some embodiments, a pH responsive polymer component includes one or more amine moieties that become protonated upon exposure to a relevant pH or pH chance.

Probiotic: As used herein, the term “probiotic” is used to refer to compositions that are or include a live microorganism (e.g., bacterium, fungus, virus, or bacteriophage) that is not harmful to certain animals (e.g., ruminants and/or humans) so that it can safely be ingested thereby. Probiotics may take the form of compositions, preparations, and/or particles, as described in further detail in the present disclosure. In some embodiments, a probiotic is reported or known to provide one or more health benefits when ingested, consumed, or otherwise administered.

Reference: As used herein describes a standard or control relative to which a comparison is made. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Residual solvent: As used herein, the term “residual solvent” refers to a solvent that remains in a material after manufacture or processing of the material. In some embodiments, level of residual solvent is assessed by HPLC, mass spec, NMR, FTIR, and/or gas chromatography.

Stable: The term “stable,” when applied to compositions herein, means that the compositions maintain (e.g., as determined by one or more analytical assessments) one or more aspects of their physical structure and/or performance characteristic(s) (e.g., activity) over a period of time and/or under a designated set of conditions. When an assessed composition is a particle composition, in some embodiments, as will be clear from context to those skilled in the art, the term “stable” refers to maintenance of a characteristic such as average particle size, maximum and/or minimum particle size, range of particle sizes, and/or distribution of particle sizes (i.e., the percentage of particles above a designated size and/or outside a designated range of sizes) over a period of time and/or under a designated set of conditions. For probiotics, stable often refers to maintenance or preservation of viability and/or colony forming units.

Temperature-responsive: As used herein, the term “temperature-responsive” is used to refer to certain polymer component(s) as described herein, and in particular means that the relevant polymer component is characterized in that one or more aspects of its structure or arrangement is altered when exposed to a change in temperature condition (e.g., to a particular temperature and/or to a temperature change of particular magnitude). In some embodiments, a polymer component is considered to be “temperature-responsive” if, when the relevant polymer component is associated with a payload component in a particle preparation as described herein, amorphous regions of the polymer component experience a transition from a rigid state (e.g., glassy state) to a more fluid-like flexible state (e.g., more conducive to flow), at a temperature close to the point of transition from the solid state to rubbery state (e.g., glass transition).

Viability: As used herein, the term “viability” is used to refer to cellular payload component(s) as described herein, and in particular means that the relevant cellular payload component is characterized by the ability to reproduce under favorable conditions, as most commonly measured by a spread plate enumeration method as described herein and reported in terms of colony forming units (CFU), CFU/g, CFU/mL, log(CFU/g) or log(CFU/mL).

Viability Loss: As used herein, the term “viability loss” is used to refer to a change in viability (as described herein, reported in terms of colony forming units (CFU), (CFU/g), (CFU/mL), log(CFU), log(CFU/g), and/or log(CFU/mL)) of a composition after a period of incubation in an environment (e.g., exposure to time, temperature, water, heat, light, shear, pressure). As provided herein, viability loss refers to the difference in enumerated log(CFU) before and after exposure to the environmental condition for the proscribed incubation period.

Water activity: As used herein, “water activity” of a material is an indication (e.g., a measurement) of how much free (i.e., available to bind or react) water is present in the material, and is typically determined as the ratio of the vapor pressure of water in a material (p) to the vapor pressure of pure water (po) at the same temperature. For example, a water activity of 0.80 means the vapor pressure is 80 percent of that of pure water. Water activity typically increases with temperature. Those skilled in the art will be familiar with three basic water activity measurement systems: Preventive Electrolytic Hygrometers (REH), Capacitance Hygrometers, and Dew Point Hygrometers (sometimes called chilled mirror).

Disclosed herein, among other things, are compositions and methods for manufacture, maintenance (e.g., storage) and/or use (e.g., administration or delivery) of a probiotic composition (e.g., a particle preparation comprising a microbe comprising at least one of Lactobacillus rhamnosus, Lactobacillus acidophilus, Bifidobacterium lactis, Bifidobacterium animalis, food ingredient(s) comprising microbes, and/or mineral(s) such as calcium carbonate).

In some embodiments, a nutraceutical is or comprises a probiotic ingredient, or probiotic food ingredient, or probiotic supplement ingredient. In some aspects, the disclosure provides a formulation of a nutraceutical probiotic food ingredient, probiotic ingredient, or probiotic supplement ingredient for improving health.

In some embodiments, a nutraceutical is or comprises a probiotic ingredient. In some aspects, the disclosure provides a formulation of a nutraceutical probiotic ingredient for improving health.

In some embodiments, one or more lipid (e.g., one or more lipid components) are used to provide a barrier to a nutraceutical (e.g., a nutraceutical payload component). In some embodiments, one or more lipid (e.g., one or more lipid components) are used to provide a barrier to a nutraceutical or to barrier materials used to formulate a nutraceutical.

In some embodiments, one or more carbohydrates (e.g., one or more carbohydrate components) are used to encapsulate a nutraceutical and/or barrier material(s) used to formulate a nutraceutical.

In some embodiments, one or more hydrophobic carbohydrates (e.g., one or more hydrophobic carbohydrate components) are used to encapsulate a nutraceutical and/or barrier material(s) used to formulate a nutraceutical.

In some embodiments, one or more polymers (e.g., one or more polymer components) are used to encapsulate a nutraceutical and/or barrier material(s) used to formulate a nutraceutical.

In some embodiments, one or more hydrophobic polymers (e.g., one or more hydrophobic polymer components) are used to encapsulate a nutraceutical and/or barrier material(s) used to formulate a nutraceutical.

In some embodiments, one or more proteins (e.g., one or more protein components) are used to encapsulate a nutraceutical and/or barrier material(s) used to formulate a nutraceutical.

In some embodiments, one or more hydrophobic proteins (e.g., one or more hydrophobic protein components) are used to encapsulate a nutraceutical and/or barrier material(s) used to formulate a nutraceutical.

In some embodiments, the formulated nutraceuticals and barrier material(s) are characterized as particle preparations. In some embodiments, a hydrophobic wax-based barrier material having low water activity and moisture content is used to encapsulate and stabilize a nutraceutical in foods and/or beverages.

In some embodiments, a hydrophobic carbohydrate, polymer, or protein further reduces water activity and moisture content and/or further stabilizes a probiotic composition.

In some embodiments, the method of manufacturing the provided probiotic compositions is biocompatible. Provided technologies provide benefits over existing products, because, among other things, protection of nutraceuticals from moisture content substantially prolongs shelf-life and/or viability, and facilitates incorporation of nutraceuticals into food and/or beverage products.

In some aspects, the present disclosure provides a method of enumerating microbes (e.g., probiotics) encapsulated within a probiotic composition. In some embodiments, microbes are encapsulated within a moisture-accessible portion of the probiotic composition. In some embodiments, microbes are encapsulated within a moisture-inaccessible portion of the probiotic composition. In some embodiments of the disclosure, a method of extracting (e.g., peptone extraction) microbes from the moisture-accessible portion of the probiotic composition is provided. In some embodiments of the disclosure, a method of extracting microbes from the moisture-inaccessible portion (e.g., oil extraction) of the probiotic composition is provided. In some embodiments, the method of extracting microbes from the moisture-inaccessible portion of a probiotic composition uses or may utilize a suitable oil (e.g., oil component), moderate- to low-HLB surfactants (e.g., surfactant component), and/or a suitable salt solution (e.g., salt solution component) to extract microbes from a hydrophobic phase to an aqueous phase. In some embodiments, standard microbiology techniques (e.g., spread plate enumeration) are utilized on dilutions of the moisture-accessible and/or moisture-inaccessible extracts. Provided technologies provide benefits over existing products, among other things, by substantially improving the biocompatibility of the extraction process of microbes from hydrophobic phases, thereby enabling enumeration of hydrophobic cell encapsulation, viability, and/or enumeration of total microbes in a probiotic composition.

In some aspects, the disclosure provides a method of manufacture for a probiotic composition for improving health. In some embodiments, a nutraceutical payload component is reduced to a size (e.g., size reduction) amenable to homogenous incorporation into a liquid matrix. In some embodiments, a nutraceutical payload component is reduced to a size (e.g., size reduction) amenable to mitigate any sensory aspects (e.g., texture, grit, taste, etc.). In some embodiments, nutraceutical payload components of reduced size are incorporated (e.g., incorporation process) into a molten non-aqueous matrix (e.g., probiotic composition). In some embodiments, the present disclosure provides methods of atomizing (e.g., size reduction) probiotic compositions. In some embodiments, the present disclosure provides methods of coating (e.g., coating process) probiotic compositions. Size reduction and atomization of nutraceutical payload components (for example, probiotic compositions) are often accompanied by exposure to environmental factors (e.g., excessive heat, light, oxidation, moisture, shear, pressure) that accelerate degradation of a nutraceutical payload component. The present disclosed embodiments provide conditions of size reduction and atomization to preserve viability of nutraceutical payload component(s).

In some embodiments, the present disclosure provides probiotic compositions that may be or comprise particles (e.g., microparticles) for the stabilization of microbes (e.g., probiotics), microbe food ingredients, and/or other nutraceuticals. In some embodiments, the stabilization of nutraceutical payload components is achieved via encapsulation within barrier materials. The present embodiments provide lipid materials demonstrating prolonged stability in high moisture and acidic environments. Several compositions for the encapsulation and stabilization of nutraceutical payload components (e.g., probiotics) that utilize carbohydrate (e.g., sodium alginate, pectin) or protein (e.g., gelatin) barrier materials may suffer from rapid degradation in environments with high moisture and/or water activity. Some compositions that are intended to encapsulate and stabilize nutraceutical payload components (e.g., probiotics) utilizing lipid materials (e.g., cocoa butter, vegetable oil, phospholipids) demonstrate poor stability (e.g., loss of >2 log(CFU/g) within 2 weeks) at temperatures above about 0° C. The compositions and methodologies of probiotic compositions comprising a nutraceutical payload component (e.g., probiotics, probiotic ingredients, probiotic supplement ingredients, probiotic food ingredients) disclosed herein help to address the challenges disclosed herein through manufacturing process(es) capable of preserving cell viability, through enumeration process(es) to determine viability in lipid matrices, and through compositions that improve cell viability and stability, even at 37° C. Furthermore, the disclosed compositions demonstrate surprising stability and nearly complete resistance to moisture uptake, as a result of the unique manufacturing process, when dispersed in aqueous and/or high water activity environments (e.g., milk, milk powder, yogurt, sachet, powdered supplements, soda, seltzer, alcoholic beverages, Gatorade, sports drinks, water, simulated intestinal fluid, simulated gastric fluid, aqueous solution).

### B. Probiotic Compositions Comprising Nutraceutical Payload Component(s) and/or Barrier Material(s) for Improving Health

### 1. Particle Preparations

Among other things, the present disclosure provides particle preparations (e.g., probiotic compositions, e.g. probiotic particle preparations). For example, in some embodiments, probiotic compositions are or comprise particles (e.g., particle preparations, e.g., probiotic particle preparations). For example, particles may comprise a payload component (e.g., cellular payload component) and/or a carrier component.

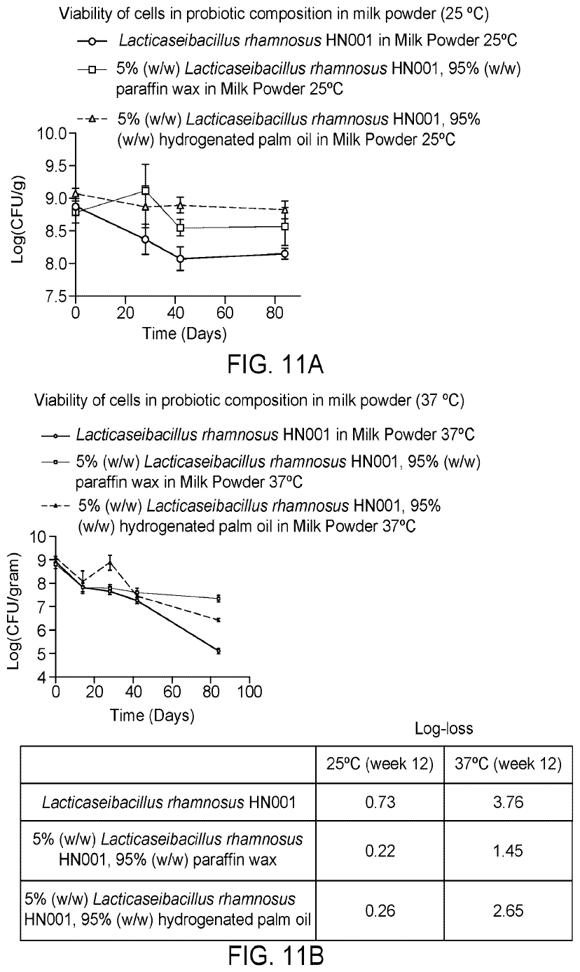

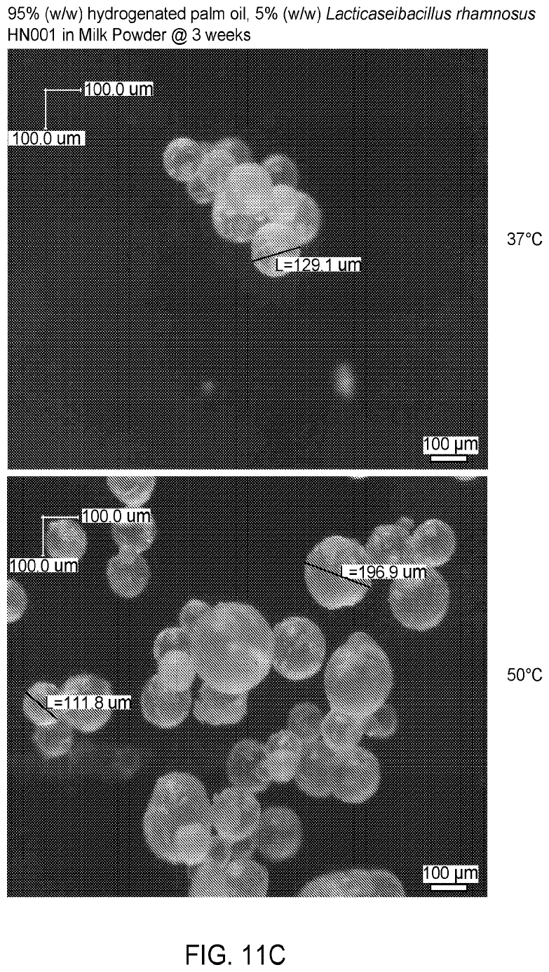

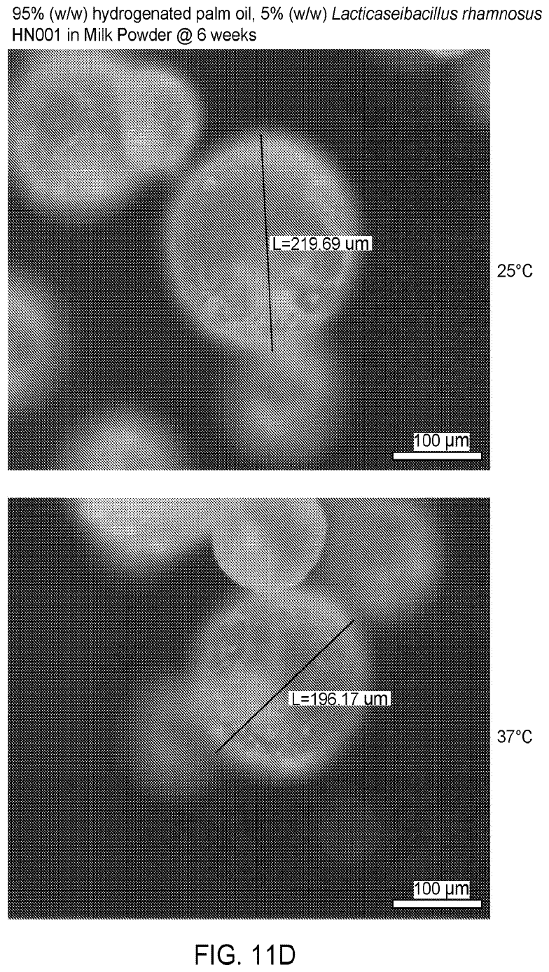

As depicted in a non-limiting schematic in FIG. 11, in some embodiments, an exemplary particle preparation may comprise a particle 100 comprising at least one carrier component 110, at least one payload component 120, at least one excipient component 130, or a combination thereof.

Additionally or alternatively, an exemplary particle preparation may comprise a particle 100 comprising at least one carrier component 110, at least one payload component 120, at least one excipient component 130, at least one matrix component 140, or a combination thereof.

In some embodiments, at least one matrix component 140 may be or comprises an at least one carrier component 110. In some embodiments, at least one matrix component 140 is at least one carrier component 110.

In some embodiments, at least one payload component 120 and/or an at least one excipient component 130 may be described as being dispersed within (e.g., embedded within) at least one matrix component 140.

In some embodiments, at least one carrier component 110 may be described as encapsulating (i) at least one payload component 120, and/or at least one excipient component 130.

In some embodiments, at least one payload component 120, at least one excipient component 130, at least one matrix component 140, or a combination thereof may be described as being dispersed within (e.g., encapsulated in) at least one carrier component 110.

### 2. Particle-in-Particle Preparations

In certain embodiments, particle preparations disclosed herein may be or comprise particles comprising particle-in-particle structures (e.g., particle-in-particle preparations), as illustrated in a non-limiting embodiment of a particle 101 in FIG. 11. For example, particle-in-particle preparations may comprise particles of exemplary particle preparations 100 further dispersed in (e.g., encapsulated in) one or more carrier components 110.

Additionally or alternatively, particle-in-particle preparations may be or comprise particles of exemplary particle preparations 100 further dispersed in (e.g., embedded in) at least one matrix component 140.

In some embodiments, exemplary particles 100 of particle preparations may be homogenously or non-homogeneously (e.g., heterogeneously) dispersed in (e.g., encapsulated in, e.g., embedded in) exemplary particles 101.

In many embodiments, nutraceutical payload components are physically associated with (e.g., encapsulated) carrier components(s) such that nutraceutical payload components are protected from environmental factors.

In certain embodiments, particle preparations comprising nutraceutical payload components and one or more carrier components(s) are physically associated with (e.g., encapsulated in) one or more carrier components. In some embodiments, particle preparations encapsulated in one or more carrier component(s) (e.g., particle-in-particle structures) are further protected from environmental factors (e.g., water, light, heat).

In some embodiments, particle-in-particle structures comprise particle preparations comprising nutraceutical payload components and one or more carrier components encapsulated within carrier components that protect a nutraceutical payload component (e.g., microbe, probiotic, bacteria) from the same environmental factor (e.g., water, light, heat).

In other embodiments, particle-in-particle structures comprise particle preparations comprising nutraceutical payload components and one or more carrier components encapsulated within carrier components that protect a nutraceutical payload component (e.g., microbe, probiotic, bacteria) from a different environmental factor (e.g., water, light, heat).

In some instances, a carrier component is or comprises biocompatible material(s) comprising at least one of sugar, polysaccharide, carbohydrate, oil, fat, wax, protein, polymer, or a combination thereof. In some embodiments, one or more bacterial species are embedded in a carrier component.

### 3. Size and Shape of Particles and Particle Preparations

In some embodiments (e.g., FIG. TA-1H), the present disclosure provides particle preparations in which particles have a particular shape or form, for example, having a cross-section shape of a circle, an oval, a triangle, a square, a hexagon, or an irregular shape. In some embodiments, a preparation includes particles of different shapes or forms. In some embodiments, most or substantially all or all particles in a preparation have a common shape.

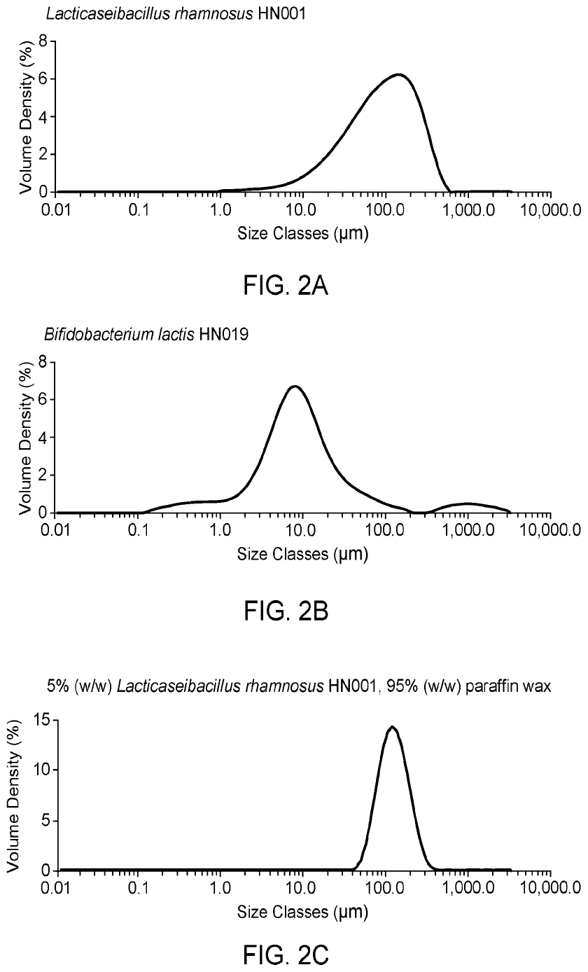

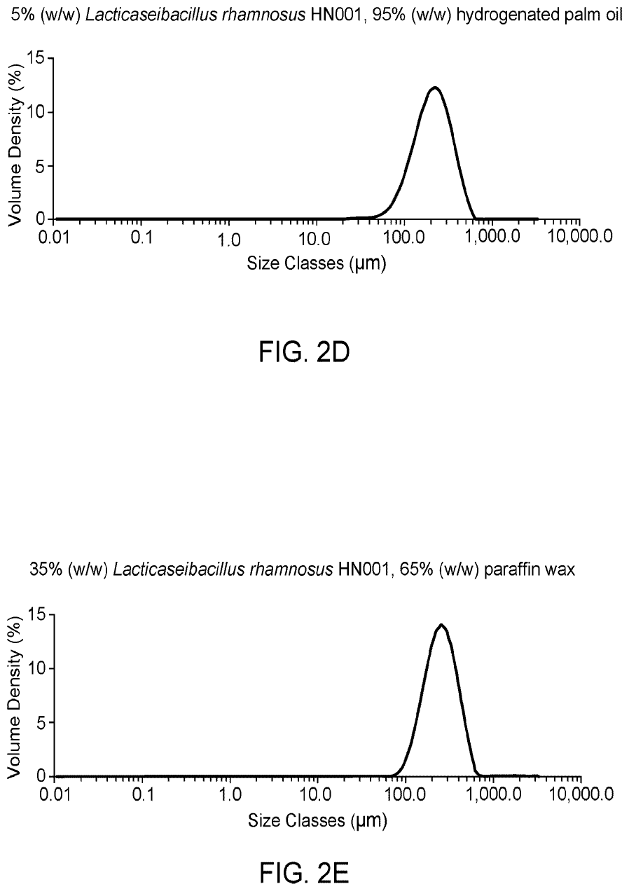

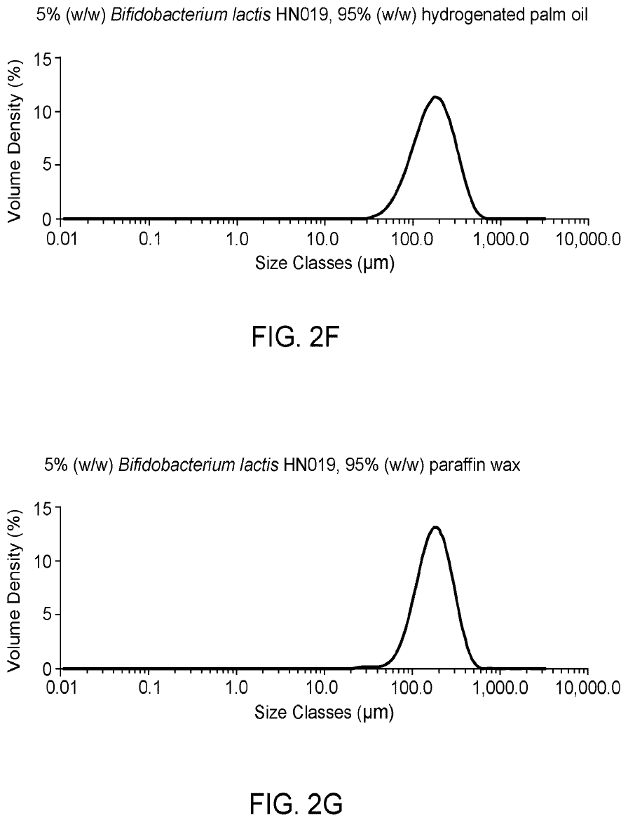

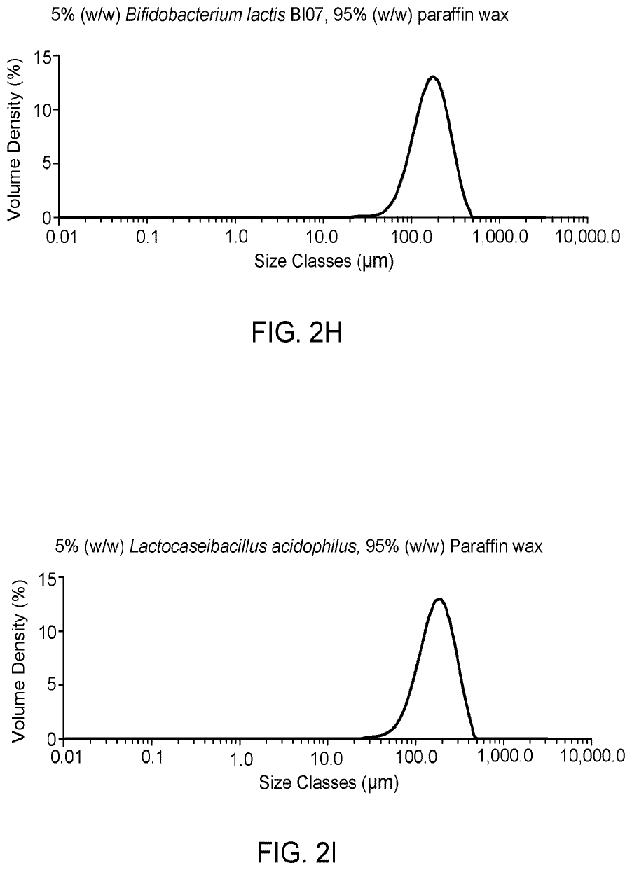

In some embodiments (e.g., FIG. 2A-2I), particles (e.g., lipid microparticles) in a provided particle preparation may have a distribution of diameters (e.g., Dv(10), Dv(20), Dv(30), Dv(40), Dv(50), Dv(60), Dv(70), Dv(80), Dv(90), Dv99, etc.). In some embodiments, particles (e.g., lipid microparticles) in a provided particle preparation may have an average diameter (e.g., D[3.2], D[4.3], etc.). Regardless of the shape of the particle, the “diameter” (i.e., size) of a particle is the longest distance from one end of the particle to another end of the particle.

In some instances (e.g., FIG. 2A-2I), particles in a particle preparation as described and/or utilized herein may have a distribution of diameters (e.g., Dv(10), Dv(20), Dv(30), Dv(40), Dv(50), Dv(60), Dv(70), Dv(80). Dv(90), Dv(99), etc.) of up to about 3000 μm, up to about 2000 μm, up to about 1000 μm, up to about 500 μm, up to about 400 μm, up to about 300 μm, up to about 200 μm, up to about 100 μm, up to about 50 μm, up to about 40 μm, up to about 30 μm, up to about 20 μm, up to about 10 μm, or up to about 5 μm.

In some embodiments, provided probiotic compositions (e.g., particle preparations) are or comprise particles with an average diameter (e.g., D[3,2], D[4,3], etc.) of particles in the range of about 5-3000 m, about 5-2000 μm, about 5-1000 μm, about 5-500 μm, about 5-250 μm, about 100-250 μm, about 5-175 μm, about 5-100 μm, about 5-50 μm, about 5-10 μm, and/or about 4-6 μm.

In some instances, particle preparations comprise particles (e.g., wax microparticles comprising a nutraceutical payload component) characterized by an average particle diameter (e.g., D[3,2], D[4,3], etc.) within a range of about 5 μm to about 1000 μm. In some instances, particle preparations comprise particles (e.g., lipid microparticles comprising a nutraceutical payload component) characterized by an average particle diameter (e.g., D[3,2], D[4,3], etc.) within a range of about 5 μm to about 400 μm.

In some instances, particle preparations comprise particles (e.g., lipid microparticles comprising a nutraceutical payload component) characterized by an average particle diameter (e.g., D[3,2], D[4,3], etc.) within a range of about 60 μm to about 200 μm.

In some instances, particle preparations comprise particles (e.g., lipid microparticles comprising a payload component) characterized by an average particle diameter (e.g., D[3,2], D[4,3], etc.) within a range of about 100 μm to about 300 μm.

In some instances, particle preparations comprise particles (e.g., lipid microparticles comprising a payload component) characterized by an average particle diameter (e.g., D[3,2], D[4,3], etc.) within a range of about 60 μm to about 300 μm.

In some instances, particle preparations comprise particles (e.g., lipid microparticles comprising a nutraceutical payload component) characterized by an average particle diameter (e.g., D[3,2], D[4,3], etc.) within a range of about 100 μm to about 1000 μm.

In some instances, particle preparations (e.g., probiotic compositions) comprise particles (e.g., lipid microparticles comprising nutraceutical payload components) characterized by an average particle diameter (e.g., D[3,2], D[4,3], etc.) within arange of about 5 μm to about 60 μm.

In some instances, particle preparations (e.g., probiotic compositions) comprise particles (e.g., lipid microparticles comprising a nutraceutical payload component) characterized by an average particle diameter (e.g., D[3,2], D[4,3], etc.) within a range of about 5 μm to about 300 μm.

### 4. Particle-in-Particle Structures

In certain embodiments, probiotic compositions (e.g., particle preparations) comprise nutraceutical payload components and one or more barrier material(s). In many embodiments, nutraceutical payload components are physically associated with (e.g., encapsulated) barrier materials(s) such that nutraceutical payload components are protected from environmental factors. In some embodiments, nutraceutical payload components encapsulated in one or more barrier material(s) are a particle preparation.

In certain embodiments, particle preparations comprising nutraceutical payload components and one or more barrier materials(s) are physically associated with (e.g., encapsulated in) one or more barrier materials. In some embodiments, particle preparations encapsulated in one or more barrier material(s) (e.g., particle-in-particle structures) are further protected from environmental factors (e.g., water, light, heat).

In certain embodiments, particle preparations comprising nutraceutical payload components and one or more barrier materials(s) are physically associated with (e.g., encapsulated in) one or more barrier materials. In some embodiments, particle preparations encapsulated in one or more barrier material(s) (e.g., particle-in-particle structures) are further protected from environmental factors encountered during processing and manufacturing approaches (e.g., shear, heat, pressure).

In some embodiments, particle preparations physically associated with (e.g., encapsulated) within barrier material(s) (e.g., particle-in-particle structures) are or may be dispersed homogeneously within barrier material(s). In some embodiments, particle preparations physically associated with (e.g., encapsulated) within barrier material(s) (e.g., particle-in-particle structures) are or may be dispersed non-homogeneously within barrier material(s).

In some embodiments, particle-in-particle structures comprise particle preparations comprising nutraceutical payload components and one or more barrier materials(s) encapsulated within barrier materials that protect a nutraceutical payload component (e.g., microbe, probiotic, bacteria, mineral, carotenoid, and/or combinations thereof) from the same environmental factor (e.g., water, light, heat, shear, pressure, acid). In other embodiments, particle-in-particle structures comprise particle preparations comprising nutraceutical payload components and one or more barrier materials(s) encapsulated within barrier materials that protect a nutraceutical payload component (e.g., microbe, probiotic, bacteria, mineral, carotenoid, and/or combinations thereof) from a different environmental factor (e.g., water, light, heat, shear, pressure, acid).

In some embodiments, particle-in-particle structures may be further encapsulated in one or more barrier material(s).

### 5. Nutraceutical Payload Components

In many embodiments, a nutraceutical payload component may be useful (e.g., may be beneficial) to an aspect of human/animal health or behavior, or otherwise a feature of an environment to which a provided composition is applied/in which a provided composition is utilized.

For example, in some embodiments, a nutraceutical payload component is or comprises at least one of: antioxidants, macronutrients, micronutrients, minerals, prebiotics, probiotics, vitamins, or combinations thereof. For example, in many instances, a nutraceutical payload component may comprise only a probiotic component. In another example, a nutraceutical payload component may comprise a formulated probiotic component (e.g., probiotic food ingredient, probiotic ingredient, formulated probiotic, encapsulated probiotic). In another example, a nutraceutical payload component may comprise only a mineral component. In some embodiments, a nutraceutical payload component may include only non-probiotic, non-mineral payload component(s).

In some embodiments, a payload component (e.g., nutraceutical payload component) is fat soluble. In some instances, a payload component (e.g., nutraceutical payload component) is water soluble. In some embodiments, a payload component (e.g., nutraceutical payload component) is both fat soluble and water soluble. In some embodiments, a payload component (e.g., nutraceutical payload component) is partially fat soluble. In some instances, a payload component (e.g., nutraceutical payload component) is partially water soluble. In some embodiments, a payload component (e.g., nutraceutical payload component) is both partially fat soluble and partially water soluble.

In some embodiments, a payload component (e.g., nutraceutical payload component) is or comprises at least one micronutrient. In some instances, a micronutrient is or comprises at least one vitamin. For example, a vitamin is or comprises vitamin B1, vitamin B2, vitamin B3, vitamin B5, vitamin B6, vitamin B7, vitamin B9, vitamin B12, vitamin A, vitamin C, vitamin D, vitamin E, vitamin K, or a combination thereof. In some instances, a payload component is or comprises vitamin D. In some instances, a micronutrient is or comprises at least one carotenoid compound. For example, a carotenoid is or comprises alpha-lipoic acid, astaxanthin, adonixanthin, adonirubin, beta-carotene, coenzyme Q10, lutein, lycopene, zeaxanthin, meso-zeaxanthin, and/or combinations thereof.

In some embodiments, a payload component (e.g., nutraceutical payload component) is or comprises at least one macronutrient. In some instances, a macronutrient is or comprises at least one carbohydrate, at least one fat, at least one protein, or a combination thereof.

In some embodiments, a payload component (e.g., nutraceutical payload component) is or comprises at least one mineral and/or element. In some instances, a mineral is or comprises iron, zinc, calcium, magnesium, manganese, phosphorus, cobalt, potassium, sodium, oxide, carbonate, chloride, iodine, sulfur, copper, fluoride, selenium, or a combination thereof.

In some embodiments, a payload component (e.g., nutraceutical payload component) is or comprises at least one short chain fatty acid. In some instances, a short chain fatty acid is or comprises acetate, propionate, and butyrate, or a combination thereof.

In some embodiments, a payload component (e.g., nutraceutical payload component) is or comprises at least one probiotic species. In some instances, a probiotic is or comprises at least one species of yeast, at least one species of fungus, at least one species of bacteria, or a combination thereof.

In some instances, a payload component (e.g., nutraceutical payload component) is or comprises at least one probiotic species. In some instances, a probiotic is or comprises at least one species of fungus. In some instances, at least one species of fungus is or comprises Saccharomyces cerevisiae and/or Saccharomyces boulardii.

In some instances, a payload component (e.g., nutraceutical payload component) is or comprises at least one probiotic species. In some instances, a probiotic is or comprises at least one species of bacteria. In some instances, at least one species of bacteria is or comprises Bacillus coagulans, Bacillus licheniformis, Bacillus subtilis, Bifidobacterium angulatum, Bifidobacterium animalis, Bifidobacterium bijidum, Bijidobacterium breve, Bifidobacterium infantis, Bijidobacterium Bifidobacterium longum, Enterococcus foecium, Enterococcus faecalis, Lactobacillus acidophilus, Lactobacillus amylovorus, Lactobacillus alimentarius, Lactobacillus bulgaricus, Lactobacillus casei subsp. casei, Lactobacillus casei Shirota, Lactobacillus curvatus, Lactobacillus delbrueckii subsp lactis, Lactobacillus fermentum, Lactobacillus farciminis, Lactobacillus gasseri, Lactobacillus helveticus, Lactobacillus johnsonii, Lactobacillus lacti, Lactobacillus paracasei, Lactobacillus pentosaceus, Lactobacillus plantarum, Lactobacillus reuteri, Lactobacillus rhamnosus (Lactobacillus GG), Lactobacdlus sake, Lactobacillus salivarius, Lactococcus lactis, Micrococcus varians, Pediococcus acidilactici Pediococcus pentosaceus, Pediococcus acidilactici, Pediococcus halophilus, Streptococcus faecalis, Streptococcus thermophilus, Staphylococcus carnosus, or Staphylococcus xylosus Lactobacillus acidophilus, Lactobacillus bulgaricus, Lactobacillus rhamnosus, Lactobacillus reuteri, Streptococcus thermophilus, Bifidobacterium animalis, Bifidobacterium bifidum, Bifidobacterium lactis, Bacillus subtilis, and/or a combination thereof.

In some embodiments, a payload component (e.g., nutraceutical payload component) is or comprises at least one probiotic species that is considered a spore forming species. In some instances, a probiotic species is or comprises Bacillus coagulans, Bacillus lichenjbrmis, Bacillus subtilis, and/or a combination thereof.

In some embodiments, a payload component (e.g., nutraceutical payload component) is or comprises at least one prebiotic. In some instances, at least one prebiotic is or comprises non-digestible fibers (e.g., inulin), bacteriophage, or a combination thereof.

In some embodiments, a payload component (e.g., nutraceutical payload component) is or comprises at least one probiotic ingredient that is provided as a commercial product (e.g., probiotic ingredient, probiotic food ingredient). In some instances, a probiotic ingredient is or comprises probiotics that are encapsulated, comprising cryoprotectants, encapsulated with cryoprotectants, mixed with cryoprotectants, comprising flow-aids or anti-caking agents (e.g., dry-flo, starch, microcrystalline cellulose), encapsulated with flow-aids or anti-caking agents, mixed with flow-aids or anti-caking agents, comprising desiccants, encapsulated with desiccants, mixed with desiccants, and/or a combination thereof.

In some embodiments, a probiotic ingredient comprises one or more probiotics in the absence of a cryoprotectant.

In some embodiments, a microbe component may comprise an amount of at least one microbe characterized by CFUs. For example, a microbe component may comprise about 10⁴-10¹⁶ CFUs. In some embodiments, a microbe component may comprise about 10⁴-10¹² CFUs. In some embodiments, a microbe component may comprise about 10⁹-10¹⁶ CFUs. In some embodiments, a microbe component may comprise about 10⁴-10⁹ CFUs. In certain embodiments, it is of convenience to express the enumeration of the microbe component in logarithmic units (i.e., log(CFU)).

In some embodiments, a microbe component may comprise an amount of at least one microbe characterized by CFUs/gram or per unit mass. For example, a microbe component may comprise about 10⁴-10⁶ CFUs/gram. In some embodiments, a microbe component may comprise about 10⁴-10¹² CFUs/gram. In some embodiments, a microbe component may comprise about 10⁹-10⁶ CFUs/gram. In some embodiments, a microbe component may comprise about 10⁴-10⁹ CFUs/gram. In certain embodiments, it is of convenience to express the enumeration of the microbe component in logarithmic units (i.e., log(CFU/gram)).

In some embodiments, a microbe component may comprise an amount of at least one microbe characterized by CFUs/ml or per unit volume. For example, a microbe component may comprise about 10⁴-10¹⁶ CFUs/ml. In some embodiments, a microbe component may comprise about 10⁴-10¹² CFUs/ml. In some embodiments, a microbe component may comprise about 10⁹-10¹⁶ CFUs/ml. In some embodiments, a microbe component may comprise about 10⁴-10⁹ CFUs/ml. In certain embodiments, it is of convenience to express the enumeration of the microbe component in logarithmic units (i.e., log(CFU/ml)).

It is contemplated that, in some embodiments, probiotics are or may be encapsulated inside particles in a particle preparation as described herein. Alternatively or additionally, one or more probiotics can be combined with a particle preparation as described herein. In some embodiments, the probiotics are or may be encapsulated inside particles that are an ingredient or a food ingredient or a supplement ingredient comprising formulated microbes (e.g., probiotics).

It is contemplated that, in some embodiments, probiotics are or may be encapsulated inside particles in a particle preparation as described herein. Alternatively or additionally, one or more probiotics can be combined with a particle preparation as described herein (e.g., where particles of the preparation include a nutraceutical such as for example, a mineral compound (e.g., iron oxide, calcium carbonate, and/or a combination thereof) or a carotenoid (e.g., lutein)).

In some embodiments, at least one probiotic species is at least about 90 wt %, at least about 85 wt %, at least about 80 wt %, at least about 75 wt %, at least about 70 wt %, at least about 65 wt %, at least about 60 wt %, at least about 55 wt %, at least about 50 wt %, at least about 45 wt %, at least about 40 wt %, at least about 35 w-t %, at least about 30 wt %, at least about 25 wt %, at least about 20 wt %, at least about 15 wt %, at least about 10 wt %, at least about 5 wt %, at least about 1 wt %, at least about 0.8 wt %, at least about 0.5 wt %, at least about 0.1 wt % of a particle preparation (i.e., probiotic composition).

### 6. Carrier Components

In many embodiments, a nutraceutical payload component is encapsulated within a barrier material. In a typical embodiment, barrier materials are chosen to exclude one or more environmental agents (e.g., water, moisture, humidity, water activity, acidity, light, heat, oxygen). In some embodiments, it is advantageous to exclude the encapsulated nutraceutical payload components from environmental water (e.g., moisture barrier materials). In some embodiments, it is advantageous to exclude the encapsulated nutraceutical payload components from acidity (e.g., pH responsive materials). In some embodiments, it is advantageous to exclude the encapsulated nutraceutical payload components from molecular oxygen, oxygen radicals, or combinations thereof (e.g., oxygen scavenging materials). In some embodiments, it is advantageous to protect the encapsulated nutraceutical payload components from physical forces or environmental conditions (e.g., elevated heat, elevated pressure, shear), or combinations thereof.

In some embodiments, a nutraceutical payload component is encapsulated in a range of 1-15, 1-10, 1-8, 1-6, 1-4, and/or 1-2 distinct barrier materials. In some embodiments, a nutraceutical payload component is encapsulated in 1 barrier material. In other embodiments, a nutraceutical payload component is encapsulated in 2 barrier materials.

In certain embodiments, a nutraceutical payload component is encapsulated in a range of 1-15, 1-10, 1-8, 1-6, 1-4, and/or 1-2 barrier materials that are homogeneously blended. In certain embodiments, the nutraceutical payload component is encapsulated in a range of 1-15, 1-10, 1-8, 1-6, 1-4, and/or 1-2 barrier materials that are subsequently encapsulated in a range of 1-15, 1-10, 1-8, 1-6, 1-4, and/or 1-2 barrier materials.

In certain embodiments, barrier materials are selected to achieve the exclusion of one or more environmental agents that accelerate degradation and/or decrease viability and/or decrease CFUs of the encapsulated nutraceutical payload component. In some embodiments, the nutraceutical payload component is encapsulated in one or more barrier materials that exclude environmental water, acidity, and/or molecular oxygen, oxygen radicals, or combinations thereof. In some embodiments, the barrier materials exclude only environmental water; in other cases, barrier materials exclude environmental water and/or acidity.

In certain embodiments, barrier materials are selected to achieve the protection of the encapsulated nutraceutical payload component by mitigating/protecting against degradation and/or mitigating/protecting decreases viability and/or mitigating/protecting against decreases in CFUs when exposed to one or more environmental physical forces. In some embodiments, the nutraceutical payload component is encapsulated in one or more barrier materials that protect against heat, shear, elevated pressure, vacuum, or combinations thereof. In some embodiments, the barrier materials protect against only shear: in other cases, barrier materials protect against shear and/or heat; in other cases, barrier materials exclude environmental water and/or acidity and/or also protect against shear.

In some embodiments, at least one barrier material is at least about 99 wt %, 95 wt % 90 wt %, at least about 85 wt %, at least about 80 wt %, at least about 75 wt %, at least about 70 wt %, at least about 65 wt %, at least about 60 wt %, at least about 55 wt %, at least about 50 wt %, at least about 45 wt %, at least about 40 wt %, at least about 35 wt %, at least about 30 wt %, at least about 25 wt %, at least about 20 wt %, at least about 15 wt %, at least about 10 wt %, at least about 5 wt %, at least about 1 wt %, at least about 0.8 wt %, at least about 0.5 wt %, at least about 0.1 wt % of a particle preparation (i.e., probiotic composition).

### 7. Lipid Components

Typically, as described herein, utilized lipid component(s) is or are characterized by hydrophobicity (i.e., is a hydrophobic lipid component). In some instances, lipid component(s) is or are characterized by melting point (i.e., is a room temperature solid, low melt temperature lipid component).

As provided herein, a lipid component may be or comprises at least one lipid. In some instances, lipid components can be a combination of lipids, each of which may or may not be individually hydrophobic and/or room temperature solids with low melt temperature.

In some instances, a lipid component may be or comprise one or more waxes, fats, fatty acids, fatty alcohols, glycerol ethers, glycol ethers, glycerol esters, glycol esters, natural oils, processed oils, sterols, or combinations thereof.

In some instances, lipid component(s) comprise one or more waxes. For example, in some embodiments, wax(es) may comprise paraffin wax, montan wax, microcrystalline wax, polyethylene wax, petrolatum wax, ozokerite wax, ceresin wax, beeswax, lanolin wax, spermaceti wax, tallow wax, lac wax, Chinese insect wax, ambergris wax, soy wax, camauba wax, candelilla wax, coconut wax, palm kernel wax, rice bran wax, or combinations thereof.

In some instances, lipid component(s) comprise one or more hydrogenated plant oils. For example, in some embodiments, wax(es) may comprise fatty acid monoglyceride esters, fatty acid diglyceride esters, fatty acid triglyceride esters, coconut oil, cottonseed oil, palm oil, soybean oil, sunflower oil, fully hydrogenated palm oil, partially hydrogenated palm oil, fully hydrogenated sunflower oil, partially hydrogenated sunflower oil, fully hydrogenated soybean oil, partially hydrogenated soybean oil, fully hydrogenated vegetable oil, partially hydrogenated vegetable oil, fully hydrogenated cottonseed oil, partially hydrogenated cottonseed oil, or combinations thereof.

In some instances, lipid component(s) comprise one or more fatty acids. For example, in some embodiments, wax(es) may comprise butyric acid, pentanoic acid, hexanoic acid, heptanoic acid, caprylic acid, nonanoic acid, capric acid, lauric acid, myristic acid, palmitic acid, stearic acid, and/or arachidonic acid, or combinations thereof.

In some instances, one or more lipid component(s) melt(s) at a temperature of >28° C., >35° C., >45° C., >55° C., >65° C., >75° C., >85° C., and/or >95° C.

In some instances, one or more lipid component(s) melt(s) at a temperature of <105° C., <95° C., <85° C., <75° C., <65° C., <55° C., <45° C., and/or <35° C.

In some embodiments, one or more hydrophobic lipid component(s) and/or room temperature solid lipid component(s) is or are associated (e.g., encapsulating) with one or more nutraceuticals in a particle preparation (e.g., probiotic composition) as described herein.

In some embodiments, at least one lipid component is at least about 99 wt %, at least about 95 wt %, at least about 90 wt %, at least about 85 wt %, at least about 80 wt %, at least about 75 wt %, at least about 70 wt %, at least about 65 wt %, at least about 60 wt %, at least about 55 wt %, at least about 50 wt %, at least about 45 wt %, at least about 40 wt %, at least about 35 wt %, at least about 30 wt %, at least about 25 wt %, at least about 20 wt %, at least about 15 wt %, at least about 10 wt %, at least about 5 wt %, at least about 2.5 wt %, or at least about 1 wt % of a particle preparation (i.e., probiotic composition).

In some embodiments, at least one lipid component is about 0.25 wt % to about 99.75 wt %, about 0.5 wt % to about 99.75 wt %, about 0.75 wt % to about 99.75 wt %, about 1 wt % to about 99.75 wt %, about 2.5 wt % to about 99.75 wt %, about 5 wt % to about 99.75 wt %, about 10 wt % to about 99.75 wt %, about 25 wt % to about 99.75 wt %, about 50 wt % to about 99.75%, about 75 wt % to about 99.75%, about 0.25 wt % to about 75 wt %, about 0.5 wt % to about 75 wt %, about 0.75 wt % to about 75 wt %, about 1 wt % to about 75 wt %, about 2.5 wt % to about 75 wt %, about 5 wt % to about 75 wt %, about 10 wt % to about 75 wt %, about 25 wt % to about 75 wt %, about 50 wt % to about 75%, about 0.25 wt % to about 50 wt %, about 0.5 wt % to about 50 wt %, about 0.75 wt % to about 50 wt %, about 1 wt % to about 50 wt %, about 2.5 wt % to about 50 wt %, about 5 wt % to about 50 wt %, about 10 wt % to about 50 wt %, about 25 wt % to about 50 wt %, about 0.25 wt % to about 25 wt %, about 0.5 wt % to about 25 wt %, about 0.75 wt % to about 25 wt %, about 1 wt % to about 25 wt %, about 2.5 wt % to about 25 wt %, about 5 wt % to about 25 wt %, about 10 wt % to about 25 wt %, about 0.25 wt % to about 10 wt %, about 0.5 wt % to about 10 wt %, about 0.75 wt % to about 10 wt %, about 1 wt % to about 10 wt %, about 2.5 wt % to about 10 wt %, about 5 wt % to about 10 wt %, about 0.25 wt % to about 5 wt %, about 0.5 wt % to about 5 wt %, about 0.75 wt % to about 5 wt %, about 1 wt % to about 5 wt %, or about 2.5 wt % to about 5 wt %.

### 8. Carbohydrate Components

Typically, as described herein, utilized carbohydrate component(s) is or are characterized by hydrophobicity (i.e., is a hydrophobic carbohydrate component, is a hydrophilic carbohydrate component). In some instances, carbohydrate component(s) is or are characterized by negative charge at physiological pH (i.e., pH 7.4) (i.e., is a negatively charged carbohydrate).

As provided herein, a carbohydrate component may be or comprises at least one carbohydrate. In some instances, carbohydrate components can be a combination of carbohydrates, each of which may or may not be individually hydrophobic and/or negatively charged at physiological pH (i.e., pH 7.4).

In some instances, a carbohydrate component may be or comprise one or more starches, cellulose, starch derivatives, cellulose derivatives, anionic carbohydrates, pH-responsive carbohydrates, mucoadhesive carbohydrates, polysaccharides, dietary fiber, or combinations thereof.

In some instances, carbohydrate component(s) comprise one or more starches and/or starch derivatives. For example, in some embodiments, starches may comprise amylose, amylopectin, and/or combinations thereof.

In some embodiments, carbohydrate component(s) comprise one or more celluloses and/or cellulose derivatives. For example, in some embodiments, celluloses may comprise cellulose, methyl cellulose, ethyl cellulose, hydroxypropyl cellulose, hydroxypropyl methyl cellulose, hydroxypropyl ethyl cellulose, cellulose triacetate, cellulose acetate succinate, cellulose acetate butyrate, cellulose acetate phthalate, hydroxypropyl methylcellulose acetate succinate, and/or combinations thereof.

In some embodiments, carbohydrate component(s) comprise one or more anionic carbohydrate derivatives. For example, in some embodiments, anionic derivatives may comprise sodium alginate, potassium alginate, calcium alginate, magnesium alginate, zinc alginate, sodium pectinate, potassium pectinate, calcium pectinate, zinc pectinate, sodium hyaluronate, potassium hyaluronate, calcium hyaluronate, magnesium hyaluronate, zinc hyaluronate, and/or combinations thereof.

In some embodiments, carbohydrate component(s) are considered pH-responsive carbohydrates. In certain embodiments, pH-responsive carbohydrates are carbohydrate materials that are characterized by their water solubility at a predetermined pH. In some embodiments, pH-responsive carbohydrates are characterized by their water solubility at low pH (e.g., pH<about 5, pH<about 4, pH<about 3, pH<about 2, pH<about 1). In some embodiments, pH-responsive carbohydrates exhibit low water solubility at low pH and higher water solubility at moderate (e.g., pH of about 5.5, about 6, about 6.5, about 7, about 7.5, about 8) to high (e.g., pH>8, pH>9, pH>10, pH>11, pH>12) pH. In other embodiments, pH-responsive carbohydrates exhibit higher water solubility at low pH and lower water solubility at moderate to high pH.

For example, in some embodiments, pH-responsive carbohydrate component(s) may comprise sodium alginate, potassium alginate, calcium alginate, magnesium alginate, zinc alginate, sodium pectinate, potassium pectinate, calcium pectinate, zinc pectinate, sodium hvaluronate, potassium hyaluronate, calcium hyaluronate, magnesium hyaluronate, zinc hyaluronate, cellulose acetate succinate, cellulose acetate butyrate, cellulose acetate phthalate, hydroxypropyl methylcellulose acetate succinate, heparin sodium, sodium carboxymethylcellulose, chitosan, and/or combinations thereof.

In some embodiments, carbohydrate component(s) are considered mucoadhesive carbohydrates. In certain embodiments, mucoadhesive carbohydrates are carbohydrate materials that are characterized by their ability to interact with the mucosal interface (e.g., mucus, mucins, glycocalyx, proteoglycans, cell membrane, phospholipids). Without wishing to be bound by any particular theory, mucoadhesive carbohydrates may utilize a combination of hydrogen bonding, charge-charge interaction, and hydrophobic effect to prolong residence time of formulations (e.g., particle preparations) on a mucosal surface.

For example, in some embodiments, mucoadhesive carbohydrate component(s) may comprise sodium alginate, potassium alginate, calcium alginate, magnesium alginate, zinc alginate, sodium pectinate, potassium pectinate, calcium pectinate, zinc pectinate, sodium hyaluronate, potassium hyaluronate, calcium hyaluronate, magnesium hyaluronate, zinc hyaluronate, sodium carboxymethylcellulose, chitosan, and/or combinations thereof.

In some instances, carbohydrate component(s) comprise one or more polysaccharides. For example, in some embodiments, polysaccharides may comprise hyaluronic acid, chitosan, glycol chitosan, alginate, sodium alginate, pectin, guar gum, alginic acid, agarose, dextran, and/or combinations thereof.

In some instances, carbohydrate component(s) comprise one or more dietary fiber. For example, in some embodiments, dietary fiber may comprise inulin, pectin, amylopectin, and/or combinations thereof.

In some embodiments, one or more carbohydrate component(s) is or are associated (e.g., encapsulating) with one or more nutraceuticals in a particle preparation (e.g., probiotic composition) as described herein.

In some embodiments, one or more carbohydrate components(s) is or are associated (e.g., encapsulating) with one or more barrier materials in a particle preparation (e.g., probiotic composition) as described herein.

In some embodiments, one or more carbohydrate components(s) is or are at least one prebiotic. In some instances, at least one prebiotic is or comprises non-digestible fibers (e.g., inulin, pectin, etc.),

In some embodiments, at least one carbohydrate material is at least about 50 wt %, at least about 45 wt %, at least about 40 wt %, at least about 35 wt %, at least about 30 wt %, at least about 25 wt %, at least about 20 wt %, at least about 15 wt %, at least about 10 wt %, at least about 5 wt %, at least about 1 wt %, at least about 0.8 wt %, at least about 0.5 wt %, at least about 0.1 wt %, at least about 0 wt % of a particle preparation (i.e., probiotic composition).

In some embodiments, probiotic compositions of the present disclosure do not include octenyl succinic anhydride starch. In some embodiments, probiotic compositions of the present disclosure do not include chitosan.

### 9. Protein Components

Typically, as described herein, utilized protein component(s) is or are characterized by hydrophobicity (i.e., is a hydrophobic protein component, is a hydrophilic protein component).

As provided herein, a protein component may be or comprises at least one protein. In some instances, protein components can be a combination of proteins, each of which may or may not be individually hydrophobic.

In some embodiments, a protein component may be or comprise one or more globulin, albumin, prolamin, zein, whey, casein, and/or combinations thereof.

In some embodiments, protein component(s) comprise one or more globulins. For example, in some embodiments, globulins may comprise whey protein, β-lactoglobulin, α-lactalbumin, casein, and/or combinations thereof.

In some embodiments, protein component(s) comprise one or more albumins. For example, in some embodiments, albumins may comprise bovine serum albumin, ovalbumin. and/or combinations thereof.

In some embodiments, protein component(s) comprise one or more prolamins. For example, in some embodiments, prolamins may comprise zein, hordein, gliadin, secalin, kafirin, avenin, and/or combinations thereof.

In some embodiments, one or more protein component(s) is or are associated (e.g., encapsulated) with one or more nutraceuticals in a particle preparation (e.g., probiotic composition) as described herein.

In some embodiments, one or more protein components(s) is or are associated (e.g., encapsulated) with one or more barrier materials in a particle preparation (e.g., probiotic composition) as described herein.

In some embodiments, at least one protein component is at least about 50 wt %, at least about 45 wt %, at least about 40 wt %, at least about 35 wt %, at least about 30 wt %, at least about 25 wt %, at least about 20 wt %, at least about 15 wt %, at least about 10 wt %, at least about 5 wt %, at least about 1 wt %, at least about 0.8 wt %, at least about 0.5 wt %, at least about 0.1 w-t %, at least about 0 wt % of a particle preparation (i.e., probiotic composition).

### Polymer Components

Typically, as described herein, utilized polymer component(s) is or are characterized by charge at physiological pH (i.e., pH 7.4) (i.e., is an anionic polymer, is a neutral polymer, is a zwitterionic polymer, is a cationic polymer).

As provided herein, a polymer component may be or comprise at least one polymer. In some instances, polymer components can be a combination of polymers, each of which may or may not be individually charged at physiological pH (i.e., pH 7.4).

In some instances, a polymer component may comprise acidic groups. For example, in some embodiments, anionic polymers may comprise carboxylic acids (—COOH), sulfonic acids (—SO3H), phosphonic acids, or boronic acids. In some instances, a polymer component may be characterized as exhibiting mucoadhesivity. For example, polymer component(s) may be poly(methyl methacrylate), poly(vinyl acetate succinate), poly(methacrylic acid), poly(acrylic acid), and/or poly(vinyl acetate).

In some instances, a polymer component may be or comprises a copolymer comprising methacrylate. For example, a polymer component (e.g., a pH-responsive polymer component) may comprise butyl methacrylate, 2-dimethylaminoethyl methacrvlate, methyl methacrylate. In some instances, a polymer component may be or comprises poly(butylmethacrylate-co-(2-dimethylaminoethyl)methacrylate-co-methylmethacrylate).

### 10. Excipient Components

In some embodiments, a probiotic composition (e.g., particle preparation) may further comprise an excipient component.

In some embodiments, an excipient component utilized in accordance with the present disclosure is or comprises components that are not payload components and/or are not barrier components or carrier components.

In some embodiments, an excipient component is or comprises at least one anti-caking component, anti-agglomerating component, anti-clumping component, anti-aggregating component, a surfactant component, a plasticizing component, an acid scavenger, an oxygen scavenger, a moisture scavenger, a water scavenger, a desiccant, or a combination thereof. In some embodiments, an excipient component is or comprises one or more starch, cellulose, and/or sugar compounds. In some embodiments, an excipient component imparts a benefit (e.g., reduced caking, reduced agglomeration, reduced clumping, increased stability, increased biocompatibility) on a probiotic composition. In some embodiments, an excipient component imparts a change to the environment within the particle preparation (e.g., pH change, oxygen concentration change, water concentration change) for a probiotic composition. In some embodiments, an excipient component imparts a change (e.g., pH change, oxygen concentration change, water concentration change) to the local environment (e.g., stomach, food matrix, beverage) where the probiotic composition resides at a point in time.

Excipient components exhibiting one or more of anti-caking, anti-agglomerating, anti-clumping, anti-aggregating, surfactant, and/or plasticizing properties may comprise substance(s) identified by one or more governing bodies as safe (e.g., generally regarded as safe and/or food additives). In some instances, those skilled in the art will appreciate that excipient component(s) are or may be selected from those excipient(s) recognized as Generally Regarded as Safe (i.e., GRAS) by the U.S. Food and Drug Administration. In some instances, those skilled in the art will appreciate that excipient component(s) are or may be selected from those excipient(s) recognized in 21 C.F.R. 184. In some instances, those skilled in the art will appreciate that excipient component(s) are or may be selected from those excipient(s) recognized in GB2760-2014 by the National Health and Family Planning Commission of the People's Republic of China.

In some embodiments, an excipient component is or comprises at least one starch (e.g., Dry-Flo®), one cellulose (e.g., microcrystalline cellulose), or one sugar (maltodextrin). In some instances, an excipient component can comprise multiple excipients and combinations thereof.

In some embodiments, excipients are added to barrier material(s) and/or nutraceutical payload components during a manufacturing process. In some embodiments, excipients are present in barrier material(s) and/or nutraceutical payload components prior to mixing during a manufacturing process.

In some embodiments, an excipient component is at least about 99 wt %, at least about 90 wt %, at least about 85 wt %, at least about 80 wt %, at least about 75 wt %, at least about 70 wt %, at least about 65 wt %, at least about 60 wt %, at least about 55 wt %, at least about 50 wt %, at least about 45 wt %, at least about 40 wt %, at least about 35 wt %, at least about 30 wt %, at least about 25 wt %, at least about 20 wt %, at least about 15 wt %, at least about 10 wt %, at least about 5 wt %, at least about 1 Wt %, at least about 0.8 wt %, at least about 0.5 wt %, at least about 0.1 wt % of a probiotic composition (i.e., a particle preparation).

In some embodiments, an excipient component can lower water activity of particle preparations.

In some embodiments, an excipient component can lower residual solvent content of particle preparations.

In some embodiments, an excipient component can affect pH-responsiveness and alter payload release profile.

In some embodiments, an excipient component can alter pH within the particle preparation (e.g., probiotic composition).

In some embodiments, an excipient component can alter oxygen concentration within the particle preparation (e.g., probiotic composition).

In some embodiments, an excipient component can alter water or moisture concentration within the particle preparation (e.g., probiotic composition).

In some embodiments, an excipient component can alter pH within the microenvironment (e.g., stomach, food matrix, beverage) where the particle preparation (e.g., probiotic composition) resides.

In some embodiments, an excipient component can alter oxygen concentration within the microenvironment (e.g., stomach, food matrix, beverage) where the particle preparation (e.g., probiotic composition) resides.

In some embodiments, an excipient component can alter water or moisture concentration within the microenvironment (e.g., stomach, food matrix, beverage) where the particle preparation (e.g., probiotic composition) resides.

In some embodiments, an excipient component affects response of the probiotic composition to heat. Additionally or alternatively, an excipient component alters the glass transition temperature of the probiotic composition. In some embodiments, this may enable or facilitate methods of formulating or manufacturing probiotic compositions. In one non-limiting example, an excipient component comprising calcium carbonate reduces the glass transition temperature of a lipid component.

In some embodiments, an excipient component affects response of the probiotic composition to shear.

In some embodiments, an excipient component affects response of the probiotic composition to elevated pressure.

In some embodiments, an excipient component can affect stability in water, against light, in milk, in yogurt, in milk powder, in high humidity environments, in high moisture environments, or at elevated temperatures.

### 11. Moisture Content

In some embodiments, provided probiotic compositions are characterized by low moisture content. In some embodiments, the present disclosure provides technologies for preparing and/or characterizing probiotic compositions comprising low moisture content.

In some embodiments, the present disclosure provides probiotic compositions (e.g., particle preparations) with low moisture content. Disclosed technologies provide benefits over existing products because high moisture content formulations may lead to rapid degradation of microbes.

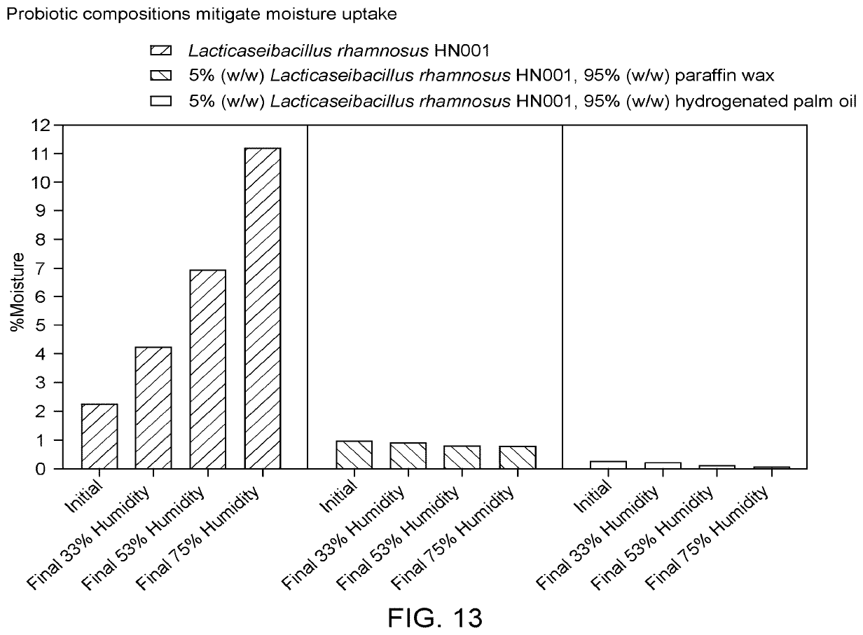

In some embodiments, the present disclosure provides probiotic compositions (e.g., particle preparations) with low moisture content. In some instances (e.g., FIG. 13), provided probiotic compositions (e.g., particle preparations) may have a moisture content of <8 wt %, <6 wt %, <4 wt %, <2 wt %, <1 wt %, or <0.5 wt %.

In some embodiments, provided probiotic compositions are characterized by resistance or mitigation of water absorption or moisture absorption when exposed to high humidity or moisture content as demonstrated in FIG. 13. In some embodiments, the present disclosure provides technologies for preventing uptake of water or moisture.

In some embodiments, the present disclosure provides probiotic compositions (e.g., particle preparations) that resist or mitigate moisture absorption when exposed to high humidities or moisture. In some instances, provided probiotic compositions (e.g., particle preparations) resist absorption of less than about 0.25%, less than about 0.5%, less than about 1%, and/or less than about 5% (w/w) moisture content, as compared to initial moisture content, after incubation in relative humidities of about 33%, about 53%, and/or about 75%.

In some embodiments, particle preparations (e.g., probiotic compositions) with low moisture content are particularly useful for combination with microbes (e.g., microbes sensitive to loss of colony forming units when exposed to high-moisture agents). In some embodiments, probiotic compositions (e.g., particle preparations) may further comprise a probiotic. In some embodiments, probiotic compositions (e.g., particle preparations) may further comprise a probiotic, one or more minerals, and/or one or more micronutrients.

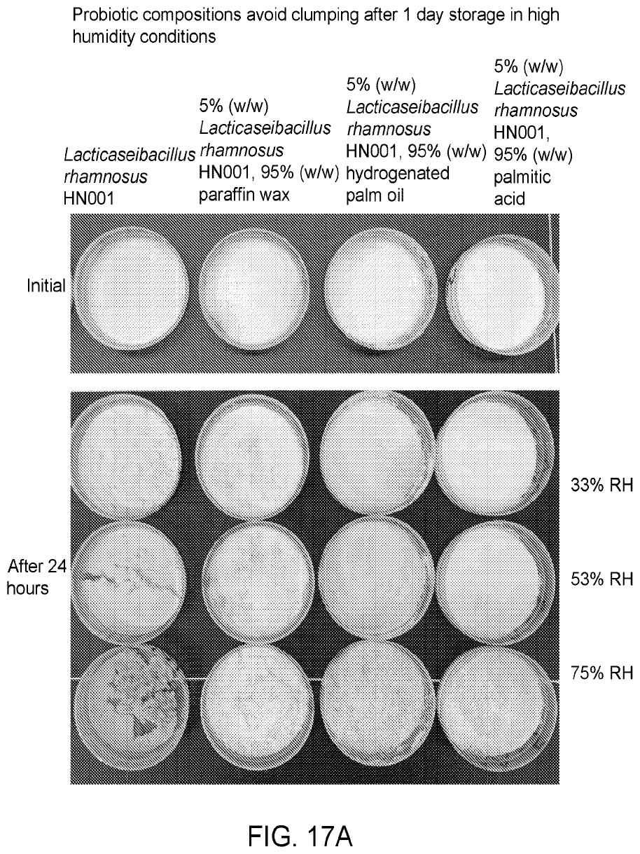

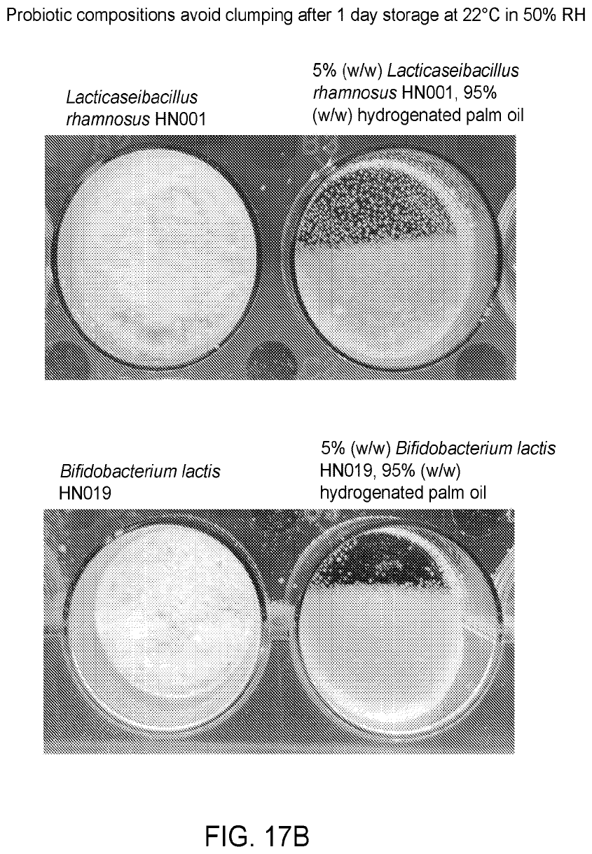

In an unexpected result (e.g., FIG. 17A-B), certain disclosed particle preparations (e.g., probiotic compositions) exhibiting low moisture content resist caking in environments of both low humidity as well as in elevated humidity. In certain embodiments, disclosed particle preparations (e.g., probiotic compositions) that resist caking additionally or alternatively exhibit improved flowability, relative to un-encapsulated nutraceutical payload component (e.g., probiotic cells), following storage for a predetermined period of time (e.g., incubation period) in an environment of at least about 50% relative humidity. In certain cases, improved flowability is or may be observed following a 24 hour incubation period in an environment of 50% relative humidity.

### 12. Water Activity

Nutraceutical payload component(s) often exhibit poor stability in environments with high water activity. In certain aspects of the present embodiments, a probiotic composition of low water activity exhibits a water activity of <about 0.4, <about 0.3, <about 0.2, and/or <about 0.1.

In certain embodiments, the disclosed invention provides probiotic compositions (e.g., particle preparations) of water activity <about 0.4, <about 0.3, <about 0.2, and/or <about 0.1. In certain embodiments, the disclosed invention provides probiotic compositions of low water activity. In some embodiments, the present disclosure provides technologies for preparing and/or characterizing probiotic compositions comprising low water activity.

In some embodiments, the present disclosure provides probiotic compositions (e.g., particle preparations) with low water activity. Disclosed technologies provide benefits over existing products because high water activity formulations lead to rapid degradation of microbes.

In some embodiments, particle preparations (e.g., probiotic compositions) with low water activity are particularly useful for combination with microbes (e.g., microbes sensitive to loss of colony forming units when exposed to high-water-reactivity agents). In some embodiments, probiotic compositions (e.g., particle preparations) may further comprise a probiotic.

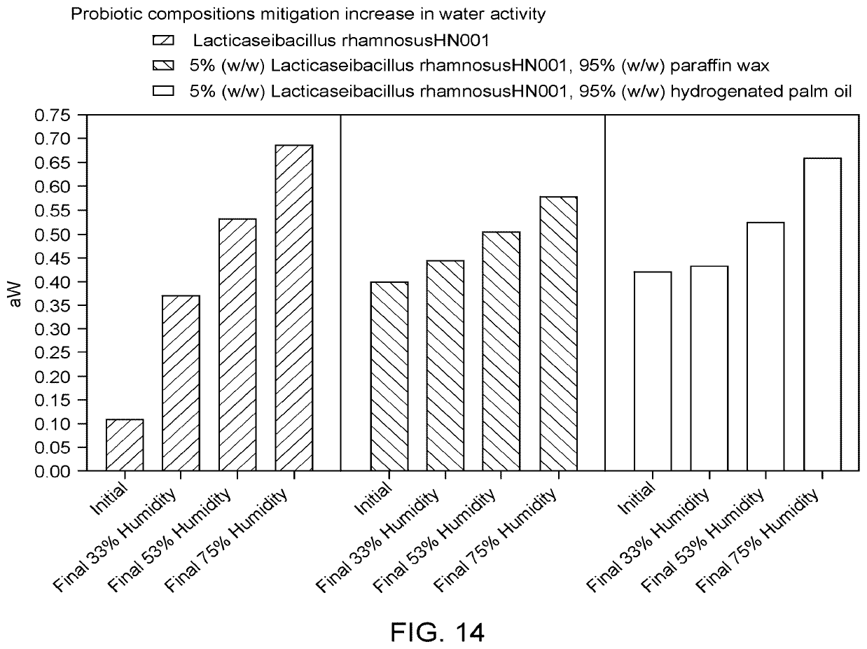

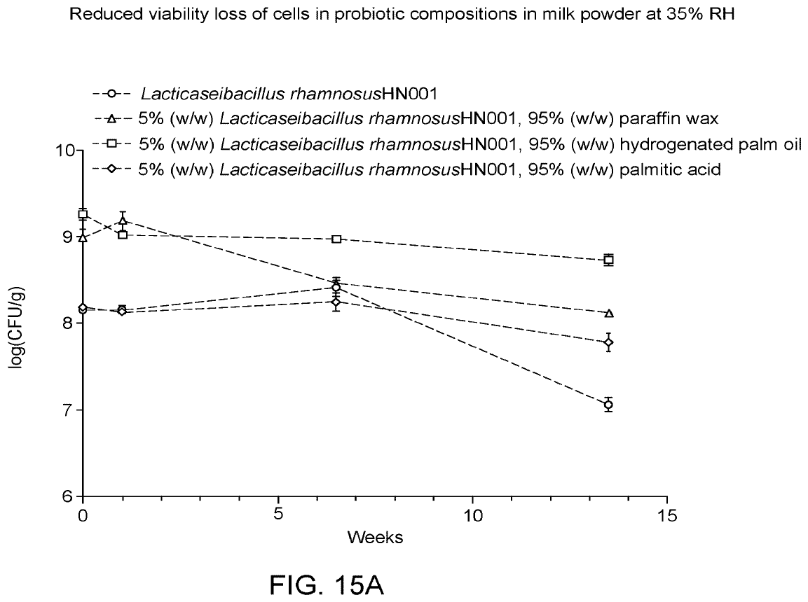

In an unexpected result, the disclosed invention provides for the stability of a nutraceutical payload component in environments with high water activity. In certain embodiments, the probiotic compositions provided herein (e.g., FIG. 14) exhibit a water activity of >about 0.3, >about 0.4, >about 0.5, and/or >about 0.6.

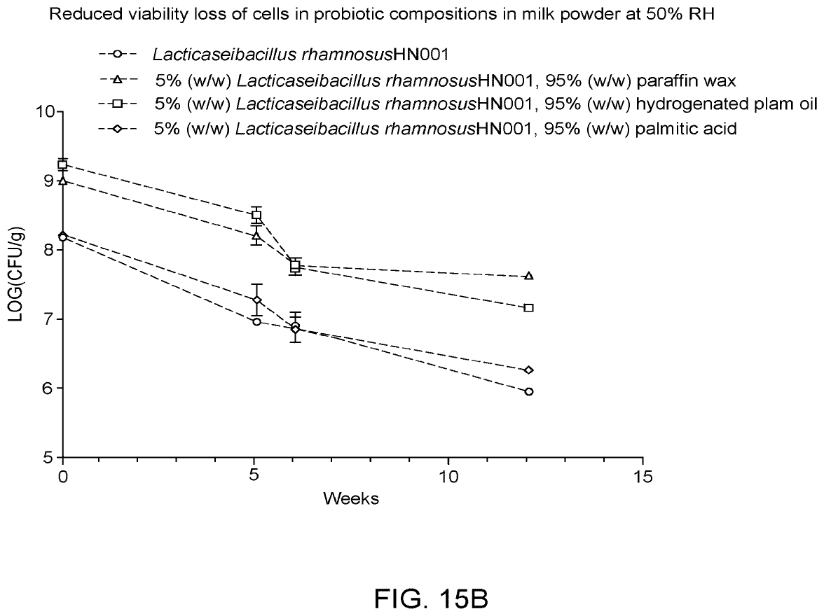

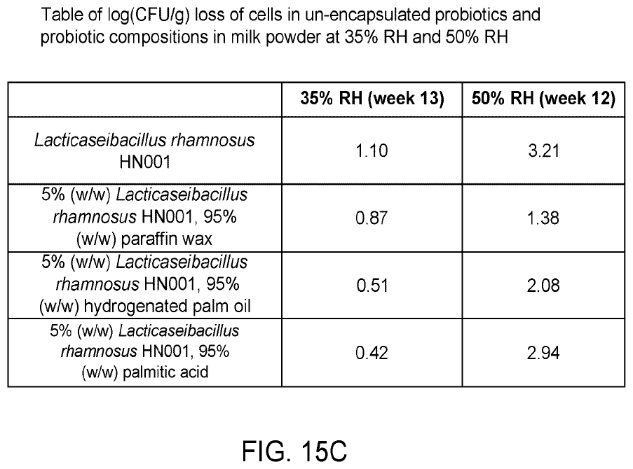

In certain embodiments, provided probiotic compositions with a payload component (e.g., probiotic cells, minerals, carotenoid compounds) exhibit high water activity. In certain embodiments (e.g., FIG. 15A-15B), high water activity probiotic compositions with a probiotic payload exhibit stability, enumerated by log(CFUs), over at least 1, at least 3, at least 6, and/or at least 12 weeks.

It is contemplated that the unexpected result described herein improves on previous methods by providing stability of a payload component (e.g., a probiotic component) in high water activity environments. It is further contemplated that the improvement described herein is a consequence of one or more selected barrier material(s) comprised in the probiotic composition. In certain embodiments, one or more selected barrier material(s) may or are further encapsulated within one or more selected barrier material(s) to reduce water activity. In certain embodiments, one or more selected barrier material(s) may be or are further encapsulated within one or more selected barrier material(s) to increase water activity.

### 13. Release of Payload Component

In some embodiments, probiotic compositions (e.g., particle preparations) disclosed herein provide for controlled release of payload components.

In many embodiments, release is physical and/or chemical dissociation of a payload (e.g., nutraceutical payload component) from a probiotic composition (e.g., particle preparation). In certain embodiments, release of a nutraceutical payload component occurs in a predetermined manner. In certain embodiments, release of a nutraceutical payload component occurs in response to an environmental factor (e.g., heat, light, water, humidity, water activity, mechanical forces, shear, pressure, chemical triggers, or combinations thereof).

In certain embodiments, release of a payload (e.g., nutraceutical payload component) occurs in response to an environmental factor (e.g., heat, light, water, humidity, water activity, mechanical forces, chemical triggers, or combinations thereof) to promote the utility (e.g., effectiveness of the nutraceutical payload component) of the probiotic composition.

In certain embodiments, control of release is the prevention of physical and/or chemical dissociation of a payload (e.g., nutraceutical payload component) from a probiotic composition (e.g., particle preparation). In preferred embodiments, control of release is prevention of physical and/or chemical dissociation of a payload (e.g., nutraceutical payload component) from a probiotic composition (e.g., particle preparation) in aqueous environments. In further preferred embodiments, release of a nutraceutical payload component is mitigated in acidic environments (e.g., simulated gastric fluid).

In certain embodiments, control of release of a nutraceutical payload component (e.g., microbe, bacteria, probiotic, mineral, carotenoid compound) is achieved by the use of one or more barrier material(s). In some embodiments, a barrier material erodes (e.g., dissolves, degrades, decomposes) only in the small intestine. In some embodiments, control of release of a nutraceutical payload component is release of the payload only in the small intestine. In some embodiments, a barrier material erodes (e.g., dissolves, degrades, decomposes) only in the large intestine. In some embodiments, control of release of a nutraceutical payload component is release of the payload only in the large intestine. In some embodiments, a barrier material erodes (e.g., dissolves, degrades, decomposes) only in the stomach. In some embodiments, control of release of a nutraceutical payload component is release of the payload only in the stomach. In some embodiments, a barrier material erodes (e.g., dissolves, degrades, decomposes) in one or more of the stomach, small intestine, and/or large intestine. In some embodiments, control of release of a nutraceutical payload component is release of the payload in one or more of the stomach, small intestine, and/or large intestine.

In some instances, less than about 10% log(CFU), less than about 9% log(CFU), less than about 8% log(CFU), less than about 7% log(CFU), less than about 6% log(CFU), less than about 5% log(CFU), less than about 4% log(CFU), less than about 3% log(CFU), less than about 2% log(CFU), less than about 1% log(CFU), less than about 0.5% log(CFU), less than about 0.1% log(CFU), less than about 0.05% log(CFU), and/or less than about 0.01% log(CFU) of probiotic (e.g., microbe payload, probiotic payload, etc.) is released from probiotic compositions (e.g., nutraceutical particle preparations) after soaking in water or simulated gastric fluid for 72 hours at about 25° C.

In some instances, less than about 10% log(CFU), less than about 9% log(CFU), less than about 8% log(CFU), less than about 7% log(CFU), less than about 6% log(CFU), less than about 5% log(CFU), less than about 4% log(CFU), less than about 3% log(CFU), less than about 2% log(CFU), less than about 1% log(CFU), less than about 0.5% log(CFU), less than about 0.1% log(CFU), less than about 0.05% log(CFU), and/or less than about 0.01% log(CFU) of probiotic (e.g., microbe payload, probiotic payload, etc.) is released from probiotic compositions (e.g., nutraceutical particle preparations) after soaking in water or simulated gastric fluid for 72 hours at about 37° C.

### 14. Protection of Payload Component

In some embodiments, probiotic compositions (e.g., nutraceutical particle preparations) disclosed herein provide for stability of payload components (e.g., nutraceutical payload components).

In several embodiments, stability of a payload component is provided by maintaining one or more physical, chemical, and/or biological properties over a predetermined period of time (e.g., incubation period) with exposure to one or more environmental stimuli (e.g., water, acid, heat, oxygen, light, pasteurization, physical perturbation, high pressure, or combinations thereof).

In certain embodiments, probiotic compositions (e.g., nutraceutical particle preparations) are stored for predetermined periods of time constituting an incubation period. In some embodiments, an incubation period is >about 1 second, >about 5 seconds, >about 10 seconds, >about 30 seconds, >about 1 minute, >about 5 minutes, >about 10 minutes. In some embodiments, an incubation period is >about 1 hour, >about 3 hours, >about 6 hours, >about 12 hours, >about 24 hours. In some embodiments, an incubation period is >about 1 day, >about 2 days, >about 3 days, >about 5 days, >about 7 days. In some embodiments, an incubation period is >about 1 week, >about 2 weeks, >about 4 weeks, and/or >about 6 weeks. In some embodiments, an incubation period is >about 1 month, >about 2 months, >about 3 months, >about 6 months, >about 12 months. In some embodiments, an incubation period is >about 1 year, >about 2 years, >about 3 years, >about 4 years, >about 5 years. In certain embodiments, a physical, chemical, and/or biological parameter is observed (e.g., measured) prior to and following a predetermined period of time (e.g., incubation period) to determine a change. In certain embodiments, a change in a physical, chemical, and/or biological parameter is expressed as a ratio, or percentage, of said change versus the original (e.g., prior to incubation period) value of the parameter.

For example, a physical parameter may comprise particle diameter, particle morphology, particle dispersity, particle density, particle agglomeration, particle porosity, or combinations thereof.

In some embodiments, a physical parameter is measured using at least one of: brightfield microscopy, electron microscopy, laser diffraction particle sizing, dynamic light scattering, and/or analytical ultracentrifugation.

For example, a chemical parameter may comprise visible or ultraviolet absorption spectrum, molecular weight, mass spectrum, hydrophobicity, partition coefficient, nuclear magnetic resonance spectrum, or combinations thereof.

In some embodiments, a chemical parameter is measured using at least one of: spectrophotometry, mass spectrometry, liquid chromatography, fluorescence spectrophotometry, and/or nuclear magnetic resonance.

For example, a biological parameter may comprise metabolic activity, colony forming units (CFUs), log colony forming units (log(CFUs)), turbidity, or combinations thereof.

In some embodiments, a biological parameter is measured using at least one of lactic acid assay, live/dead assay, spread plate enumeration on agar, enumeration on agar, spectrophotometry, and/or microscopy.

In some embodiments, maintenance of a measured physical, chemical, and/or biological property is characterized by a<about 40%, <about 30%, <about 20%, <about 10%, <about 5%, <about 2.5%, <about 1%, and/or <about 0.5% change following an incubation period. In some embodiments, maintenance of a measured physical, chemical, and/or biological property is characterized by a negligible change following an incubation period.

In several embodiments, stability of a payload component is provided by maintaining one or more physical, chemical, and/or biological properties over a predetermined period of time (e.g., incubation period). In some embodiments, stability of a payload component is provided by maintaining one physical, chemical, and/or biological property over an incubation period. In some embodiments, stability of a payload component is provided by maintaining multiple physical, chemical, and/or biological properties over an incubation period.

Nutraceutical payload components, as described herein, may be susceptible to instability over prolonged incubation to environmental stimuli (e.g., water, acid, heat, oxygen, light, pasteurization, physical perturbation, high pressure, or combinations thereof). Providing stability of a nutraceutical payload component in an aqueous environment may present particular difficulty. In some embodiments, the disclosed probiotic compositions (e.g., nutraceutical particle preparations) provide for the stability of a nutraceutical payload component upon exposure to one or more environmental stimuli, including, but not limited to, incubation in an aqueous environment.

In certain embodiments, stability of nutraceutical payload component(s) that is or are comprise microbes (e.g., probiotic cells) is characterized by maintenance of colony forming units (CFUs) or log colony forming units (log(CFUs)). In certain embodiments, stability of nutraceutical payload component(s) that is or are comprise a mineral (e.g., calcium carbonate) is characterized by maintenance of titration relative to an acid. In certain embodiments, stability of nutraceutical payload component(s) that is or are comprise a vitamin and/or carotenoid (e.g., retinoic acid, lutein, zeaxanthin) is characterized by maintenance of hydrophobicity, ultraviolet absorption spectrum, or combinations thereof.

In certain embodiments, stability of one or more nutraceutical payload components is provided over a period of time (e.g., incubation period) within an aqueous environment. In some embodiments, an aqueous environment comprises distilled water, saline solution, simulated intestinal fluid, simulated gastric fluid, juice, buttermilk, soda, Gatorade, sports drinks, Vitamin drink, seltzer, alcoholic beverage and/or coffee. In some embodiments, stability of one or more nutraceutical payload components within an aqueous environment is provided for at least 1, at least 3, at least 6, at least 12, and/or at least 24 hours.

In certain embodiments, stability of a microbes (e.g., probiotic) is provided over a period of time (e.g., incubation period) within an aqueous environment. In some embodiments, an aqueous environment comprises distilled water, saline solution, simulated intestinal fluid, simulated gastric fluid, juice, buttermilk, soda, Gatorade, sports drinks. Vitamin drink, seltzer, alcoholic beverage and/or coffee. In some embodiments, an aqueous environment comprises simulated gastric fluid. In some embodiments, stability of a probiotic cell is measured by change in log(CFUs) following an incubation period.

In some embodiments, a probiotic payload component exhibits <about 2 log(CFU), <about 1 log(CFU), <about 0.5 log(CFU), and/or <about 0.25 log(CFU) loss following incubation in aqueous environment (e.g., distilled water, saline solution, simulated intestinal fluid, simulated gastric fluid, juice, buttermilk, soda, Gatorade, sports drinks, Vitamin drink, seltzer, alcoholic beverage) at 25° C. for at least 1 hour. In some embodiments, a probiotic payload component exhibits <about 2 log(CFU), <about 1 log(CFU), <about 0.5 log(CFU), and/or <about 0.25 log(CFU) loss following incubation in aqueous environment at 25° C. for at least 3 hours. In some embodiments, a probiotic payload component exhibits <about 2 log(CFU), <about 1 log(CFU), <about 0.5 log(CFU), and/or <about 0.25 log(CFU) loss following incubation in aqueous environment at 25° C. for at least 24 hours.

In some embodiments, a probiotic payload component exhibits <about 2 log(CFU), <about 1 log(CFU), <about 0.5 log(CFU), and/or <about 0.25 log(CFU) loss following incubation in aqueous environment (e.g., distilled water, saline solution, simulated intestinal fluid, simulated gastric fluid, juice, buttermilk, soda, Gatorade, sports drinks. Vitamin drink, seltzer, alcoholic beverage) at 37° C. for at least 1 hour. In some embodiments, a probiotic payload component exhibits <about 2 log(CFU), <about 1 log(CFU), <about 0.5 log(CFU), and/or <about 0.25 log(CFU) loss following incubation in aqueous environment at 37° C. for at least 3 hours. In some embodiments, a probiotic payload component exhibits <about 2 log(CFU), <about 1 log(CFU), <about 0.5 log(CFU), and/or <about 0.25 log(CFU) loss following incubation in aqueous environment at 37° C. for at least 24 hours.

In certain embodiments, stability of a microbes (e.g., probiotic) is provided over a period of time (e.g., incubation period) within an acidic environment. In some embodiments, an acidic environment comprises pH<about 5, pH<about 4, pH<about 3, pH<about 2, and/or pH<about 1. In some embodiments, an acidic environment comprises simulated gastric fluid. In some embodiments, stability of a probiotic cell is measured by change in log(CFUs) following an incubation period.

In some embodiments, a probiotic payload component exhibits <about 2 log(CFU), <about 1 log(CFU), <about 0.5 log(CFU), and/or <about 0.25 log(CFU) loss following incubation in acidic environment (e.g., simulated gastric fluid) at 25° C. for at least 1 hour. In some embodiments, a probiotic payload component exhibits <about 2 log(CFU), <about 1 log(CFU), <about 0.5 log(CFU), and/or <about 0.25 log(CFU) loss following incubation in acidic environment at 25° C. for at least 3 hours. In some embodiments, a probiotic payload component exhibits <about 2 log(CFU), <about 1 log(CFU), <about 0.5 log(CFU), and/or <about 0.25 log(CFU) loss following incubation in acidic environment at 25° C. for at least 24 hours.

In certain embodiments, stability of a microbes (e.g., probiotic) is provided over a period of time (e.g., incubation period) at elevated temperature. In some embodiments, an elevated temperature is >about 25° C., >about 30° C. and/or >about 37° C. In some embodiments, stability of a probiotic cell is measured by change in log(CFUs) following an incubation period.

In some embodiments, a probiotic payload component exhibits <about 2 log(CFU), <about 1 log(CFU), <about 0.5 log(CFU), and/or <about 0.25 log(CFU) loss following incubation at a temperature of at least −20° C. for at least 6 months, at least 1 year, at least 2 years, and/or at least 3 years. In some embodiments, a probiotic payload component exhibits <about 2 log(CFU), <about 1 log(CFU), <about 0.5 log(CFU), and/or <about 0.25 log(CFU) loss following incubation at a temperature of at least 20° C. for at least 1 week, at least 3 weeks, at least 6 weeks, and/or at least 12 weeks. In some embodiments, a probiotic payload component exhibits <about 2 log(CFU), <about 1 log(CFU), <about 0.5 log(CFU), and/or <about 0.25 log(CFU) loss following incubation at a temperature of at least 37° C. for at least 1 week, at least 3 weeks, at least 6 weeks, and/or at least 12 weeks.

In certain embodiments, stability of a microbes (e.g., probiotic) is provided over a period of time (e.g., incubation period) at elevated relative humidity. In some embodiments, an elevated relative humidity is >about 35%, >about 42%, and/or >about 50%. In some embodiments, stability of a probiotic cell is measured by change in log(CFUs) following an incubation period.

In some embodiments, a probiotic payload component exhibits <about 2 log(CFU), <about 1 log(CFU), <about 0.5 log(CFU), and/or <about 0.25 log(CFU) loss following incubation at a relative humidity of at least 35% for at least 3 weeks, at least 6 weeks, at least 12 weeks, and/or at least 36 weeks. In some embodiments, a probiotic payload component exhibits <about 2 log(CFU), <about 1 log(CFU), <about 0.5 log(CFU), and/or <about 0.25 log(CFU) loss following incubation at a relative humidity of at least 42% for at least 3 weeks, at least 6 weeks, at least 12 weeks, and/or at least 36 weeks. In some embodiments, a probiotic payload component exhibits <about 2 log(CFU), <about 1 log(CFU), <about 0.5 log(CFU), and/or <about 0.25 log(CFU) loss following incubation at a relative humidity of at least 50% for at least 3 weeks, at least 6 weeks, at least 12 weeks, and/or at least 36 weeks.

In certain embodiments, stability of a microbe (e.g., probiotic) is provided over a period of time (e.g., incubation period) within a high pressure environment. In some embodiments, a high pressure environment is characterized by a pressure of at least 100 bar, at least 200 bar, at least 400 bar, at least 800 bar, at least 1200 bar, at least 1600 bar, at least 2000 bar, at least 3000 bar, at least 4000 bar, at least 6000 bar and/or at least 8000 bar. In some embodiments, stability of a probiotic cell is measured by change in log(CFUs) following an incubation period in a high pressure environment. In some embodiments, stability of a probiotic cell is measured by change in log(CFUs) following an incubation period in a high pressure environment.

In certain embodiments, stability of a microbe (e.g., probiotic) is provided over a period of time (e.g., incubation period) in high water activity environments (e.g., high water activity powders, a gummy product, etc.). In some embodiments, high water activity environments are water activities >about 0.20, >about 0.25, >about 0.30, >about 0.55, >about 0.60, >about 0.65 and/or >about 0.70. In some embodiments, stability of a probiotic cell is measured by change in log(CFUs) following an incubation period.

In some embodiments, a probiotic payload component exhibits <about 2 log(CFU), <about 1 log(CFU), <about 0.5 log(CFU), and/or <about 0.25 log(CFU) loss following incubation in a high pressure environment at about 40 bar for at least 1 minute. In some embodiments, a probiotic payload component exhibits <about 2 log(CFU), <about 1 log(CFU), <about 0.5 log(CFU), and/or <about 0.25 log(CFU) loss following incubation in a high pressure environment at about 800 bar for at least 30 seconds. In some embodiments, a probiotic payload component exhibits <about 2 log(CFU), <about 1 log(CFU), <about 0.5 log(CFU), and/or <about 0.25 log(CFU) loss following incubation in a high pressure environment at about 3000 bar for at least 10 seconds. In some embodiments, a probiotic payload component exhibits <about 2 log(CFU), <about 1 log(CFU), <about 0.5 log(CFU), and/or <about 0.25 log(CFU) loss following incubation in a high pressure environment at about 8000 bar for at least 5 seconds. In some embodiments, stability of a probiotic cell is measured by change in log(CFUs) following an incubation period in a high pressure environment.

In some embodiments, a probiotic payload component exhibits <about 2 log(CFU), <about 1 log(CFU), <about 0.5 log(CFU), and/or <about 0.25 log(CFU) loss following incubation in a high temperature environment at about 37° C. for at least 5 minutes. In some embodiments, a probiotic payload component exhibits <about 2 log(CFU), <about 1 log(CFU), <about 0.5 log(CFU), and/or <about 0.25 log(CFU) loss following incubation in a high temperature environment at about 55° C. for at least 5 minutes. In some embodiments, a probiotic payload component exhibits <about 2 log(CFU), <about 1 log(CFU), <about 0.5 log(CFU), and/or <about 0.25 log(CFU) loss following incubation in a high temperature environment at about 75° C. for at least 20 seconds. In some embodiments, a probiotic payload component exhibits <about 2 log(CFU), <about 1 log(CFU), <about 0.5 log(CFU), and/or <about 0.25 log(CFU) loss following incubation in a high temperature environment at about 130° C. for at least 2 seconds. In some embodiments, a probiotic payload component exhibits <about 2 log(CFU), <about 1 log(CFU), <about 0.5 log(CFU), and/or <about 0.25 log(CFU) loss following incubation in a high temperature environment at about 150° C. for at least 4 seconds. In some embodiments, stability of a probiotic cell is measured by change in log(CFUs) following an incubation period in a high temperature environment.

In certain embodiments, stability of a microbes (e.g., probiotic) is provided over a period of time (e.g., incubation period) within a high shear environment. In some embodiments, a high shear environment is characterized by a mixing rate of at least 200 rpm, at least 500 rpm, at least 1000 rpm, at least 2000 rpm, at least 4000 rpm, at least 10000 rpm, at least 25000 rpm, at least 40000 rpm, and/or at least 50000 rpm. In some embodiments, stability of a probiotic cell is measured by change in log(CFUs) following an incubation period in a high shear environment.

In some embodiments, a probiotic payload component exhibits <about 2 log(CFU), <about 1 log(CFU), <about 0.5 log(CFU), and/or <about 0.25 log(CFU) loss following incubation in a high shear environment at about 500 rpm for at least 1 minute. In some embodiments, a probiotic payload component exhibits <about 2 log(CFU), <about 1 log(CFU), <about 0.5 log(CFU), and/or <about 0.25 log(CFU) loss following incubation in a high shear environment at about 1000 rpm for at least 30 seconds. In some embodiments, a probiotic payload component exhibits <about 2 log(CFU), <about 1 log(CFU), <about 0.5 log(CFU), and/or <about 0.25 log(CFU) loss following incubation in a high shear environment at about 10000 rpm for at least 10 seconds. In some embodiments, a probiotic payload component exhibits <about 2 log(CFU), <about 1 log(CFU), <about 0.5 log(CFU), and/or <about 0.25 log(CFU) loss following incubation in a high shear environment at about 25000 rpm for at least 10 seconds. In some embodiments, a probiotic payload component exhibits <about 2 log(CFU), <about 1 log(CFU), <about 0.5 log(CFU), and/or <about 0.25 log(CFU) loss following incubation in a high shear environment at about 50000 rpm for at least 10 seconds.

In some embodiments, probiotic composition(s) of the present disclosure have high probiotic loading (e.g., greater than 35%, greater than 40%, greater than 45%, greater than 50%, greater than 55%, greater than 60%, greater than 65%, greater than 70%, or greater than 75% (w/w)) and low fat, lipid, wax, and/or protein content (e.g., less than 50%, less than 45%, less than 40%, less than 35%, less than 30%, or less than 25% (w/w)). In some embodiments, this formulation has improved dispersion in liquids, achieves a higher probiotic dose for a given unit mass of particle formulation, and/or reduces material costs due to use of fewer/less encapsulating materials.

### C. Method of Enumeration of Microbes in a Probiotic Composition

In many embodiments of the probiotic compositions (e.g., particle preparations) provided in this disclosure, it is necessary to enumerate microbes either encapsulated or not encapsulated within barrier materials, for example, to determine encapsulation efficiency, protection from the environment, cell viability, cell growth capacity, and/or combinations thereof. In some embodiments, the enumeration of microbes either encapsulated or not encapsulated within barrier materials serves as a measure of batch quality. In some embodiments, the enumeration of microbes either encapsulated or not encapsulated within barrier materials serves as a measure of the protective ability of barrier material(s). In some embodiments, batches that do not meet designated criteria may be discarded or not further utilized.

In certain embodiments, the disclosed probiotic compositions (e.g., that are or comprise particle preparations) comprise microbes encapsulated within one or more barrier material(s). In some instances, the barrier material(s) serve as a barrier to moisture (e.g., moisture-resistant materials). In some instances, nutraceutical payload components (e.g., microbes, probiotics, bacteria) encapsulated within barrier material(s) that are characterized as being moisture-resistant are retained upon dispersal of particle preparations within an aqueous (e.g., water, suitable salt solution component, simulated gastric fluid, yogurt, smoothie, milk) system.

### 1. Aqueous Extraction

In some embodiments, the disclosed probiotic compositions (e.g., that are or comprise particle preparations) comprise microbes encapsulated within one or more lipid barrier material(s). In some instances, nutraceutical payload components (e.g., microbes, probiotics, bacteria) encapsulated within barrier material(s) comprising lipid components are retained upon dispersal of particle preparations within an aqueous (e.g., water, suitable salt solution component, simulated gastric fluid, yogurt, smoothie, milk) bath.

In some embodiments, the disclosed probiotic compositions (e.g., that are or comprise particle preparations) comprise microbes loosely adhered, on the surface, freely unincorporated, or combinations thereof within one or more lipid barrier material(s). In some instances, nutraceutical payload components (e.g., microbes, probiotics, bacteria) unincorporated within barrier material(s) comprising lipid components are lost upon dispersal of particle preparations within an aqueous (e.g., water, suitable salt solution component, simulated gastric fluid, yogurt, smoothie, milk) bath.

In some embodiments, nutraceutical payload components (e.g., microbes, probiotics, bacteria) unincorporated and/or loosely incorporated within barrier material(s) are collected in an aqueous (e.g., water, suitable salt solution component) bath. In certain embodiments, probiotic compositions (e.g., particle preparations) are added to an aqueous bath in a proportion of less than about 10%, less than about 5%, less than about 1%, less than about 0.2%, and/or less than about 0.05%. In certain embodiments, probiotic compositions (e.g., particle preparations) dispersed in an aqueous bath are subjected to a homogenization process. In certain embodiments a homogenization process may comprise, but is not limited to, overhead stirrer, manual stirring, stir bar, high pressure homogenization, low pressure homogenization, sonication, ultrasonication, vortexing, and/or combinations thereof.

In certain embodiments, probiotic compositions (e.g., particle preparations) are dispersed in an aqueous bath and subjected to a homogenization process as provided herein. In some embodiments, the aqueous bath, comprising nutraceutical payload components (e.g., microbes, probiotics, bacteria) is collected.

In some embodiments, nutraceutical payload components (e.g., microbes, probiotics, bacteria) lost upon dispersal of particle preparations within an aqueous (e.g., water, suitable salt solution component, simulated gastric fluid, yogurt, smoothie, milk) bath are enumerated using standard microbiology techniques (e.g., enumeration on agar).

### 2. Oil Extraction

In some embodiments, the disclosed probiotic compositions (e.g., that are or comprise particle preparations) comprise microbes encapsulated within one or more lipid barrier material(s). In some instances, nutraceutical payload components (e.g., microbes, probiotics, bacteria) encapsulated within barrier material(s) comprising lipid components are released upon dispersal of particle preparations within a suitable oil bath (e.g., oil component). In certain embodiments, the oil bath (e.g., oil component) is warmed to facilitate melting and dispersal of probiotic compositions (e.g., particle preparations) comprising microbes encapsulated within lipid barrier material(s).

In some embodiments, nutraceutical payload components (e.g., microbes, probiotics, bacteria) released upon dispersal of particle preparations within a suitable oil component are recovered by forming an emulsion with a suitable surfactant and suitable salt solution.

In a preferred embodiment, an oil component is added in excess relative to a predetermined weight of probiotic composition. In some embodiments, an oil component is added to at least 1, 2, 3, 4, 5, 10, 20, 30, 50, 75, and/or 100-fold by mass relative to a predetermined weight of probiotic composition.

In some embodiments, a suitable oil component is warmed to facilitate melting and dispersal of a probiotic composition. In certain cases, the oil component is warmed to at least >20° C., >30° C., >40° C., >50° C., >60° C., >70° C. and/or >80° C.

In certain aspects, a suitable surfactant component is added to a warmed oil bath comprising a dispersed probiotic composition (e.g., particle preparation). In certain embodiments, the suitable surfactant is added to an excess quantity relative to the mass of warmed oil in the oil bath (e.g., oil component). In preferred embodiments, the suitable surfactant is added to at least 1, 2, 3, 4, 5, 10, and/or 20-fold by mass relative to the weight of oil in the oil bath (e.g., oil component).

In certain aspects, a suitable salt solution component is added to a warmed oil bath comprising a dispersed probiotic composition (e.g., particle preparation) with suitable surfactant (e.g., surfactant component). In certain embodiments, the suitable salt solution component is added to an excess quantity relative to the weight of liquid in the oil bath (e.g., oil component and surfactant component). In preferred embodiments, the suitable salt solution is added to at least 3, 4, 5, 10, 20, 50, 100 and/or 200-fold by mass relative to the weight of liquid in the oil bath (e.g., oil component and surfactant component).

In certain aspects, an aqueous portion of a combined bath comprising a probiotic composition, oil component, surfactant component, and salt solution component is collected for enumeration using standard microbiology techniques (e.g., enumeration).

### 3. Oil Component

Typically, as described herein, utilized oil component(s) is or are characterized by melting point (e.g., liquid oil at room temperature).

As provided herein, an oil component may be or comprises at least one oil. In some instances, oil components can be a combination of oils, each of which may or may not be individually liquids at room temperature.

For example, as described herein, oil component(s) liquid at room temperature may comprise, but are not limited to, vegetable oil, castor oil, avocado oil, sunflower oil, rapeseed oil, mineral oil, palm oil, or combinations thereof.

### 4. Surfactant Components

Typically, as described herein, utilized surfactant component(s) is or are characterized by HLB value (e.g., intermediate- to low-HLB surfactants). In some instances, surfactant component(s) is or are characterized by charge (i.e., non-ionic surfactants).

As provided herein, a surfactant component may be or comprises at least one surfactant. In some instances, surfactant components can be a combination of surfactants, each of which may or may not be individually intermediate- to low-HLB and/or non-ionic surfactants.

For example, in some embodiments, surfactant components characterized as intermediate- to low-HLB surfactants may comprise, but are not limited to, cetearyl alcohol, cetearyl glucoside, cetyl alcohol, emulsifying wax, glyceryl stearate, PEG-40 hydrogenated castor oil, polyoxyethylene glycol sorbitan alkyl esters, polysorbates, propanediol, safflower oleosomes, sorbitan alkyl esters, and/or combinations thereof.

For example, in some embodiments, surfactant components characterized as non-ionic surfactants may comprise, but are not limited to, cetearyl alcohol, cetearvl glucoside, cetyl alcohol, emulsifying wax, glyceryl stearate, PEG-40 hydrogenated castor oil, polyoxyethylene glycol sorbitan alkyl esters, polysorbates, propanediol, safflower oleosomes, sorbitan alkyl esters, and/or combinations thereof

### 5. Salt Solution Components

Typically, as described herein, utilized salt solution component(s) is or are characterized by tonicity (e.g., balanced salt solutions).

In some embodiments, a suitable salt solution component may be or comprise, but are not limited to, peptone water, saline solution, phosphate buffer saline solution, dulbecco's phosphate buffer saline solution, HEPES buffer saline solution, Earl's balanced salt solution, Hank's balanced salt solution, and/or combinations thereof.

### 6. Enumeration

In certain embodiments, enumeration of microbes in a provided probiotic composition (e.g., particle preparations) is a measure of the encapsulation efficiency of a payload component into a probiotic composition. In certain embodiments, enumeration of microbes in provided probiotic compositions (e.g., particle preparations) is a measure of the protection of a payload component from environmental moisture, water, humidity, and/or water activity. In certain embodiments, enumeration of microbes in a provided probiotic composition (e.g., particle preparations) is a measure of the growth capacity of an encapsulated payload component. In certain embodiments, enumeration of microbes is used to measure one or more of encapsulation efficiency, protection, and growth capacity of the payload component in a probiotic composition.

In some embodiments, encapsulation efficiency of the provided probiotic composition (e.g., particle preparations) is determined by measuring the enumerated viability (e.g., CFU/g) relative to the intended loading during the manufacturing process. In some embodiments, encapsulation efficiency of the provided probiotic composition (e.g., particle preparations) is determined by measuring the aqueous extraction viability (e.g., CFU/g) relative to the enumerated viability in the oil extraction.

In some embodiments, standard microbiology techniques used for the enumeration of microbes may be or comprise spread plate enumeration on agar, optical turbidity measurement, microscopy, and/or combinations thereof.

### D. Methods of Manufacturing Probiotic Compositions

In some embodiments, probiotic compositions provided and/or utilized in accordance with the present disclosure are or comprise particles (e.g., lipid microparticles). Some aspects of the present disclosure provide technologies making and/or characterizing particle preparations—e.g., that are or comprise barrier materials described herein, and/or compositions that include them.

### 1. Methods of Altering Size Distribution of Freeze-Dried Cells

In certain embodiments, a method of reducing the size of a nutraceutical payload component is provided. In some embodiments, the nutraceutical payload component to be reduced in size is or may be a liquid. In some embodiments, the nutraceutical payload component to be reduced in size is or comprises a solid. In certain embodiments, the nutraceutical payload component is or are particle(s). In some embodiments, particle(s) is or are characterized as having a particle size distribution, as described herein. Alternatively, or additionally, particle(s) is or are characterized as having an average particle diameter, as described herein.

In certain embodiments, a nutraceutical payload component is or are particle(s) characterized as having an average particle diameter. In some embodiments, the average particle diameter of a nutraceutical payload component (e.g., particle(s)) is reduced by mechanical means (e.g., method of size reduction). It is contemplated that reduction of the particle size of a nutraceutical payload component facilitates homogeneous incorporation into the probiotic compositions described herein.

As provided herein, methods of size reduction may be or comprise a single method of size reduction. In some instances, methods of size reduction may be or comprise at least 1, 2, or 3 successive methods of size reduction. In some instances, methods of size reduction may be or comprise several successive methods of size reduction.

For example, in some embodiments, methods of size reduction may comprise, but are not limited to, spray drying, lyophilization/milling, planetary milling, ball milling, burr milling, roller milling, media milling, impact milling, jet milling, high-pressure homogenization, cryo milling, hammer milling, conical milling, hand screening, and/or granulation/extrusion.

Methods for reducing size may exert environmental strain (e.g., excessive heat, light, oxidation, moisture, or combinations thereof) on payload components (e.g., vitamins, probiotics, minerals). In some embodiments, the provided size reduction methods preserve stability of the nutraceutical payload component. In certain embodiments, process parameters are selected to maximize stability of the encapsulated nutraceutical payload component.

In some embodiments, process parameters pertinent to the stability of the encapsulated nutraceutical payload component include, but are not limited to, processing temperature, milling speed, and/or combinations thereof.

In some embodiments, the temperature of a size reduction is selected in order to preserve stability of a nutraceutical payload component. In some instances, the temperature of the size reduction process is or may be <70° C.<60° C., <50° C., <40° C., <30° C., <20° C., <10° C., <0° C., <−10° C., <−20° C., <−30° C., and/or <−40° C. In certain embodiments, the temperature of the size reduction process is between about −20° C. and about 10° C.

In some embodiments, the milling speed of a size reduction is selected in order to preserve stability of a nutraceutical payload component. In some instances, the milling speed of the size reduction process is or may be <50000 rpm, <20000 rpm, <10000 rpm, <5000 rpm, <2000 rpm, and/or <1000 rpm. In certain embodiments, the milling speed of the size reduction process is between about 5000 rpm and about 20000 rpm.

In certain embodiments, size reduction employed to reduce the size of nutraceutical payload component(s) is or may be characterized by the ratio of average particle diameter measured after size reduction to average particle diameter measured before size reduction. In some embodiments, the average particle diameter of a nutraceutical payload component is measured using a Malvern Mastersizer. In some embodiments, other suitable particle size analyzers may be used.

In certain embodiments, a nutraceutical payload component is characterized by average particle diameter. In some embodiments, the nutraceutical payload component is characterized as having an average particle diameter of <1000 m, <500 μm, <250 μm, <125 μm, <50 μm, <20 μm, <5 μm, <1 μm, and/or <0.5 μm. In certain preferred embodiments, the nutraceutical payload component is characterized as having an average particle diameter between about 1 μm-100 μm. In certain preferred embodiments, the nutraceutical payload component is characterized as having an average particle diameter between about 10 μm-200 μm. In certain preferred embodiments, the nutraceutical payload component is characterized as having an average particle diameter in a range from about 5 μm-50 μm.

### 2. Methods of Dispersing Freeze-Dried Cells in a Molten Non-Aqueous Matrix

In certain embodiments, a method of dispersing a nutraceutical payload component within a molten non-aqueous matrix (e.g., probiotic composition) is provided. In some embodiments, the nutraceutical payload component to be reduced in size is or may be a liquid. In some embodiments, the nutraceutical payload component to be reduced in size is or may be a solid. In certain embodiments, the nutraceutical payload component is or are particle(s). In some embodiments, particle(s) is or are characterized as having a particle size distribution, as described herein. Alternatively, or additionally, particle(s) is or are characterized as having an average particle diameter, as described herein.

In certain embodiments, a nutraceutical payload component is dispersed within a molten non-aqueous matrix. In some embodiments, the molten non-aqueous matrix is or may comprise a lipid, a carbohydrate, or a protein, as described herein. In certain embodiments, the molten non-aqueous matrix is or may comprise a material characterized as having a low melting point. In many embodiments, components utilized in a molten non-aqueous matrix are characterized to melt <80° C., <70° C., <60° C., <50° C., <40° C., <30° C.

As provided herein, a molten non-aqueous matrix may be or comprises at least one lipid, one carbohydrate, one surfactant, and/or one protein. In some instances, a molten non-aqueous matrix can be a combination of lipids, carbohydrates, surfactants, and/or proteins.

For example, in some embodiments, lipid components as suitable for a molten non-aqueous matrix may comprise, but are not limited to, paraffin wax, montan wax, microcrystalline wax, polyethylene wax, petrolatum wax, ozokerite wax, ceresin wax, beeswax, lanolin wax, spermaceti wax, tallow wax, lac wax, Chinese insect wax, ambergris wax, soy wax, camauba wax, candelilla wax, coconut wax, palm kernel wax, rice bran wax, butyric acid, n-butanol, pentanoic acid, n-pentanol, hexanoic acid, n-hexanol, heptanoic acid, n-heptanol, caprylic acid, n-octanol, nonanoic acid, n-nonanol, capric acid, n-decanol, lauric acid, n-dodecanol, myristic acid, n-tetradecanol, palmitic acid, n-hexadecanol, stearic acid, n-octadecanol, arachidonic acid, n-icosanol, fatty alcohol monoglyceride ethers, fatty acid monoglyceride esters, fatty alcohol diglyceride ethers, fatty acid diglyceride esters, fatty alcohol triglyceride ethers, fatty acid triglyceride esters, fatty alcohol glycol monoether, fatty acid glycol monoesters, fatty alcohol glycol diethers, fatty acid glycol diesters, fatty alcohol poly(glycerol) ethers, fatty acid poly(glycerol) esters, fatty alcohol poly(glycol) ethers, fatty acid poly(glycol) esters, coconut oil, corn oil, cottonseed oil, olive oil, palm oil, peanut oil, rapeseed oil, safflower oil, sesame oil, soybean oil, sunflower oil, almond oil, pine nut oil, cashew oil, fully hydrogenated palm oil, partially hydrogenated palm oil, fully hydrogenated sunflower oil, partially hydrogenated sunflower oil, fully hydrogenated soybean oil, partially hydrogenated soybean oil, fully hydrogenated vegetable oil, partially hydrogenated vegetable oil, fully hydrogenated cottonseed oil, partially hydrogenated cottonseed oil, cholesterol, cholenic acid, ursolic acid, betulinic acid, and/or combinations thereof.

Methods of incorporating nutraceutical payload components within molten matrices (e.g., incorporation process) may exert environmental strain (e.g., excessive heat, light, oxidation, moisture, shear, pressure, or combinations thereof) on payload components (e.g., vitamins, probiotics, minerals). The nutraceutical payload components of the present embodiments, therefore, may help to preserve stability of the nutraceutical payload component. In certain embodiments, process parameters are selected to maximize stability of the encapsulated nutraceutical payload component.

In certain embodiments, incorporation of a nutraceutical payload component is achieved by homogenization techniques (e.g., homogenization process). In some embodiments, homogenization techniques may comprise, but are not limited to, overhead stirrer, manual stirring, stir bar, high pressure homogenization, low pressure homogenization, sonication, ultrasonication, vortexing, and/or combinations thereof.

In some embodiments, process parameters pertinent to the stability of the encapsulated nutraceutical payload component includes, but are not limited to, processing temperature, homogenization speed, and/or combinations thereof.

In some embodiments, the temperature of an incorporation process is selected in order to preserve stability of a nutraceutical payload component. In some instances, the temperature of the incorporation process is or may be <80° C., <70° C., <60° C., <50° C., <40° C., <30° C., <20° C., and/or. In certain embodiments, the temperature of the incorporation process is between about 30° C. and about 60° C.

In some embodiments, the speed of a homogenization process is selected in order to preserve stability of a nutraceutical payload component. In some instances, the speed of the homogenization process is or may be <50000 rpm, <40000 rpm, <25000 rpm, <10000 rpm, <5000 rpm, <1000 rpm, <500 rpm, <250 rpm, <125 rpm, <50 rpm, and/or <10 rpm. In certain embodiments, the milling speed of the size reduction process is between about 10 rpm and about 100 rpm.

### 3. Methods of Spheronization of Probiotic Compositions

In certain embodiments, a method of size reduction of a probiotic composition is provided. In some embodiments, the probiotic composition to be reduced in size is a liquid. In some embodiments, the probiotic composition to be reduced in size is a solid. In certain embodiments, the probiotic composition is or are particle(s). In some embodiments, particle(s) is or are characterized as having a particle size distribution, as described herein. Alternatively, or additionally, particle(s) is or are characterized as having an average particle diameter, as described herein.

In certain embodiments, a probiotic composition is or are particle(s) characterized as having an average particle diameter. In some embodiments, the average particle diameter of a probiotic composition (e.g., particle(s)) is reduced by mechanical means (e.g., method of size reduction).

As provided herein, methods of size reduction may be or comprise a single method of size reduction. In some instances, methods of size reduction may be or comprise at least 1, 2, or 3 successive methods of size reduction. In some instances, methods of size reduction may be or comprise several successive methods of size reduction.

For example, in some embodiments, methods of size reduction comprise, but are not limited to, planetary milling, ball milling, burr milling, roller milling, media milling, impact milling, jet milling, high-pressure homogenization, cryo milling, hammer milling, conical milling, hand screening, or granulation/extrusion, extrusion, spray drying, fluid bed agglomeration, spray congealing, high-shear granulation, tableting, pouring, roller compaction, crosslinking, prilling, spinning disc atomization, and/or combinations thereof.

Methods of size reduction may exert environmental strain (e.g., excessive heat, light, oxidation, moisture, shear, or combinations thereof) on payload components (e.g., vitamins. probiotics, minerals). The provided method(s) of size reduction of probiotic compositions may therefore help to preserve stability of the nutraceutical payload component. In certain embodiments, process parameters are selected to maximize stability of the encapsulated nutraceutical payload component.

In some embodiments, process parameters pertinent to the stability of the encapsulated nutraceutical payload component include, but are not limited to, atomization temperature, atomization flow rate, cooling temperature, or combinations thereof.

In some embodiments, an atomization temperature is selected in order to preserve stability of a nutraceutical payload component. In some instances, the temperature of the incorporation process is or may be <80° C., <70° C., <60° C., <50° C., <40° C., <30° C., <20° C., and/or. In certain embodiments, the temperature of the incorporation process is in a range from about 40° C. to about 80° C.

In some embodiments, an atomization flow rate is selected in order to preserve stability of a nutraceutical payload component. In some instances, the speed of the homogenization process is or may be <11000 rpm, <10000 rpm, <9000 rpm, <8000 rpm, <7000 rpm, <6000 rpm, <5000 rpm, <4000 rpm, <3000 rpm, <2000 rpm, and/or <1000 rpm. In certain embodiments, the milling speed of the size reduction process is between about 3000 rpm and about 10000 rpm.

In some embodiments, cooling temperature is selected in order to preserve stability of a nutraceutical payload component. In some instances, the cooling temperature is or may be <20° C., <10° C., <0° C., <−20° C., <−50° C., <−70° C., <−80° C. In certain embodiments, the cooling temperature is between about 0° C. and about 25° C. In other embodiments, the cooling temperature is between about −80° C. and about −40° C.

In certain embodiments, a probiotic composition (e.g., particle preparation) is characterized by average particle diameter. In some embodiments, the probiotic composition is characterized as having an average particle diameter of <1000 μm, <500 μm, <250 μm, <125 μm, <50 μm, <20 μm, and/or <5 μm. In certain preferred embodiments, the nutraceutical payload component is characterized as having an average particle diameter between about 10 μm-200 μm.

In certain preferred embodiments, the nutraceutical payload component is characterized as having an average particle diameter between about 50 μm-800 μm. In certain preferred embodiments, the nutraceutical payload component is characterized as having an average particle diameter in a range from about 90 μm-400 μm.

### 4. Methods of Coating Probiotic Compositions

In certain embodiments, a method of coating probiotic composition(s) (e.g., particle preparations) is provided. In some embodiments, the probiotic composition to be coated is a solid. In certain embodiments, the probiotic composition is or are particle(s) (e.g., particle preparations). In some embodiments, particle(s) is or are characterized as having a particle size distribution, as described herein. Alternatively. or additionally, particle(s) is or are characterized as having an average particle diameter, as described herein.

As provided herein, methods of coating may be or comprise a single method of coating. In some instances, methods of coating may be or comprise at least 1, 2, or 3 successive methods of coating. In some instances, methods of coating may be or comprise several successive methods of coating.

For example, in some embodiments, methods of coating comprise, but are not limited to, spray pan coating, fluidized bed coating, dip coating, roller coating, sputter coating, or combinations thereof.

In some embodiments, a method of coating a probiotic composition (e.g., a particle preparation) uses or may utilize materials that improve (e.g., protect, or improve the functionality of) the probiotic composition. In some embodiments, a method of coating a probiotic composition improves resistance to moisture (e.g., humidity, water, water activity). In some embodiments, a method of coating a probiotic composition improves resistance to acidity (e.g., pH responsive materials). In some embodiments, a method of coating a probiotic composition reduces porosity. In some embodiments, a method of coating a probiotic composition reduces agglomeration, aggregation, and/or tackiness.

In some embodiments, materials for coating a probiotic composition are characterized by having resistance to moisture. In some embodiments, materials for coating a probiotic composition are characterized by having resistance to acidity. In some embodiments, materials for coating a probiotic composition are characterized by having resistance to moisture and acidity.

For example, in some embodiments, materials resistant to moisture comprise, but are not limited to, shellac, cellulose acetate butyrate, zein, gliadin, kafirin, avenin, and/or combinations thereof.

For example, in some embodiments, materials resistant to acidity comprise, but are not limited to, shellac, cellulose acetate butyrate, zein, gliadin, kafirin, avenin, cellulose acetate succinate, cellulose acetate phthalate, hydroxypropyl methylcellulose acetate succinate, and/or combinations thereof.

### 5. Method of Drying Probiotic Composition

In certain embodiments, a method of drying probiotic composition(s) (e.g., particle preparations) is provided. In some embodiments, drying of a probiotic composition comprises reduction of moisture content. In some embodiments, drying of a probiotic composition comprises reduction of water activity.

The disclosed method of drying certain probiotic compositions, as provided herein, improves upon the prior art by further eliminating exposure of payload components (e.g., probiotics) to moisture and/or presence of water.

In certain embodiments, drying of certain probiotic composition(s) is achieved by the use of chemical drying agents, elevated temperature, vacuum, or combinations thereof.

For example, in some embodiments, drying of probiotic composition(s) is achieved by use of drierite, heating, vacuum, molecular sieves, sodium sulfate, magnesium sulfate, calcium carbonate, calcium chloride, or combinations thereof.

### E. Uses of Probiotic Compositions

In certain embodiments, the disclosed probiotic compositions (e.g., that are or comprise particle preparations) provide protection against degradation (e.g., oxidation, hydrolysis, isomerization, fragmentation, lysis, or a combination thereof) of payload component(s). In some embodiments, the disclosed probiotic compositions comprise particle preparations wherein nutraceutical payload components are protected from environmental factors (e.g., water, humidity, moisture, water activity, light, heat, and/or acid).

It is contemplated that provided particle preparations (e.g., probiotic compositions) disclosed herein are suitable for use in varying consumable compositions (e.g., a food product, a beverage product, an animal-consumable product). It is further contemplated that provided particle preparations (e.g., probiotic compositions) disclosed herein are suitable for use in consumable compositions of high water activity. In some instances, disclosed particle preparations (e.g., probiotic compositions) provide for stability of barrier materials (e.g., lipids, proteins, and/or carbohydrates), payload component (e.g., nutraceutical payload component), or a combination thereof when used with consumable compositions (e.g., a food product, a beverage product, an animal-consumable product).

Further, this disclosure provides for probiotic compositions (e.g., particle preparations) which may improve health. Provided technologies provide benefits over existing products because (i) in some embodiments, provided probiotic compositions (e.g., particle preparations) maintain intended cell viability when packaged as compared to previous technologies, and (ii) there have been no feasible technologies (e.g., cost-efficient, time-efficient, physically and/or chemically-capable) which suitably protect nutraceutical payload components from environmental factors to (e.g., water, high water activity, humidity, moisture, water activity, light, heat, and/or acid) to protect said viability.

Some aspects of the current disclosure provide methods of promoting health or longevity in an animal, comprising providing an effective amount of particle preparations (e.g., probiotic compositions) described herein in combination with a consumable composition (e.g., a food product, a beverage product, an animal-consumable product, etc.) to an animal. In some embodiments, consumable compositions comprise particle preparations (e.g., probiotic compositions).

In some embodiments, an animal is a human, for example, an adult, an elder, a teenager, an adolescent, or an infant. In some embodiments, an animal is an agricultural animal, for example, a horse, a cow, a pig, a sheep, a goat, a domesticated bird (e.g., chicken, duck, goose), a non-domesticated (e.g., wild) bird, etc. In some embodiments, an animal is a pet animal, for example, a dog, a cat, a rabbit, and/or a fish.

Some aspects of the current disclosure provide consumable compositions (e.g., food products, beverage product, animal-consumable compositions) comprising disclosed particle preparations (e.g., probiotic compositions). In some embodiments, consumable compositions comprising particle preparations (e.g., probiotic compositions) is or comprises a food product. In some embodiments, a food product is characterized by high water activity. In some embodiments, a food product is or comprises at least one of agricultural seed, baby formula, bread, candy, capsule, cake, cereal, chip, cookie, dry powder, fertilizer, food additive, ice cream, kefir, nutrition supplement, packaged food, pet feed, pet food, protein bar, protein powder, sachet, salad dressing, smoothie, spice, sprinkle packet, tablet, and/or yogurt. In some embodiments, consumable compositions comprising particle preparations (e.g., probiotic compositions) are provided to an animal in a mixture with a food or food ingredient.

Some aspects of the current disclosure provide non-consumable compositions that are applied for agricultural applications (e.g., agricultural seed, fertilizer). In some embodiments, non-consumable compositions comprising particle preparations (e.g., probiotic compositions) is or comprises an agricultural product for plant growth or plant nutrient delivery. In some embodiments, an agricultural product is characterized by high water activity. In some embodiments, non-consumable compositions comprising particle preparations (e.g., probiotic compositions) are provided to seeds or plants in a mixture with a seed or fertilizer or plant ingredient.

Some aspects of the current disclosure provide consumable compositions (e.g., food products, beverages, animal-consumable compositions) comprising disclosed particle preparations (e.g., probiotic compositions). In some embodiments, consumable compositions comprising particle preparations (e.g., probiotic compositions) is or comprises a beverage product. In some embodiments, a beverage product is characterized by high water activity. In some embodiments, a beverage product is or comprises at least one of liquid supplement formulation, beer, seltzer, kefir, coffee, juice, liquid pharmaceutical formulation, milk, soda, sports drink (e.g., Gatorade, sports drinks, Vitamin beverage), tea, water, and/or wine. In some embodiments, the formulation is provided to an animal in a mixture with a beverage or beverage ingredient.

Some aspects of the current disclosure provide powder-based supplement, food, and/or beverage-mix products comprising particle preparations (e.g., probiotic compositions) disclosed herein. In some embodiments, the powder-based supplement, food, and/or beverage-mix products are characterized by high water activity. In some embodiments, the powder-based supplement, food, and/or beverage-mix products is a pre-workout powder, post-workout powder or pill, pre-workout capsule/pill, baby formula, whey powder, milk powder, protein powder, drink powder mix (e.g., Kool-Aid type mix), or a powder-based supplement, food, or beverage-mix products.

1. Incorporation of Probiotic composition into Food and/or Beverage Products

In certain embodiments, the current disclosure provides for the incorporation of probiotic composition(s) into food and/or beverage products.

In some embodiments, probiotic composition(s) are incorporated into food and/or beverage products in the food and/or beverage manufacturing process. In some embodiments, probiotic composition(s) are incorporated into food and/or beverage products and/or gummy products in the food and/or beverage and/or gummy packaging process. In some embodiments, probiotic composition(s) are incorporated prior to pasteurization of a food and/or beverage product. In some embodiments, probiotic composition(s) are incorporated prior to mixing of a food and/or beverage and/or gummy product. In some embodiments, probiotic composition(s) are incorporated into finished food and/or beverage products and/or gummy products. In some embodiments, probiotic composition(s) are incorporated into food and/or beverage products and/or gummy products immediately prior to consumption.

In certain embodiments, incorporation of probiotic composition(s) (e.g., particle preparations) into food and/or beverage products utilizes size reduction techniques and/or homogenization. In some embodiments, size reduction techniques are applied to probiotic composition(s) (e.g., particle preparations) prior to incorporation. Alternatively or additionally, size reduction techniques are applied to food and/or beverage products during incorporation of the probiotic composition(s). Alternatively or additionally, size reduction techniques are applied to food and/or beverage products after incorporation of the probiotic composition(s). The present disclosure provides for size reduction using, for example, planetary milling, ball milling, burr milling, roller milling, media milling, impact milling, jet milling, high-pressure homogenization, cryo milling, hammer milling, conical milling, hand screening, or granulation/extrusion, extrusion, spray drying, lyophilization/milling, fluid bed agglomeration, spray congealing, high-shear granulation, tableting, pouring, roller compaction, crosslinking, prilling, spinning disc atomization, and/or combinations thereof.

In certain embodiments, homogenization is applied to probiotic composition(s) following incorporation into food and/or beverage products. The present disclosure provides for homogenization using, for example, overhead stirrer, manual stirring, stir bar, high pressure homogenization, low pressure homogenization, sonication, ultrasonication, vortexing, or combinations thereof.

In some embodiments, probiotic composition(s) of the present disclosure homogeneously distribute in food and/or beverage products having low pH. In some embodiments, probiotic composition(s) of the present disclosure homogeneously distribute in food and/or beverage products having high pH. In some embodiments, probiotic composition(s) of the present disclosure homogeneously distribute in food and/or beverage products having low protein content. In some embodiments, probiotic composition(s) of the present disclosure homogeneously distribute in food and/or beverage products having high protein content. In some embodiments, probiotic composition(s) of the present disclosure homogeneously distribute in food and/or beverage products having low fat content. In some embodiments, probiotic composition(s) of the present disclosure homogeneously distribute in food and/or beverage products having high fat content. In some embodiments, probiotic composition(s) of the present disclosure homogeneously distribute in food and/or beverage products having low viscosity. In some embodiments, probiotic composition(s) of the present disclosure homogeneously distribute in food and/or beverage products having high viscosity.

In some embodiments, when homogenously distributed in a transparent or translucent liquid food and/or beverage product, the food and/or beverage products maintain an opacity below about 50%, below about 45%, below about 40%, below about 35%, below about 30%, below about 25%, below about 20%, below about 15%, below about 10%, or below about 5%.

In certain embodiments, incorporation of probiotic composition(s) into food and/or beverage products significantly affects the visual appearance, texture, and/or taste of the food and/or beverage products. In other embodiments, incorporation of probiotic composition(s) into food and/or beverage products minimally affects the visual appearance, texture, and/or taste of the food and/or beverage products.

In some embodiments, disclosed particle preparations minimally affect visual appearance, texture, and/or taste when incorporated, as provided herein, into milk powder. In some instances, disclosed particle preparations minimally affect visual appearance, texture, and/or taste when incorporated, as provided herein, into dehydrated peanut butter. In some instances, disclosed particle preparations minimally affect visual appearance, texture, and/or taste when incorporated, as provided herein, into a MRE (i.e., meal ready-to-eat).

### 2. Stability of Nutraceutical Payload Component in Food and or Beverage Products

Incorporation of probiotic composition(s) into food and/or beverage products may be associated with significant reduction in the stability of nutraceutical payload component(s) (e.g., probiotic cells). The present disclosure therefore may help to improve on previous methods by providing stability in food and/or beverage matrices at a predetermined temperature, a predetermined humidity, and/or a predetermined period of time (e.g., incubation period). In some instances, a probiotic composition provides for stability of a payload component (e.g., a microbe component) in a liquid (e.g., water, SGF, SIF, SRF), food and/or beverage product(s) (e.g., sachet, yogurt, milk powder, seltzer, alcoholic beverage, vitamin beverage, sprinkle packet) or environment (e.g., elevated humidity, temperature).

In some embodiments, particle preparations may be or are effective at protecting payload component (e.g., microbes payload component) against a physical change, a chemical change, a biological change, or combinations thereof (e.g., degradation, oxidation, hydrolysis, lysis, isomerization, fragmentation, or a combination thereof). In some instances, a physical, chemical, or biological change may be induced by one or more of heat, light, shear, or water.

In some embodiments, remaining microbes in probiotic composition refers to the remaining CFUs after a period of storage at −20° C. and/or 4° C. and/or 25° C. and/or 30° C. and/or 35° C. and/or 37° C. and/or 50° C., at 33%, 53%, and/or 75% relative humidity (e.g., for 1 month and/or 2 months and/or 3 months and/or 6 months and/or 1 year and/or 2 years and/or 3 years). In certain embodiments, remaining CFUs after a period of storage are expressed as a percentage of initially loaded log(CFU/g) (e.g., % remaining log(CFU)).

In some embodiments, stability in solids is defined as there being at least 10¹⁶ CFU/g remaining. In some embodiments, stability in solids is defined as there being at least 10¹⁵ CFU/g remaining. In some embodiments, stability in solids is defined as there being at least 10¹⁴ CFU/g remaining. In some embodiments, stability in solids is defined as there being at least 10¹³ CFU/g remaining. In some embodiments, stability in solids is defined as there being at least 10¹² CFU/g remaining. In some embodiments, stability in solids is defined as there being at least 10¹¹ CFU/g remaining. In some embodiments, stability in solids is defined as there being at least 10¹⁰ CFU/g remaining. In some embodiments, stability in solids is defined as there being at least 10⁹ CFU/g remaining. In some embodiments, stability in solids is defined as there being at least 10⁸ CFU/g remaining. In some embodiments, stability in solids is defined as there being at least 10⁶ CFU/g remaining. In some embodiments, stability in solids is defined as there being at least 10⁵ CFU/g remaining. In some embodiments, stability in solids is defined as there being at least 10⁴ CFU/g remaining. In some embodiments, stability in solids is defined as there being at least 10³ CFU/g remaining.

In some embodiments, stability in liquids is defined as there being at least 10¹⁶ CFU/ml remaining. In some embodiments, stability in liquids is defined as there being at least 10¹⁵ CFU/ml remaining. In some embodiments, stability in liquids is defined as there being at least 10¹⁴ CFU/ml remaining. In some embodiments, stability in liquids is defined as there being at least 10¹³ CFU/ml remaining. In some embodiments, stability in liquids is defined as there being at least 10¹² CFU/ml remaining. In some embodiments, stability in liquids is defined as there being at least 10¹¹ CFU/ml remaining. In some embodiments, stability in liquids is defined as there being at least 10¹⁰ CFU/ml remaining. In some embodiments, stability in liquids is defined as there being at least 10⁹ CFU/ml remaining. In some embodiments, stability in liquids is defined as there being at least 10⁸ CFU/ml remaining. In some embodiments, stability in liquids is defined as there being at least 10⁶ CFU/ml remaining. In some embodiments, stability in liquids is defined as there being at least 10⁵ CFU/ml remaining. In some embodiments, stability in liquids is defined as there being at least 10⁴ CFU/ml remaining. In some embodiments, stability in liquids is defined as there being at least 10³ CFU/ml remaining.

In some embodiments, cell viability is stable in a provided composition (e.g., as described above), e.g., over a period of time at a particular environmental condition. In some embodiments, viability is assessed after 6 months at ambient temperature. In some such embodiments, remaining probiotic viability is >99.99%, >95%, >90%, >85%, >80%, >75%, >70%, >65%, and/or >60 relative to initial loading).

In some embodiments, a provided particle preparation is stable in that viability loss (e.g., log(CFU)) of a majority of a payload component it includes is minimized after passage of a period of time (e.g., at least about 1, 2, 3, 4, 5, 6, 7, or 8 weeks) under a particular environmental condition (e.g., ambient temperature). In some embodiments, stability is a viability loss of <about 2, <about 1, <about 0.5, <about 0.25, and/or <about 0.1 log(CFU) of a nutraceutical payload component (e.g., microbes) is observed over a period of time under the environmental condition. In some embodiments, the period of time is up to about 8 weeks and the environmental condition is or comprises ambient temperature. In some embodiments, the period of time is up to about 2 weeks and the environmental condition is or comprises presence of water (e.g., in aqueous solution). In some embodiments, the period of time is up to about 72 hours and the environmental condition is or comprises exposure to light at elevated temperatures (e.g., about 37° C.): in some such embodiments, at least about 80%, at least about 85%, at least about 90%, or at least about 95% or more of a payload component retains its integrity (e.g., log(CFU)) over the period of time under the environmental condition.

In some embodiments, a provided particle preparation is stable in that viability loss (e.g., log(CFU)) of a majority of a payload component it includes is minimized after passage of a period of time (e.g., at least about 1, 2, 3, 6, 9, 12, 24, or 36 months) under a particular environmental condition (e.g., ambient temperature). In some embodiments, stability is a viability loss of <about 2, <about 1, <about 0.5, <about 0.25, and/or <about 0.1 log(CFU) of a nutraceutical payload component (e.g., microbes) is observed over a period of time under the environmental condition. In some embodiments, the period of time is up to about 36 months and the environmental condition is or comprises ambient temperature. In some embodiments, the period of time is up to about 12 months and the environmental condition is or comprises presence of food product (e.g., in a mixture with a food product). In some embodiments, the period of time is up to about 1 month and the environmental condition is or comprises exposure to a food product (e.g., in a mixture with yogurt); in some such embodiments, at least about 70%, at least about 75%, at least about 80%, at least about 85%, at least about 90%, and/or at least about 95% or more of a payload component retains its integrity (e.g., log(CFU)) over the period of time under the environmental condition.

In some embodiments, a payload component (e.g., microbes payload component) may be or is protected against lysis upon standing in a food. In some instances, protection is <3 log(CFU) loss observed for at least 1 year at >−20° C., >0° C., >20° C., >25° C., >30° C., and/or >35° C. In some instances, protection is <2 log(CFU) loss observed for at least 1 year at >−20° C., >0° C., >20° C., >25° C., >30° C. and/or >35° C. In some instances, protection is <1 log(CFU) loss observed for at least 1 year at >−20° C., >0° C., >20° C., >25° C., >30° C., and/or >35° C.

In some embodiments, stability in gummy products is defined as there being at least 10¹⁶ CFU/g remaining. In some embodiments, stability in gummy products is defined as there being at least 10¹⁵ CFU/g remaining. In some embodiments, stability in gummy products is defined as there being at least 10¹⁴ CFU/g remaining. In some embodiments, stability in gummy products is defined as there being at least 10¹³ CFU/g remaining. In some embodiments, stability in gummy products is defined as there being at least 10¹² CFU/g remaining. In some embodiments, stability in gummy products is defined as there being at least 10¹¹ CFU/g remaining. In some embodiments, stability in gummy products is defined as there being at least 10¹⁰ CFU/g remaining. In some embodiments, stability in gummy products is defined as there being at least 10⁹ CFU/g remaining. In some embodiments, stability in gummy products is defined as there being at least 10⁸ CFU/g remaining. In some embodiments, stability in gummy products is defined as there being at least 10⁶ CFU/g remaining. In some embodiments, stability in gummy products is defined as there being at least 10⁵ CFU/g remaining. In some embodiments, stability in gummy products is defined as there being at least 10⁴ CFU/g remaining. In some embodiments, stability in gummy products is defined as there being at least 10³ CFU/g remaining.

In some embodiments, a payload component (e.g., microbes payload component) may be or is protected against lysis upon standing in a food. In some instances, protection is <3 log(CFU) loss observed for at least >1, >2, and/or >3 years at >−20° C., >0° C., >20° C., >25° C., >30° C., and/or >35° C. In some instances, protection is <2 log(CFU) loss observed for at least >1, >2, and/or >3 years at >−20° C., >0° C., >20° C., >25° C., >30° C., and/or >35° C. In some instances, protection is <1 log(CFU) loss observed for at least >1, >2, and/or >3 years at >−20° C., >0° C., >20° C., >25° C., >30° C., and/or >35° C.

In some embodiments, particles disclosed herein are effective to protect against permeation of water (e.g., aqueous liquids, soda, seltzer, Gatorade, sports drinks, vitamin beverage, water). In some instances, a payload component (e.g., microbe payload component) may be or is protected against lysis upon standing in a beverage. In some instances, protection is <6 log(CFU) loss observed for at least 1 year at >−20° C., >0° C., >20° C., >25° C., >30° C., and/or >35° C. In some instances, protection is <3 log(CFU) loss observed for at least 1 year at >−20° C., >0° C., >20° C., >25° C., >30° C. and/or >35° C. In some instances, protection is <1 log(CFU) loss observed for at least 1 year at >−20° C., >0° C., >20° C., >25° C., >30° C., and/or >35° C.

In some embodiments, particles disclosed herein are effective to protect against permeation of fluids (e.g., aqueous liquids, soda, seltzer, Gatorade, sports drinks, vitamin beverage, water). In some instances, a payload component (e.g., microbes payload component) may be or is protected against lysis upon standing in a beverage. In some instances, protection is <6 log(CFU) loss observed for >1, >2, and/or >3 years at >−20° C., >0° C., >20° C., >25° C., >30° C., and/or >35° C. In some instances, protection is <3 log(CFU) loss observed for at least 1 year at >−20° C., >0° C., >20° C., >25° C., >30° C., and/or >35° C. In some instances, protection is <1 log(CFU) loss observed for at least 1 year at >−20° C., >0° C., >20° C., >25° C., >30° C., and/or >35° C.

In some instances, viability of the payload component (<about 3 log(CFU) loss) is maintained after storage in a solid food (e.g., bread, rice, baked goods, etc.) at ambient temperatures for time periods between 0-1 week, 0-1 month, 0-1 years, or 1-5 years of storage.

In some instances, viability of the payload component (<about 3 log(CFU) loss) is maintained after storage in a dry powder (e.g., supplement powder, milk powder, baby formula, flour, etc.) at ambient temperatures for time periods between 0-1 week, 0-1 month, 0-1 years, or 1-5 years of storage.

In some instances, viability of the payload component (<about 3 log(CFU) loss) is maintained after storage in a liquid beverage (e.g., coffee, drinkable yogurt, water, soda, Gatorade, sports drinks, etc.) at ambient temperatures for time periods between 0-1 week, 0-1 month, 0-1 years, or 1-5 years of storage.

In some embodiments, disclosed particle preparations are stable (<about 2 log(CFU) loss) up to 2 weeks, up to 1 month, up to 6 months, up to 1 year, up to 2 years, up to 5 years, etc. in water at ambient temperature.

In some embodiments, disclosed particle preparations are stable (<about 2 log(CFU) loss) up to 2 weeks in yogurt at ambient temperature.

In some embodiments, disclosed particle preparations are stable (<about 2 log(CFU) loss) up to 2 weeks in milk powder at ambient temperature.

In some instances, viability of the payload component (<about 3 log(CFU) loss) is maintained after storage in a gummy product (e.g., gummy bear, gummy worm, gelatin-based gummy product, pectin-based gummy product, etc.) at ambient temperatures for time periods between 0-1 week, 0-1 month, 0-1 years, or 1-5 years of storage.

In some embodiments, disclosed particle preparations are stable (<about 2 log(CFU) loss) up to 2 weeks in baby formula at ambient temperature.

In some embodiments, disclosed particle preparations are stable (<about 2 log(CFU) loss) up to 2 weeks in whole milk powder instant at ambient temperature.

In some embodiments, disclosed particle preparations are stable (<about 2 log(CFU) loss) up to 2 weeks in high fat milk powder at ambient temperature.

In some embodiments, disclosed particle preparations are stable (<about 2 log(CFU) loss) up to 2 weeks in a sachet at ambient temperature.

In some embodiments, particle preparations are stable (<about 2 log(CFU) loss) up to 2 weeks when combined with animal feed (e.g., total meal ration, animal feed pellets, etc.) at ambient temperature.

In some embodiments, particle preparations may be effective to protect payload component(s) against humidity-induced degradation. In some instances, payload component(s) dispersed in food product(s) is or are stable (<about 2 log(CFU) loss) when exposed to ambient humidity (e.g., 30% relative humidity) at ambient temperatures (e.g., 25° C.) for up to 6 weeks.

In some embodiments, particle preparations may be effective to protect payload component(s) against humidity-induced degradation. In some instances, payload component(s) dispersed in food product(s) is or are stable (<about 2 log(CFU) loss) when exposed to low humidity (e.g., 20% relative humidity) at ambient temperatures (e.g., 25° C.) for up to 6 weeks.

In some embodiments, particle preparations may be effective to protect payload component(s) against humidity-induced degradation. In some instances, payload component(s) dispersed in food product(s) is or are stable (<about 2 log(CFU) loss) when exposed to ambient humidity (e.g., 30% relative humidity) at elevated temperatures (e.g., 40° C.) for up to 6 weeks.

In some embodiments, particle preparations may be effective to protect payload component(s) against humidity-induced degradation. In some instances, payload component(s) dispersed in food product(s) is or are stable (<about 2 log(CFU) loss) when exposed to low humidity (e.g., 20% relative humidity) at elevated temperatures (e.g., 40° C.) for up to 6 weeks.

In some embodiments, probiotic compositions are incorporated into a food and/or beverage product in the presence of humidity (e.g., water, moisture content, water activity). In some instances, particle preparations may be effective to protect payload component(s) against humidity-induced degradation. In some instances, payload component(s) is or are stable (<about 2 log(CFU) loss) when exposed to >15%, >20%, >25%, and/or >30% relative humidity, at >−20° C. and/or >4° C. and/or >25° C. and/or >30° C. and/or >35° C. and/or >37° C. and/or >50° C., for >1, >2, >3, >4, >6, and/or >8 weeks.

In some embodiments, particle preparations may be effective to protect payload component(s) against humidity-induced degradation. In some instances, payload component(s) is or are stable (<about 2 log(CFU) loss) when exposed to high humidity (e.g., 75% relative humidity) at ambient temperatures (e.g., 25° C.) for up to 6 weeks.

In some embodiments, probiotic compositions are incorporated into a food and/or beverage product in the presence of humidity (e.g., water, moisture content, water activity). In some instances, particle preparations may be effective to protect payload component(s) against humidity-induced degradation. In some instances, payload component(s) is or are stable (<about 2 log(CFU) loss) when exposed to >50%, >55%, >60%, >65%, >70% and/or >75% relative humidity, at >−20° C. and/or >4° C. and/or >25° C. and/or >30° C. and/or >35° C. and/or >37° C. and/or >50° C., for >1, >2, >3, >4, >6, and/or >8 weeks.

In some instances, viability of the payload component (<about 3 log(CFU) loss) is maintained after storage in a freezer (−85° C. to 0° C.), a refrigerator (1-10° C.), or atmospheric temperature (−10° C.-40° C.) for time periods between 0-1 week, 0-1 month, 0-1 years, or 1-5 years of storage.

In some instances, protection against oxygen, heat, light, and water of payload component is maintained after storage in a freezer (−85° C. to 0° C.), a refrigerator (1-10° C.), or atmospheric temperature (−10° C.-40° C.) for time periods ranging from 0-1 week, 0-1 month, 0-1 year, and/or 1-5 years of storage.

In some embodiments, particle compositions of the present disclosure do not comprise are prepared in the absence of any cryoprotectant. For example, in some embodiments, payload component(s) (e.g. probiotics) of particle compositions maintain viability when incorporated in a food and/or beverage product stored at temperatures below 0° C. (e.g., −10° C., −15° C., −20° C., −25° C., −30° C., −35° C., −40° C., −45° C., −50° C., −55° C., −60° C., −65° C., −70° C., −75° C., −80° C., −85° C., −90° C., −95° C. or −100° C.) in the absence of any cryoprotectant.

In some embodiments, particle compositions of the present disclosure do not comprise are prepared in the absence of any cryoprotectant. For example, in some embodiments, payload component(s) (e.g., probiotics) of particle compositions in the absence of any cryoprotectant, maintain viability when incorporated in a food and/or beverage product stored at temperatures below 0 to −100° C., −10 to −100° C., −20 to −100° C., −30 to −100° C., −40 to −100° C., −50 to −100° C., −60 to −100° C., −70 to −100° C., −80 to −100° C., −90 to −100° C., 0 to −90° C., −10 to −90° C., −20 to −90° C., −30 to −90° C., −40 to −90° C., −50 to −90° C., −60 to −90° C., −70 to −90° C., −80 to −90° C., 0 to −80° C., −10 to −80° C., −20 to −80° C., −30 to −80° C., −40 to −80° C., −50 to −80° C., −60 to −80° C., −70 to −80° C., 0 to −70° C., −10 to −70° C., −20 to −70° C., −30 to −70° C., −40 to −70° C., −50 to −70° C., −60 to −70° C., 0 to −60° C., −10 to −60° C., −20 to −60° C., −30 to −60° C., −40 to −60° C., −50 to −60° C., 0 to −50° C., −10 to −50° C., −20 to −50° C., −30 to −50° C., −40 to −50° C., 0 to −40° C., −10 to −40° C., −20 to −40° C., −30 to −40° C., 0 to −30° C., −10 to −30° C., −20 to −30° C., −30 to −30° C., −40 to −30° C., −50 to −30° C., −60 to −30° C., −70 to −30° C., −80 to −30° C., −90 to −30° C., or −10 to −20° C.

In some embodiments of the present disclosure, payload component(s) (e.g., probiotics) of particle compositions maintain viability when incorporated in a food and/or beverage product stored at temperatures above 25-100° C. 30-100° C., 35-100° C., 40-100° C., 45-100° C., 50-100° C., 55-100° C., 60-100° C., 65-100° C., 70-100° C., 75-100° C., 75-100° C., 80-100° C., 85-100° C., 90-100° C., 95-100° C., 25-90° C., 30-90° C., 35-90° C., 40-90° C., 45-90° C., 50-90° C., 55-90° C. 60-90° C. 65-90° C. 70-90° C. 75-90° C., 75-90° C., 80-90° C., 85-90° C., 25-80° C., 30-80° C.35-80° C.40-80° C., 45-80° C., 50-80° C., 55-80° C., 60-80° C., 65-80° C., 70- 80° C., 75-80° C., 75-80° C., 25-70° C., 30-70° C., 35-70° C., 40-70° C., 45-70° C., 50-70° C., 55-70° C., 60-70° C., 65-70° C., 25-60° C., 30-60° C., 35-60° C., 40-60° C., 45-60° C., 50-60° C., 55-60° C., 25-50° C., 30-50° C., 35-50° C., 40-50° C., 45-50° C., 25-40° C., 30-40° C., 35-40° C., or 25-30° C.

In some embodiments, in an unencapsulated form, payload component(s) (e.g., probiotics) of the present disclosure may cause a food and/or beverage product to spoil, less palatable, and/or less visually appealing. However, when in an encapsulated form, payload component(s) (e.g., probiotics) of the present disclosure do not cause a food and/or beverage product to spoil, less palatable, and/or less visually appealing.

In some embodiments, probiotic composition(s) of the present disclosure maintain viability when incorporated into and/or stored in food and/or beverage products having low pH. In some embodiments, probiotic composition(s) of the present disclosure maintain viability when incorporated into and/or stored in food and/or beverage products having high pH. In some embodiments, probiotic composition(s) of the present disclosure maintain viability when incorporated into and/or stored in food and/or beverage products having low protein content. In some embodiments, probiotic composition(s) of the present disclosure maintain viability when incorporated into and/or stored in food and/or beverage products having high protein content. In some embodiments, probiotic composition(s) of the present disclosure maintain viability when incorporated into and/or stored in food and/or beverage products having low fat content. In some embodiments, probiotic composition(s) of the present disclosure maintain viability when incorporated into and/or stored in food and/or beverage products having high fat content. In some embodiments, probiotic composition(s) of the present disclosure maintain viability when incorporated into and/or stored in food and/or beverage products having low viscosity. In some embodiments, probiotic composition(s) of the present disclosure maintain viability when incorporated into and/or stored in food and/or beverage products having high viscosity.

### 3. Characterizing Compositions and or Components Thereof

In some embodiments, provided composition(s), and/or component(s) thereof, are subjected to one or more assessments, for example to characterize one or more structural features and/or functional properties thereof (e.g., for quality control and/or after storage under particular conditions and for a particular period of time). In some embodiments, batches that do not meet designated criteria may be discarded or not further utilized.

### EXEMPLARY ENUMERATED EMBODIMENTS

The following numbered embodiments, while non-limiting are exemplary of certain aspects of the present disclosure:

1) A probiotic composition comprising microbes and one or more barrier materials, wherein the one or more barrier materials comprise, on a dry weight basis:
about 40% to about 99% (w/w) lipid; and
about 0% to about 59% (w/w) of:
   a carbohydrate;
   a protein;
   a polymer; or
   combinations thereof.

2) The composition of embodiment 1, wherein the composition is a particle preparation.

3) The composition of embodiment 2, wherein the particles are characterized to be about 1-10000 μm in diameter.

4) The composition of embodiment 2, wherein the particles are characterized to be about 2-5000 μm in diameter.

5) The composition of embodiment 2, wherein the particles are characterized to be about 3-1000 μm in diameter.

6) The composition of embodiment 2, wherein the particles are characterized to be about 10-1000 μm in diameter.

7) The composition of any one of embodiments 2-6, wherein the dispersity of particles is <about 0.4, <about 0.3, <about 0.2, <about 0.1.

8) The composition of any one of embodiments 2-7, wherein the diameter and dispersity are measured using a Malvem Mastersizer.

9) The composition of any one of embodiments 2-7, wherein the diameter and dispersity are measured using Scanning Electron Microscopy.

10) The composition of any one of embodiments 2-9, wherein particles of the preparation are characterized in having a water activity of <about 0.6, <about 0.3, <about 0.2, and/or <about 0.1.

11) The composition of any one of embodiment 10, wherein the water activity is measured using a TDL2 water activity meter.

12) The composition of any one of embodiments 2-11, wherein the particle comprises core materials encapsulated by a shell materials.

13) The composition of embodiment 12, wherein the core materials comprise, on a dry weight basis, about 40% to about 99% (w/w) of the particle composition.

14) The composition of embodiment 13, wherein the core materials comprise microbes and one or more barrier materials.

15) The composition of embodiment 14, wherein the microbes are a dry powder comprising a single species or a mixture of species.

16) The composition of embodiment 15, wherein the powder is characterized to be about 0.01-4000 μm in diameter.

17) The composition of embodiment 16, wherein the powder is characterized to be about 0.05-1000 μm in diameter.

18) The composition of embodiment 17, wherein the powder is characterized to be about 0.06-200 μm in diameter.

19) The composition of embodiment 18, wherein the powder is characterized to be about 1-100 μm in diameter.

20) The composition of embodiment 19, wherein the diameter is measured using a Malvern Mastersizer.

21) The composition of any one of embodiments 15-20, wherein the microbes are dispersed within the core materials

22) The composition of any one of embodiments 15-21, wherein a density of one or more core materials of the microbes is between about 1×10⁵ CFU/g and about 1×10¹⁴ CFU/g.

23) The composition of any one of embodiments 15-21, wherein a density of one or more core materials of the microbes is between about 1×10⁷ CFU/g and about 1×10¹³ CFU/g.

24) The composition of any one of embodiments 15-21, wherein a density of one or more core materials of the microbes is between about 1×10⁹ CFU/g and about 1×10¹² CFU/g.

25) The composition of any one of embodiments 15-24, wherein the microbes are probiotic bacteria.

26) The composition of any one of embodiments 15-25, wherein the probiotic bacteria are selected from the group comprising: Bacillus coagulans, Bacillus licheniformis, Bacillus subtilis, Bifidobacterium angulatum, Bifidobacterium animalis, Bifidobacterium bifidum, Bifidobacterium breve, Bifidobacterium infantis, Bifidobacterium Bifidobacterium longum, Enterococcus faecium, Enterococcus faecalis, Lactobacillus acidophilus, Lactobacillus amylovorus, Lactobacillus alimentarius, Lactobacillus bulgaricus, Lactobacillus casei subsp. casei, Lactobacillus casei Sinrota, Lactobacillus curvatus, Lactobacillus delbrueckii subsp lactis, Lactobacillus fermentum, Lactobacillus farciminis, Lactobacillus gasseri, Lactobacillus helveticus, Lactobacillus johnsonii, Lactobacillus lacti, Lactobacillus paracasei, Lactobacillus pentosaceus, Lactobacillus plantarum, Lactobacillus reuteri, Lactobacillus rhamnosus (Lactobacillus GG), Lactobacillus sake, Lactobacillus salivarius, Lactococcus lactis, Micrococcus varians, Pediococcus acidilactici Pediococcus pentosaceus, Pediococcus acidilactici, Pediococcus halophilus, Streptococcus faecalis. Streptococcus thermophilus. Staphylococcus camosus, and Staphylococcus xylosus.

27) The composition of any one of embodiments 15-26, wherein >40%, >60%, and/or >80% of probiotic introduced during the manufacturing process is entrapped within the core materials.

28) The composition of any one of embodiments 15-26, wherein the barrier material is a solid at 25° C.

29) The composition of any one of embodiments 15-26, wherein the barrier material is moisture resistant.

30) The composition of any one of embodiments 15-26, wherein the moisture resistant material is characterized to melt between 30-90° C.

31) The composition of any one of embodiments 15-26, wherein the moisture resistant material is characterized to melt between 36-70° C.

32) The composition of any one of embodiments 15-26, wherein the moisture resistant material is characterized to melt between 40-60° C.

33) The composition of any one of embodiments 29-32, wherein the moisture resistant material is a lipid.

34) The composition of embodiment 33, wherein the lipid is a wax.

35) The composition of embodiment 34, wherein the wax comprises paraffin wax, montan wax, microcrystalline wax, polyethylene wax, petrolatum wax, ozokerite wax, ceresin wax, beeswax, lanolin wax, spermaceti wax, tallow wax, lac wax, chinese insect wax, ambergris wax, soy wax, carnauba wax, candelilla wax, coconut wax, palm kernel wax, rice bran wax, or combinations thereof.

36) The composition of embodiment 33, wherein the lipid is a plant oil.

37) The composition of embodiment 36, wherein the plant oil comprises fatty acid monoglyceride esters, fatty acid diglyceride esters, fatty acid triglyceride esters, coconut oil, cottonseed oil, palm oil, soybean oil, sunflower oil, fully hydrogenated palm oil, partially hydrogenated palm oil, fully hydrogenated sunflower oil, partially hydrogenated sunflower oil, fully hydrogenated soybean oil, partially hydrogenated soybean oil, fully hydrogenated vegetable oil, partially hydrogenated vegetable oil, fully hydrogenated cottonseed oil, partially hydrogenated cottonseed oil, or combinations thereof.

38) The composition of embodiment 33, wherein the lipid is a fatty acid.

39) The composition of embodiment 38, wherein the fatty acid comprises butyric acid, pentanoic acid, hexanoic acid, heptanoic acid, caprylic acid, nonanoic acid, capric acid, lauric acid, myristic acid, palmitic acid, stearic acid, and/or arachidonic acid, or combinations thereof.

40) The composition of embodiment 12, wherein the shell materials comprise, on a dry weight basis, about 0% to about 50% (w/w) of the particle preparation.

41) The composition of embodiment 40, wherein the shell materials comprise a carbohydrate, a protein, or combinations thereof.

42) The composition of embodiment 41, wherein the carbohydrate comprises: amylose, amylopectin, cellulose, methyl cellulose, ethyl cellulose, hydroxypropyl cellulose, hydroxypropyl methyl cellulose, hydroxypropyl ethyl cellulose, cellulose triacetate, cellulose acetate succinate, cellulose acetate butyrate, cellulose acetate phthalate, hydroxypropyl methylcellulose acetate succinate, sodium alginate, potassium alginate, calcium alginate, magnesium alginate, zinc alginate, sodium pectinate, potassium pectinate, calcium pectinate, zinc pectinate, sodium hyaluronate, potassium hyaluronate, calcium hyaluronate, magnesium hyaluronate, zinc hyaluronate, or combinations thereof.

43) The composition of embodiment 41, wherein the protein comprises: whey protein, β-lactoglobulin, α-lactalbumin, casein, bovine serum albumin, ovalbumin, zein, hordein, gliadin, secalin, kafirin, avenin, or combinations thereof.

44) The composition of any one of embodiments 2-43, wherein the water activity following incubation for 96 hours at 75% humidity at 25° C. is <about 0.7, <about 0.5, and/or <0.3.

45) The composition of any one of embodiments 2-43, wherein the water activity following incubation for 96 hours at 53% humidity at 25° C. is <about 0.6, <about 0.5, and/or <0.3.

46) The composition of any one of embodiments 2-43, wherein the water activity following incubation for 96 hours at 33% humidity at 25° C. is <about 0.5, <about 0.4, and/or <0.3.

47) The composition of any one of embodiments 2-46, wherein the moisture content following incubation for 96 hours at 75% humidity at 25° C. is <about 8%, <about 4%, and/or <about 2% (w/w).

48) The composition of any one of embodiments 2-46, wherein the moisture content following incubation for 96 hours at 53% humidity at 25° C. is <about 8%, <about 4%, and/or <about 2% (w/w).

49) The composition of any one of embodiments 2-46, wherein the moisture content following incubation for 96 hours at 33% humidity at 25° C. is <about 6%, <about 4%, and/or <about 1.5% (w/w).

50) The composition of any one of embodiments 2-49, wherein the particle preparation is effective to protect against degradation of the microbes.

51) The composition of any one of embodiments 2-50, wherein degradation comprises loss of log colony forming units (log(CFUs)), changes to particle morphology, changes to particle diameter, or combinations thereof.

52) The composition of any one of embodiments 2-51, wherein the particle preparation is effective to protect against moisture-induced degradation (e.g., presence of water, humidity, water activity, moisture content or combinations thereof), heat-induced degradation, acid-induced degradation (e.g., presence of simulated gastric fluid), degradation as a result of storage in a food and/or beverage product, or combinations thereof.

53) The composition of any one of embodiments 2-52, wherein the particle preparation is effective to protect against moisture-induced degradation in aqueous media at 37° C. for at least 1 hour, 2 hours, 3 hours, 6 hours, 12 hours, and/or 24 hours.

54) The composition of embodiment 53, wherein the loss of log(CFU) is <about 2, <about 1, and/or <about 0.5.

55) The composition of embodiment 53, wherein the loss of log(CFU) relative to untreated particle preparation is <about 30%, <about 20%, <about 10%.

56) The composition of embodiment 53, wherein the particle diameter is within about 30%, within about 20%, and/or within about 10% of the untreated particle diameter.

57) The composition of any one of embodiments 2-56, wherein the particle preparation is effective to protect against moisture-induced degradation at elevated relative humidity for at least 1 day, 2 days, 3 days, 6 days, 8 days, or 14 days at 25° C.

58) The composition of embodiment 57, wherein the particle preparation is effective to protect against moisture-induced degradation at about 35% relative humidity.

59) The composition of embodiment 2-58, wherein the particle preparation is effective to protect against moisture-induced degradation at about 50% relative humidity.

60) The composition of any one of embodiments 57-59, wherein the loss of log(CFU) is <about 2, <about 1, and/or <about 0.5.

61) The composition of any one of embodiments 57-59, wherein the loss of log(CFU) relative to untreated particle preparation is <about 30%, <about 20%, and/or <about 10%.

62) The composition of any one of embodiments 2-61, wherein the particle preparation is effective to protect against degradation at >about −20° C., >about 4° C., >about 25° C., and/or >about 37° C.

63) The composition of embodiment 62, wherein the particle preparation is effective to protect against degradation for at least 1 month, 2 months, 6 months, 1 year, and/or 3 years.

64) The composition of any one of embodiments 62-63, wherein the loss of log(CFU) is <about 2, <about 1, and/or <about 0.5.

65) The composition of any one of embodiments 62-63, wherein the loss of log(CFU) relative to untreated particle preparation is <about 30%, <about 20%, <about 10%.

66) The composition of any one of embodiments 62-63, wherein the particle diameter is within about 40%, within about 30%, and/or within about 20% of the untreated particle diameter.

67) The composition of any one of embodiments 2-66, wherein the particle preparation is effective to protect against acid-induced degradation (i.e., simulated gastric fluid) at 37° C. up to 24 hours, up to 48 hours, up to 96 hours, and/or up to 192 hours.

68) The composition of any one of embodiments 2-67, wherein the particle preparation is effective to protect against degradation at a pH<about 5, a pH<about 4, a pH<about 3, a pH<about 2.

69) The composition of any one of embodiments 2-68, wherein the particle preparation is effective to protect against simulated gastric fluid.

70) The composition of embodiment 69, wherein the loss of log(CFU) is <about 2, <about 1, and/or <about 0.5.

71) The composition of embodiment 69, wherein the loss of log(CFU) relative to untreated particle preparation is <about 30%, <about 20%, and/or <about 10%.

72) The composition of any one of embodiments 2-71, wherein the particle preparation is effective to protect against degradation as a result of storage in a food and/or beverage product.

73) The composition of embodiment 72, wherein the food product is comprised at least of: agricultural seed, baby formula, bread, candy, capsule, cake, cereal, chip, cookie, dry powder, fertilizer, food additive, ice cream, kefir, nutrition supplement, packaged food, pet feed, pet food, protein bar, protein powder, sachet, salad dressing, smoothie, spice, sprinkle packet, tablet, yogurt, or combinations thereof.

74) The composition of embodiment 72, wherein the beverage product is comprised at least of beer, kefir, coffee, juice, liquid pharmaceutical formulation, milk, soda, sports drink, tea, water, wine, or combinations thereof.

75) The composition of any one of embodiments 2-74, wherein the particle preparation is effective to protect against degradation at >about −20° C., >about 4° C., >about 25° C., and/or >about 37° C.

76) The composition of any one of embodiments 2-75, wherein the particle preparation is effective to protect against degradation for at least 1 week, 2 weeks, 1 month, 2 months, 6 months, 1 year, and/or 3 years.

77) The composition of embodiment 73, wherein the particle preparation is dispersed within a milk powder.

78) The composition of embodiment 77, wherein the loss of log(CFU) after 4 weeks at 25° C. and about 35% relative humidity is <about 2, <about 1, and/or <about 0.5.

79) The composition of embodiment 77, wherein the loss of log(CFU) after 8 weeks at 25° C. and about 35% relative humidity is <about 2, <about 1, and/or <about 0.5.

80) The composition of embodiment 77, wherein the loss of log(CFU) after 12 weeks at 25° C. and about 35% relative humidity is <about 2, <about 1, and/or <about 0.5.

81) The composition of embodiment 77, wherein the loss of log(CFU) after 4 weeks at 25° C. and about 50% relative humidity is <about 3, <about 2, and/or <about 1.

82) The composition of embodiment 77, wherein the loss of log(CFU) after 8 weeks at 25° C. and about 50% relative humidity is <about 3, <about 2, and/or <about 1.

83) The composition of embodiment 77, wherein the loss of log(CFU) after 12 weeks at 25° C. and about 50% relative humidity is <about 3, <about 2, and/or <about 1.

84) The composition of embodiment 77, wherein the loss of log(CFU) after 4 weeks at 37° C. and about 35% relative humidity is <about 2, <about 1, and/or <about 0.5.

85) The composition of embodiment 77, wherein the loss of log(CFU) after 8 weeks at 37° C. and about 35% relative humidity is <about 2, <about 1, and/or <about 0.5.

86) The composition of embodiment 77, wherein the loss of log(CFU) after 12 weeks at 37° C. and about 35% relative humidity is <about 2, <about 1, and/or <about 0.5.

87) The composition of embodiment 73, wherein the particle preparation is dispersed within a yogurt.

88) The composition of embodiment 87, wherein the loss of log(CFU) after 2 weeks at 37° C. and about 35% relative humidity is <about 4, <about 2, and/or <about 1.

89) The composition of embodiment 87, wherein the loss of log(CFU) after 4 weeks at 37° C. and about 35% relative humidity is <about 4, <about 2, and/or <about 1.

90) The composition of embodiment 87, wherein the loss of log(CFU) after 8 weeks at 37° C. and about 35% relative humidity is <about 4, <about 2, and/or <about 1.

91) A method for enumerating microbes in probiotic compositions comprising a step of:
i) weighing 2 portions of formulated probiotic compositions:
ii) adding the first portion of formulated probiotic compositions to a warmed, stirred oil bath;
iii) sequentially adding emulsifier and amenable salt solution to the aforementioned stirring oil bath;
iv) serially diluting an aliquot of the aforementioned emulsion;
v) performing spread plate enumeration on the aforementioned dilutions;
vi) adding the second portion of formulated probiotic compositions directly to an aqueous salt solution;
vii) mixing the aforementioned aqueous suspension salt and formulated probiotic compositions;
viii) serially diluting the aforementioned aqueous solution; and
ix) performing spread plate enumeration on the aforementioned dilutions.

92) The method of embodiment 91, wherein the microbes are bacteria.

93) The method of embodiment 92, wherein the bacteria are probiotics.

94) The method of embodiment 93, wherein the probiotics are selected from the group comprising, Bacillus coagulans, Bacillus licheniformis, Bacillus subtilis, Bifidobacterium angulatum, Bifidobacterium animalis, Bifidobacterium bifidum, Bifidobacterium breve. Bifidobacterium infantis, Bifidobacterium Bifidobacterium longum, Enterococcus faecium, Enterococcus faecalis, Lactobacillus acidophilus, Lactobacillus amylovorus, Lactobacillus alimentarius, Lactobacillus bulgaricus, Lactobacillus casei subsp. casei, Lactobacillus casei Sinrota, Lactobacillus curvatus, Lactobacillus delbrueckii subsp lactis, Lactobacillus fermentum, Lactobacillus farciminis, Lactobacillus gasseri, Lactobacillus helveticus, Lactobacillus johnsonii. Lactobacillus lacti, Lactobacillus paracasei, Lactobacillus pentosaceus, Lactobacillus plantarum, Lactobacillus reuteri, Lactobacillus rhamnosus (Lactobacillus GG), Lactobacillus sake, Lactobacillus salivarius, Lactococcus lactis, Micrococcus varians, Pediococcus acidilactici Pediococcus pentosaceus, Pediococcus acidilactici, Pediococcus halophilus, Streptococcus faecalis, Streptococcus thermophilus, Staphylococcus camosus, or Staphylococcus xylosus.

95) The method of any one of embodiments 91-94, wherein the oil is at least one of vegetable oil, castor oil, avocado oil, sunflower oil, rapeseed oil, mineral oil, or palm oil.

96) The method of embodiment 95, wherein the mass of oil in the warmed, stirred oil bath is between about 1 to about 100 fold the measured mass of formulated probiotic compositions.

97) The method of embodiment 95, wherein the mass of oil in the warmed, stirred oil bath is between about 2 to about 75 fold the measured mass of formulated probiotic compositions.

98) The method of embodiment 95, wherein the mass of oil in the warmed, stirred oil bath is between about 5 to about 50 fold the measured mass of formulated probiotic compositions.

99) The method of any one of embodiments 95-98, wherein the temperature of the oil bath is between about 20° C. and about 90° C.

100) The method of any one of embodiments 95-98, wherein the temperature of the oil bath is between about 35° C. and about 80° C.

101) The method of any one of embodiments 91-100, wherein the emulsifier is characterized as having an HLB value <18.

102) The method of embodiment 101, wherein the emulsifier comprises at least one of Cetearyl Alcohol, Cetearyl Glucoside, Cetyl Alcohol, Emulsifying Wax, Glyceryl Stearate, PEG-40 Hydrogenated Castor Oil, Polyoxyethylene glycol sorbitan alkyl esters, Polysorbates, Propanediol, Safflower Oleosomes, and Sorbitan alkyl esters.

103) The method of any one of embodiments 101-102, wherein the mass of emulsifier is between about 1 to about 20 fold relative to the mass of oil in the oil bath.

104) The method of any one of embodiments 101-103, wherein the amenable salt solution is at least one of Peptone water, saline solution, Phosphate buffer saline solution, Dulbecco's phosphate buffer saline solution, HEPES buffer saline solution. Earl's balanced salt solution, or Hank's balanced salt solution.

105) The method of any one of embodiments 101-104, wherein the mass of salt solution is added such that the final concentration of emulsifier in the emulsion is between about 0.5% and about 30% (w/w).

106) The method of any one of embodiments 101-105, wherein the mixing rate is between about 50 and about 500 RPM.

107) The method of any one of embodiments 101-105, wherein the mixing rate is between about 100 and about 400 RPM.

108) The method of any one of embodiments 106-107, wherein the mixing time is between about 5 and about 240 minutes.

109) The method of any one of embodiments 106-107, wherein the mixing time is between about 30 and about 120 minutes.

110) The method of any one of embodiments 91-109, wherein the emulsions are diluted between about 0 and about 12 10-fold dilutions prior to spread plate enumeration.

111) A method of manufacturing a probiotic composition comprising microbes and a barrier material comprising a step of
i) milling a freeze-dried microbes solution, forming milled microbes;
ii) dispersing the milled microbes within a liquid, thereby forming a suspension;
iii) homogenizing the suspension of microbes a in liquid matrix;
iv) atomizing the homogenized liquid matrix;
v) air-cooling the atomized liquid matrix, forming cooled compositions;
vi) collecting the cooled compositions;
vii) coating the collected cooled compositions forming coated compositions; and
viii) drying the coated compositions.

112) The method of embodiment 111, wherein the probiotic composition is a particle preparation.

113) The method of embodiment 112, wherein the particles are characterized to be about 1-10000 μm in diameter.

114) The method of embodiment 112, wherein the particles are characterized to be about 2-5000 μm in diameter.

115) The method of embodiment 112, wherein the particles are characterized to be about 3-1000 μm in diameter.

116) The method of embodiment 112, wherein the particles are characterized to be about 10-1000 μm in diameter.

117) The method of any one of embodiments 112-116, wherein the dispersity of particles is <about 0.4, <about 0.3, <about 0.2, and/or <about 0.1.

118) The method of any one of embodiments 112-117, wherein the diameter and dispersity are measured using a Malvem Mastersizer.

119) The method of any one of embodiments 112-117, wherein the diameter and dispersity are measured using Scanning Electron Microscopy.

120) The method of any one of embodiments 112-119, wherein particles of the preparation are characterized in having a water activity of <about 0.6, <about 0.3, <about 0.2, <about 0.1.

121) The method of any one of embodiments 112-120, wherein the water activity is measured using a TDL2 water activity meter.

122) The method of any one of embodiments 112-121, wherein the particle comprises core materials encapsulated by a shell materials.

123) The method of any one of embodiments 111-122, wherein the microbes are bacteria.

124) The method of embodiment 123, wherein the bacteria are probiotics.

125) The method of embodiment 124, wherein the probiotics are selected from the group comprising Bacillus coagulans, Bacillus licheniformis, Bacillus subtilis, Bifidobacterium angulatum, Bifidobacterium animalis, Bifidobacterium bifidum, Bifidobacterium breve, Bifidobacterium infantis, Bifidobacterium Bifidobacterium longum, Enterococcus faecium, Enterococcus faecalis, Lactobacillus acidophilus, Lactobacillus amylovorus, Lactobacillus alimentarius. Lactobacillus bulgaricus, Lactobacillus casei subsp. casei, Lactobacillus casei Sinrota, Lactobacillus curvatus, Lactobacillus delbrueckii subsp lactis, Lactobacillus fermentum, Lactobacillus farciminis, Lactobacillus gasseri, Lactobacillus helveticus, Lactobacillus johnsonii, Lactobacillus lacti, Lactobacillus paracasei, Lactobacillus pentosaceus, Lactobacillus plantarum, Lactobacillus reuteri, Lactobacillus rhamnosus (Lactobacillus GG), Lactobacillus sake, Lactobacillus salivarius, Lactococcus lactis, Micrococcus varians, Pediococcus acidilactici Pediococcus pentosaceus, Pediococcus acidilactici, Pediococcus halophilus, Streptococcus faecalis, Streptococcus thermophilus, Staphylococcus camosus, or Staphylococcus xylosus.

126) The method of any one of embodiments 111-124, wherein the barrier material is a solid at 25° C.

127) The method of embodiment 126, wherein the barrier material is moisture resistant.

128) The method of any one of embodiments 126-127, wherein the barrier material is characterized to melt between 30-90° C.

129) The method of any one of embodiments 126-127, wherein the barrier material is characterized to melt between 36-70° C.

130) The method of any one of embodiments 126-127, wherein the barrier material is characterized to melt between 40-60° C.

131) The composition of any one of embodiments 126-130, wherein the moisture resistant material is a lipid.

132) The composition of embodiment 131, wherein the lipid comprises, on a dry weight basis, between about 40% to about 99% (w/w) of the total mass of probiotic composition.

133) The composition of any one of embodiments 131-132, wherein the lipid is a wax.

134) The composition of embodiment 133, wherein the wax comprises paraffin wax, montan wax, microcrystalline wax, polyethylene wax, petrolatum wax, ozokerite wax, ceresin wax, beeswax, lanolin wax, spermaceti wax, tallow wax, lac wax, Chinese insect wax, ambergris wax, soy wax, camauba wax, candelilla wax, coconut wax, palm kernel wax, rice bran wax, or combinations thereof.

135) The composition of any one of embodiments 131-132, wherein the lipid is a plant oil.

136) The composition of embodiment 133, wherein the plant oil comprises fatty acid monoglyceride esters, fatty acid diglyceride esters, fatty acid triglyceride esters, coconut oil, cottonseed oil, palm oil, soybean oil, sunflower oil, fully hydrogenated palm oil, partially hydrogenated palm oil, fully hydrogenated sunflower oil, partially hydrogenated sunflower oil, fully hydrogenated soybean oil, partially hydrogenated soybean oil, fully hydrogenated vegetable oil, partially hydrogenated vegetable oil, fully hydrogenated cottonseed oil, partially hydrogenated cottonseed oil, or combinations thereof.

137) The composition of any one of embodiments 131-132, wherein the lipid is a fatty acid.

138) The composition of embodiment 133, wherein the fatty acid comprises butyric acid, pentanoic acid, hexanoic acid, heptanoic acid, caprylic acid, nonanoic acid, capric acid, lauric acid, myristic acid, palmitic acid, stearic acid, and/or arachidonic acid, or combinations thereof.

139) The method of any one of embodiments 111-138, wherein milling is achieved using at least one of the following methods: planetary milling, ball milling, burr milling, roller milling, media milling, impact milling, jet milling, high-pressure homogenization, cryo milling, hammer milling, conical milling, hand screening, or granulation/extrusion.

140) The method of embodiment 139, wherein milling is achieved using extrusion.

141) The method of any of embodiments 139-140, wherein a processing aid is included.

142) The method of any of embodiments 139-140, wherein the processing aid comprises: calcium carbonate, calcium phosphate, calcium hydroxide, calcium hydroxyapatite, zinc oxide, titanium oxide, silicon oxide, or combinations thereof.

143) The method of any one of embodiments 111-142, wherein dispersal of microbes within liquid is achieved using overhead stirrer, manual stirring, stir bar, high pressure homogenization, low pressure homogenization, sonication, ultrasonication, vortexing, or combinations thereof.

144) The method of any one of embodiments 111-143, wherein atomization is achieved using planetary milling, ball milling, burr milling, roller milling, media milling, impact milling, jet milling, high-pressure homogenization, crvo milling, hammer milling, conical milling, hand screening, or granulation/extrusion, extrusion, spray drying, fluid bed agglomeration, spray congealing, high-shear granulation, tableting, roller compaction, crosslinking, pouring, prilling, spinning disc atomization, or combinations thereof.

145) The method of any one of embodiments 111-144, wherein atomization is achieved using spinning disc atomization.

146) The method of embodiment 145, wherein the disc speed is between about 2000 and about 6000 rpm.

147) The method of embodiment 145, wherein the disc speed is between about 4000 and about 5000 rpm.

148) The method of any one of embodiments 145-147, wherein the disc temperature is between about 50° C. and about 90° C.

149) The method of any one of embodiments 145-148, wherein the cooling air temperature is between about 20° C. and about 25° C.

150) The method of embodiments 145-149, wherein the cooled compositions are collected on a powder bed.

151) The method of embodiment 150, wherein the powder is a material that reduces particle agglomeration.

152) The method of embodiment 151, wherein the powder is at least one of: spray dried starch, spray dried lactose, magnesium stearate, zinc stearate, stearic acid, silicon dioxide, zinc oxide, titanium oxide, aluminum oxide, or combinations thereof.

153) The method of any one of embodiments 111-152, wherein the relative composition of living cell to barrier material is between about 0.01% and 40% (w/w).

154) The method of any one of embodiments 111-152, wherein the relative composition of living cell to barrier material is between about 5% and 25% (w/w).

155) The method of any one of embodiments 111-154, wherein coating is achieved using at least one of the following methods: spray pan coating, fluidized bed coating, dip coating, roller coating, or sputter coating.

156) The method of any one of embodiments 111-155, wherein the coating is a material selected from at least one of the following: a carbohydrate, a protein, or combinations thereof.

157) The method of embodiment 156, wherein the carbohydrate comprises: amylose, amylopectin, cellulose, methyl cellulose, ethyl cellulose, hydroxypropyl cellulose, hydroxypropyl methyl cellulose, hydroxypropyl ethyl cellulose, cellulose triacetate, cellulose acetate succinate, cellulose acetate butvrate, cellulose acetate phthalate, hydroxvpropyl methylcellulose acetate succinate, sodium alginate, potassium alginate, calcium alginate, magnesium alginate, zinc alginate, sodium pectinate, potassium pectinate, calcium pectinate, zinc pectinate, sodium hvaluronate, potassium hyaluronate, calcium hyaluronate, magnesium hyaluronate, zinc hyaluronate, or combinations thereof.

158) The method of embodiment 156, wherein the protein comprises: whey protein, β-lactoglobulin, α-lactalbumin. casein, bovine serum albumin, ovalbumin, zein, hordein, gliadin, secalin, kafirin, avenin, or combinations thereof.

159) The method of any one of embodiments 111-158, wherein the coating material comprises, on a dry weight basis, between about 0% to about 59% (w/w) of the total mass of probiotic composition.

160) The method of any one of embodiments 111-159, wherein drying of the probiotic composition is achieved using at least one of the following methods: drierite, heating, vacuum, molecular sieves, sodium sulfate, magnesium sulfate, calcium carbonate, calcium chloride, or combinations thereof.

161) The method of embodiment 160, wherein water activity is reduced by >about 10%, >about 20%, and/or >about 30%.

162) A probiotic composition comprising microbes and one or more barrier materials wherein the barrier materials comprise, on a dry weight basis:
about 40% to about 99% (w/w) lipid, the lipid comprising at least one of a wax, a plant oil, and a fatty acid; and
about 1% to about 59% (w/w) of
   a carbohydrate;
   a protein;
   a polymer;
   or combinations thereof,
   wherein the microbes comprise probiotic bacteria.

163) The composition of embodiment 162, wherein the probiotic bacteria comprises at least one of Bacillus coagulans, Bacillus licheniformis, Bacillus subtilis, Bifidobacterium angulatum, Bifidobacterium animalis, Bifidobacterium bifidum, Bifidobacterium breve. Bifidobacterium infantis, Bifidobacterium Bifidobacterium longum, Enterococcus faecium, Enterococcus faecalis, Lactobacillus acidophilus, Lactobacillus amylovorus, Lactobacillus alimentarius, Lactobacillus bulgaricus, Lactobacillus casei subsp. Casei, Lactobacillus casei Sinrota, Lactobacillus curvatus, Lactobacillus delbrueckii subsp lactis, Lactobacillus fermentum, Lactobacillus farciminis, Lactobacillus gasseri, Lactobacillus helveticus, Lactobacillus johnsonii. Lactobacillus lacti, Lactobacillus paracasei, Lactobacillus pentosaceus, Lactobacillus plantarum, Lactobacillus reuteri, Lactobacillus rhamnosus (Lactobacillus GG), Lactobacillus sake, Lactobacillus salivarius, Lactococcus lactis, Micrococcus varians, Pediococcus acidilactici Pediococcus pentosaceus, Pediococcus acidilactici, Pediococcus halophilus, Streptococcus faecalis, Streptococcus thermophilus, Staphylococcus camosus, and Staphylococcus xylosus.

164) The composition of embodiment 163, comprising a carbohydrate, wherein the carbohydrate comprises: amvlose, amylopectin, cellulose, methyl cellulose, ethyl cellulose, hydroxypropyl cellulose, hydroxypropyl methyl cellulose, hydroxypropyl ethyl cellulose, cellulose triacetate, cellulose acetate succinate, cellulose acetate butyrate, cellulose acetate phthalate, hydroxypropyl methylcellulose acetate succinate, sodium alginate, potassium alginate, calcium alginate, magnesium alginate, zinc alginate, sodium pectinate, potassium pectinate, calcium pectinate, zinc pectinate, sodium hyaluronate, potassium hyaluronate, calcium hyaluronate, magnesium hyaluronate, zinc hyaluronate, or combinations thereof.

165) The composition of embodiment 163, comprising a protein, wherein the protein comprises: whey protein, β-lactoglobulin, (α-lactalbumin, casein, bovine serum albumin, ovalbumin, zein, hordein, gliadin, secalin, kafirin, avenin, or combinations thereof.

166) The composition of embodiment 163, wherein the composition comprises a particle preparation comprising the microbes, wherein a diameter of the composition is about 10% to about 30% larger than a diameter of the particle preparation.

167) The composition embodiment 166, wherein particles of the particle preparation are characterized in comprising a water activity from about 0.1 to about 0.3.

168) The composition of embodiment 167, wherein particles of the particle preparation comprise a dispersity from about 0.1 to about 0.4.

169) The composition of embodiment 168, comprising an excipient component comprising calcium carbonate.

170) The composition of embodiment 163, wherein the probiotic bacteria comprises at least one of Lacticaseibacillus rhamnosus (HN001), Bifidobacterium lactis (HN019), Bifidobacterium lactis (BI-07), and Lactobacillus acidophilus.

171) The composition of embodiment 170, wherein the probiotic bacteria comprises at least one of Lacticaseibacillus rhamnosus (HN001) and Bifidobacterium lactis (HN019).

172) The composition of embodiment 162, wherein the lipid comprises at least one of paraffin wax, hydrogenated palm oil, and palmitic acid.

173) The composition of embodiment 170, comprising about 5% (w/w) of the probiotic bacteria and about 95% (w/w) of the lipid, wherein the lipid comprises at least one of paraffin wax, hydrogenated palm oil, and palmitic acid.

174) The composition of embodiment 170, comprising about 35% (w/w) of the probiotic bacteria and about 65% (w/w) of the lipid, wherein the lipid comprises at least one of paraffin wax, hydrogenated palm oil, and palmitic acid.

175) The composition of embodiment 170, comprising from about 5% to about 35% (w/w) of the probiotic bacteria, and from about 65% to about 95% (w/w) of the lipid, wherein the lipid comprises at least one of paraffin wax, hydrogenated palm oil, and palmitic acid.

176) A particle comprising the composition of embodiment 162, wherein the particle comprises a diameter in a range from about 60 μm to about 300 μm.

177) The particle of embodiment 176, wherein the particle comprises a diameter in a range from about 100 μm to about 250 μm.

178) An extrudate comprising:
about 85% (w/w) plant oil;
about 10% (w/w) excipient component; and
about 5% (w/w) probiotic bacteria.

179) The extrudate of embodiment 178, wherein the plant oil comprises hydrogenated palm oil.

180) The extrudate of embodiment 179, wherein the excipient component comprises CaCO3.

181) The extrudate of embodiment 179, wherein the probiotic bacteria comprises Bifidobacterium lactis (HN019).

182) The composition of embodiment 162, wherein the barrier material comprises:
a first inner layer comprising at least one of a hydrophilic material and a water soluble material; and
a second outer layer comprising at least one of a hydrophobic material and a fat soluble material.

183) The composition of embodiment 162, wherein the barrier material comprises a polymer component comprising at least one of a bile-responsive polymer, a pH-responsive polymer, and a microbiome-responsive polymer.

184) The extrudate of embodiment 178, wherein the excipient component comprises at least one of microcrystalline cellulose, a starch, and maltodextrin.

185) The composition of embodiment 162, comprising an excipient component comprising at least one of anti-caking component, anti-agglomerating component, anti-clumping component, anti-aggregating component, a surfactant component, a plasticizing component, an acid scavenger, an oxygen scavenger, a moisture scavenger, a water scavenger, a desiccant, and/or a combination thereof.

186) The composition of embodiment 162, wherein the probiotic bacteria is encapsulated with at least one of a desiccant and a cryoprotectant.

187) The composition of embodiment 162, comprising a carotenoid comprising at least one of alpha-lipoic acid, astaxanthin, adonixanthin, adonirubin, beta-carotene, coenzyme Q10, lutein, lycopene, zeaxanthin, and meso-zeaxanthin.

188) The composition of embodiment 162, wherein the probiotic bacteria comprises at least one spore forming species.

189) The method of embodiment 111, wherein the liquid into which the milled microbes are dispersed comprises a surfactant comprising sodium dodecyl sulfate.

190) The composition of embodiment 185, wherein the excipient imparts a change to at least one of
(i) an environment within the particle composition; and
(ii) a local environment in which the probiotic composition resides;
wherein a change to the environment within the particle composition comprises at least one of a pH change, an oxygen concentration change, and a water concentration change, and
wherein local environment comprises at least one of a stomach, a food matrix, and a beverage.

191) A food product coated with the probiotic composition of embodiment 162, the food product comprising at least one of a gelatin-based matrix and a pectin-based matrix.

192) The food product of embodiment 191, wherein the at least one gelatin-based matrix and/or pectin-based matrix comprises a water activity in a range from 0.50 plus or minus 0.01 to 0.70 plus or minus 0.01.

### EXEMPLIFICATION

The following examples are intended to illustrate but not limit the disclosed embodiments. The following examples are useful to confirm aspects of the disclosure described above and to exemplify certain embodiments of the disclosure.

These non-limiting examples demonstrate particular features and advantages of provided technologies—e.g., of provided probiotic compositions comprising microbes and barrier materials.

Among other things, provided probiotic compositions may be characterized by significant improvements, including, for example, improved physicochemical stability, cell viability, controlled release, anti-caking, anti-agglomeration, anti-clumping, anti-aggregation, and/or amenability to combination with other component(s) of a product (e.g., a nutraceutical product that may in many embodiments be a consumable product). As exemplified herein, provided probiotic compositions achieve one or more of the following advantages: (i) amenability to combination (e.g., mixing) with other components or materials, which enables payload components to be combined with and/or incorporated into complex foods and/or beverages (e.g., milk) and/or ingredients (e.g., sachets); (ii) maintenance of cell viability in these food and/or beverage matrices, humid storage environments, or digestive environments; (iii) low water content, even when characterized by high water activity: (iv) technological modularity that permits control over particle size characteristic(s) (e.g., average particle [e.g., microparticle] size and/or size distribution), loading, and/or release; and (vi) anti-caking, anti-agglomeration, anti-clumping, and/or anti-aggregation.

### A. Example 1: Morphology of Exemplary Particle Preparations

The morphology of non-limiting exemplary embodiments of the disclosed probiotic compositions is depicted by brightfield and scanning electron micrographs.

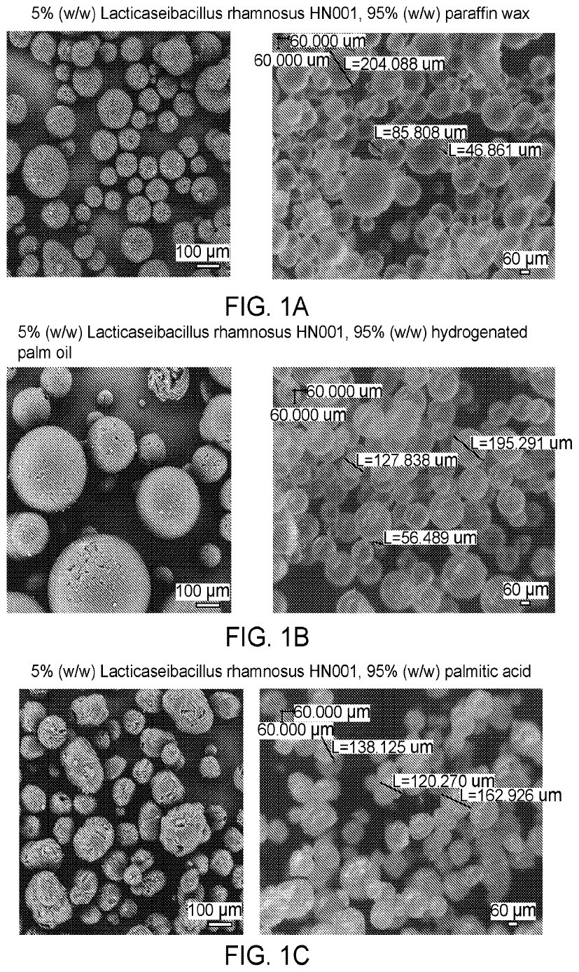

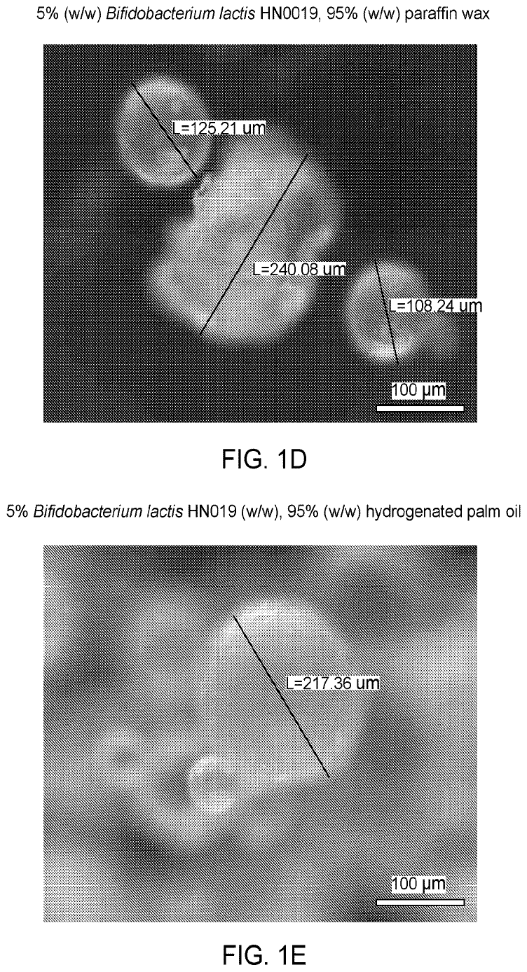

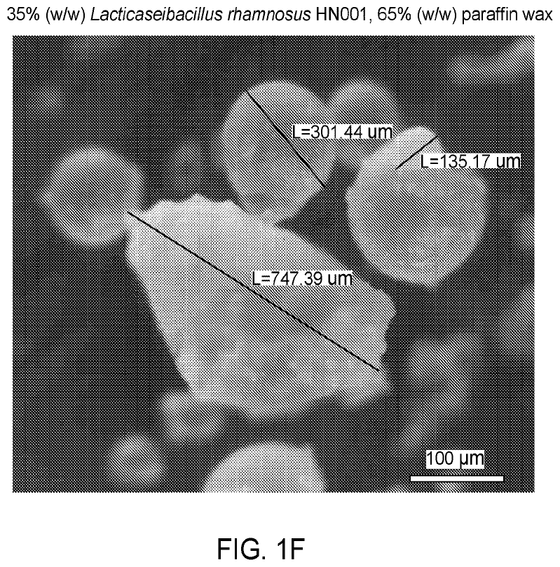

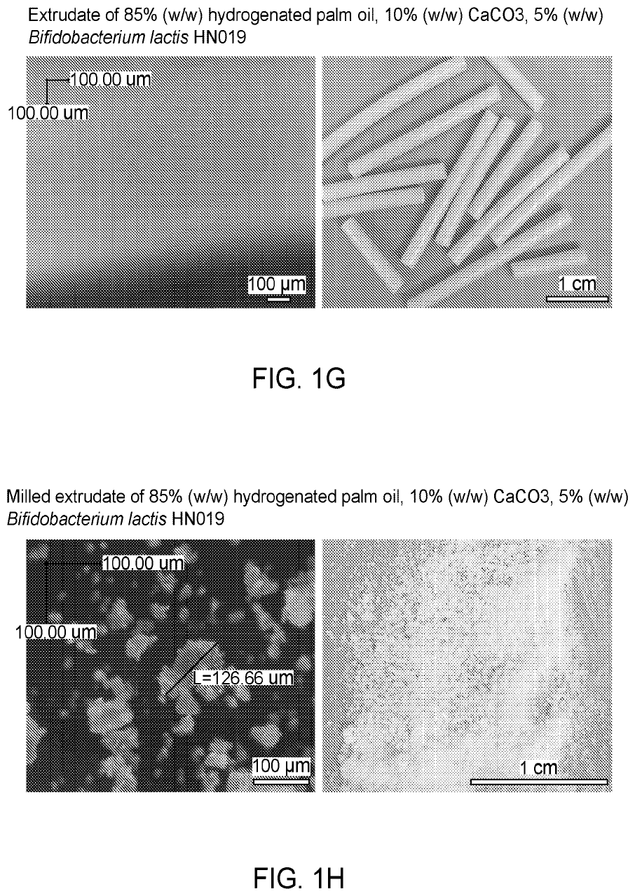

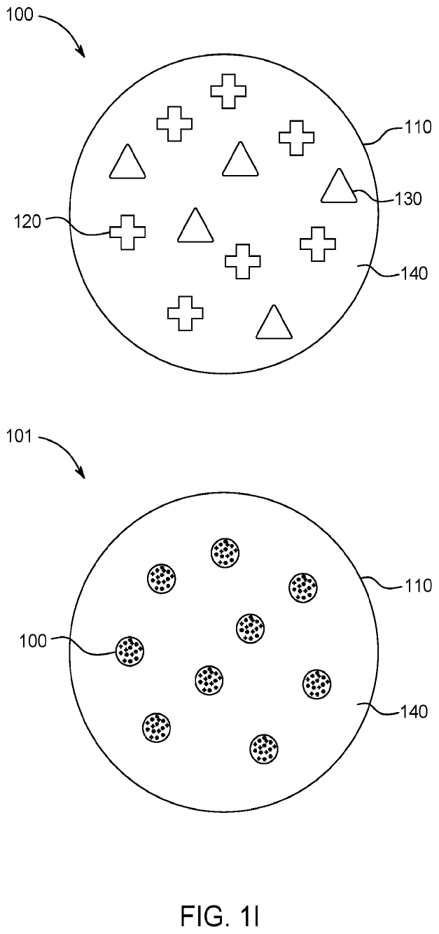

Brightfield micrographs, FIG. 1A-H, and Scanning Electron Micrographs, FIG. 1A-C, depict exemplary payload-containing particles as provided by the present disclosure. The provided probiotic compositions comprise various shapes (e.g., spherical particles, circular particles, disc-shaped particles, irregular-shaped particles, etc.) of consistent or various size distribution, with smooth or rough surfaces. Brightfield micrographs in FIG. 1A-H are taken with a compound microscope. As shown in FIGS. 1A-1C (right images) probiotic particles and/or preparations of the present embodiments may include translucence and even transparence, which in some embodiments provides the additional benefit of not needing to be colored in order to visually blending in with food and beverage products into which they are being mixed.

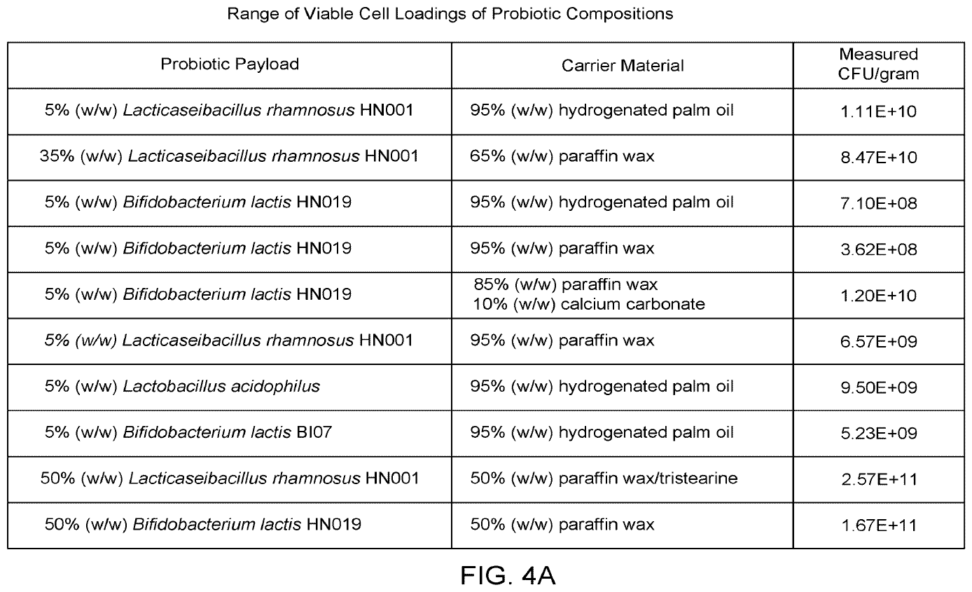

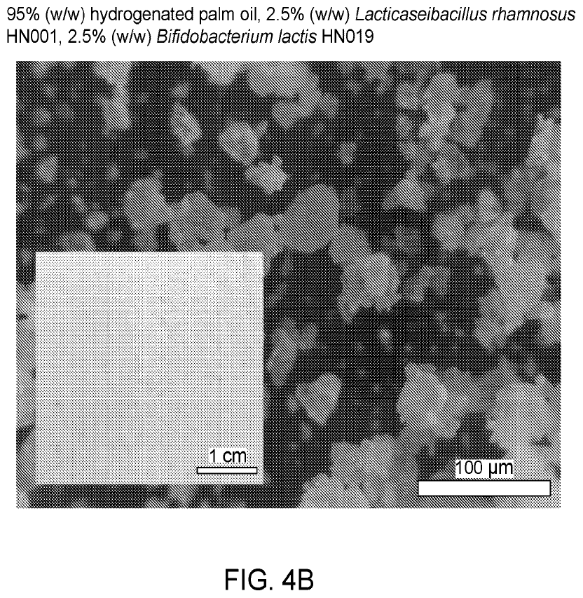

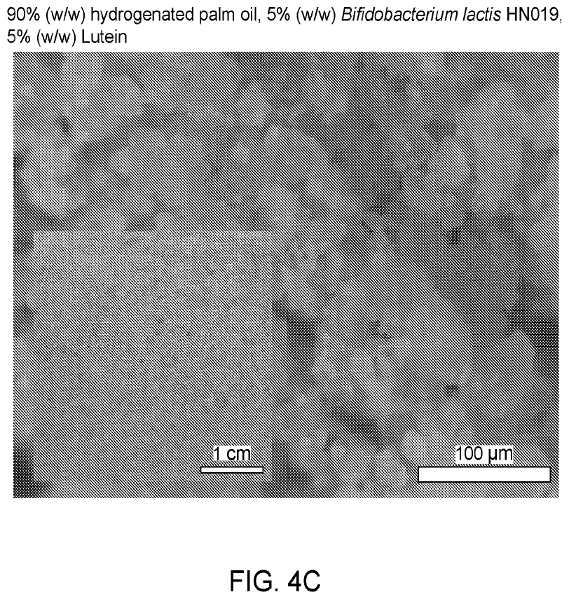

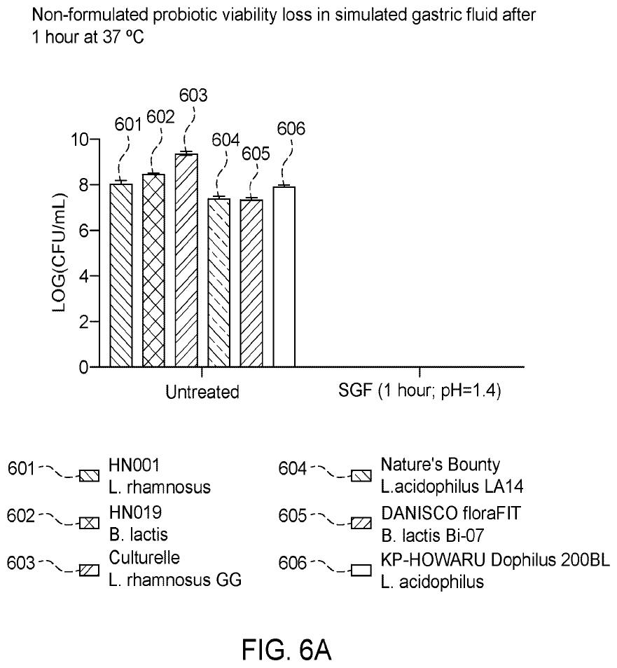

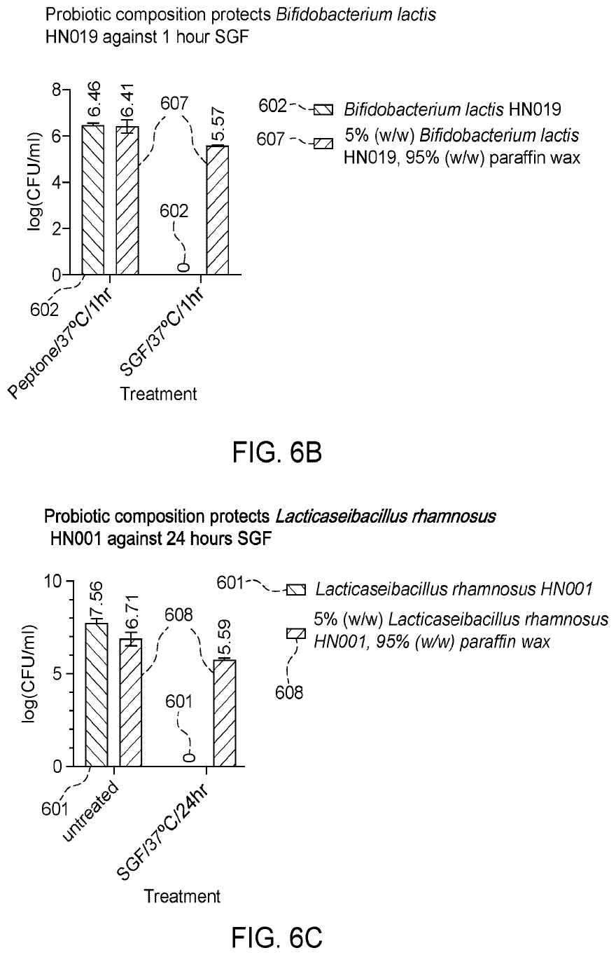

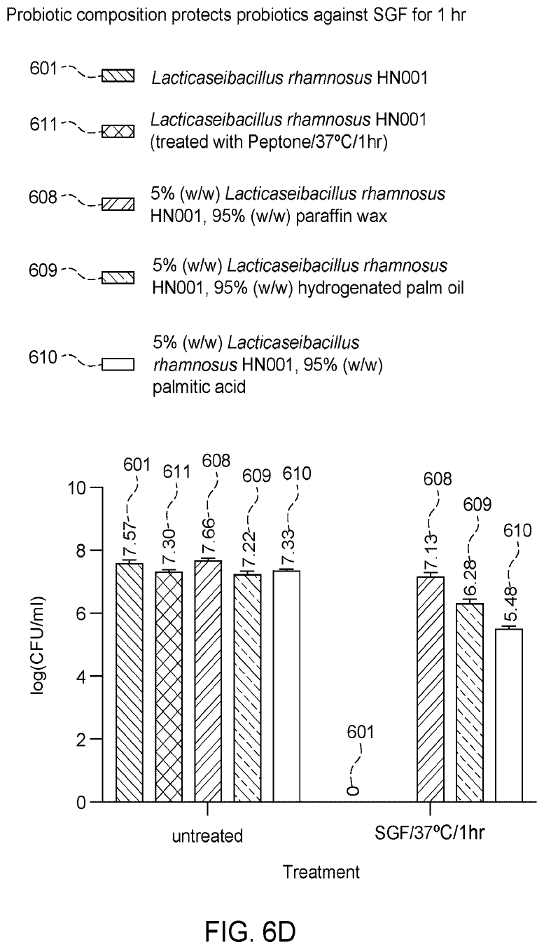

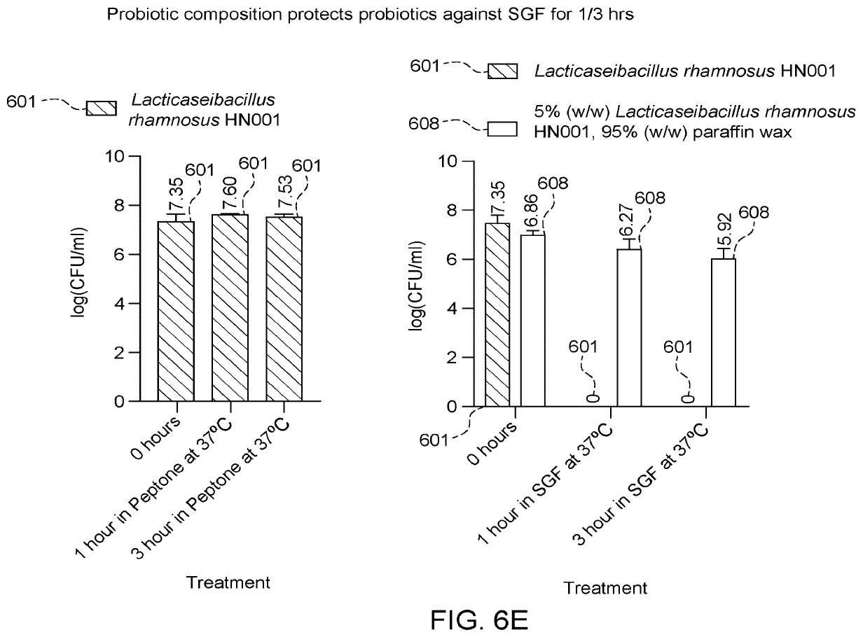

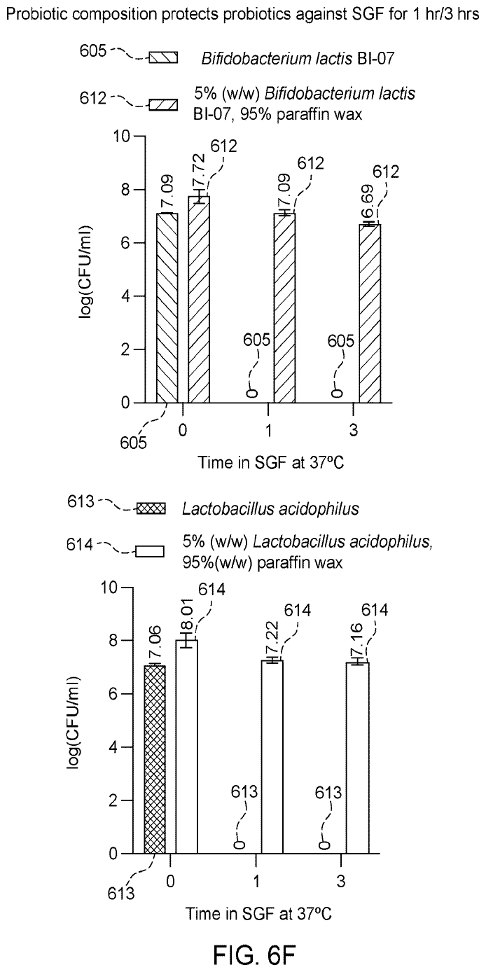

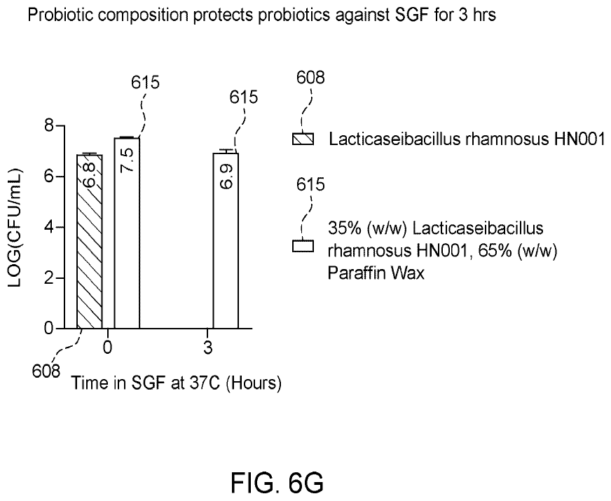

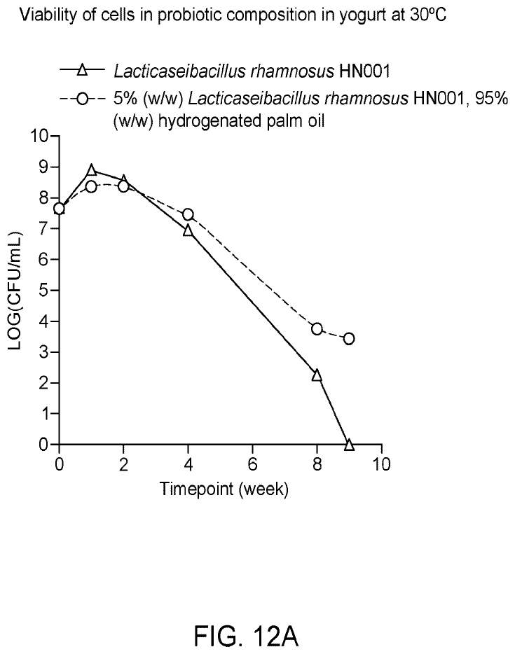

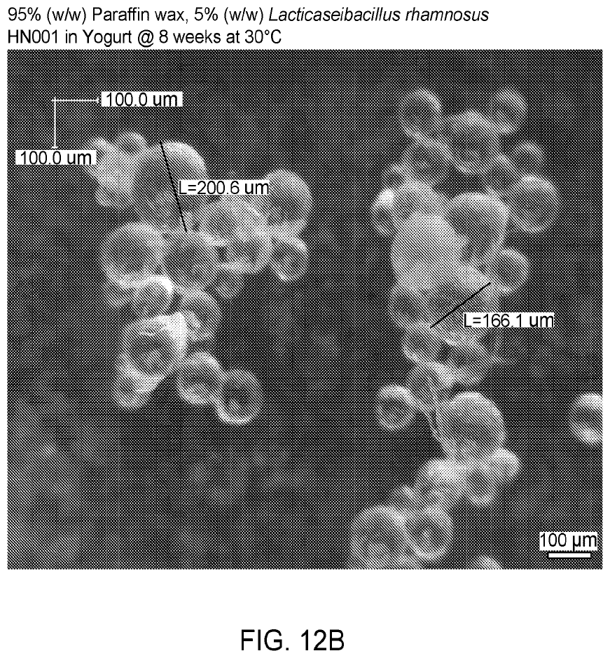

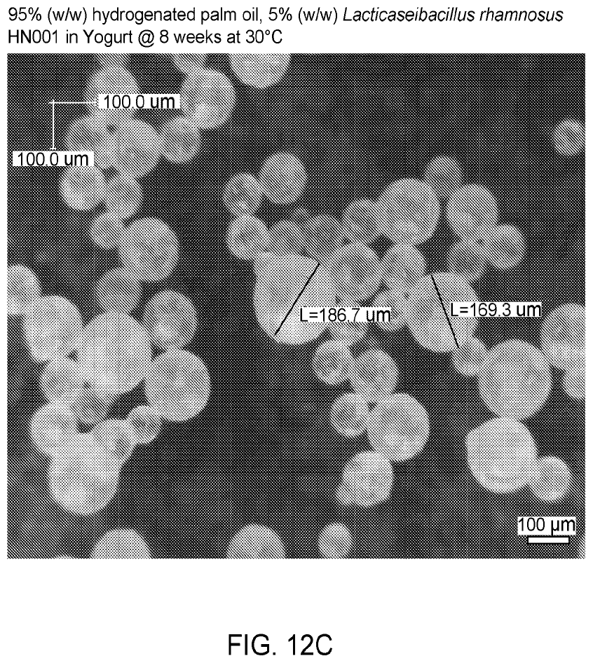

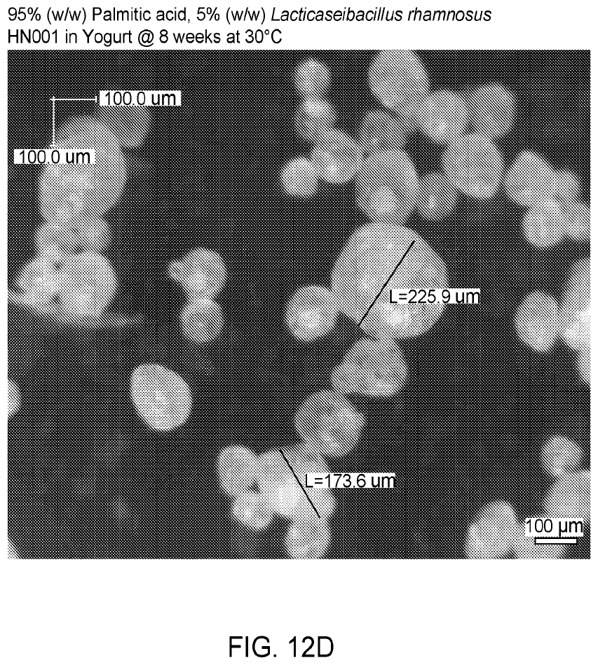

These particular exemplified probiotic compositions comprise either paraffin wax or hydrogenated palm oil as lipid components. These non-limiting exemplary embodiments of barrier materials confer several performance advantages to an encapsulated microbes (e.g. lactobacillus) versus a non-encapsulated payload (e.g. lactobacillus) in terms of controlled loading (FIG. 4A), stability and enhanced survival in simulated gastric fluid (FIG. 6A-6G), stability in milk powder (FIG. 11A-11B), stability in yogurt (FIG. 12A), and/or stability in humid environments (FIG. 15A-15B).

### B. Example 2: Preparation of Particle Preparations Via Prilling

This example describes a process of producing probiotic particles via prilling, by utilizing meltable properties of lipid materials. FIG. 1F depicts an exemplary probiotic particle that has been produced via this method.

In an exemplary protocol of manufacture, paraffin wax, or other lipid component, is melted with an induction heater at a temperature 5-15° C. above the melting point (which in some embodiments may be in a range from about 22° C. to about 80° C.). A spinning disk is prewarmed to a temperature about 5° C. above the induction heater temperature, and set at a speed between 4500-5000 rpm. The probiotic component is added to the molten lipid and mixed in completely, with care to minimize time in the molten lipid. When completely mixed, the molten mixture is fed onto the center of the spinning disc, during which atomized particles are dispersed to the edges and solidified upon cooling. The resulting particles (for example, the 35% Lacticaseibacillus rhamnosus HN001/65% paraffin wax particles shown in FIG. 1F) may be collected and immediately analyzed for particle size, water activity, etc.

### C. Example 3: Preparation of Encapsulated Probiotic Particles Via Extrusion

In an exemplary protocol of manufacture, probiotic compositions are prepared via extrusion and milling. FIGS. 1G and 1H illustrate example particle preparations that have been coated with a secondary coating via this method.

Preparing payload component: Optionally, the payload is micronized prior to extrusion. A DynoMill Multi-Lab bead mill is used to pre-micronize payload component with the following settings: (i) 3000 rpm, (ii) 250 mL/min pump/feed rate, and (iii) N2 purging over the beaker headspace. (Other suitable milling equipment may also be used). 500 mL deionized water is purged with N2 for 30 minutes and is combined with: (i) up to 0.5 g of excipient, (ii) 50 g of a nutraceutical payload (e.g., a probiotic), and (iii) 400 mL of 0.65 mm beads.

Preparing particles comprising payload component: Lipid material or other polymer component is combined with the dried payload component and the combination is mixed. In some embodiments, plasticizers such as calcium carbonate, soybean oil, vitamin E, or other vegetable oils are optionally added up to 20% w/w and mixed with a mechanical mixer. The resulting mixture of polymer component and payload component is extruded using a Thermo Haake Minilab II at a temperature in the range of 40° C. up to 75° C. and a screw speed between 30-90 RPM. The resulting extrudate is milled (e.g., via cryo mill, jet mill, or other mill, or multiple in sequence) to produce a particle preparation (i.e., probiotic composition) comprising particles of 2-400 m in size with a payload component loading of about 1-25%.

### D. Example 4: Particle Size Distribution in Exemplary Particle Preparations

Size distributions, generated by a Malvern Laser Diffraction Particle Sizer, of non-limiting exemplary embodiments of provided probiotic compositions are described in the following example.

Particle size and particle size distributions influence sensory experience (e.g., mouth feel), ease of mixing with food and/or beverage products, ease of mixing with other formulation constituents, other constituents during formulation, and/or rate of release of payloads.

Several size distributions of the probiotic compositions provided in this disclosure may be obtained using the method of manufacture further outlined in the disclosure. In the following preferred non-limiting exemplified embodiments, a cone-milling process was utilized to modify the size distribution of a lyophilized cake of microbes. Additionally, a spinning disc atomization procedure, with disc speed and temperature of 4500 rpm and 78° C., respectively, was used to modify the size distribution of compositions comprising a lipid barrier material encapsulating a microbes. In some embodiments, cone-milling processes may be substituted with other milling processes including, but not limited to, planetary milling, ball milling, roller milling, media milling, impact milling, jet milling, high-pressure homogenization, cryo milling, hammer milling, hand screening, or extrusion. The same milling processes may be used to select or further refine the size distribution of compositions comprising a lipid barrier material and microbes. The speed and temperature of the spinning disc apparatus are parameters that control the resulting size distribution of the composition. In some embodiments, atomization, and resulting size distribution, can be achieved through alternate techniques including, but not limited to, prilling, electrostatic spray, high pressure spray, fluid flow, pouring, or ultrasonic spray.

FIG. 2A-2B illustrate particle size distributions of a dispersed lyophilized cake of viable probiotic bacteria, either before (FIG. 2A) or after (FIG. 2B) a cone milling process. FIG. 2C-2E illustrate particle size distributions of exemplary compositions of Lactobacillus rhamnosus with a barrier material of either paraffin wax or hydrogenated palm oil. These exemplary compositions of 5% Lacticaseibacillus rhamnosus HN001 in paraffin wax (FIG. 2C), 5% Lacticaseibacillus rhamnosus HN001 in hydrogenated palm oil (FIG. 2D), 35% Lacticaseibacillus rhamnosus HN00 in paraffin wax (FIG. 2E), 5% Bifidobacterium lactis HN019 in hydrogenated palm oil (FIG. 2F), 5% Bifidobacterium lactis HN019 in paraffin wax (FIG. 2G), 5% Bifidobacterium lactis BI-07 in paraffin wax (FIG. 2H), and 5% Lactobacillus acidophilus in paraffin wax (FIG. 2I) exhibit unimodal size distribution, with typical diameter between about 100 μm to about 250 μm. Particle diameters greater than 500 μm alter the mouth-feel of a food, beverage, and/or pharmaceutical product; advantageous embodiments of the provided compositions, as a result, exhibit average particle diameter less than about 500 μm. Table 1 shown in Example 16 provides a summary of particle diameters for the measured particles shown in the Figures. All of the measured particles included a diameter in a range from about 40 μm to about 750 μm. All but one of the measured particles included a diameter in a range from about 40 μm to about 310 μm. A plurality of the remaining measured particles included a diameter in a range from about 40 μm to about 260 μm, or from about 60 μm to about 260 μm, or from about 100 μm to about 250 μm.

### E. Example 5: Preparation of Secondary Coated Probiotic Particles Via Pan Coating

This example describes a process of creating a secondary coat around probiotic particles via benchtop pan coating, by creating an organic film over the original probiotic particle.

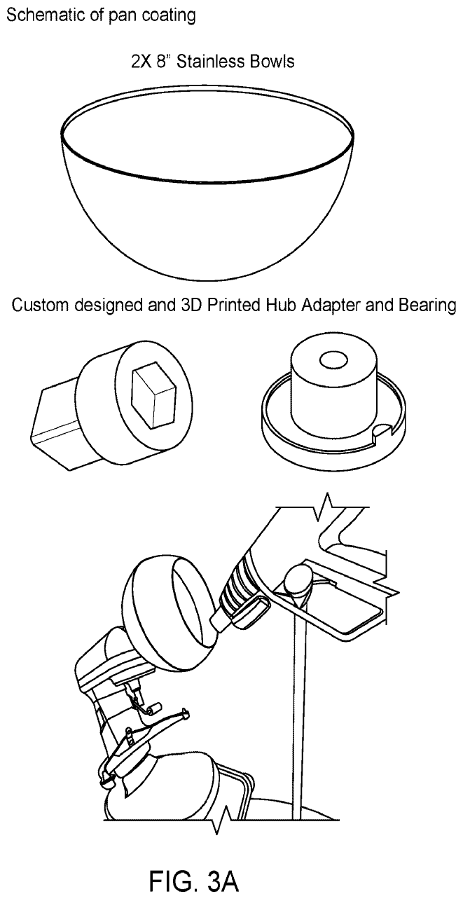

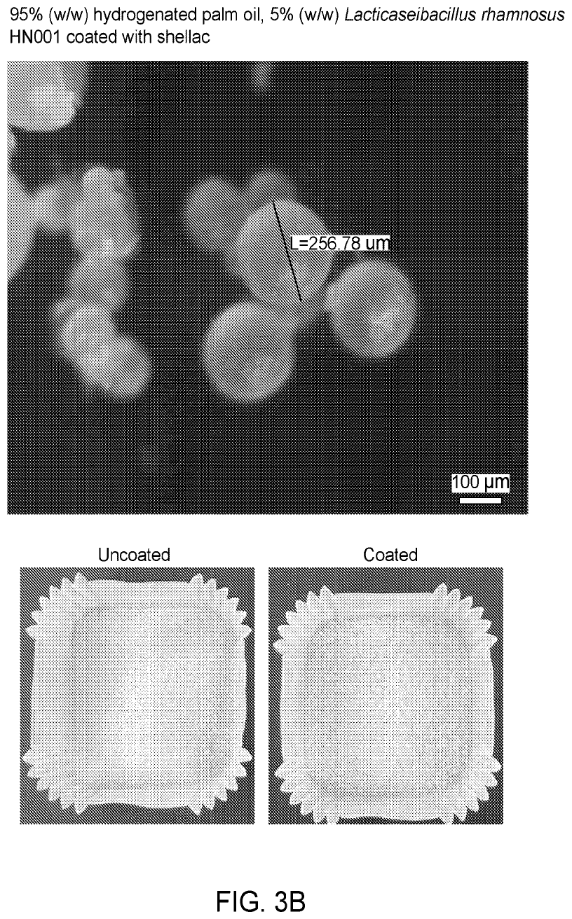

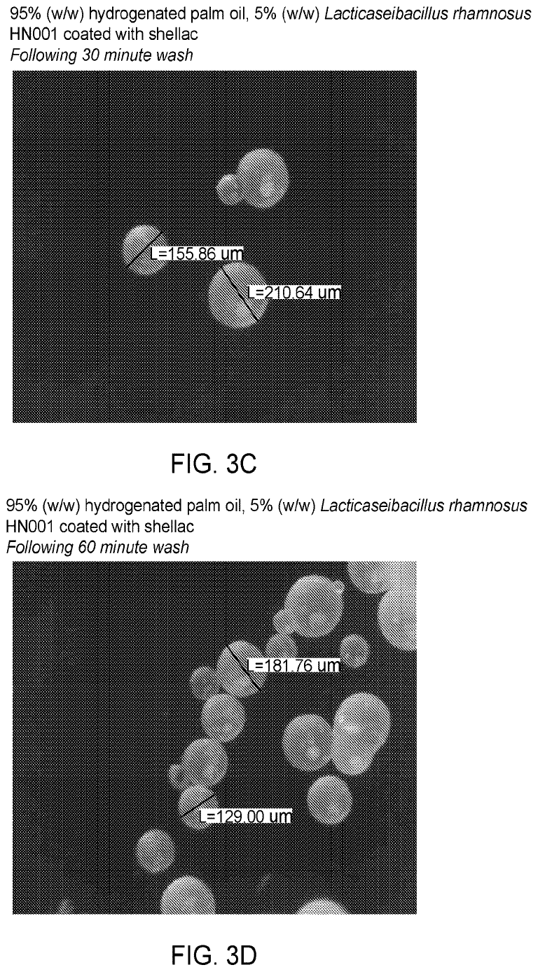

A round pan is adapted to fit a KitchenAid® stand mixer. The mixer is tilted backwards roughly 30° and a heat gun is positioned to point towards the bottom of the pan. A visual representation of the setup is depicted in FIG. 3A. Particles are rotated at a rate of 0.2-2 Hz with an inlet temperature of 30° C.-50° C. The particles are sprayed with an atomized solution of coating polymer, dissolved in a suitable solvent (e.g., ethanol), allowing for the solvent to evaporate after adding several sprays. Once application of the coating is complete, particles are subjected to inlet air temperature at 30° C.-50° C. until the coating is completely dry. Coated particles are exemplified in brightfield micrographs FIG. 3B and remain visibly intact after washing in an aqueous peptone solution for 30 (FIG. 3C) and 60 (FIG. 3D) minutes.

### F. Example 6: Payload Component Loading of Particle Preparations

The example presented below demonstrates that the ratio of microbes (e.g., probiotic) to lipid component is selected during the manufacturing process to control the relative loading of cells within the composition. Relative cell loading within a probiotic composition may influence cost, environmental exposure, and dose selection. In certain preferred embodiments, the relative composition of microbes within the probiotic composition is maximized to reduce waste and required dose.

FIG. 4A includes a table of cell loadings achieved within certain exemplary probiotic compositions comprising Lacticaseibacillus rhamnosus, Bifidobacterium lactis, and Lactobacillus acidophilus embedded within matrices of paraffin wax, fully hydrogenated palm oil (with and without calcium carbonate), or palmitic acid. Relative loading of microbes in these exemplary embodiments, determined using the oil extraction method for cell enumeration described in Example 10, were in a range from about 0.1% (w/w) to about 50% (w/w).

Probiotic compositions as described herein can homogeneously incorporate more than one payload at once. For example, FIG. 4B is a micrograph depicting an exemplary probiotic composition that comprises 2.5% of both Lactobacillus rhamnosus and Bifidobacterium lactis. Further, probiotic compositions as described herein can homogeneously incorporate compatible nutraceutical particles as a payload component. FIG. 4C presents a micrograph of an exemplary probiotic composition that comprises hydrogenated palm oil, Bifidobacterium lactis at 5% loading, and a nutraceutical compound (e.g., Lutein) at 5% loading.

### G. Example 7: Preparation of Probiotic Particles Via Melt-Emulsion

In an exemplary protocol of manufacture, probiotic compositions are prepared via benchtop melt-emulsion. FIG. 4B depicts an exemplary probiotic composition produced via the following method.

In one exemplary protocol of benchtop melt-emulsion, probiotic bacteria in powder form are warmed to room temperature and weighed to a desired quantity (e.g., 500 mg) with an analytical balance. In a separate aluminum dish or bowl, a desired quantity of lipid (for e.g. 9.5 g) is weighed with an analytical balance. (The aluminum dish or bowl may also be made of other suitable materials such as steel, glass, Teflon, and other similar non-fouling surfaces). The dish with lipid is heated to 5-10° C. above the melting point of the wax (e.g., any wax, lipid, hydrogenated palm oil, and/or encapsulating material as described herein) which in some embodiments may be in a range from about 46° C. to about 68° C.) until the solid has completely melted. The temperature of the hot plate is subsequently reduced to the melting point of the lipid (which in some embodiments, may be in a range from about 22° C. to about 40° C.) and the weighed quantity of bacteria powder is added to the molten matrix in proportion. The suspension is vigorously mixed by spatula to disperse clumps and uniformly distribute the probiotics for 1-2 minutes. The aluminum dish is removed from heat under continuous mechanical mixing. The blend is allowed to cool to room temperature (about 22° C., +/−2° C.), followed by incubation in a −20° C. freezer to accelerate cooling. Particles are generated from the solidified formulation via burr milling.

Alternatively, a hotplate with temperature control is heated to 5-15° C. above. Wax material was heated until completely molten. Probiotics are weighed into a small weigh boat and then slowly added (with stirring) into the molten wax. A metal spatula is used to mix the wax and probiotic material. Probiotic amount is calculated based on a desired loading percentage. A plastic beaker or metal container with small holes is placed into a liquid nitrogen dewar and then the dewar is filled with liquid nitrogen. The wax and probiotic mixture is rapidly poured into the liquid nitrogen dewar. The plastic beaker or metal container is raised and allows liquid nitrogen to flow out. The plastic beaker or metal container is removed and the resulting probiotic preparation is collected. Probiotic particles are generated from the solidified formulation via burr milling.

### H. Example 8: Viability of Cells within Provided Probiotic Compositions

Those skilled in the art will recognize that cell viability is often lost during the encapsulation process due to factors including, but not limited to, temperature, shear forces, moisture content, or combinations thereof. The following example demonstrates the novelty of the probiotic compositions provided in this disclosure by encapsulation of cells without a substantial reduction in viability. In certain embodiments, the barrier materials of the probiotic composition are solid in a range from about 25° C. to about 37° C. yet yield free-flowing liquids amenable to incorporation of living cells upon mild heating to between about 45° C. and about 65° C. In further embodiments, the barrier materials are known to those skilled in the art as hydrophobic.

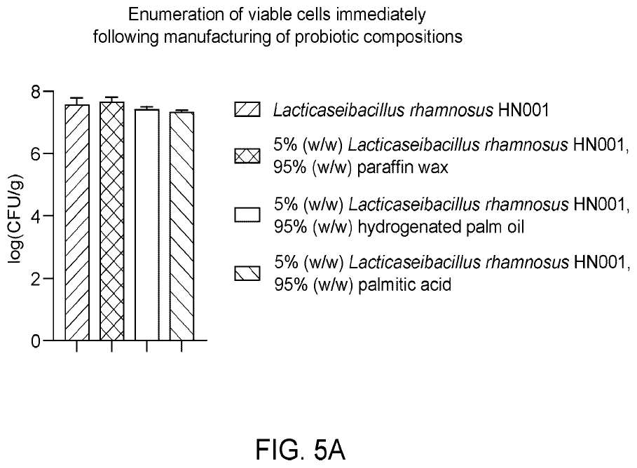

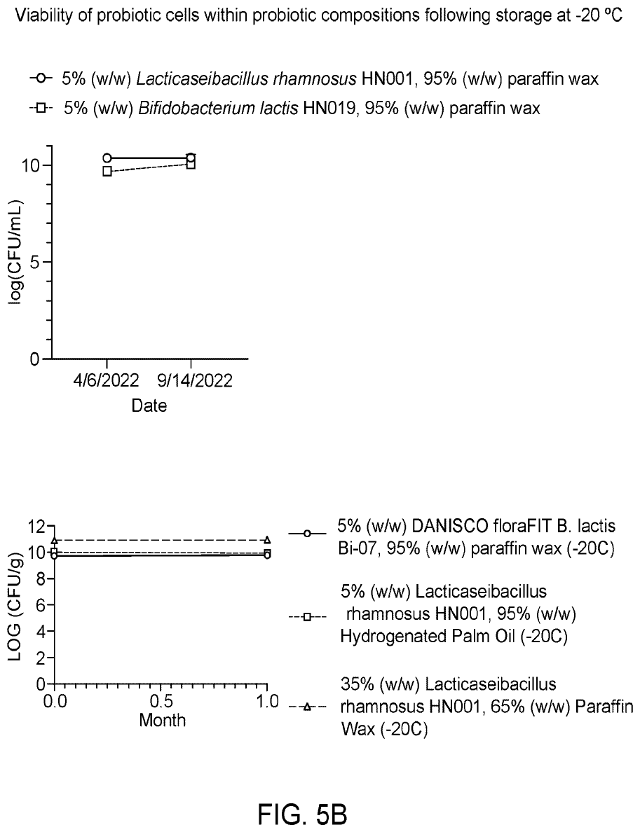

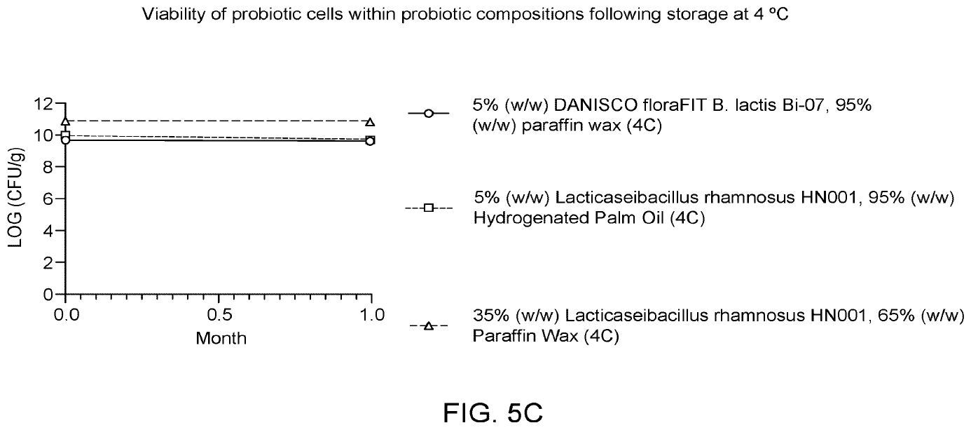

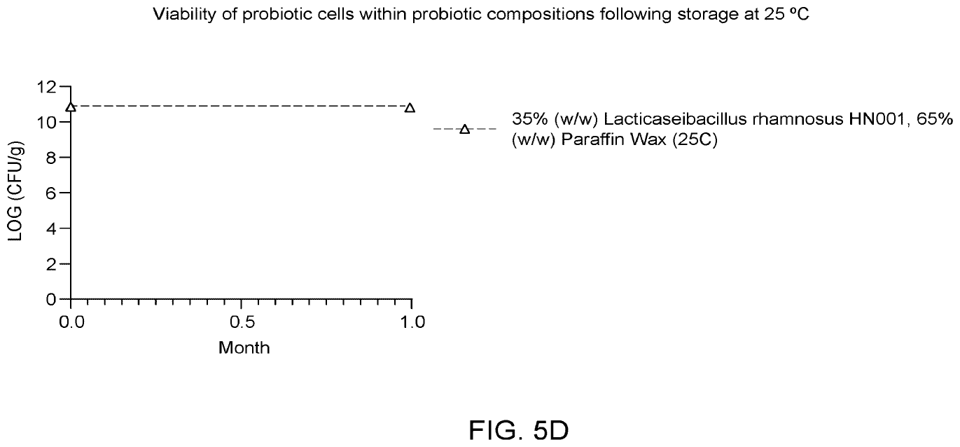

In a non-limiting exemplary embodiment of the invention, as shown in FIG. 5A, a probiotic composition comprising Lactobacillus rhamnosus encapsulated within a barrier material of paraffin wax retains cell viability relative to unformulated Lactobacillus rhamnosus (i.e., there is no statistically significant difference in the viability between encapsulated compositions of the present disclosure, and un-encapsulated compositions). This demonstrates that manufacturing approaches do not inhibit, damage and/or kill the nutraceutical payload.

In certain embodiments of the invention, probiotic compositions are stored at a predetermined temperature for a predetermined time, under a predetermined head gas being further characterized by the viability of the encapsulated living cells under these conditions. Alternatively or additionally, in some such embodiments, the storage conditions may comprise high temperature (e.g., up to or above about 50° C.), presence of water/humidity, acidic liquid medium (e.g., simulated gastric fluid), and/or presence of a dairy product. In some such embodiments, a stored composition maintains at least about 50% of one or more payload components in relation to the starting amount (100%) and/or at least about 10⁹ colony forming units of probiotics. For example, in some embodiments, provided probiotic particles are characterized in that probiotic viability is maintained after incorporation into the lipid-based particle (FIG. 5A) or after storage for 1-5 months at −20° C. (FIG. 5B), or after storage for 1 month at 4° C. (FIG. 5C), or after storage for 1 month at 25° C. (FIG. 5D).

### I. Example 9: Stability of Particles and Payload Components in Fluids

The following exemplary embodiments of the invention illustrate the ability of the disclosed nutraceutical particles to preserve encapsulated cell viability when dispersed in simulated biological fluids (e.g., simulated gastric fluid (SGF), simulated intestinal fluid (SIF), simulated rumen fluid (SRF) and/or tryptic soy broth (TSB)).

In one exemplary instance, shown in FIG. 6A-6E, probiotic compositions (expressed as % w/w) of 5% Bifidobacterium lactis HN019 in paraffin wax (FIG. 6B), 5% Lacticaseibacillus rhamnosus HN00l in paraffin wax (FIG. 6C, 6D, 6E), 5% Lacticaseibacillus rhamnosus HN001 in hydrogenated palm oil (FIG. 6D), 5% Lacticaseibacillus rhamnosus HN001 in palmitic acid (FIG. 6D), 5% Bifidobacterium lactis BI-07 in paraffin wax (FIG. 6F). 5% Lactobacillus acidophilus in paraffin wax (FIG. 6F), and 35% Lacticaseibacillus rhamnosus HN001 in paraffin wax exhibit <25% log(CFU/g) loss in microbes following incubation in simulated gastric fluid (pH 1.4) at 37° C. up to 24 hours versus nearly 100% log(CFU/g) loss for unformulated bacteria following even <1 h incubation.

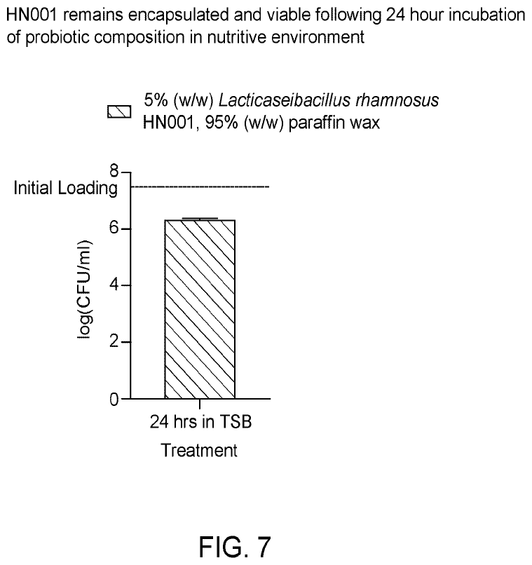

FIG. 7 demonstrates that Lactobacillus rhamnosus in probiotic particles remain encapsulated and viable after being subjected to a nutritive environment in trvptic soy broth for 24 hours, and subsequently washed before extraction and enumeration. Probiotics in the particles were retained and stabilized in the particle, and did not undergo a typical growth cycle that might occur in the presence of nutrients and water.

An exemplary protocol for exposing probiotic compositions to simulated biological fluids (e.g. simulated gastric fluid (SGF)) involves preparing a solution of SGF (hydrochloric acid, sodium chloride. Pepsin) at pH 1.4, followed by sterilization via 0.22 μm filtration. Treatment vessels may include 100 mL sample cups, to which 50 mL of SGF solution is added, and a maximum of 1% w/w or w/v of probiotic composition is added. The pH after sample addition is measured to confirm that it remains below 1.5. Treatment vessels are covered and incubated on a shaker at 30 rpm and 37° C. Following a 1 hour incubation, treatment vessels are cooled to 25° C. and neutralized by the addition of 25 mL of a buffered peptone solution. The neutralized solution is then filtered via Buchner funnel to isolate particulates for bacterial enumeration as described in Example 10. Bacterial enumeration of aqueous controls is achieved by sampling 100 μL directly from the neutralized solution (75 mL), dilution, and enumeration as described in Example 10.

An exemplary protocol for determining the resistance of probiotic compositions to simulated intestinal fluid and/or other simulated biological fluid (e.g., simulated intestinal fluid with bile) involves making/obtaining stock solutions of fluid and filter sterilizing. For each sample, a sheet of filter paper is placed into an aluminum pan and dried in an oven for at least 2 hours at 105° C., the mass of which is taken immediately prior to collection of probiotic compositions. To 800 mg of each sample, in a 15 mL polypropylene falcon tube, is added 3.08 mL of simulated gastric fluid or 7.20 mL of simulated intestinal fluid with bile. The resulting suspension is placed in a rotisserie to rotate at 40 rpm and 37° C. Following incubation for a predetermined period of time, samples are removed, filtered, and placed in an aluminum dish for drying overnight at 105° C. (The aluminum dish may also be made of other suitable materials such as steel, glass, Teflon, and other similar non-fouling surfaces). After drying, the final weight of the pan, paper, and samples is measured in order to calculate percent recovery and percent loss.

### J. Example 10: Enumeration of Microbes within Provided Probiotic Compositions

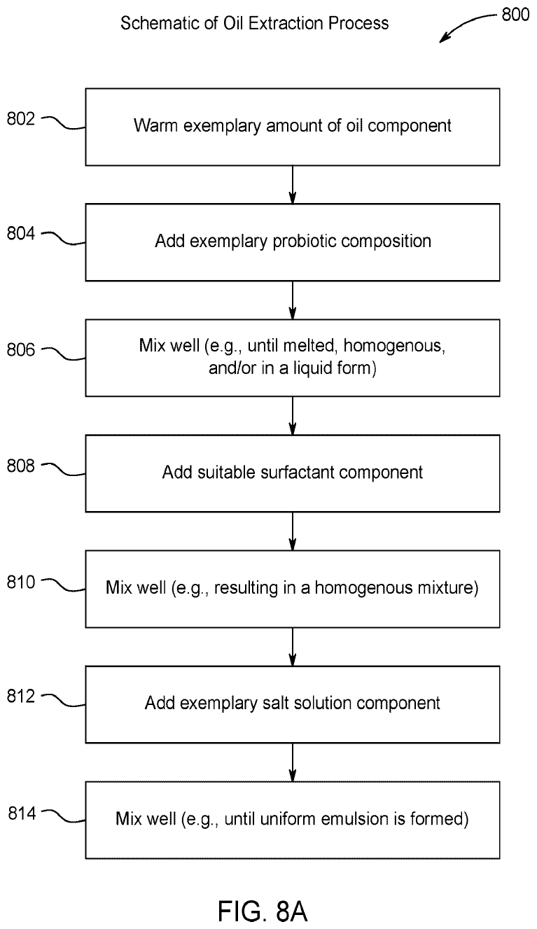

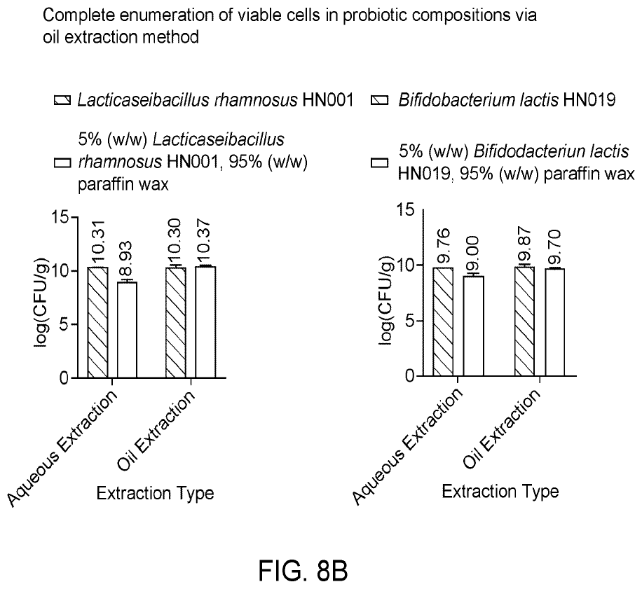

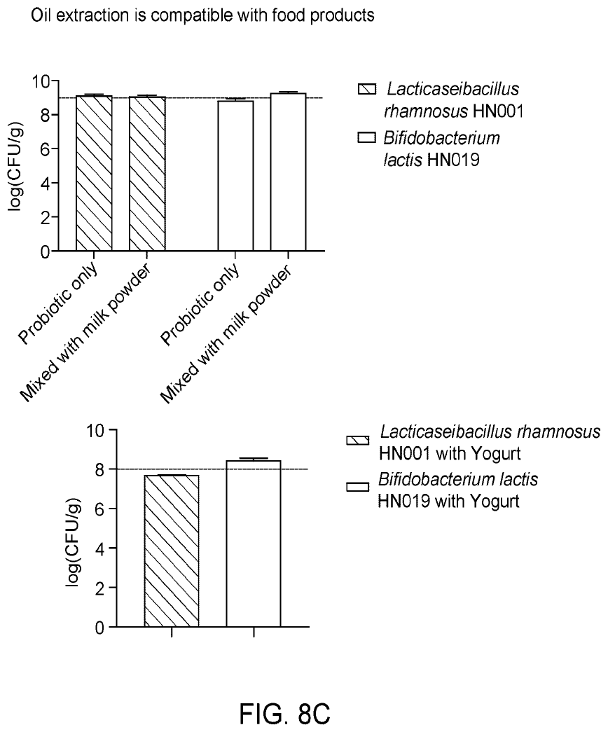

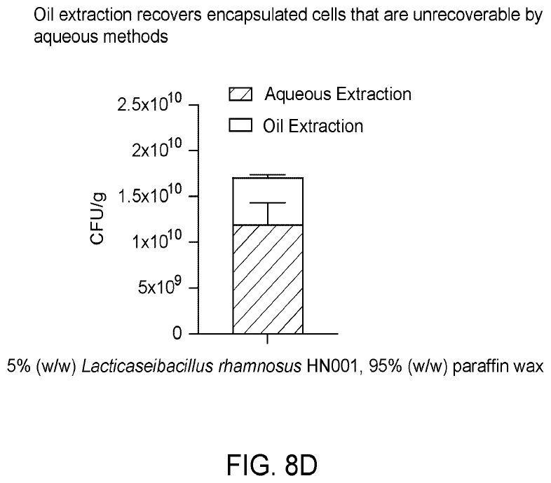

An exemplary protocol 800 for enumerating colony forming units from probiotic compositions is described herein (“oil extraction”), the process of which is exemplified in FIG. 8A. In warming step 802, an oil component (e.g., vegetable, castor, avocado, sunflower, rapeseed, mineral, palm oil) is added to a glass beaker in an amount of 5× to 50× the measured weight of a portion of probiotic composition of and warmed at a temperature ranging from about 35° C.-80° C. according to the probiotic composition and type of oil used. In step 804, the weighed portion of probiotic composition is then added to the prewarmed oil. Next, the product of step 804 is mixed until melted and homogenous in a liquid form in mixing step 806. In step 808, a specified amount (1×-20×the mass of oil) of biocompatible surfactant(s)/solubilizer(s) (e.g., Emulsifying Wax, Cetearyl Glucoside and Cetearyl Alcohol, Glyceryl Stearate SE, Cetyl Alcohol NF, Glycerol Monostearate, Polyoxyethylene Glycol Sorbitan Alkyl Esters, Sorbitan Alkyl Esters, Polysorbates, PEG-40 Hydrogenated Castor Oil, Safflower Oleosomes, and/or Propanediol), selected according to the type and amount of probiotic composition and oil component used, is added to the product of step 806. Next, the product of step 808 is then mixed well at a temperature ranging from about 35° C.-80° C. in step 810. Next, in step 812 an amenable salt solution is added (e.g., saline, phosphate buffered saline, peptone solution, maximum recovery diluent, 134ulbecco's phosphate buffered saline, HEPES buffered saline, Hank's balanced salt solution, Earl's balanced salt solution, and/or other similar balanced salt solutions used to maintain osmolality and pH in biological applications) to the product of mixing step 810 in a volume sufficient for a final emulsifier concentration of 0.5-30% w/w), selected according to the composition and amount of probiotic composition, oil and emulsifier. The product of mixing step 814 is then mixed for a specified time at an RPM between 100-400 rpm, until a uniform emulsion is formed. This product of step 814 may then be serially diluted and used to perform a spread plate enumeration method.

The oil extraction described herein improves upon the recovery achieved with a traditional aqueous method, and achieves full recovery of all cells incorporated in exemplary probiotic particles relative to an equivalent amount of free cells, as demonstrated in FIG. 8B on particles of 5% Lacticaseibacillus rhamnosus HN001 in paraffin wax and 5% Bifidobacterium lactis HN019.

The exemplary protocols for enumeration of microbes within probiotic compositions provided herein are, in some embodiments, applicable to the enumeration of microbes dispersed within food and/or beverage matrices. In one non-limiting example, the enumeration of microbes (e.g., probiotic cells) is achieved following dispersion in milk powder and/or yogurt matrices. As FIG. 8C demonstrates, the measured colony forming units of Bifidobacterium lactis or Lacticaseibacillus rhamnosus dispersed within milk powder and yogurt, determined using the oil extraction method provided herein, matches the intended colony forming units provided by unmixed probiotic cells in the absence of milk powder and yogurt.

An exemplary protocol for enumerating colony forming units from the moisture-accessible surface of probiotic compositions (aqueous extraction) involves weighing 1 g of the probiotic composition into a 15 mL conical tube. To the tube is added 9 mL of 45° C. prewarmed peptone buffer (peptone, milliQ water), which is then vortexed and rotated on a rotisserie for 10 minutes. This solution may then be serially diluted and used to perform a spread plate enumeration method. However, through sequentially collecting the undissolved probiotic composition following an aqueous extraction and performing a secondary oil extraction on the collected material, as performed in FIG. 8D, it is demonstrated that while the aqueous protocol can be effective in enumerating the surface accessible portion of a payload, it does not fully capture all microbes, and that the oil extraction protocol is necessary to release and enumerate the remaining, inaccessible, fully encapsulated payload (microbes). Notably, if an oil extraction is performed, a separate aqueous extraction is not needed to enumerate moisture accessible portions. By performing both the aqueous and the oil extraction protocol, the amount of payload (microbes) in both the moisture accessible and inaccessible portions can be enumerated separately, and thus a ratio of encapsulated and un-encapsulated microbes can be determined (encapsulation efficiency).

An exemplary protocol for enumerating an exemplary probiotic (e.g., Lactobacillus, Bifidobacterium) powder or solution involves first weighing out a specified amount of the sample. Probiotics are removed from storage and allowed to warm to room temperature. A specified amount (e.g., 1 g) of sample is transferred to a conical tube and diluted with a specified amount (e.g., 9 mL) of sterile peptone buffer (peptone, milliQ water). This tube is vortexed and serially diluted further in 1:10 dilutions down to the appropriate final dilutions according to the initial amount of probiotics in the sample. The relevant dilutions are then spread plate onto pre-prepared MRS agar plates (for Lactobacillus strains, comprising: Protease, Peptone, Beef extract, Yeast extract, Dextrose, Polysorbate 80, Ammonium Citrate, Sodium Acetate, Magnesium Sulfate, Manganese Sulfate, Dipotassium Phosphate, Magnesium Sulfate Heptahydrate. Agar. Distilled/Deionized Water) or pre-prepared MRS+L-cysteine agar plates (for Bifidobacterium strains, comprising: Protease, Peptone, Beef extract, Yeast extract, Dextrose, Polysorbate 80, Ammonium Citrate, Sodium Acetate, Magnesium Sulfate, Manganese Sulfate, Dipotassium Phosphate, Magnesium Sulfate Heptahydrate, Agar, L-cysteine, Distilled/Deionized Water) using a sterile L-spreader. Dilutions are plated in triplicate. Plates are allowed to dry, inverted, placed into a sealed anaerobic chamber with anaerobic packs to remove oxygen, and finally stored in an incubator set at 37° C. for 24-72 hours. After incubation and removal from the incubator, individual colonies are enumerated. To determine the initial amount of colony forming units in the sample, the number of counts is multiplied by a factor of the amount of dilution from the original sample. Triplicate colony forming unit enumerations are then averaged together.

### K. Example 11: Growth Compatibility of Probiotic Particles with Probiotics

The following example illustrates that the material compositions disclosed herein do not inhibit cell growth and/or metabolism prior to release in a nutritive and/or desired final environment.

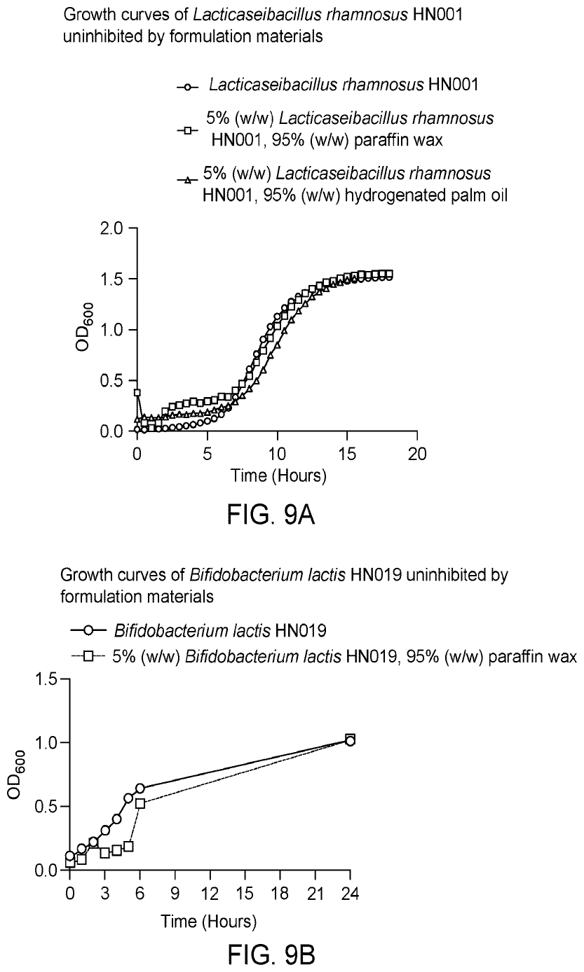

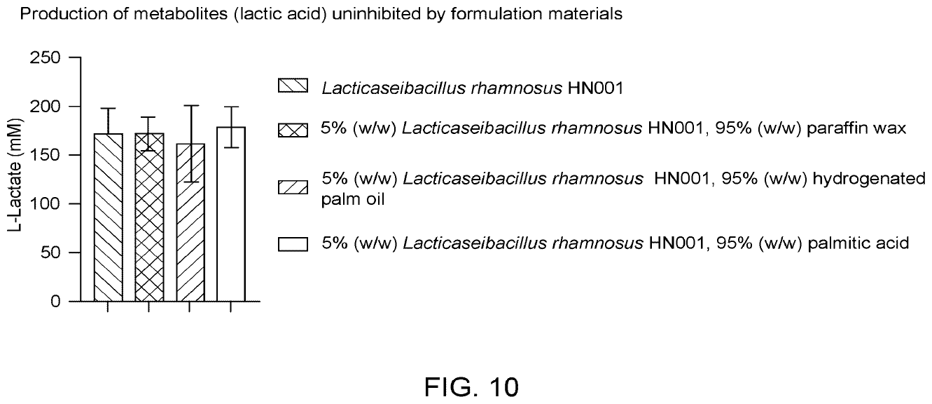

In one exemplary embodiment, probiotic compositions (e.g., particle preparations) encapsulating probiotics demonstrate minimal interference toward cell growth following incubation in MRS broth. As shown in FIG. 9, the growth of loosely encapsulated (e.g., aqueous-accessible) Lactobacillus rhamnosus HN001 (FIG. 9A) or Bifidobacterium lactis HN019 (FIG. 9B) probiotics comprised within exemplary probiotic compositions are not inhibited by the presence of particle preparations (i.e., relative to probiotic cells grown in MRS broth free of particle preparations). Similarly, FIG. 10 illustrates that when probiotics were encapsulated in various lipid components (paraffin wax, hydrogenated palm oil, palmitic acid) and incubated in MRS broth, probiotic production of a lactic acid metabolite was not inhibited as compared to an un-encapsulated probiotic (Lactobacillus rhamnosus HN001, in this example).

An exemplary protocol for monitoring production of lactic acid (e.g., FIG. 10) and/or overall growth involves preparing an overnight culture by weighing 100 mg of a probiotic powder or liquid into 50 mL of a suitable nutrient broth (MRS or MRS+L-cysteine). The tube is mixed followed by incubation at 37° C. for 18-24 hours. OD600 measurements are taken at 0.5-1 hr intervals using a spectrophotometer during the incubation period to construct a manual growth curve; alternatively, a plate reader can be used with volumes <300 μL. Aliquots are sampled at predetermined time points or at assay completion and centrifuged at 6000×g to pelletize the bacteria. The resulting supernatant is collected for use in EnzyChrom™ L-Lactate Assay Kit (ECLC-100) to determine lactic acid content.

### L. Example 12: Probiotic Composition Resists Degradation when Incorporated into Food and/or Beverage Products

Food and/or beverage products are, in some instances, complex matrices that promote the degradation of pharmaceutical and probiotic compositions via the presence of water, enzymes, and/or acidic/basic environments. The following example demonstrates the novelty of the disclosed invention in preserving cell viability when probiotic compositions are incorporated into milk powder, yogurt, and other dairy products.

In a non-limiting exemplary embodiment, probiotic compositions are shown (FIG. 11A-11B) to protect Lactobacillus rhamnosus from degradation and/or viability loss following incorporation and incubation in milk powder for up to 12 weeks at either 25° C. or 37° C. relative to unformulated bacteria. At 12 weeks following incubation at 25° C., bacteria within probiotic compositions exhibit <0.3 log(CFU/g) loss in viability versus 0.73 log(CFU/g) for unformulated bacteria in milk powder. At 12 weeks following incubation at 37° C., bacteria within probiotic compositions exhibit <2.7 log(CFU/g) loss in viability versus 3.76 log(CFU/g) for unformulated bacteria in milk powder.

FIG. 11C-11D are brightfield micrographs depicting probiotic compositions incorporated and stored within milk powder, then subsequently rinsed, filtered, and imaged with a compound microscope. Size and morphology of compositions following incubation in milk powder are comparable to those of the freshly prepared compositions depicted in FIG. 1A-1H.

In addition to stabilizing probiotic bacteria in milk powder, certain instances of the provided probiotic compositions are capable of protecting bacteria in a yogurt matrix, as described in FIG. 12A. Encapsulated Lactobacillus rhamnosus exhibits about 1000-fold greater viability following incorporation and incubation in yogurt for 9 weeks at 30° C. relative to unformulated bacteria.

FIG. 12B-12D are brightfield micrographs depicting probiotic compositions incorporated and stored within yogurt, then subsequently rinsed, filtered, and imaged with a compound microscope. Size and morphology of compositions following incubation in yogurt are comparable to those of the freshly prepared compositions depicted in FIG. 1A-1H.

An exemplary protocol for isolating probiotic composition from milk powder or yogurt involves weighing out an excess amount of milk powder/yogurt/probiotic composition mixture into a large beaker. With constant mixing at 300 RPM, between 5×-20× the weight of milk/yogurt/probiotic composition mixture of water is added until a colloidal solution is formed. Floating particles are skimmed off the surface of the colloidal suspension using a spatula, and transferred to a second, clean beaker. The contents of the second beaker with water are rinsed and the skimming and collection processes are repeated as necessary. The rinsed particles are then filtered using a Buchner funnel and vacuum. Particles are subsequently air dried before enumeration according to the oil extraction process detailed in Example 10.

An exemplary protocol for preparing probiotic compositions for long term storage studies with or without dairy products (e.g., milk powder) is performed as follows. A specified amount of probiotic composition is weighed and placed into a set of metallized bags (various sizes). For studies designed to represent storage in anoxic conditions, the bag atmosphere is evacuated and flushed with nitrogen, taking care not to displace the solid contents. The bags are then sealed using a vacuum sealer or non-vacuum heat sealer to represent storage in vacuumed or non-vacuumed bags respectively, and stored at specified temperatures (e.g., −4° C., 25° C., 35° C., 37° C., 50° C.). For long term storage studies, separate bags are created for individual time-points in which samples are meant to be taken and enumerated.

### M. Example 13: Probiotic Composition Resists Moisture Uptake and Resists Viability Loss in High Moisture Environments

The presence of water and/or water activity is a common factor underlying the loss of viability in cell-containing probiotic compositions. The following example illustrates the ability of the barrier materials in the provided probiotic compositions to retain integrity in high-moisture conditions, resist water uptake, and thereby mitigate degradation of the cellular component included therein.

For example, FIG. 13 demonstrates that the probiotic compositions in this disclosure do not gain moisture content, even when exposed to controlled relative humidities of 33%, 53%, or 75% for 4 days. Un-encapsulated probiotic powder and dehydrated milk powder, on the other hand, demonstrate a 2-5 fold increase in moisture content. FIG. 14 reveals that probiotic compositions encapsulating probiotic cells exhibit a smaller increase in water activity as compared to un-encapsulated probiotics. For example, even when the initial level of water activity is higher, as shown in FIG. 14, the encapsulated probiotic compositions of the present embodiments demonstrated a lower level of water activity increase when exposed to increasing amounts of humidity. As such, even when exposed to 75% relative humidity, the water activity of the encapsulated probiotic compositions of the present embodiments demonstrated lower water activity levels than un-encapsulated probiotics, which had a much lower initial water activity (i.e., a much lower starting point).

The protection from degradation afforded by the provided probiotic compositions is further exemplified in FIGS. 15A-C, where the viability of encapsulated cells is compared to that of un-encapsulated cells following roughly 12 weeks of controlled exposure within dehydrated milk powder to a relative humidity of 35% or 50%. After 13.5 weeks in a relative humidity of 35%, probiotic compositions exhibit 0.22-0.68 log(CFU/g) improvement in viability relative to un-encapsulated cells and <0.9 log(CFU/g) total loss in viability. After 12 weeks in relative humidity of 50%, probiotic compositions exhibit 0.12-0.82 log(CFU/g) improvement in viability relative to un-encapsulated cells and <2.08 log(CFU/g) total loss in viability. All enumerations were performed as described in Example 10.

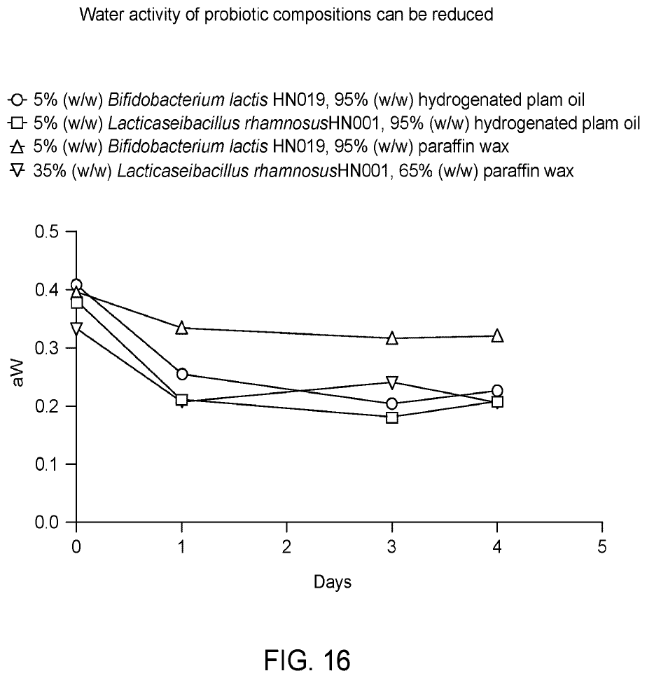

FIG. 16 demonstrates that probiotic particles can be dried (e.g., just prior to bagging for long term storage or packaging), using a moisture absorber such as Drierite®. Using this method, water activity was reduced by up to around 50%. Other methods to dry include vacuum, ovens at elevated temperatures, typically between 50° C. and 70° C., and/or vacuum ovens.

Those skilled in the art will appreciate that increased humidity stability can improve, for example, shelf-life and shelf-storage of both the particle itself and moisture sensitive payload components (e.g., probiotics). Furthermore, the ability of provided technologies to limit moisture uptake and increase stability of included payload compounds enables such compounds to be incorporated into or included in water-based compositions (e.g., water-based food products or other edible compositions) or other materials whose moisture/water content would otherwise destroy or negatively impact the payload compound(s). Thus, the present disclosure provides compositions that include payload component(s) (e.g., probiotics) formulated within barrier materials as described herein, specifically including where such payload component(s) are or comprise agent(s) or material(s) that are otherwise not stable to humidity/water exposure.

An exemplary protocol for determining moisture content from probiotic particles involves weighing an amount of probiotic particles in an aluminum dish. Subsequently, the aluminum dish and particles are dried in an oven at 105° C., for 24 hours. The sample is weighed again, and the loss in weight is attributed to loss of all the moisture content that has dried off, thereby allowing for a calculation of % moisture content in the original sample. Water activity (aw) is measured using a METER aqualab TDL-2 water activity meter.

It is contemplated that embodiments of provided probiotic particles enable storage of moisture-sensitive component(s) (e.g., probiotics). Without wishing to be bound by particular theory, the present disclosure proposes that provided particles may limit transport of water (e.g., from particle(s) to the environment or vice versa), and thus may confer benefit to (e.g., may improve stability of) other component(s) or material(s) with which they are combined or otherwise associated, particularly to the extent that such other component(s) or material(s) may otherwise display sensitivity to water. In some particular embodiments, such water-sensitive component(s) or material(s) may be probiotic(s). In some embodiments, a provided probiotic particle comprises a lipid component (e.g., a wax) and a payload component (e.g., a probiotic); alternatively or additionally, in some embodiments, a provided probiotic particle includes a nutraceutical particle preparation that itself includes (e.g., incorporates and/or encapsulated) a nutraceutical, which may confer protective benefits to other components that probiotic particles may comprise or be combined with and that are often sensitive to water.

### N. Example 14: Anti-Caking/Anti-Agglomerating/Anti-Aggregating/Anti-Clumping Particles

The following exemplary embodiment illustrates the ability of the disclosed probiotic compositions to improve the anti-caking, anti-clumping, anti-agglomerating, anti-aggregation properties of the cellular component.

As shown in FIG. 17A, exemplary probiotic compositions comprise Lactobacillus rhamnosus encapsulated in either paraffin wax, hydrogenated palm oil, or palmitic acid exhibit reduced caking, agglomeration, aggregation, and/or clumping versus unformulated bacteria. Petri dishes with probiotic compositions incubated at 25° C. in either 33%, 53%, or 75% relative humidity lack the >2 mm diameter clumps present in the dishes containing unformulated bacteria. Moreover, a single inversion is sufficient to displace all of the probiotic composition from the surface of the petri dish, while unformulated bacteria remain adhered to the material surface. Similarly, as shown in FIG. 17B, exemplary probiotic compositions comprise Lactobacillus rhamnosus or Bifidobacterium lactis encapsulated in hydrogenated palm oil, exhibit reduced caking and better flowability compared to powders of the bacteria itself, when tilted and encouraged to flow to one side of the container.

It is contemplated that the improved anti-caking, anti-agglomerating, and/or anti-aggregation and associated flowability of the probiotic compositions provided herein may confer increased stability despite storage in suboptimal conditions and improved dispersibility in food and/or beverage products.

### O. Example 15: Dispersal of Probiotic Compositions within Food and/or Beverage Products

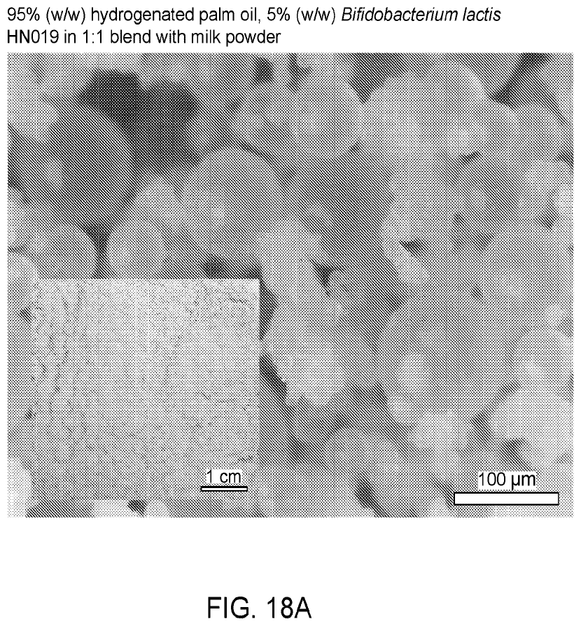

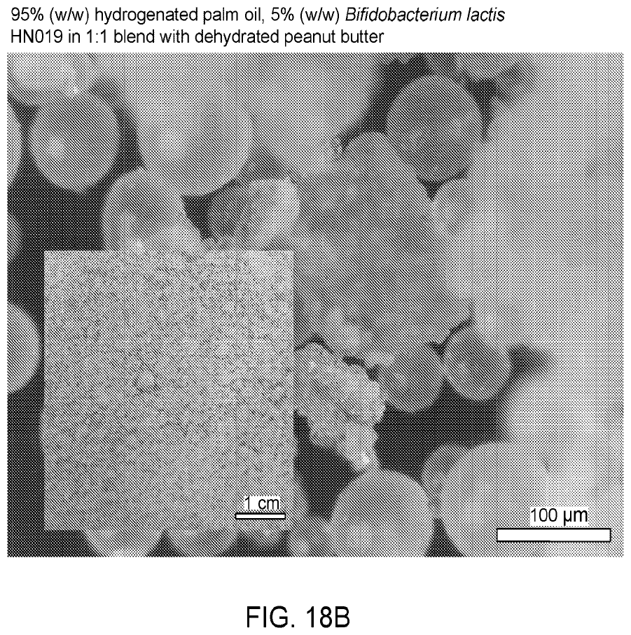

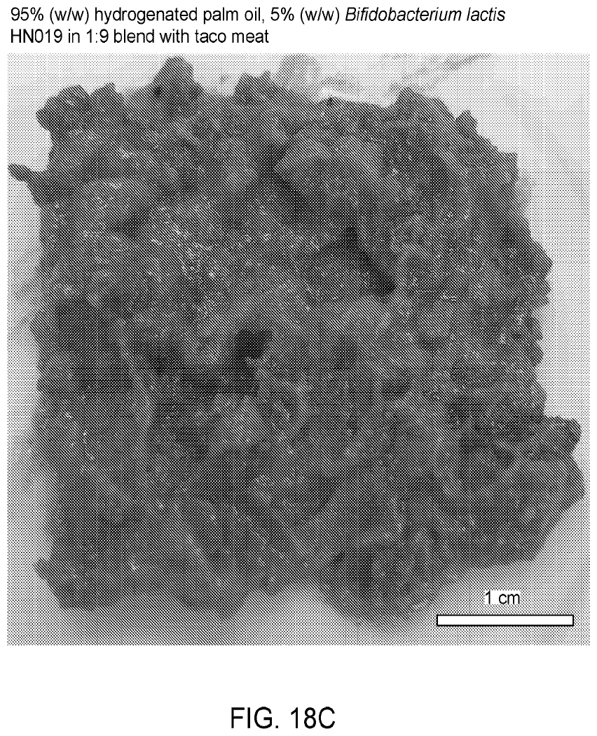

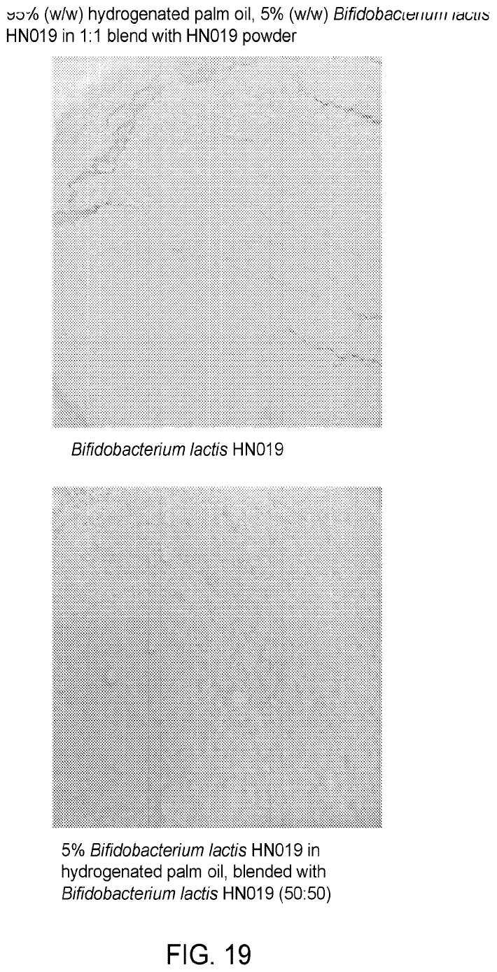

This example illustrates homogeneous mixtures of disclosed probiotic composition(s) within dairy products (e.g., dry milk powder) and/or food products (e.g., taco meat, peanut butter bar) as demonstrated in FIGS. 18A-C. It is contemplated that non-limiting exemplary embodiments of probiotic particles can be homogeneously mixed with other food products such as freeze dried powder, protein powder, solid bars (protein bars), domestic pet food (pellets), liquid shakes, pudding, etc. Homogenization can be achieved without additional processing aid or improved through addition of processing aid/excipients, through the use of mixing apparatuses such as a homogenizer, stand mixer, paddle blender, stir bar, spatula, etc. Without wishing to be bound by any particular theory, the present disclosure proposes that size characteristics and/or compositions of certain provided probiotic particles may surprisingly contribute desirable and/or useful attribute(s) to such particles, specifically including, for example, amenability to homogenous combination with other component(s), specifically including powder component(s) such as milk powder(s), and/or brightening/whitening of the overall mixture (FIG. 19).

### P. Example 16: Summary of Particle Characteristics and Performance

TABLE 1
Certain technical parameters of non-limiting exemplary particle compositions.

| | Results |
|---|---|
| | All ‘x’ increases are compared to “free” compound (i.e., |
| | not incorporated into or combined with a particle |
| Feature: Conditions | preparation as described herein) |
| Particle sizes: 5-3000 μm | Particle preparations size can be controlled between 5- |
| | 3000 μm |
| | Certain ranges are: 60 μm-300 μm, 100 μm-1000 μm, 5 |
| | μm-60 μm, 5 μm-300 μm. |
| | The following particle diameters (all in units of μm) are |
| | annotated in the following corresponding FIGS: |
| | FIG. 1A: 85.8, 46.8, 204.1 |
| | FIG. 1B: 56.5, 195.3, 127.8 |
| | FIG. 1C: 120.3, 162.9, 138.1 |
| | FIG. 1D: 125.2, 240.1, 108.2 |
| | FIG. 1E: 217.4 |
| | FIG. 1F: 747.4, 301.4, 135.2 |
| | FIG. 1H: 126.7 |
| | FIG. 3B: 256.8 |
| | FIG. 3C: 155.8, 210.6 |
| | FIG. 3D: 181.8, 129.0 |
| | FIG. 11C: 129.1, 196.9, 111.8 |
| | FIG. 11D: 219.7, 196.2 |
| | FIG. 12B: 200.6, 166.1 |
| | FIG. 12C: 186.7, 169.3 |
| | FIG. 12D: 225.9, 173.6 |
| Particle loadings: 1-50% for | HN001/HN019 loadings: 1%, 5%, 35%, 50% (w/w) |
| HN001, HN019, or combinations | |
| thereof | |
| SGF survival | <2 log(CFU/g) loss following 1, 3, 6, 12, or 24 h |
| | incubation compared to total loss of un-encapsulated |
| | probiotic |
| Survival in milk powders | <1 log (CFU/g) loss following 1, 2, 6, or 12 week |
| | incubation at 25° C. |
| | <2 log (CFU/g) loss following 1, 2, 6, or 12 week |
| | incubation at 37° C. |
| Survival in yogurt | <5 log (CFU/ml) loss following 1, 2, 4, or 9 week |
| | incubation at 37° C. |
| | <4 log (CFU/ml) loss following 1, 2, or 4 week |
| | incubation at 37° C. |
| | <1 log (CFU/ml) loss following 1, 2, or 4 week |
| | incubation at 37° C. |
| Morphology after storage in milk | Spherical morphology with some porosity (no change |
| powders | from initial particles) |
| Morphology after storage in milk | Spherical morphology with some porosity (no change |
| yogurt | from initial particles) |
| Anti-caking | Fine powders following 24 hour storage at 50% relative |
| | humidity |
| | Flowability upon 90° inversion |
| Combination with foods/beverages | No change in food and/or beverage appearance, taste, or |
| | texture |
| Water uptake in high humidity | No increase in moisture content following storage at 33%, |
| conditions | 53%, or 75% relative humidity |
| Morphology | Spherical morphology with some porosity |
| Metabolite secretion | Lactic acid metabolism uninhibited between unformulated |
| | probiotic and encapsulated probiotic |
| Growth | Growth uninhibited between unformulated probiotic and |
| | encapsulated probiotic |
| Storage | <0.1 log (CFU/g) loss following 4 months storage at −20° C. |
| | <0.1 log (CFU/g) loss following 1 month storage at 4° C. |
| | <0.1 log (CFU/g) loss following 1 month storage at 25° C. |

### Q. Example 17: Probiotic Composition Resists Degradation when Incorporated into Food and/or Beverage Products

Gummy products (e.g., gummy bears, gummy worms, gummy candies, gelatin-based gummy products, pectin-based gummy products, etc.) are complex matrices of high water activity, often including water activities between 0.55 and 0.70. As such, combining gummy products with probiotics presents a challenge due to the rapid loss of viability that probiotics experience in high water activity environments. The following example demonstrates an aspect of the present disclosed embodiments in which cell viability is preserved when probiotic compositions are incorporated alongside gummy products in sealed containers. VK-gummies are manufactured as a control group for storage study purposes to test alongside commercial-grade gummy candies. For example, VK-gummies may be formed via the following steps: heat about 186.8 g water to 100° C. on a hot plate. Prepare about 6.6% (w/w) gelatin solution by adding about 13.2 g gelatin powder to the hot water. Stir solution continuously at about 400 rpm while heated until gelatin is fully dissolved. Once gelatin is dissolved, remove gelatin solution from heat and pour into gummy template/mold. Cool at about 4° C. until gelatin is set. Slice gelatin into about 2 cm cubes and coat with a known mass of formulated (or unformulated) probiotics.

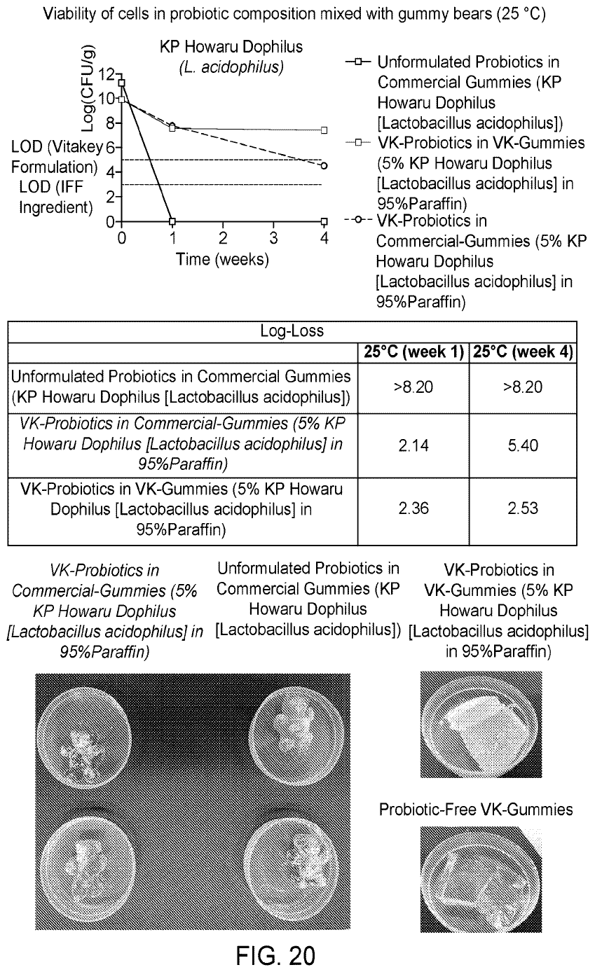

In a non-limiting exemplary embodiment, probiotic compositions are shown (FIG. 20) to protect KP Howaru Dophilus (Lactobacillus acidophilus) from degradation and/or viability loss following incorporation via coating and incubation with Black Forest brand gummy bears (water activity 0.65) for 1 week and 4 weeks at 25° C., relative to unformulated bacteria and VK-Gummies with the same probiotic composition as the Black Forest brand gummies. At 1 week following incubation at 25° C., bacteria within probiotic compositions exhibit <2.15 log(CFU/g) loss in viability versus >8.20 log(CFU/g) loss for unformulated bacteria when packaged with Black Forest gummy bears and <2.37 log(CFU/g) loss for VK-Gummies. The Black Forest coated gummy bears are represented by the black dashed line and filled circles on the data plot, the VK-Gummies are represented by the gray solid line and unfilled squares, and the unformulated bacteria are represented by the black solid line and filled squares.

Camera images depicting probiotic compositions and unformulated probiotics incorporated onto gummy bears are shown. Size and morphology of compositions following coating of gummy bears are comparable to those of unformulated probiotics coated onto gummy bears.

An exemplary protocol for isolating probiotic composition from gummy bears involved washing gummy bears coated with probiotics with 10 mL peptone, followed by plating the wash to enumerate CFUs. In some embodiments, samples coated with probiotic compositions may require oil extraction prior to plating.

An exemplary protocol for preparing probiotic compositions for long term storage studies in gummy bears may include the following steps. Commercially available gummy bears (Black Forest brand) were coated with unformulated probiotics, as well as probiotics compositions with at least 10⁹ CFU per serving and up to 10¹² CFU per serving. The gummy bears were used as-is from the bag. To control loading probiotics (both unformulated and probiotic compositions) can be cut with up to 1:10 maltodextrin. After coating gummy bears, a specified amount of gummy bears coated with probiotic composition or unformulated probiotic is weighed and placed into a set of metallized bags (various sizes). For studies designed to represent storage in anoxic conditions, the bag atmosphere is evacuated and flushed with nitrogen, taking care not to displace the solid contents. The bags are then sealed using a vacuum sealer or non-vacuum heat sealer to represent storage in vacuumed or non-vacuumed bags respectively, and stored at specified temperatures (e.g., −4° C., 25° C., 35° C., 37° C., 50° C.). For long term storage studies, separate bags are created for individual time-points in which samples are meant to be taken and enumerated.

### R. Example 18: Probiotic Compositions Incorporated into Milk Powders

The present example demonstrates incorporation of exemplary HN001 probiotic compositions and exemplary HN019 probiotic compositions into milk powders. A target probiotic concentration of 1×10⁹ CFU/g of milk powder was used for both free probiotics (non-encapsulated) and encapsulated probiotics. A mixer was used to mix probiotics and the milk powder. 20 grams of milk powder and probiotic mixture were placed into a foil or aluminum or mylar sachet/bag. A nitrogen head was optionally added to each bag and then immediately sealed using vacuum sealer. Bags were placed into incubators at appropriate temperatures (25 C, 30 C, 35 C). For free probiotics, an aqueous extraction was used to enumerate CFUs. Briefly, peptone was warmed to 45° C., 1 gram of milk powder with probiotic was added to the peptone, the mixture was allowed to rotate on a rotisserie for 15-20 minutes and serial dilutions were then plated on appropriate agar (e.g., MRS, MRS with 5% L-cysteine, etc.). For encapsulated probiotics, an oil extraction was typically used to enumerate CFUs. Briefly, an oil (e.g., mineral oil, sunflower oil, avocado oil, etc.) was warmed to 35-75 C, PEG40 or other surfactant was added and heated at the same temperature and was stirred for 5-20 minutes, then media (e.g., MRS, MRS with 5% L-cysteine, etc.) was added, and serial dilutions were performed and then plated on appropriate agar (e.g., MRS, MRS with 5% L-cysteine, etc.). Enumeration was performed as previously described.

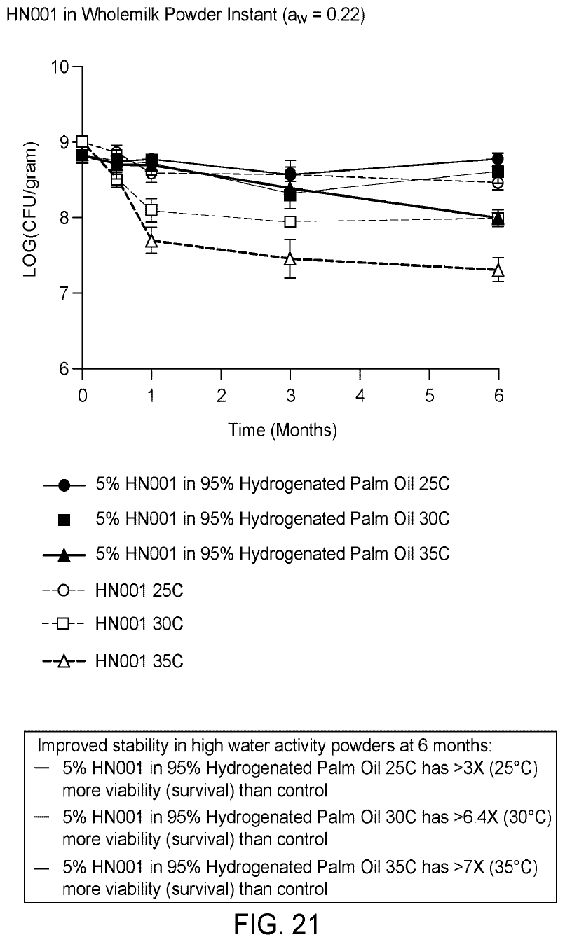

FIG. 21 shows exemplary data for HN001 (non-encapsulated and encapsulated) in whole milk powder instant with a water activity of 0.22. In this figure, all encapsulated formulations of HN001 (5% HN001 in 95% Hydrogenated Palm Oil) at 25, 30, and 35 C outperform non-encapsulated HN001 (as provided by the manufacturer). Encapsulated formulations provided a greater than 3-fold, 6.4-fold, and 7-fold higher survival (viability as determined by CFU) as compared to non-encapsulated probiotics at 25 C, 30 C, and 35 C respectively. This demonstrated that an encapsulation formulation can protect the Lacticaseibacillus rhamnosus HN001 from high water activity powders at 25, 30, and 35 C. All together, the encapsulated formulations demonstrate survival advantages over each timepoint evaluated over the 6 month period (0.5, 1, 3, 6 months).

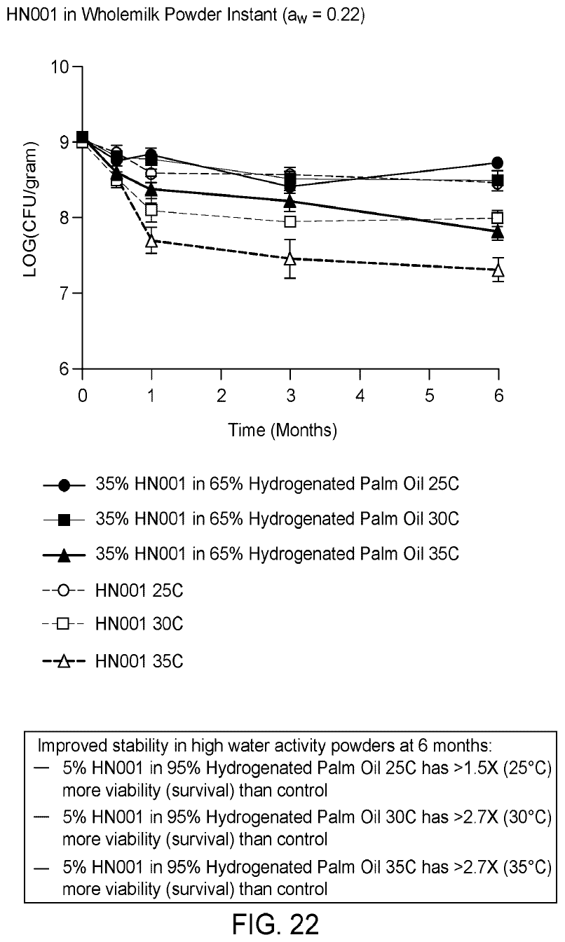

FIG. 22 shows exemplary data for HN001 (non-encapsulated and encapsulated) in whole milk powder instant with a water activity of 0.22. In this figure, all encapsulated formulations of HN001 (35% HN001 in 65% Hydrogenated Palm Oil) at 25, 30, and 35 C outperform non-encapsulated HN001 (as provided by the manufacturer). Encapsulated formulations provided a greater than 1.5-fold, 2.7-fold, and 2.7-fold higher survival (viability as determined by CFU) as compared to non-encapsulated probiotics at 25 C, 30 C, and 35 C, respectively. This demonstrates that an encapsulation formulation can protect the Lacticaseibacillus rhamnosus HN001 from high water activity powders at 25, 30, and 35C. Additionally, this data demonstrated that loading of the HN001 probiotic can be increased (From 5% to 35%) and the encapsulant decreased (from 95% to 65%) while still maintaining survival advantages during storage in milk powders. All together, the encapsulated formulations demonstrate survival advantages over each timepoint evaluated over the 6 month period (0.5, 1, 3, 6 months).

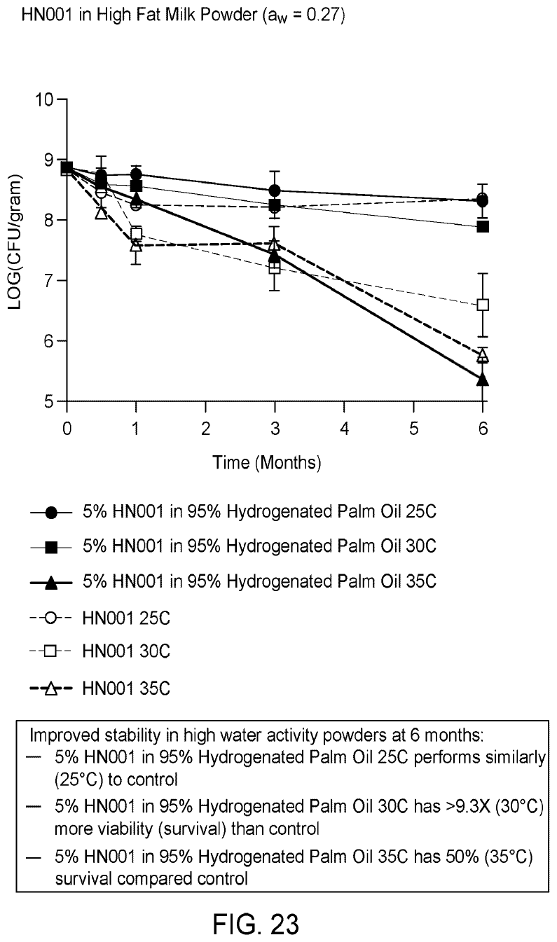

FIG. 23 shows exemplary data for HN001 (non-encapsulated and encapsulated) in high fat milk powder with a water activity of 0.27. In this figure, 2 of the encapsulated formulations of HN001 (5% HN001 in 95% Hydrogenated Palm Oil) at 25 and 30° C. are non-inferior or outperform non-encapsulated HN001 (as provided by the manufacturer). Encapsulated formulations provided a greater than 9.3-fold higher survival (viability as determined by CFU) as compared to non-encapsulated probiotics at 30 C. At 35 C, the encapsulated formulations demonstrated increased viability loss as compared to non-encapsulated; although survival advantages of encapsulated HN001 at 35 C are apparent at months 0.5 and 1. This is particularly useful since many commercial products are only intended to be used for 30 servings (e.g., 1 month if used daily). This demonstrated that an encapsulation formulation can protect the Lacticaseibacillus rhamnosus HN001 from high water activity powders at 25 C and 30 C.

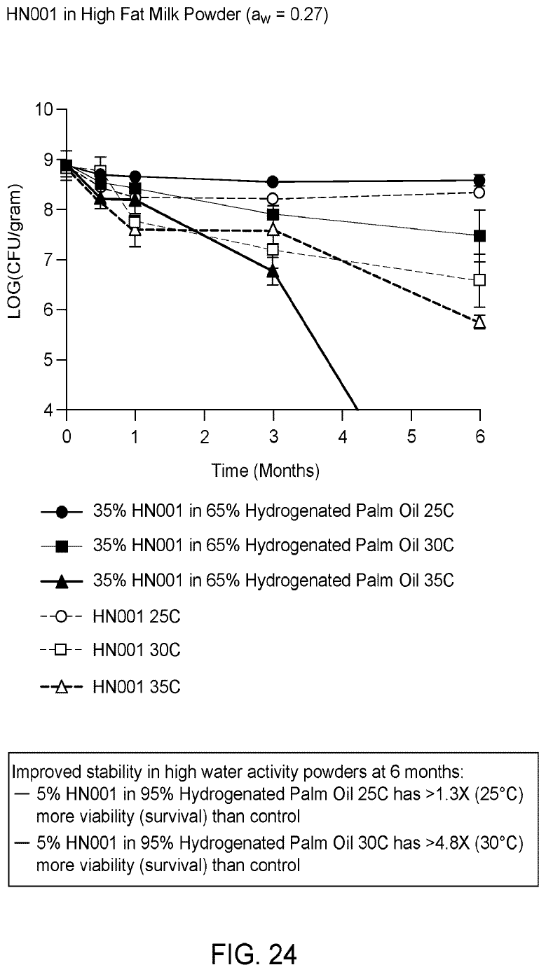

FIG. 24 shows exemplary data for HN001 (non-encapsulated and encapsulated) in high fat milk powder with a water activity of 0.27. In this figure, 2 of the encapsulated formulations of HN001 (35% HN001 in 65% Hydrogenated Palm Oil) at 25 C and 30° C. outperform non-encapsulated HN001 (as provided by the manufacturer). Encapsulated formulations provided a greater than 1.3-fold and 4.8-fold higher survival (viability as determined by CFU) as compared to non-encapsulated probiotics at 25 C and 30 C, respectively. At 35 C. the encapsulated formulations demonstrated increased viability loss as compared to non-encapsulated. At 35 C, the encapsulated formulations demonstrated increased viability loss as compared to non-encapsulated; although survival advantages of encapsulated HN001 at 35 C are apparent at months 0.5 and 1. This is particularly useful since many commercial products are only intended to be used for 30 servings (e.g., 1 month if used daily). This demonstrated that an encapsulation formulation can protect the Lacticasei bacillus rhamnosus HN001 from high water activity powders at 25 C and 30 C. Additionally, this data demonstrated that loading of the HN001 probiotic can be increased (From 5% to 35%) and the encapsulant decreased (from 95% to 65%) while still maintaining survival advantages during storage in milk powders.

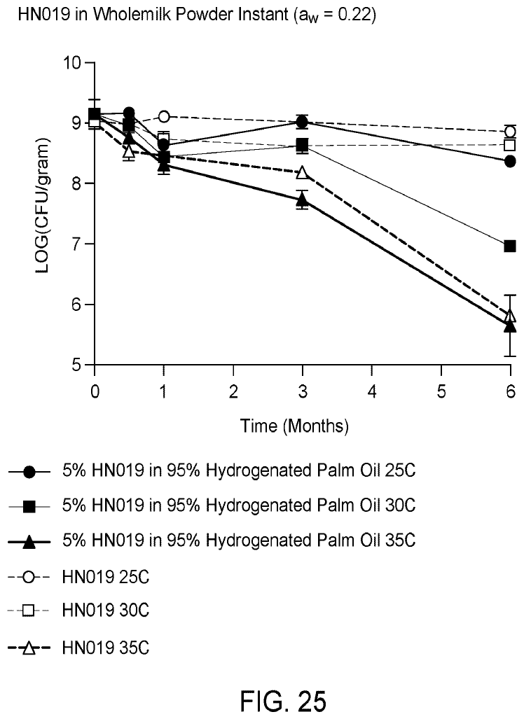

FIG. 25 shows HN019 (non-encapsulated and encapsulated) in whole milk powder instant with a water activity of 0.22. In this figure, all encapsulated formulations of HN019 (5% (w/w) Bifidobacterium lactis HN019 with 95% (w/w) hydrogenated palm oil) at 25, 30, and 35 C exhibit increased die-off or log-loss or viability loss as compared to non-encapsulated HN019 (as provided by the manufacturer). This demonstrated challenges in preparing encapsulation formulations which may be useful in protecting Bifidobacterium lactis HN019 from high water activity powders at 25, 30, and 35 C.

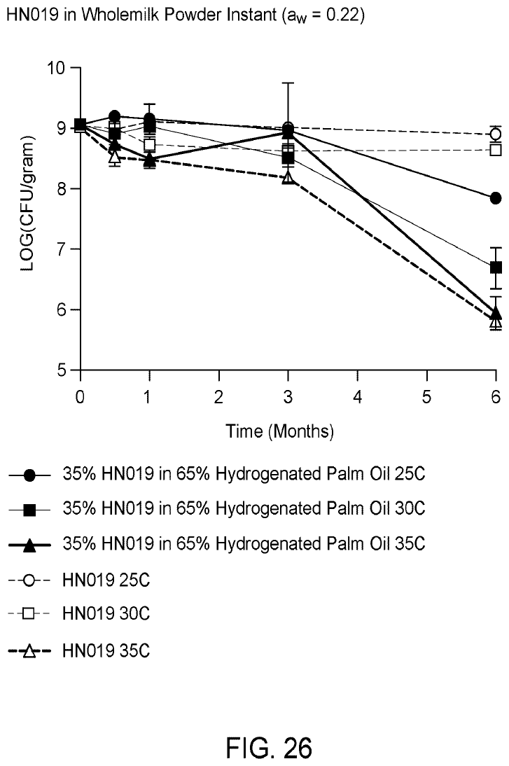

FIG. 26 shows HN019 (non-encapsulated and encapsulated) in whole milk powder instant with a water activity of 0.22. In this figure, encapsulated formulations of HN019 (35% (w/w) Bifidobacterium lactis HN019 with 65% (w/w) hydrogenated palm oil) exhibit increased die-off or log-loss or viability loss as compared to non-encapsulated HN019 (as provided by the manufacturer) at 25 C and 30 C. This demonstrated challenges in preparing encapsulation formulations which may protect Bifidobacterium lactis HN019 from high water activity powders at 25 C and 30 C. Additionally, at 35 C, non-inferiority between encapsulated and non-encapsulated HN019 was observed.

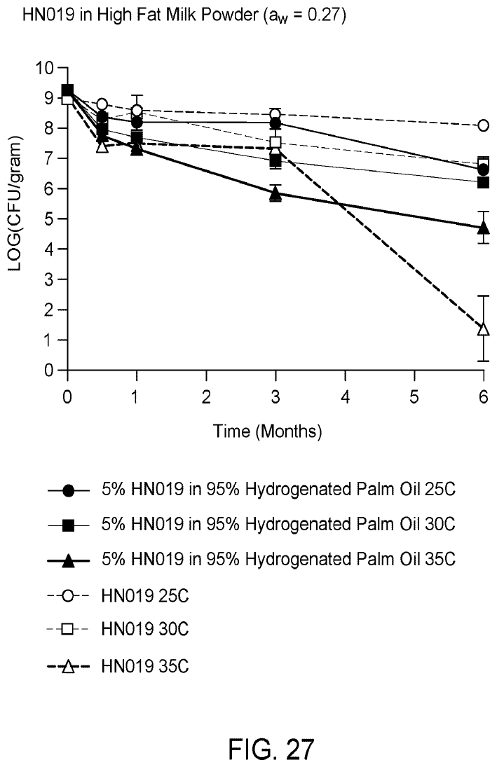

FIG. 27 shows HN019 (non-encapsulated and encapsulated) in high fat milk powder with a water activity of 0.27. In this figure, encapsulated formulations of HN019 (5% (w/w) Bifidobacterium lactis HN019 with 95% (w/w) hydrogenated palm oil) exhibit increased die-off or log-loss or viability loss as compared to non-encapsulated HN019 (as provided by the manufacturer) at 25 C and 30 C. This demonstrated challenges in preparing encapsulation formulations which may protect Bifidobacterium lactis HN019 from high water activity powders at 25 C and 30 C. Additionally, at 35 C, non-inferiority between encapsulated and non-encapsulated HN019 was observed.

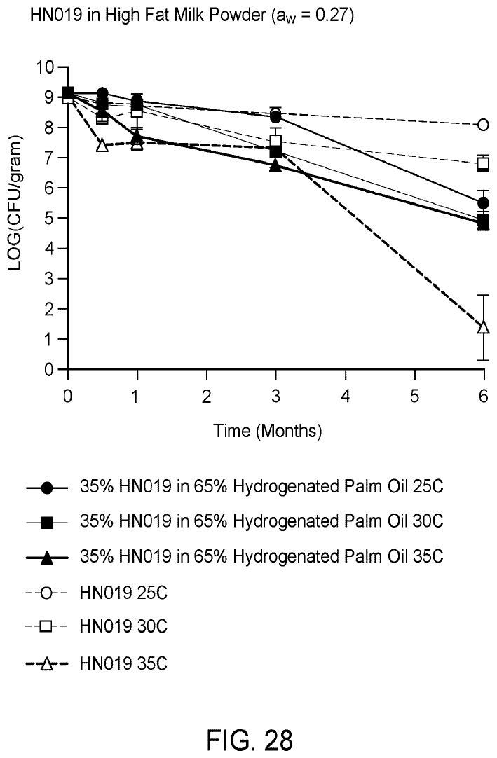

FIG. 28 shows HN019 (non-encapsulated and encapsulated) in high fat milk powder with a water activity of 0.27. In this figure, encapsulated formulations of HN019 (35% (w/w) Bifidobacterium lactis H4N019 with 65% (w/w) hydrogenated palm oil) exhibit increased die-off or log-loss or viability loss as compared to non-encapsulated HN019 (as provided by the manufacturer) at 25 C and 30 C. This demonstrated challenges in preparing encapsulation formulations which may protect Bifidobacterium lactis HN019 from high water activity powders at 25 C and 30 C. At 35 C. encapsulated HN019 outperforms non-encapsulated HN019.

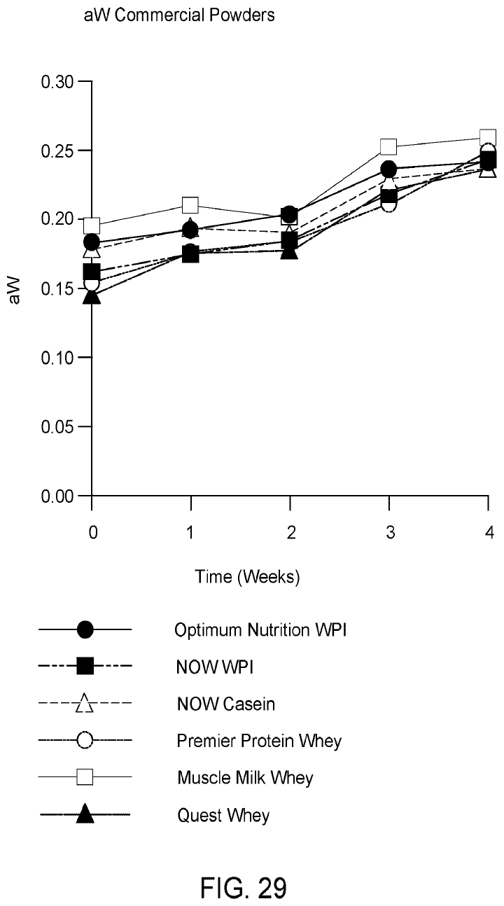

FIG. 29 shows water activity of commercial milk and/or dairy protein powders (i.e., aW) for a period of time (e.g. incubation period of 4 weeks) at room temperature. Each of these products had fewer than 30 servings per container. As such, 4 weeks of aW tracking was selected to be representative of the timeline of consumer use of these products. During simulated consumer use (e.g., opening the container 5 days out of a 7 day week to mimic consumer use by exposing the powders to ambient moisture which results in increased water activity), commercial powder products were below a water activity of 0.23 aW for the majority of the 4 week time period. This may be helpful in contextualizing results from FIGS. 21-28 since performance of encapsulated HN001 is significantly improved as compared to non-encapsulated HN001 for all temperatures at 0.22 aW and for 2 out of 3 temperatures (25 C and 30 C) at 0.27 aW. This demonstrated that encapsulation of some probiotics (e.g., HN001) in hydrogenated palm oil will meet the demands (e.g., stability of probiotic viability at 25 C, 30 C, 35 C) of commercially available protein powder products during the lifetime of the product (30 servings).

Comparing FIGS. 21-24 (encapsulated HN001 vs non-encapsulated HN001) to FIGS. 25-28 (encapsulated HN019 vs non-encapsulated HN019), demonstrates that HN001 responds differently to encapsulation as compared to HN019. Possible reasons for this include increased oxygen sensitivity of HN019 relative to HN001, increased heat sensitivity of HN019 relative to HN001 experienced during formulation, and/or other environmental or process-based challenges that are encountered during the formulation or experimentation.

### S. Example 19: Probiotic Compositions Incorporated into Yogurt

The present example demonstrates exemplary probiotic compositions incorporated into yogurt. A target probiotic concentration of 1×10⁷⁻⁹ CFU/ml of yogurt milk powder was used for both free probiotics (non-encapsulated) and encapsulated probiotics. A stomacher or mixer was used to mix probiotics and the yogurt. Between 1 and 50 ml of yogurt and probiotic mixture were placed into a sealable test tube and additionally sealed with paraffin film. Tubes were placed into incubators at appropriate temperatures (4° C., 25° C., 30° C., 35° C.). For free probiotics, an aqueous extraction was used to enumerate CFUs. Briefly, peptone was warmed to 45° C., 1 ml of yogurt with probiotic was added to the peptone, the mixture was allowed to rotate on a rotisserie for 15-20 minutes, and serial dilutions were then plated on appropriate agar (e.g., MRS, MRS with 5% L-cysteine, etc.). For encapsulated probiotics, an oil extraction was typically used to enumerate CFUs. Briefly, an oil (e.g., mineral oil, sunflower oil, avocado oil, etc.) was warmed to 35° C.-75° C., PEG40 or other surfactant was added and heated at the same temperature while being stirred for 5-20 minutes, then media (e.g., MRS, MRS with 5% L-cysteine, etc.) was added, and serial dilutions were performed and then plated on appropriate agar (e.g., MRS, MRS with 5% L-cysteine, etc.). Enumeration was performed as previously described herein.

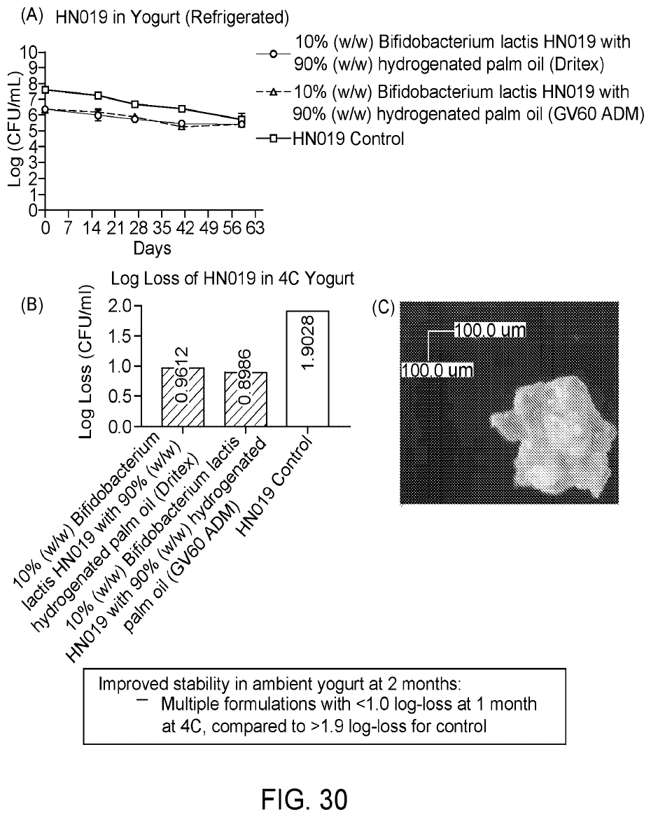

FIG. 30 demonstrates HN019 (non-encapsulated and encapsulated) in yogurt stored at 4° C. (refrigerated). In this figure, all encapsulated formulations of HN019 (10% HN019 in 90% Hydrogenated Palm Oil (both from Dritex and ADM)) at 4° C. outperformed non-encapsulated HN019 (as provided by the manufacturer). In FIG. 30A, this improved performance may not appear as clear since all groups were at approximately 6 log at 59 days; however, since the two encapsulated groups were added to yogurt at a lower concentration (CFU/ml), less log-loss or die off or viability loss was observed for the encapsulated formulations. FIG. 30B highlights these advantages by presenting the data as log loss over the 59-day period where both encapsulated formulations provided a greater than 2-fold higher survival (viability as determined by CFU) as compared to non-encapsulated probiotics at 4° C. This demonstrated that an encapsulation formulation can protect the HN019 from oxygen, moisture, etc. challenges that were encountered during storage in yogurt at 4° C. Furthermore, FIG. 30C demonstrates that the encapsulated probiotics maintain their particulate form after storage in yogurt for 28 days. Altogether, the encapsulated formulations demonstrated survival advantages over each timepoint evaluated over a 59-day period.

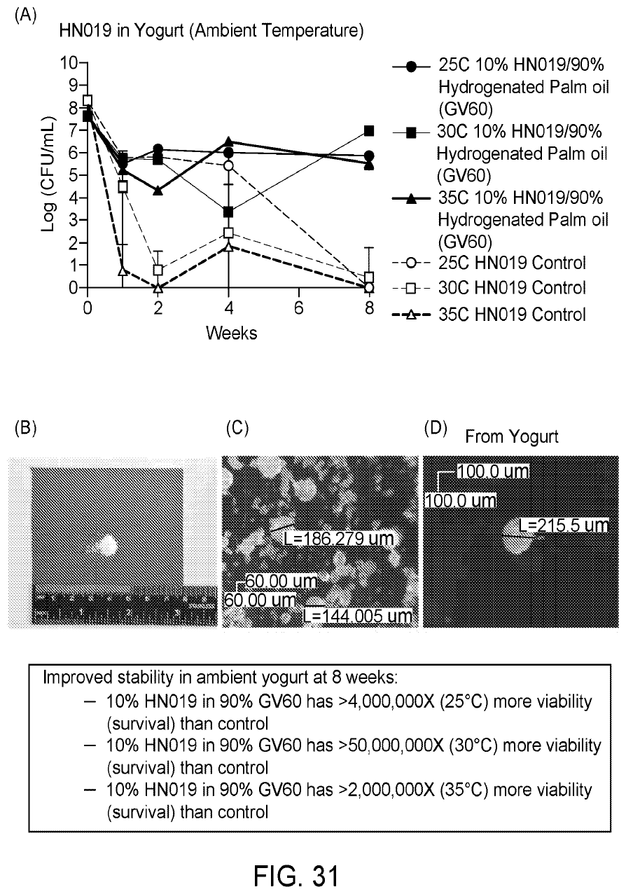

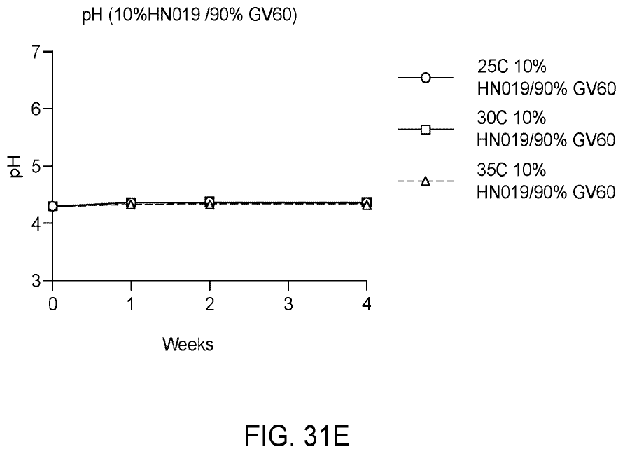

FIG. 31 demonstrates HN019 (non-encapsulated and encapsulated) in yogurt. In this figure, all encapsulated formulations of HN019 (10% HN019 in 90% Hydrogenated Palm Oil (GV60)) at 25° C. and 35° C. outperformed non-encapsulated HN019 (as provided by the manufacturer). Specifically, encapsulated formulations provided a greater than 4,000,000-fold, 50,000,000-fold, and 2,000,000-fold higher survival (viability as determined by CFU) as compared to non-encapsulated probiotics at 25° C., 30° C., and 35° C. respectively at 8 weeks. This demonstrated that an encapsulation formulation can protect the HN019 from oxygen, moisture, heat, etc. challenges that are encountered during storage in yogurt at 25° C., 30° C., and 35° C. Additionally, comparison of FIG. 31C (microscope image of the encapsulated probiotics) and FIG. 31D (microscope image of the encapsulated probiotics after storage in yogurt) demonstrate that the encapsulation system maintains its physical properties (e.g., size, shape, morphology, etc.) after storage in yogurt. As shown in FIG. 31E, the pH of the yogurt was maintained at 25° C., 30° C., and 35° C., over the 2-month period. Altogether, the encapsulated formulations demonstrate survival advantages over each timepoint evaluated over a 2-month period.

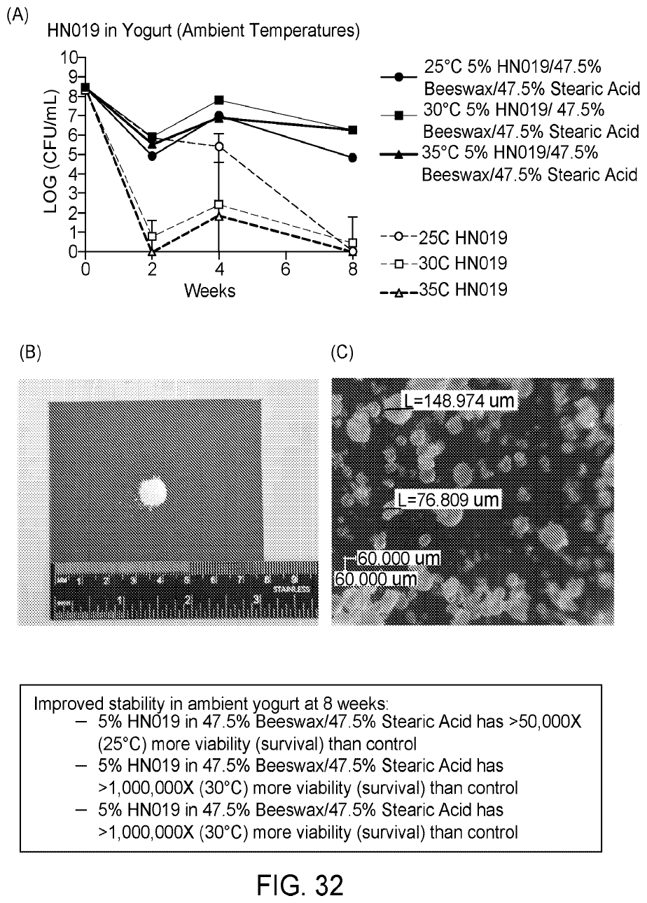

FIG. 32 demonstrates HN019 (non-encapsulated and encapsulated) in yogurt. In this figure, all encapsulated formulations of HN019 (5% HN019 in 47.5% Beeswax/47.5% Stearic Acid) at 25° C., 30° C., and 35° C. outperformed non-encapsulated HN019 (as provided by the manufacturer). Specifically, encapsulated formulations provided a greater than 50,000-fold, 1,000,000-fold, and 1,000,000-fold higher survival (viability as determined by CFU) as compared to non-encapsulated probiotics at 25° C., 30° C. and 35° C., respectively, at 8 weeks. This demonstrated that an encapsulation formulation can protect the HN019 from oxygen, moisture, heat, etc., challenges that were encountered during storage in yogurt at 25° C., 30° C., and 35° C. Altogether, the encapsulated formulations demonstrated survival advantages over each timepoint evaluated over a 2-month period.

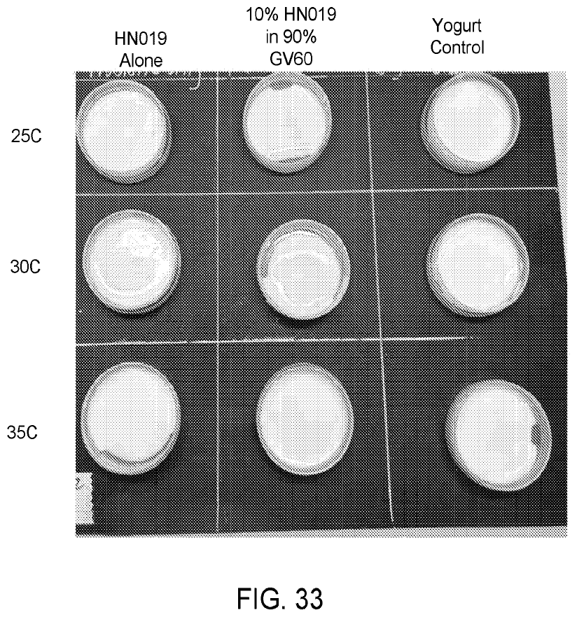

FIG. 33 shows images of yogurt, yogurt with encapsulated HN019 (10% HN019 in 90% GV60; see FIG. 21), and yogurt with HN019 probiotics (non-encapsulated) after 2 weeks storage in yogurt. Importantly, there were no observable differences (e.g., color, chunkiness, thickness, etc.) between the groups, indicating that encapsulated HN019 probiotics should not affect any of the physical sensory properties of the yogurt, after addition and storage.

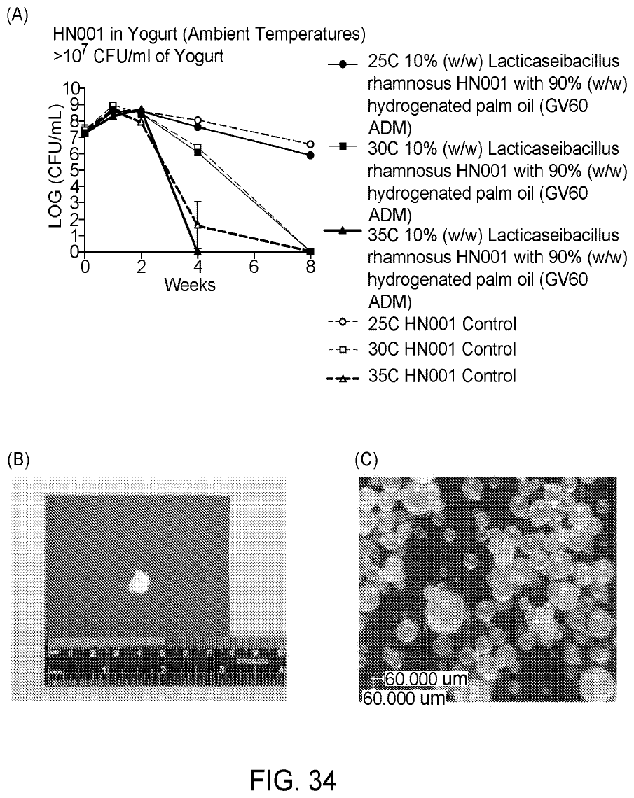

FIG. 34 demonstrates HN001 (non-encapsulated and encapsulated) in yogurt. In this figure, encapsulated formulations of HN001 (10% HN001 in 90% Hydrogenated Palm Oil (GV60 from ADM)) at 25° C., 30° C. and 35° C. exhibited similar die-off or log-loss or viability loss as compared to non-encapsulated HN001 (as provided by the manufacturer) at 25° C., 30° C., and 35° C. This demonstrated that an encapsulation formulation did not protect the HN001 from oxygen, moisture, heat, etc., challenges that were encountered during storage in yogurt at 25° C. 30° C., and 35° C. At all temperatures tested, comparable viability between encapsulated and non-encapsulated HN001 was observed.

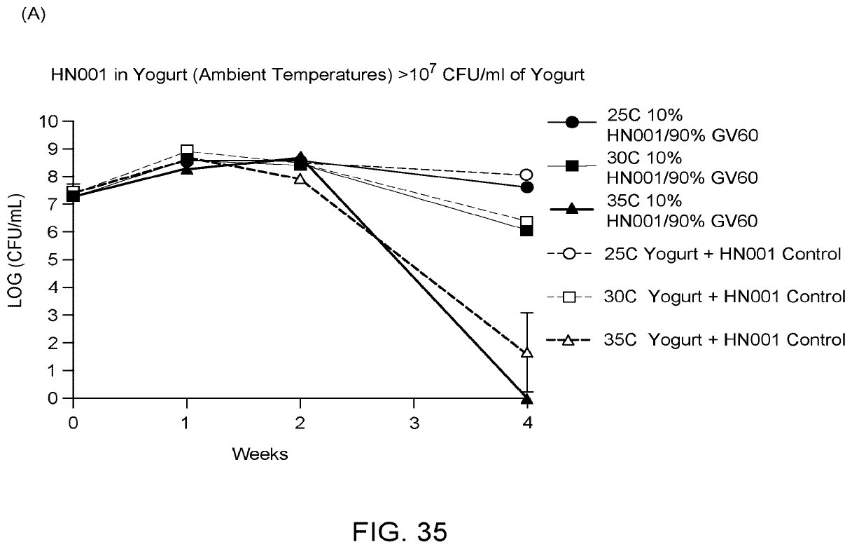

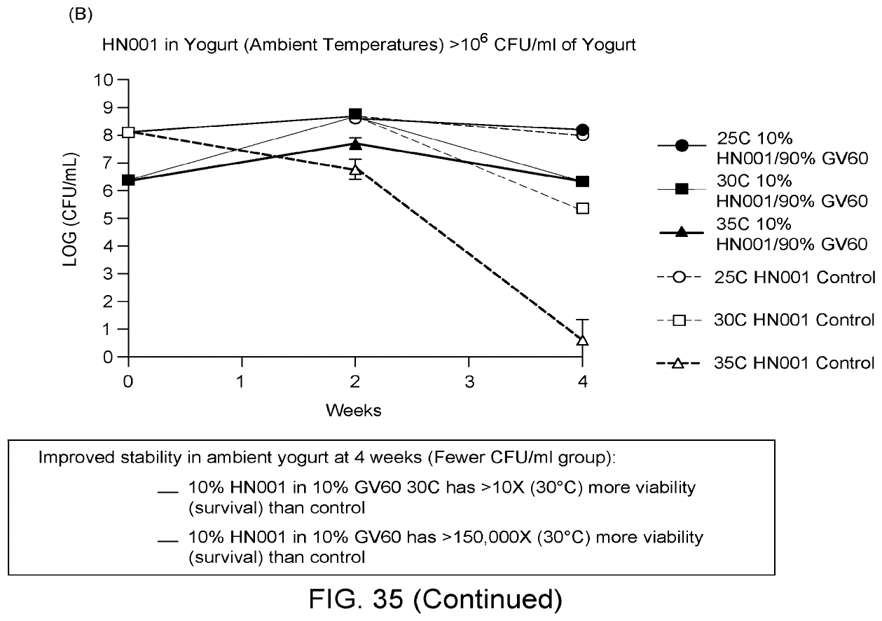

FIG. 35 demonstrates HN001 (non-encapsulated and encapsulated) in yogurt. In FIG. 35A, encapsulated formulations of HN001 (10% HN001 in 90% Hydrogenated Palm Oil (GV60 from ADM)) at 25° C., 30° C., and 35° C. exhibited similar die-off or log-loss or viability loss as compared to non-encapsulated HN001 (as provided by the manufacturer) at 25° C., 30° C., and 35° C. However, in FIG. 35B, encapsulated formulations of HN001 (10% HN001 in 90% Hydrogenated Palm Oil (GV60 from ADM)) at 30° C. and 35° C. exhibited lower die-off or log-loss or viability loss as compared to non-encapsulated HN001 (as provided by the manufacturer); at 25° C., the die-off was similar. Specifically, encapsulated formulations provided a greater than 10-fold and 150.000-fold higher survival (viability as determined by CFU) as compared to non-encapsulated probiotics at 30° C. and 35° C. respectively at 8 weeks (FIG. 35B). This demonstrated that, under some conditions, encapsulation of HN001 (10% HN001 in 90% Hydrogenated Palm Oil (GV60 from ADM)) provided survival advantages and enabled protection of HN001 from oxygen, moisture, heat, etc., challenges that were encountered during storage in yogurt at 25° C., 30° C., and 35° C. The difference between the experiments in FIG. 35A and FIG. 35B was the initial concentration of HN001 in yogurt (FIG. 35A=10⁷-10¹ CFU/ml and FIG. 35B—10⁶-10⁷ CFU/ml). This indicated that a lower concentration of encapsulated probiotic in yogurt at elevated temperatures (30° C. and 35° C.) mitigated the die-off and improved survival of HN001 in yogurt as compared to the non-encapsulated HN001 (as provided by the manufacturer).

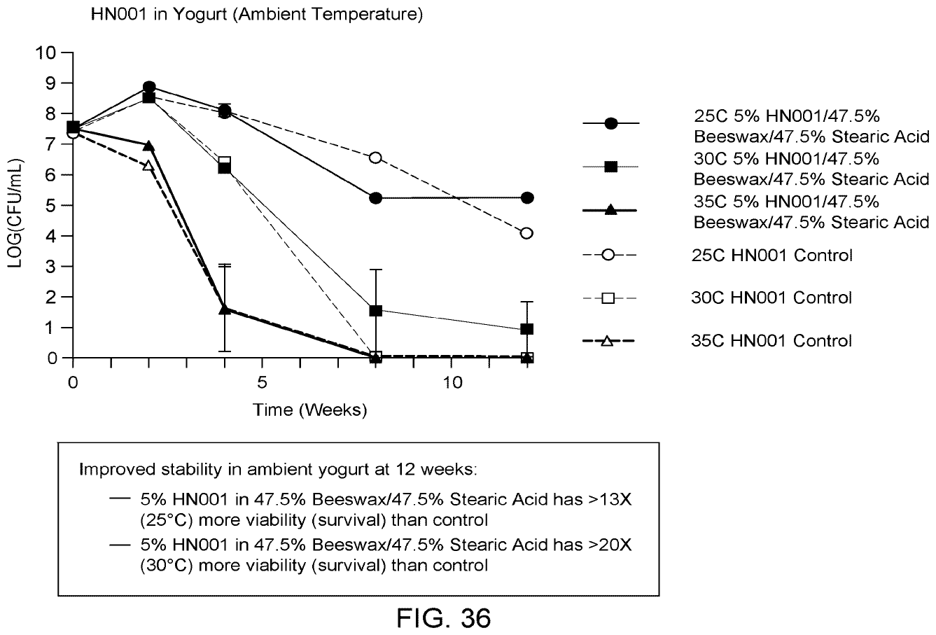

FIG. 36 demonstrates HN001 (non-encapsulated and encapsulated) in yogurt. In this figure, all encapsulated formulations of HN001 (5% HN001 in 47.5% Beeswax/47.5% Stearic Acid) at 25° C. and 30° C. outperformed non-encapsulated HN001 (as provided by the manufacturer): at 35° C., the encapsulated and non-encapsulated formulations performed similarly. Specifically, encapsulated formulations provided a greater than 13-fold and 20-fold higher survival (viability as determined by CFU) as compared to non-encapsulated probiotics at 25° C. and 30° C. respectively, at 12 weeks. This demonstrated that an encapsulation formulation can protect the HN001 from oxygen, moisture, heat, etc., challenges that were encountered during storage in yogurt at 25° C. and 30° C. Altogether, the encapsulated formulations demonstrated survival advantages for a 3-month period.

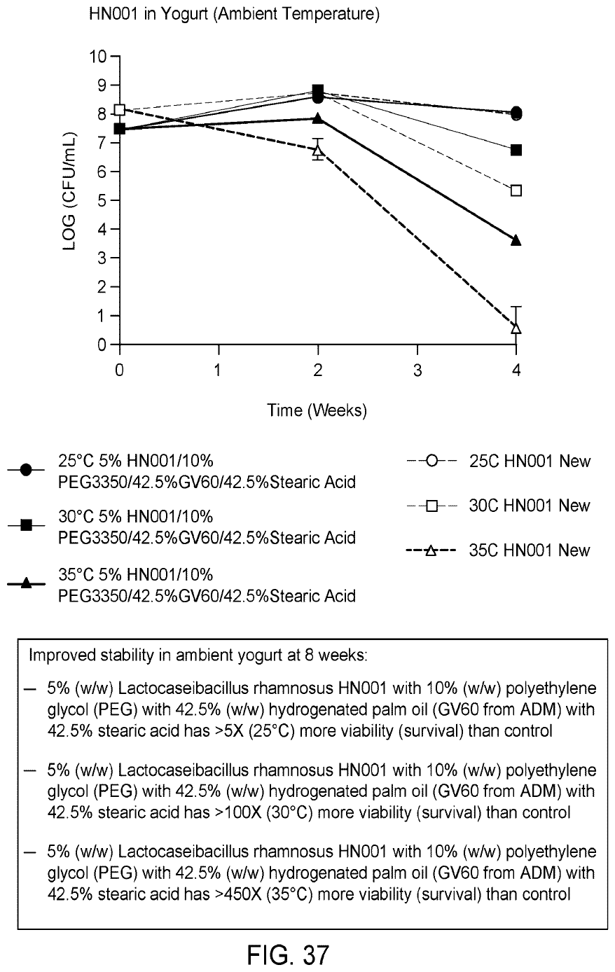

FIG. 37 demonstrates HN001 (non-encapsulated and encapsulated) in yogurt. In this figure, all encapsulated formulations of HN001 (5% (w/w) HN001 with 10% (w/w) polyethylene glycol (PEG) with 42.5% (w/w) hydrogenated palm oil (GV60 from ADM) with 42.5% stearic acid) at 25° C., 30° C., and 35° C. outperformed non-encapsulated HN001 (as provided by the manufacturer). Specifically, encapsulated formulations provided a greater than 5-fold, 100-fold, and 450-fold higher survival (viability as determined by CFU) as compared to non-encapsulated probiotics at 25° C., 30° C., and 35° C., respectively, at 4 weeks. This demonstrated that an encapsulation formulation can protect the HN001 from oxygen, moisture, heat, etc., challenges that were encountered during storage in yogurt at 25° C., 30° C., and 35 CC. Altogether, the encapsulated formulations demonstrated survival advantages over each timepoint evaluated over a 1-month period.

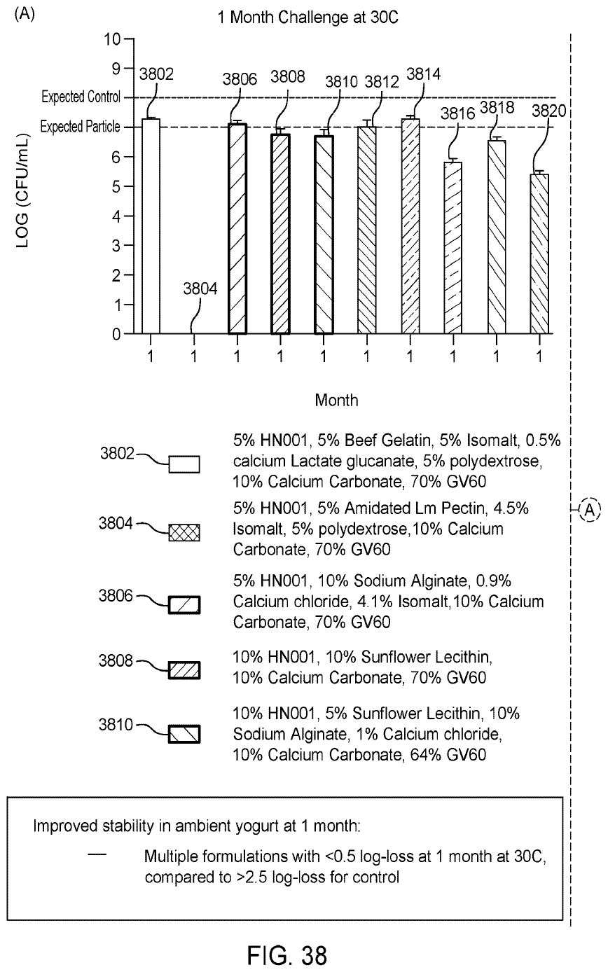

FIG. 38 demonstrates HN001 (non-encapsulated and encapsulated) in yogurt stored at 30° C. for 1 month. In this figure, 8 of the 9 encapsulated formulations of HN001 outperformed non-encapsulated HN001 (as provided by the manufacturer). This figure presents the data as both Log (CFU/ml) and log loss to highlight both the remaining CFU and the loss over a 1-month period. This figure demonstrates materials and formulations (e.g., encapsulation technologies), which may be useful in improving probiotic storage in yogurt. By evaluating materials and formulations for an exemplary condition (e.g., storage in yogurt at 30° C.) as compared to a control (e.g., non-encapsulated HN001) for multiple materials and encapsulation formulations, formulations of potential interest (e.g., <0.5 log loss over a 1 month period) may be quickly identified and evaluated further.

It can be seen by comparing FIG. 31 (encapsulated HN019 in 90% GV60 vs non-encapsulated HN019) to FIG. 34 (encapsulated HN001 in 90% GV60 vs non-encapsulated HN001), that HN001 responded differently to encapsulation as compared to HN019 when stored in yogurt at 25° C., 30° C., and 35° C. This was evidenced by survival advantages provided by encapsulation to HN019 (FIG. 31) as compared to HN001 (FIG. 34). Without wishing to be bound by any particular theory, possible reasons for this include the oxygen sensitivity of HN019 (higher than that of HN001) and/or the heat sensitivity of HN019 (higher than of HN001) experienced during storage in yogurt. Accordingly, encapsulation may provide advantages that improve storage of less-stable probiotics (HN019) in yogurt due to overcoming extreme weaknesses; whereas for HN001, there may be an existing resistance to many of the challenges encountered during storage of probiotics in yogurt. Thus, encapsulation may provide protection against challenges that HN001 is already resistant to, unlike HN019, which demonstrates severe instability against those same challenges; thus advantages of encapsulation may be more pronounced or effective for material choice (e.g., encapsulated probiotic in 90% GV60).

### T. Example 20: Probiotic Compositions Incorporated into Milk Powders or Yogurt

A target probiotic concentration of 1×10⁷⁻⁹ CFU/ml of probiotics in milk powder or yogurt was used for both free probiotics (non-encapsulated) and encapsulated probiotics. A stomacher or mixer was used to mix probiotics and the yogurt or milk powder. Between 1 and 50 ml of yogurt and probiotic mixture were placed into a sealable test tube and additionally sealed with paraffin film. Between 15 and 25 g of milk powder and probiotic mixture were placed into a aluminum or Mylar sachet and either sealed with nitrogen added to the head space or with vacuum. Tubes or sachets were placed into incubators at appropriate temperatures (25° C., 30° C., 35° C.). For free probiotics, an aqueous extraction aws used to enumerate CFUs. Briefly, peptone was warmed to 45° C., 1 ml of yogurt or milk powder with probiotic was added to the peptone, the mixture was allowed to rotate on a rotisserie for 15-20 minutes and serial dilutions were then plated on appropriate agar (e.g., MRS, MRS with 5% L-cysteine, etc.). For encapsulated probiotics, an oil extraction was typically used to enumerate CFUs. Briefly, an oil (e.g., mineral oil, sunflower oil, avocado oil, etc.) was warmed to 35-75° C., PEG40 or other surfactant was added and heated at the same temperature and stirred for 5-20 minutes, then media (e.g., MRS, MRS with 5% L-cysteine, etc.) was added, and serial dilutions were performed and then plated on appropriate agar (e.g., MRS, MRS with 5% L-cysteine, etc.). Enumeration was performed as previously described.

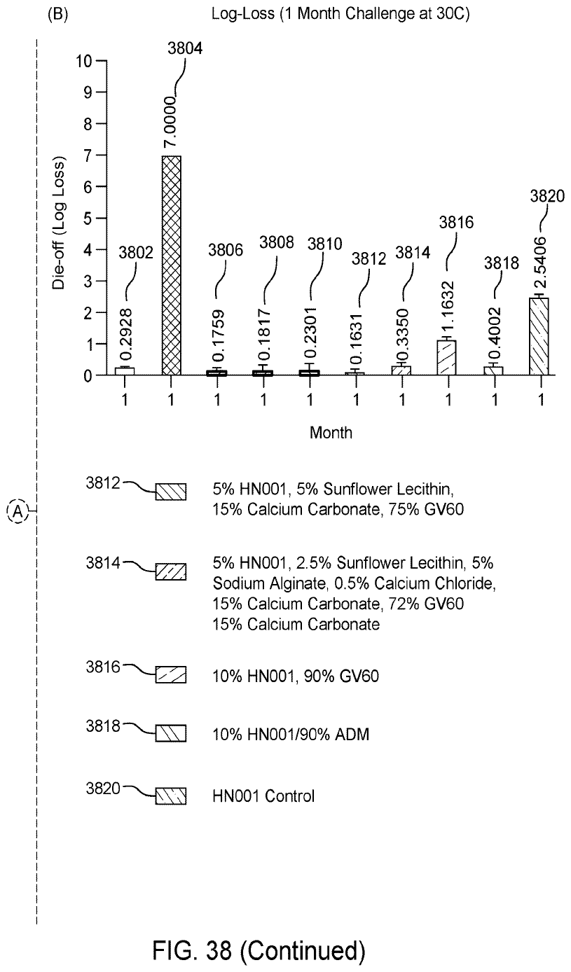

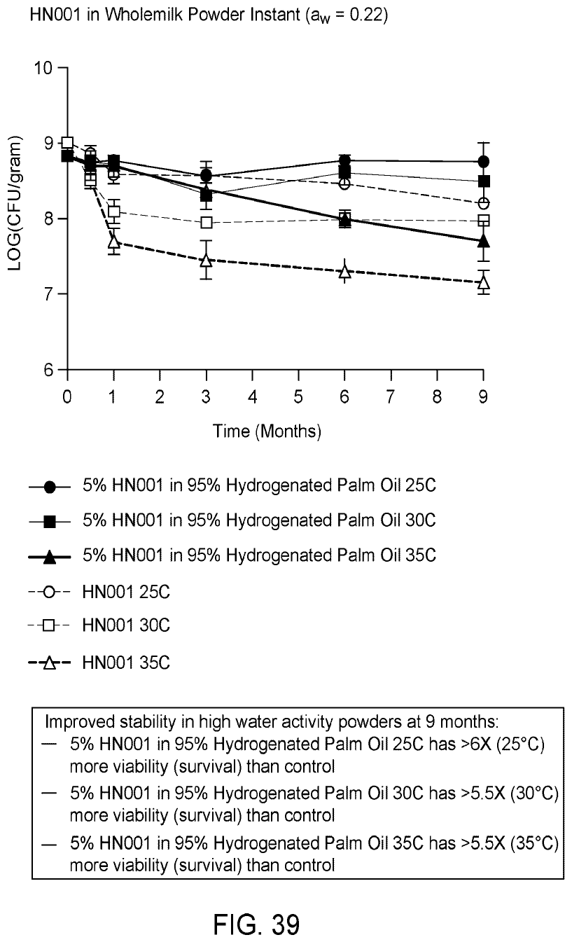

FIG. 39 presents HN001 (non-encapsulated and encapsulated) in a 0.22 water activity milk powder. In this figure, all encapsulated formulations of HN001 (5% HN001 in 95% Hydrogenated Palm Oil) at 25° C., 30° C., and 35° C. outperformed non-encapsulated HN001 (as provided by the manufacturer). Specifically, encapsulated formulations provided a greater than 6-fold, 5.5-fold, and 5.5-fold higher survival (viability as determined by CFU) as compared to non-encapsulated probiotics at 25° C., 30° C., and 35° C. respectively at 9 months. This demonstrates that the encapsulation formulation can protect the HN001 from oxygen, moisture, heat, etc. (other challenges) that are encountered during storage in milk powders of high water activity at 25° C., 30° C., and 35° C.

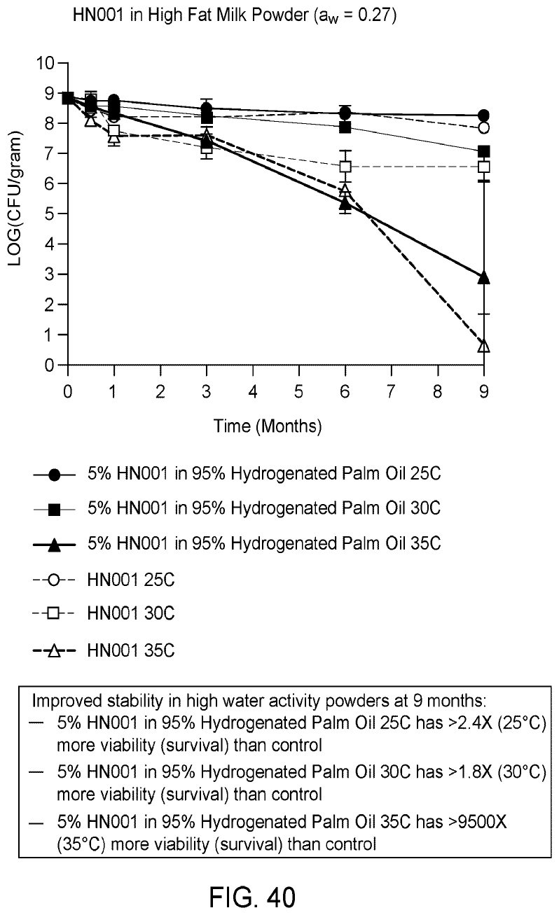

FIG. 40 presents HN001 (non-encapsulated and encapsulated) in a 0.27 water activity milk powder. In this figure, all encapsulated formulations of HN001 (5% HN001 in 95% Hydrogenated Palm Oil) at 25° C., 30° C., and 35° C. outperformed non-encapsulated HN001 (as provided by the manufacturer). Specifically, encapsulated formulations provided a greater than 2.4-fold, 1.8-fold, and 9500-fold higher survival (viability as determined by CFU) as compared to non-encapsulated probiotics at 25° C. 30° C., and 35° C. respectively at 9 months. This demonstrates that the encapsulation formulation can protect the HN001 from oxygen, moisture, heat, etc. (other challenges) that are encountered during storage in milk powders of high water activity at 25° C., 30° C., and 35° C.

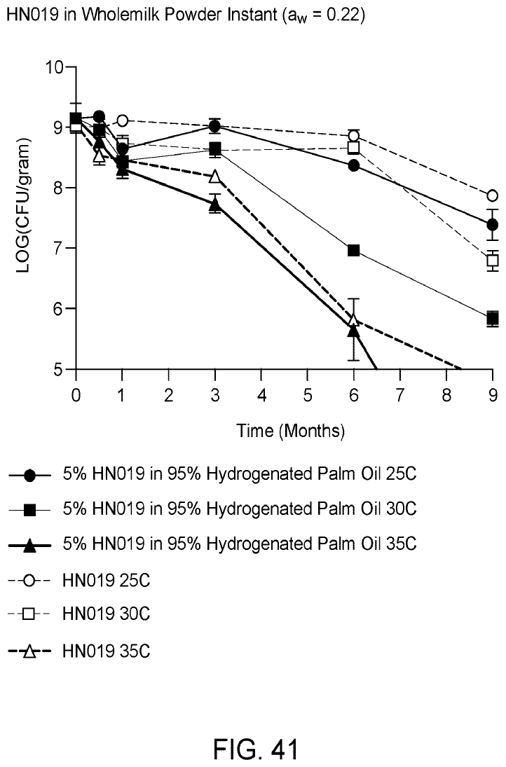

Comparing FIG. 39 (encapsulated 5% HN001 in 95% hydrogenated palm oil vs non-encapsulated HN001) to FIG. 41 (encapsulated 5% HN019 in 95% hydrogenated palm oil vs non-encapsulated HN019), it is clear that HN001 responded differently to encapsulation as compared to HN019 when stored in 0.22 water activity milk powders at 25° C., 30° C., and 35° C. This is evidenced by the survival advantages provided by encapsulation to HN001 (FIG. 39) as compared to HN019 (FIG. 41). Possible reasons for this include the oxygen sensitivity of HN019 (more so than HN001), heat sensitivity of HN019 (more so than HN00l) experienced during storage in powder, moisture stability of HN019 (more so than HN001). In this case, encapsulation may provide advantages that improve storage of more-stable probiotics (HN001l) in milk powders due to supporting existing strengths (for example, HN001 is more resistant to high moisture than HN019, as evidence by the control data in FIGS. 39 and 41). Thus, encapsulation may provide protection against challenges that HN001 is already resistant to, unlike HN019 which demonstrates severe instability against those same challenges; thus the advantages of encapsulation may be more pronounced or effective for this specific material choice or probiotic.

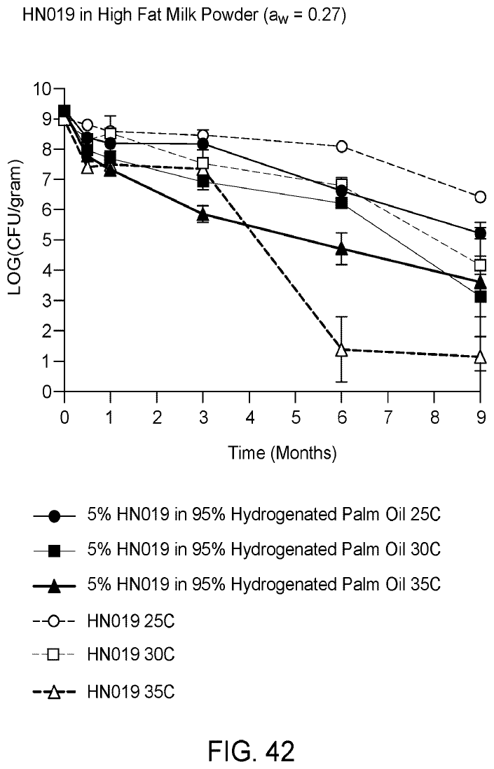

Comparing FIG. 40 (encapsulated 5% HN001 in 95% hydrogenated palm oil vs non-encapsulated HN001) to FIG. 42 (encapsulated 5% HN019 in 95% hydrogenated palm oil vs non-encapsulated HN019), it is clear that HN001 responds differently to encapsulation as compared to H14N019 when stored in 0.27 water activity milk powders at 25° C., 30° C., and 35° C. This is evidenced by the survival advantages provided by encapsulation to HN001 (FIG. 40) as compared to HN019 (FIG. 42 Possible reasons for this include the oxygen sensitivity of HN019 (more so than HN001), heat sensitivity of HN019 (more so than 14N001) experienced during storage in powder, moisture stability of HN019 (more so than HN001). In this case, encapsulation may provide advantages that improve storage of more-stable probiotics (HN001) in milk powders due to supporting existing strengths (for example, HN001 is more resistant to high moisture than HN019, as evidence by the control data in FIG. 40 and FIG. 42). Thus, encapsulation may provide protection against challenges that HN001 is already resistant to, unlike HN019 which demonstrates severe instability against those same challenges; thus the advantages of encapsulation may be more pronounced or effective for this specific material choice or probiotic.

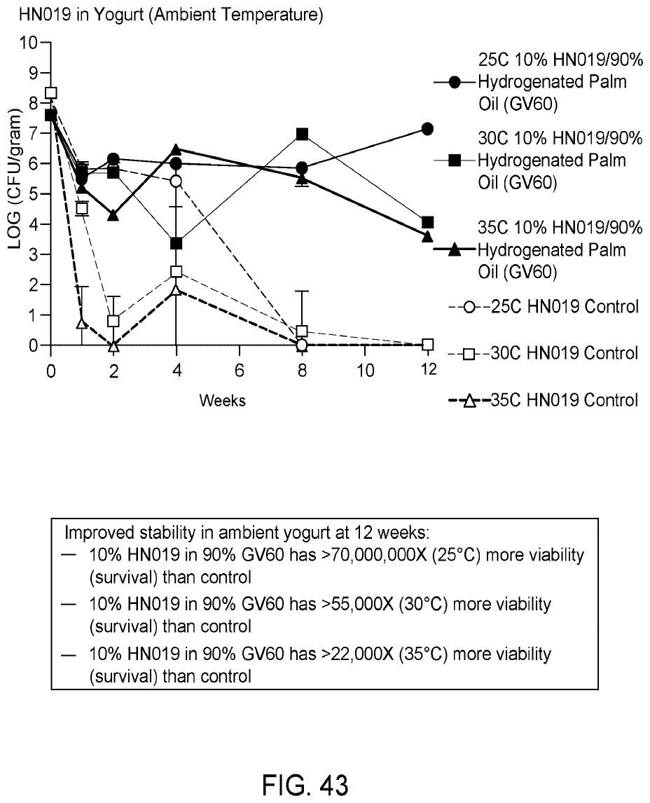

FIG. 43 presents HN019 (non-encapsulated and encapsulated) in yogurt. In this figure, all encapsulated formulations of HN019 (10% HN019 in 90% Hydrogenated Palm Oil (GV60)) at 25° C., 30° C., and 35° C. outperformed non-encapsulated HN019 (as provided by the manufacturer). Specifically, encapsulated formulations provided a greater than 70,000.000-fold, 55,000-fold, and 22,000-fold higher survival (viability as determined by CFU) as compared to non-encapsulated probiotics at 25° C., 30° C. and 35° C. respectively at 12 weeks. This demonstrates that the encapsulation formulation can protect the HN019 from oxygen, moisture, heat, etc. challenges that are encountered during storage in yogurt at 25° C., 30° C., and 35° C.

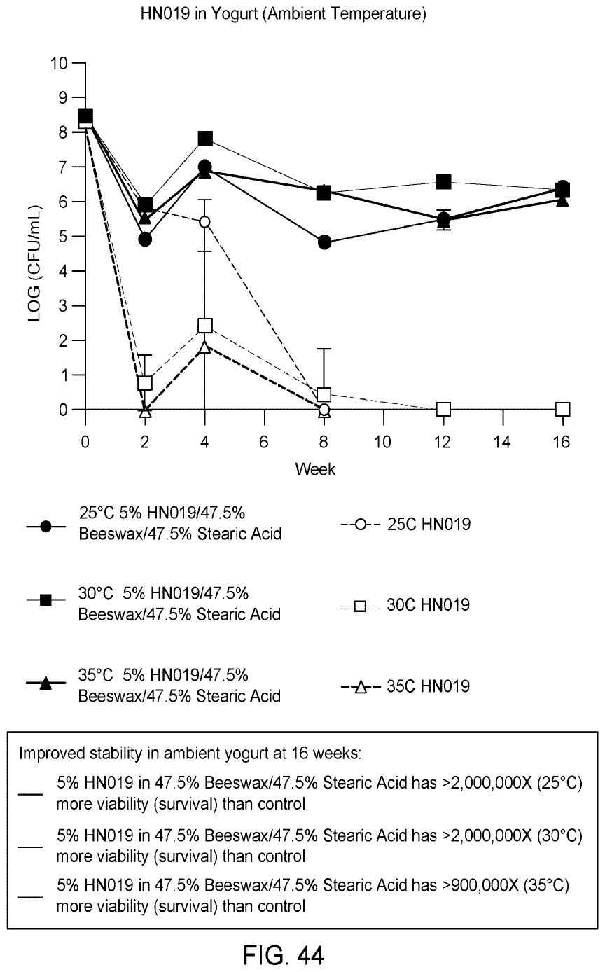

FIG. 44 presents HN019 (non-encapsulated and encapsulated) in yogurt. In this figure, all encapsulated formulations of HN019 (5% HN019 in 47.5% Beeswax/47.5% Stearic Acid) at 25° C., 30° C., and 35° C. outperformed non-encapsulated HN019 (as provided by the manufacturer). Specifically, encapsulated formulations provided a greater than 2,000,000-fold, 2,000,000-fold, and 900,000-fold higher survival (viability as determined by CFU) as compared to non-encapsulated probiotics at 25° C., 30° C., and 35° C., respectively, at 16 weeks. This demonstrates that the encapsulation formulation can protect the HN019 from oxygen, moisture, heat, etc. challenges that are encountered during storage in yogurt at 25° C., 30° C., and 35° C. Altogether, the encapsulated formulations demonstrate survival advantages over each timepoint evaluated over the 4 month period.

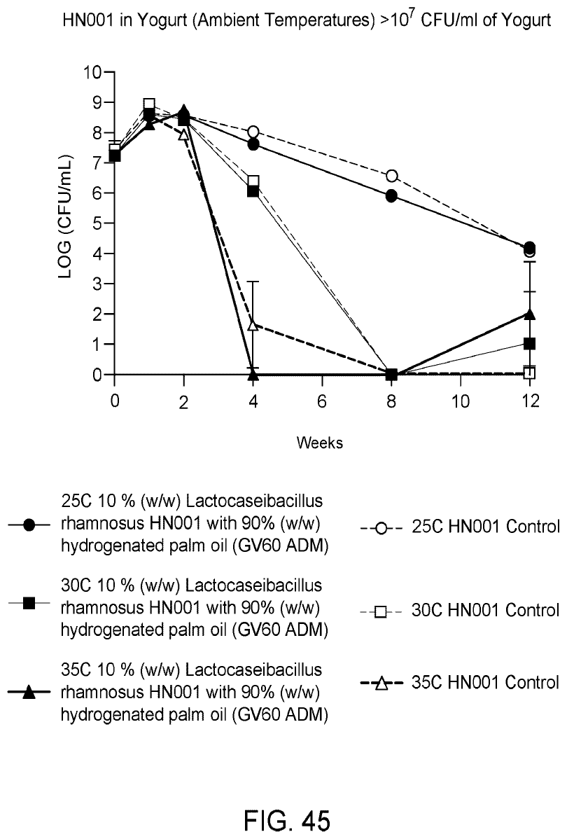

FIG. 45 presents HN001 (non-encapsulated and encapsulated) in yogurt. In this figure, encapsulated formulations of HN001 (10% HN00 l in 90% Hydrogenated Palm Oil (GV60 from ADM)) at 25° C., 30° C., and 35° C. exhibit similar die-off or log-loss or viability loss as compared to non-encapsulated HN001 (as provided by the manufacturer) at 25° C., 30° C., and 35° C. This demonstrates that the encapsulation formulation does not protect the HN001 from oxygen, moisture, heat, etc. challenges that are encountered during storage in yogurt at 25° C., 30° C., and 35° C. At all temperatures tested, non-inferiority between encapsulated and non-encapsulated HN001 was observed.

Comparing FIG. 43 (encapsulated HN019 in 90% GV60 vs non-encapsulated HN019) to FIG. 45 (encapsulated HN001 in 90% GV60 vs non-encapsulated HN001), it is clear that HN001 responds differently to encapsulation as compared to HN019 when stored in yogurt at 25° C., 30° C., and 35° C. This is evidenced by the survival advantages provided by encapsulation to HN019 (FIG. 43) as compared to HN001 (FIG. 45). Possible reasons for this include the oxygen sensitivity of HN019 (more so than HN001), heat sensitivity of HN019 (more so than HN001) experienced during storage in yogurt. In this case, encapsulation may provide advantages that improve storage of less-stable probiotics (HN019) in yogurt due to overcoming extreme weaknesses; whereas for HN001 there may be an existing resistance to many of the challenges encountered during storage of probiotics in yogurt. Thus, encapsulation may provide protection against challenges that HN001 is already-resistant to, unlike HN019 which demonstrates severe instability against those same challenges; thus the advantages of encapsulation may be more pronounced or effective for this specific material choice (encapsulated probiotic in 90% GV60).

### U. Example 21: Probiotic Compositions Incorporated into Electrolyte Beverages

The present example presents a target probiotic concentration of 1×10⁸⁻⁹ CFU/serving of probiotics in an exemplary electrolyte beverage (e.g., Gatorade) was used for both free probiotics (non-encapsulated) and encapsulated probiotics. Free and encapsulated probiotics were added to Gatorade and shaken to mix and then hermetically sealed in a plastic container. Containers were placed into incubators at appropriate temperatures (25° C.). For free probiotics, an aqueous extraction was used to enumerate CFUs. Briefly, peptone was warmed to 45 C, 1 ml of Gatorade with probiotic was added to the peptone, the mixture was allowed to rotate on a rotisserie for 15-20 minutes and serial dilutions are then plated on appropriate agar (e.g., MRS). For encapsulated probiotics, a filtering step, followed by an oil extraction was typically used to enumerate CFUs. Briefly, an oil (e.g., mineral oil, sunflower oil, avocado oil, etc.) was warmed to 35-75° C., PEG40 or other surfactant was added and heated at the same temperature and stirred for 5-20 minutes, then media (e.g., MRS) was added, and serial dilutions were performed and then plated on appropriate agar (e.g., MRS). Enumeration was performed as previously described.

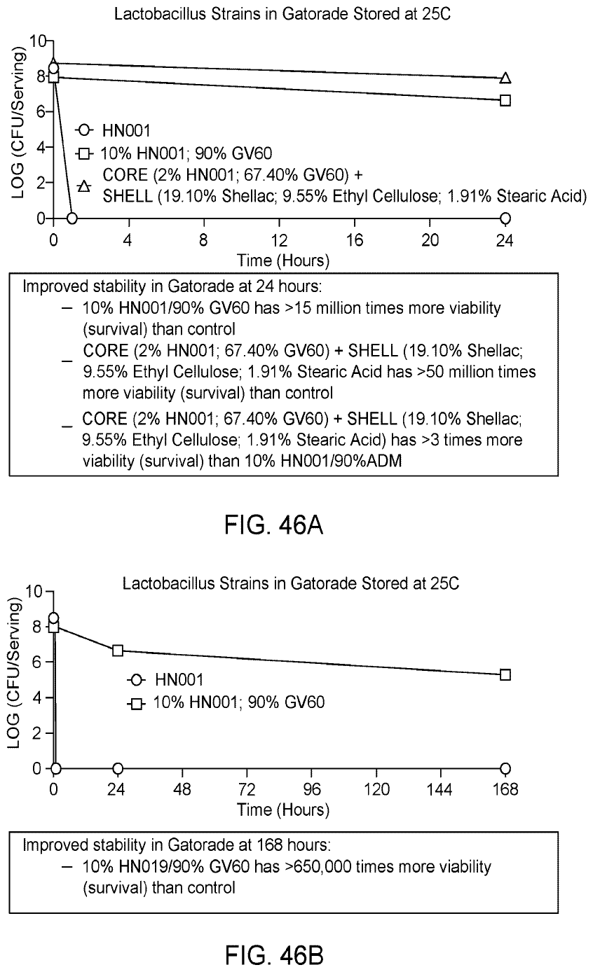

FIG. 46A describes HN001 (non-encapsulated and encapsulated) in Gatorade at 25° C. for 24 hours. In this figure, all encapsulated formulations of HN001 (10% HN001 in 90% Hydrogenated Palm Oil/GV60 and core-shell probiotic particles [CORE (2% HN001: 67.40% GV60)+SHELL (19.10% Shellac; 9.55% Ethyl Cellulose: 1.91% Stearic Acid)]) at 25° C. outperform non-encapsulated HN001 (as provided by the manufacturer). Specifically, encapsulated formulations provide a greater than 15 million-fold (10% HN001 in 90% Hydrogenated Palm Oil/GV60) and 50 million-fold (core-shell probiotic particles [CORE (2% HN001; 67.40% GV60)+SHELL (19.10% Shellac; 9.55% Ethyl Cellulose: 1.91% Stearic Acid)]) higher survival (viability as determined by CFU) as compared to non-encapsulated probiotics at 25° C. at 24 hours. The present example demonstrates that the encapsulation formulation can protect the HN001 from acid, preservatives, oxygen, moisture, etc. (other challenges) that may be encountered during storage in Gatorade at 25° C.

FIG. 46B describes HN001 (non-encapsulated and encapsulated) in Gatorade at 25° C. for 168 hours. This figure demonstrates encapsulated HN001 (10% HN001 in 90% Hydrogenated Palm Oil/GV60) at 25° C. outperformed non-encapsulated HN001 (as provided by the manufacturer). Specifically, encapsulated formulations provided a greater than 650,000-fold (10% HN001 in 90% Hydrogenated Palm Oil/GV60) higher survival (viability as determined by CFU) as compared to non-encapsulated probiotics at 25° C. at 168 hours. Accordingly, the present example further demonstrates that the encapsulation formulation can protect the HN001 from acid, preservatives, oxygen, moisture, etc. (other challenges) that are encountered during storage in Gatorade at 25° C. for 168 hours.

Core-shell probiotic particles [CORE (2% HN001; 67.40% GV60)+SHELL (19.10% Shellac; 9.55% Ethyl Cellulose; 1.91% Stearic Acid)]) as described in FIG. 46A, were manufactured via a four-step process. Step 1 involved spray congealing/prilling of HN001 (10% w/w) and hydrogenated palm oil/GV60 (90%). Step 2 involved extrusion of prilled probiotic particles with additional hydrogenated palm oil/GV60 for a w/w % of 3% HN001 and 97% GV60. Step 3 involved hammer milling extrudate into a powder. Step 4 involved fluid-bed coating to create core-shell particles where the final w/w % (considering both core and shell) consisted of CORE (2% HN001; 67.40% GV60) and SHELL (19.10% Shellac; 9.55% Ethyl Cellulose; 1.91% Stearic Acid).

### V. Example 22: Exemplary Methods of Manufacture Result in Particle Compositions Having Controllable Sizes and Morphologies

The following non-limiting example describes different manufacturing methods, repeated methods, or combinations of methods that were used to produce particle compositions of the present invention having controlled sizes and morphologies for optimized incorporation into various products (e.g., food products).

### Extrusion

Bulk material components were blended until uniform. A Haake Minilab III hot melt extruder was heated to the desired temperature and the twin screws were started at 25-50 RPM. The formulation blend was then manually fed into the extruder and pressure was added with either a manual or automatic pneumatic piston. Extrudate was then collected, weighed, and sealed under nitrogen.

Bulk materials used to form the extrudate included exemplary formulations as described in Table 2 below.

TABLE 2
Exemplary Formulations 1-5

| Sample ID | Probiotic | Wt, % | Component 1 | Wt, % | Component 2 | Wt, % |
|---|---|---|---|---|---|---|
| Formulation 1 | HN001 | 50.0% | GV60 | 40.0% | Calcium Carbonate | 10.0% |
| (830-1) | | | | | | |
| Formulation 2 | HN001 | 28.1% | GV60 | 56.3% | Calcium Carbonate | 15.6% |
| (830-2) | | | | | | |
| Formulation 3 | HN001 | 15.8% | GV60 | 65.8% | Calcium Carbonate | 18.5% |
| (830-3) | | | | | | |
| Formulation 4 | HN001 | 8.9% | GV60 | 70.4% | Calcium Carbonate | 20.7% |
| (830-4) | | | | | | |
| Formulation 5 | HN001 | 5.0% | GV60 | 73.4% | Calcium Carbonate | 21.6% |
| (830-5) | | | | | | |

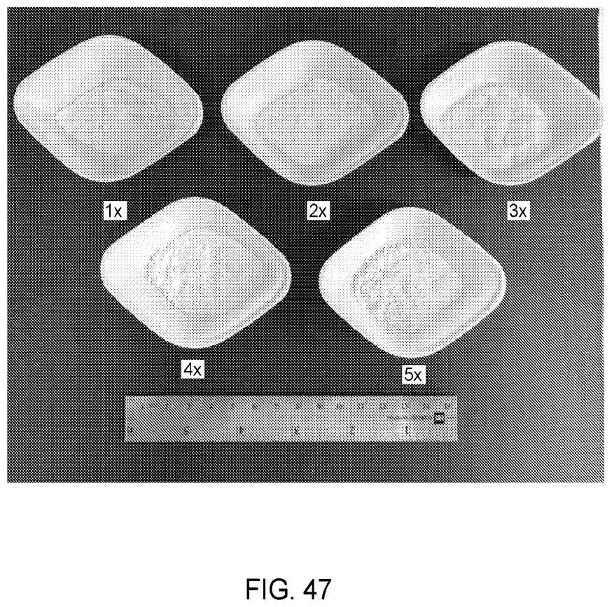

Bulk material of Formulations 1-5 was extruded or multiply extruded (e.g., Formulation 1, 1X: Formulation 2, 2X; Formulation 3, 3X; Formulation 4, 4X; or Formulation 5, 5X) then hammer milled to result in particle compositions as shown in FIG. 47.

### Milling

### Hammer Milling

An IKA Hammer Mill/Fitzpatrick SLS was assembled and equipped with the target product screen size, typically 100-500 μm. If needed, the mill was then cooled with liquid nitrogen. Next, the mill was started with a speed between 3000 and 6500 RPM. The material (e.g., bulk material or extrudate) to be milled was optionally cooled with liquid nitrogen or frozen at −80° C. in a freezer then metered in at a rate to prevent overheating of the mill. As needed, additional liquid nitrogen was added to the mill. Milled product was then collected and sealed under nitrogen.

### Jet Milling

Air or nitrogen at 5 CFM and 100 psi was allowed to flow through a Fluid Energy Jet-O-Mizer mill. The flow to the pusher and grinding nozzles were adjusted as necessary. Material to be milled (e.g., bulk material or extrudate) was added to the vibratory feeder and the speed was adjusted to ensure even flow into the mill. Milled material was collected and sealed under nitrogen.

TABLE 3
Exemplary Formulation 6

| | Formulation 6 (911-2/3) |
|---|---|
| | wt, % |
| HN001 | 2.5% |
| HN019 | 2.5% |
| LPC37 | 2.5% |
| GV60 | 92.5% |

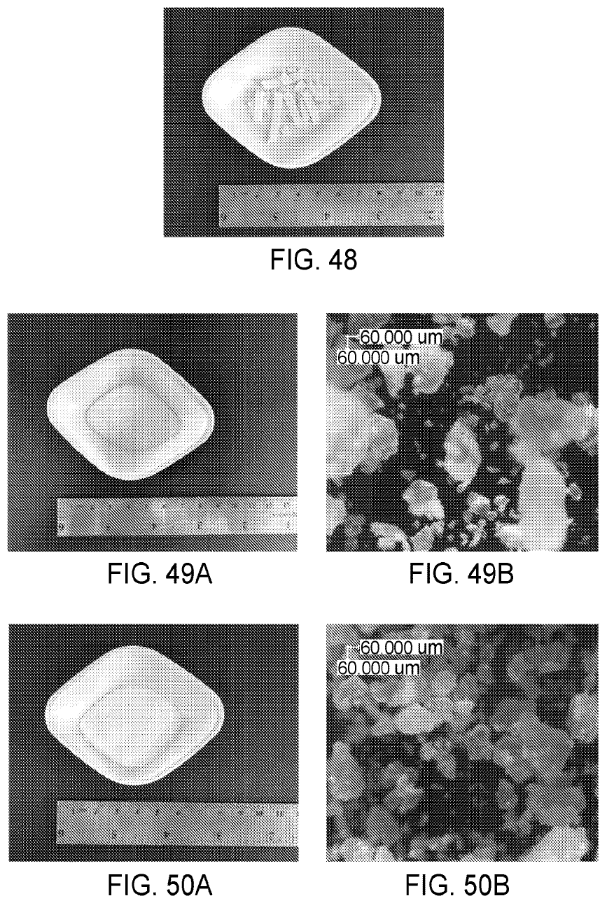

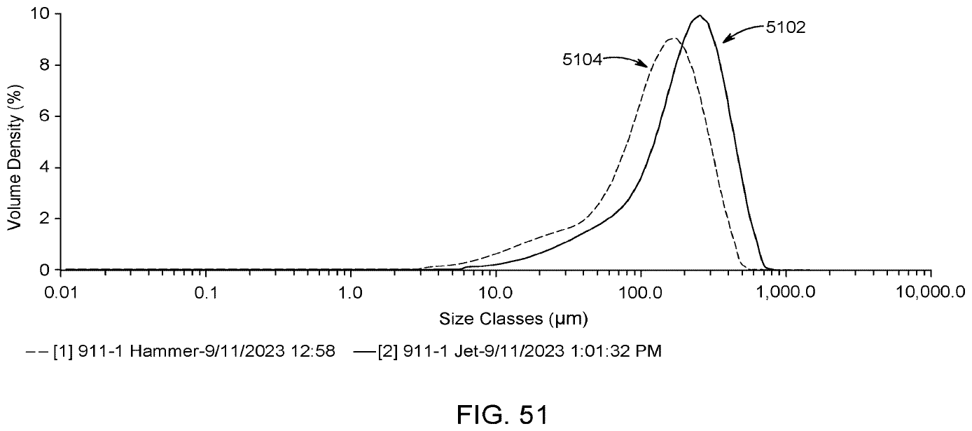

Depending on the manufacture method used, bulk material of Formulation 6 that was extruded (FIG. 48) then hammer milled (FIG. 49A and FIG. 49B) or jet milled (FIG. 50A and FIG. 50B), resulted in particle compositions having different sizes and morphologies. As further shown in the particle size distribution histograms of FIG. 51, the extruded then hammer milled particle composition 5102 had relatively uniform 10%, 50%, and 90% particle size distributions of 54.2 μm, 206 μm, and 407 m, respectively. In contrast, the extruded then jet milled particle composition 5104 had relatively uniform 10%, 50%, and 90% particle size distributions of 29.9 μm, 133 μm, and 280 μm, respectively.

### Fluid Bed Coating

A Freund Vector VFC-Micro fluid bed equipped with a Wurster insert and spray nozzle was preheated to approximately 25-80° C. Material to be coated was then added and airflow was started to fluidize prior to spraying. Spray nozzle air was started, and the required amount of coating solution was metered in using a peristatic pump. The coated material was then fluidized for an additional 10-60 minutes to ensure complete drying or curing. The fluid bed was then turned off and the particle compositions were collected and sealed under nitrogen.

TABLE 4
Exemplary Formulations 7 and 8

| | Formulation 7 (828-2) | |
|---|---|---|
| | wt, % | Formulation 8 (905-1) |
| LPC-37 | 3.0% | 2.1% |
| GV60 | 97.0% | 67.4% |
| Ethyl Cellulose | | 9.5% |
| Shellac | | 19.1% |
| Stearic Acid | | 1.9% |

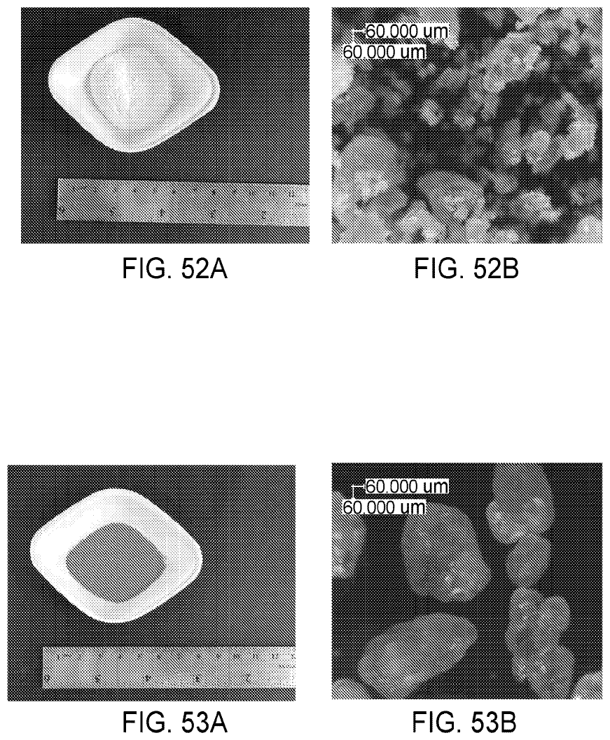

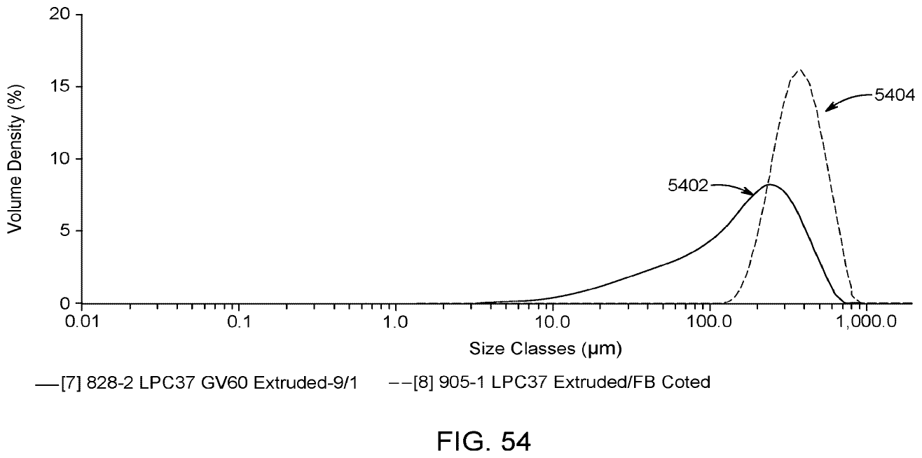

Depending on the manufacture method used, bulk material of Formulation 7 or Formulation 8 (see Table 4, above) that was extruded then hammer milled (FIG. 52A and FIG. 52B) or fluid bed coated (FIG. 53A and FIG. 53B), resulted in particle compositions having different sizes and morphologies. As shown in FIG. 53B, smaller particles agglomerated into larger particles during the fluid bed coating process. As further shown in the particle size distribution histograms of FIG. 54, the extruded then hammer milled particle composition 5402 had relatively uniform 10%, 50%, and 90% particle size distributions of 34.3 m, 170 μm, and 384 μm, respectively. In contrast, the fluid bed coated particle composition 5404 had relatively uniform 10%, 50%, and 90% particle size distributions of 228 μm, 367 μm, and 574 μm, respectively.

### Prilling

Particle compositions of the present example are manufactured by a process as previously described in Example 2. Bulk materials used to form the prilled particle compositions included exemplary formulations as described in Table 5 below.

TABLE 5
Exemplary Formulations 9 and 10

| | Formulation 9 (2201) | Formulation 10 (822-1) |
|---|---|---|
| | wt, % | wt, % |
| HN001 | 10.0% | 7.2% |
| GV60 | 90.0% | 64.5% |
| Eudraguard Biotic | | 28.3% |

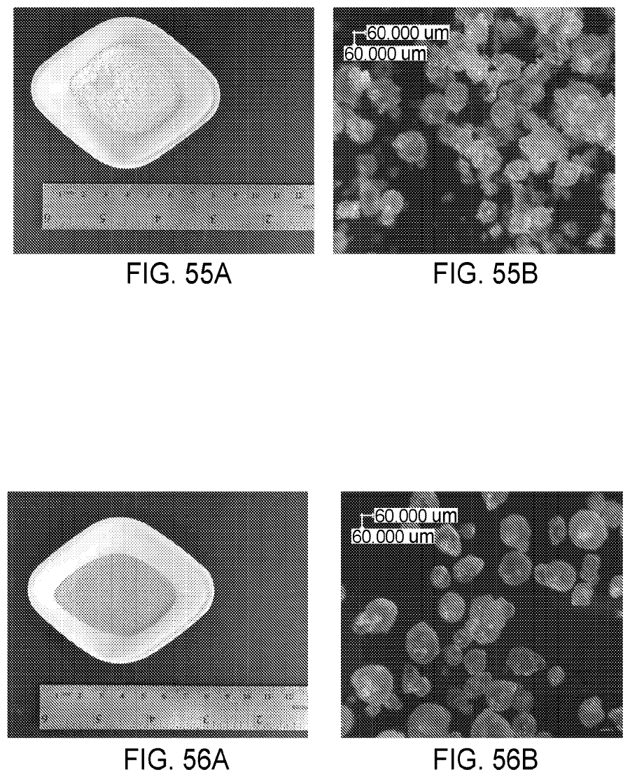

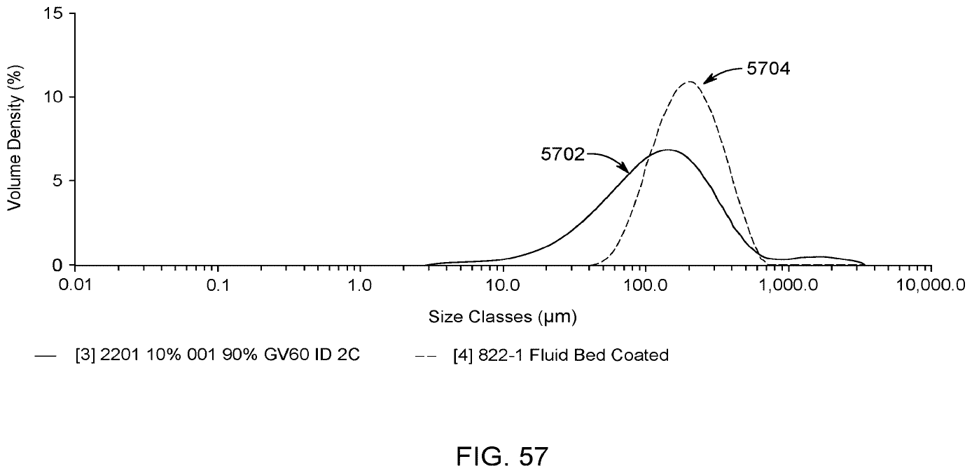

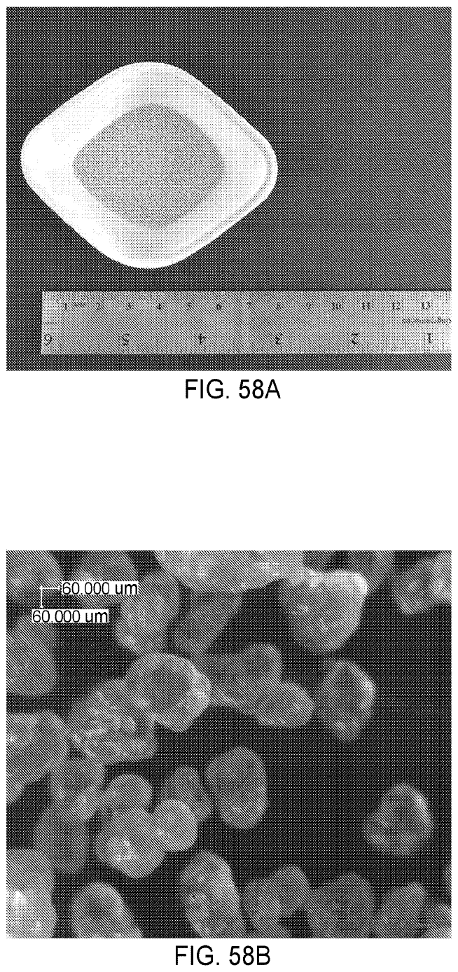

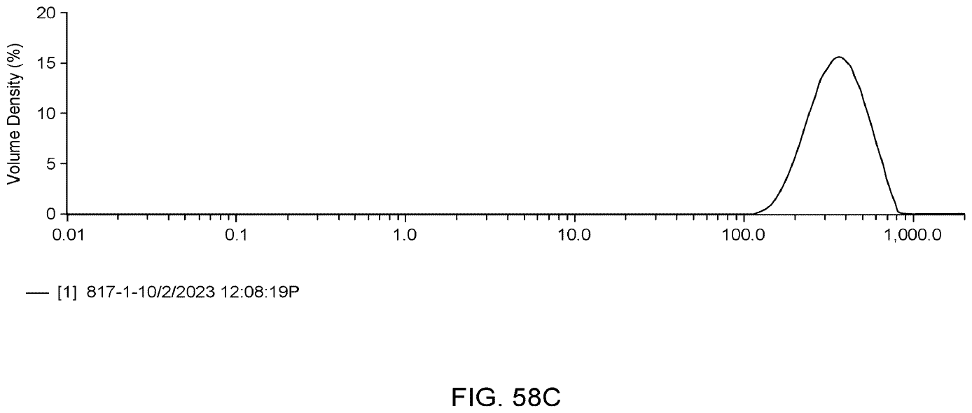

As shown in FIG. 55A-B and FIG. 56A-B, particle compositions of Formulation 9 and Formulation 10 (as described in Table 5, above) resulting from bulk material that was prilled (FIG. 58A and FIG. 58B) or prilled then fluid bed coated (FIG. 56A and FIG. 56B) had different sizes and morphologies. As further shown in the particle size distribution histograms of FIG. 57, the prilled particle composition 5702 had relatively uniform 10%, 50%, and 90% particle size distributions of 33.9 m, 124 μm, and 363 μm. The fluid bed coated particle composition 5704 had relatively uniform 10%, 50%, and 90% particle size distributions of 98.2 m, 196 μm, and 380 m, respectively.

TABLE 6
Exemplary Formulations 11 and 12

| | Formulation 11 (817-1) | Formulation 12 (901-1) |
|---|---|---|
| | wt, % | wt, % |
| LPC 37 | 2.01% | 5.00% |
| GV60 | 67.29% | |
| Ethyl Cellulose | 19.14% | |
| Shellac | 9.57% | |
| Stearic Acid | 1.91% | |
| Eudraguard Biotic | | 95.00% |

Particle compositions of Formulation 11 (as described in Table 6, above) resulting from bulk material that was prilled, extruded, milled, and fluid bed coated are shown in FIG. 58A and FIG. 58B. As further shown in the particle size distribution histograms of FIG. 58C, the prilled, extruded, milled and fluid bed coated particle composition had relatively uniform 10%, 50%, and 90% particle size distributions of 217 m, 356 μm, and 565 μm.

### Spray Drying

Particle compositions of the present disclosure can be produced by spray drying. In brief, probiotics are dispersed in encapsulation formulants forming an emulsion or dispersion, followed by homogenisation of the liquid, then atomisation of the mixture into a drying chamber. This leads to evaporation of the solvent (e.g., water) and formation of the particle compositions.

### Spray Chilling

A Buchi B-290 spray chilling chamber equipped with a spray chilling accessory was cooled and the nozzle and spray air were heated to the required temperatures. Next, the wax or lipid was weighed into a stainless-steel vessel and heated to 10° C. above the melting point. The melt was mixed using a high shear mixer or homogenizer set between 1000 and 10,000 RPM and the required amount of probiotic and any additional co-formulants were slowly added. Mixing continued until all components were fully dispersed. This mixture was then transferred to a heated funnel for transfer to the nozzle. Finally, a valve was opened allowing the mixture to move to the nozzle and be sprayed. Final particle compositions were weighed and sealed under nitrogen.

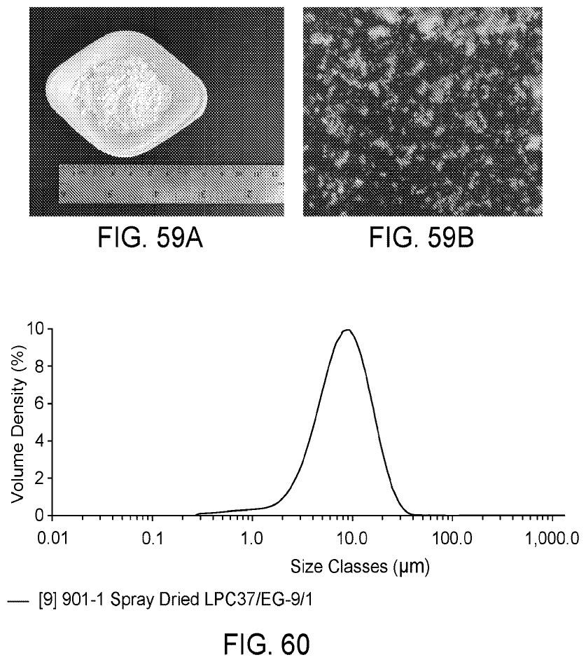

As shown in FIG. 59A and FIG. 59B, smaller particle compositions of Formulation 12 were achievable from bulk material that was spray dried.

As shown in FIG. 60, a particle composition resulting from a spray dried method had relatively uniform 10%, 50%, and 90% particle size distributions of 3.34 μm, 8.13 μm, and 16.8 μm.

### Lyophilization

Particle compositions were freeze dried under vacuum and maintained at a temperature and for a duration sufficient to completely dry the compositions.

TABLE 7
Exemplary Formulation 13

| | Formulation 13 (823-2) |
|---|---|
| | Wt, % |
| HN001 | 3.0% |
| Eudraguard Biotic | 97.0% |

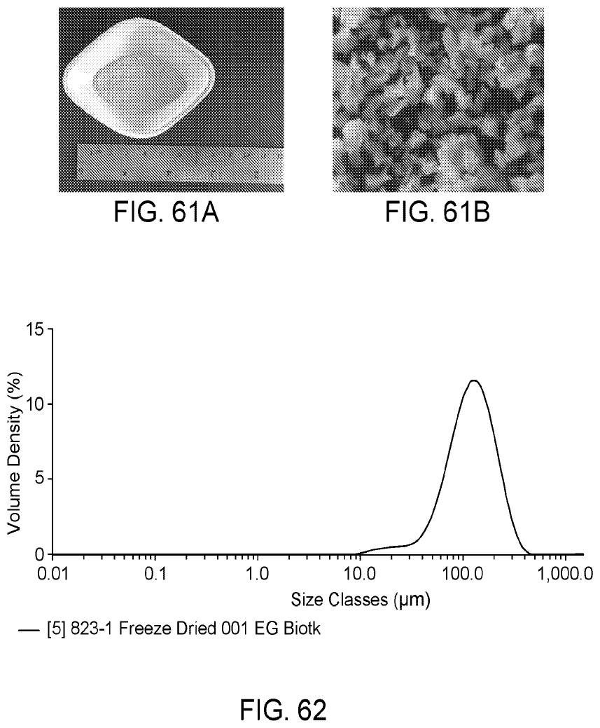

As shown in FIG. 61A and FIG. 61B, a lyophilized particle composition of Formulation 13 (as described in Table 7, above) had irregular morphology but relatively uniform 10%, 50%, and 90% particle size distributions of 57.1 μm, 121 μm, and 224 μm (FIG. 62).

These data show that particle sizes and morphology can be controlled using exemplary manufacturing methods as disclosed herein or combinations thereof.

### W. Example 23: Distribution of Particle Compositions in Liquids

The following non-limiting example describes the distribution characteristics of various particle compositions of the present invention in liquids.

Homogeneous dispersion in liquid beverages is an important characteristic that affects product quality due to appearance, accurate dosing (heterogenous dispersion of probiotic particles would lead to differential dosing with each sip/ingestion event), and stability (different areas in a given product present different challenges; for example, particles that settle to the bottom of a beverage will experience more pressure than particles at the top of a beverage; likewise, particles at the top of the beverage will have greater interaction with gasses in the headspace as compared to particles at the bottom of the beverage). As such, homogeneous distribution of probiotic particles in a beverage is desirable. Without wishing to be bound by any theory, various factors can contribute to homogenous distribution of particle compositions including the size of the particle composition, the formulation of the particle composition, and the characteristics of the liquid in which the particle composition is being dispersed, for example, but not limited to, temperature, viscosity. pH, and fat content.

TABLE 8
Exemplary Formulations 14 and 12

| | Formulation 14 (823-1) | Formulation 12 (901-1) |
|---|---|---|
| | Wt, % | Wt, % |
| LPC-37 | 74.4% | 5.00% |
| Shellac | 16.0% | |
| Ethyl Cellulose | 8.0% | |
| Stearic Acid | 1.6% | |
| Eudraguard Biotic | | 95.00% |

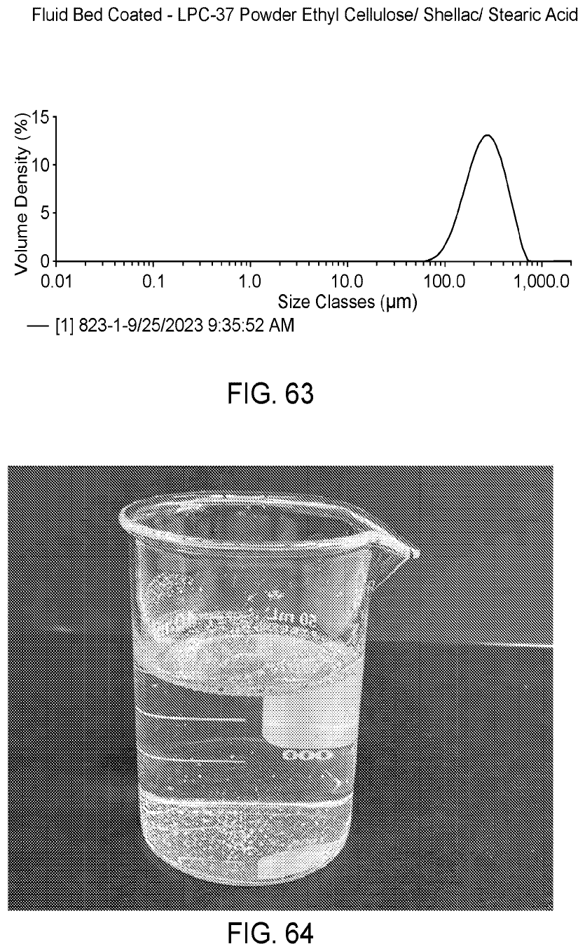

As shown in FIG. 63, a high-load probiotic particle composition of Formula 14 (as described in Table 8, above) resulting from a fluid bed coating method had relatively uniform 10%, 50%, and 90% particle size distributions of 145 m, 263 m, and 451 m. However, when 100 mg of the particle composition was added to 40 mL of water (25° C.), there was relatively poor distribution and dissolution (FIG. 64).

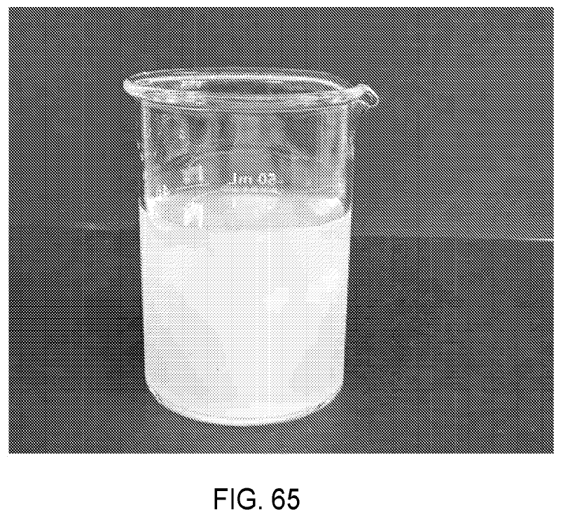

In contrast, as further shown in FIG. 65, the spray dried particle composition of Formulation 12 (as described in Table 8, above) had relatively uniform distribution when 100 mg of composition was mixed in 40 mL of water. This spray-dried particle composition does not include waxes or low-density fat- and/or lipid-based encapsulants which would typically cause particles to rise to the top of the liquid (as observed with the fluid bed coated particles of FIG. 64).

These data indicate that viable particle compositions can be produced without the use of waxes or low-density fat- and/or lipid-based encapsulants. Whereas waxes or low-density fat- and/or lipid-based encapsulants are expected to rise to the top of a liquid (e.g., a beverage), exemplary particle composition formulations of the present disclosure can distribute homogeneously.

### X. Example 24: Probiotic Viability of Particle Compositions in Enteral Feed

The following non-limiting example describes the viability of particle compositions of the present invention when mixed and stored with enteral-feed liquid formula.

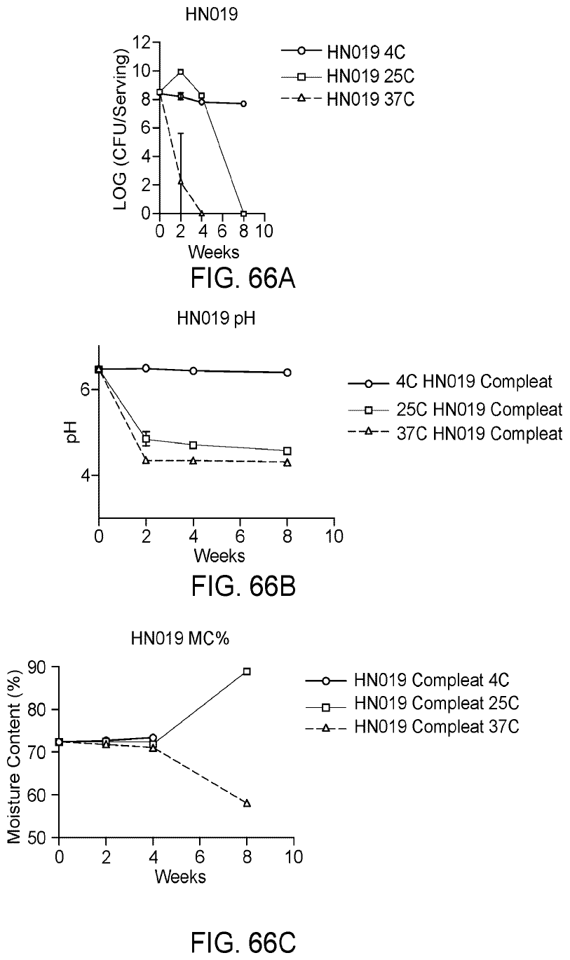

As shown in FIG. 66A, unencapsulated HN019 probiotic had decreased viability after only two weeks of storage at 25° C. (i.e., ambient temperature) and 37° C. when fully mixed with Compleat standard 1.4 plant-based tube/enteral feed liquid formula as compared to unencapsulated HN019 probiotic stored at 4° C. This reduced viability was concomitant with a lowered pH of the formula (FIG. 66B). While unencapsulated HN019 probiotic had an increase in moisture content (FIG. 66C) when maintained at 25° C. (i.e., ambient temperature) for eight weeks, unencapsulated HN019 probiotic had a decrease in moisture content when maintained at 37° C., as measured by a standard moisture balance.

TABLE 9
Exemplary Formulations 15-21

| | Formulation | Formulation | Formulation | Formulation | Formulation | Formulation | Formulation |
|---|---|---|---|---|---|---|---|
| | 15 (814-1) | 16 (815-2) | 17 (817-1) | 18 (818-1) | 19 (706-1) | 20 (717.2) | 21 (717.3) |
| | wt, % | wt, % | wt, % | wt, % | wt, % | wt, % | wt, % |
| HN019 | 5.9% | 8.35% | 2.01% | 2.1% | 8.55% | 10% | 5% |
| GV60 | 53.5% | 75.16% | 62.29% | 69.3% | 68.4% | 90% | |
| Ethyl | 12.7% | | 9.57% | | 14.5% | | |
| Cellulose | | | | | | | |
| Shellac | 25.3% | | 19.14% | | | | |
| Stearic Acid | 2.5% | | 1.91% | | | | 47.5% |
| Eudraguard | | 16.49% | | 28.6% | | | |
| Biotic | | | | | | | |
| Calcium | | | | | 8.55% | | |
| carbonate | | | | | | | |
| Beeswax | | | | | | | 47.5% |
| Processing | Prilling; | Prilling; | Prilling; | Prilling; | Extrusion; | Prilling; | Prilling |
| Method | Fluid Bed | Fluid Bed | Extrusion; | Extrusion; | Fluid Bed | Fluid Bed | |
| | Coating | Coating | Fluid Bed | Fluid Bed | Coating | Coating | |
| | | | Coating | Coating | | | |

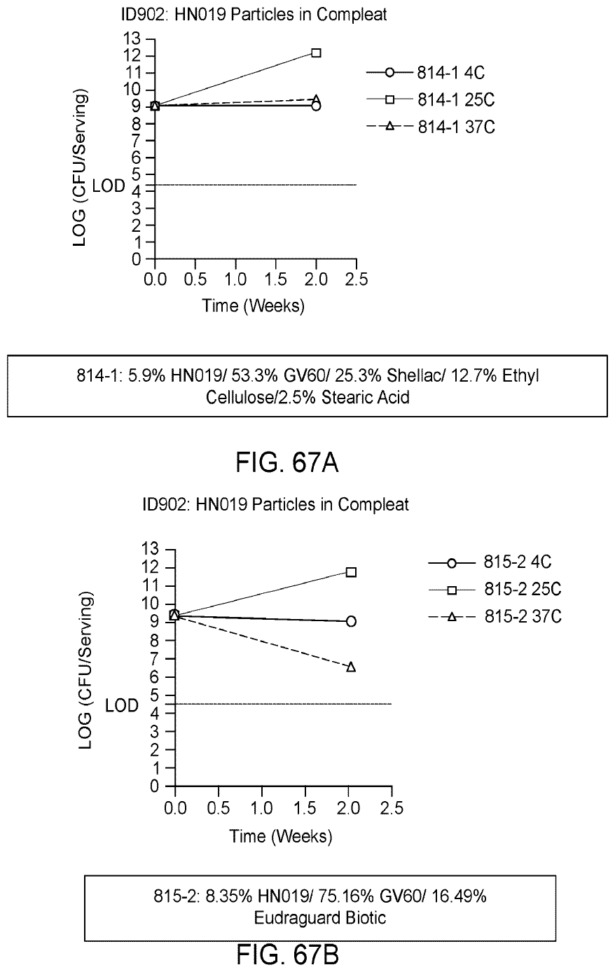

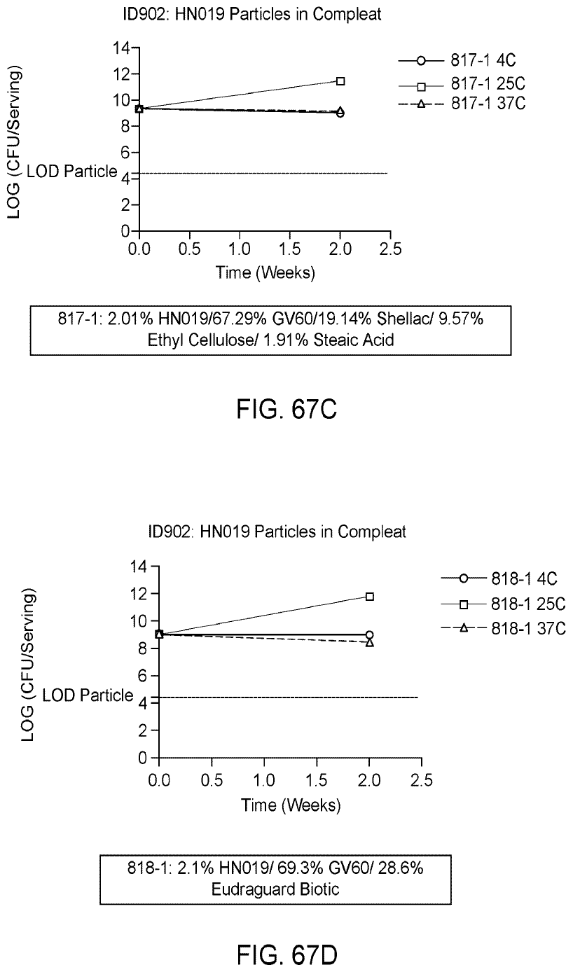

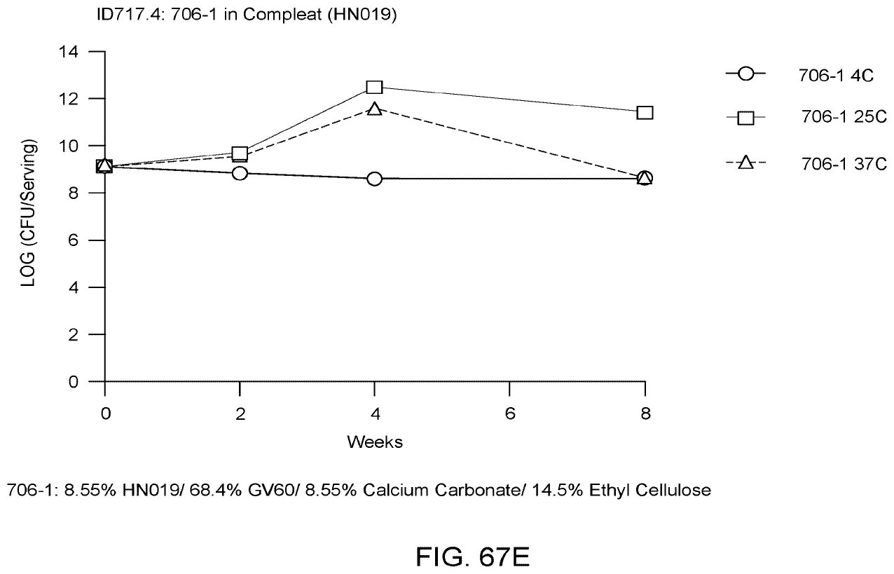

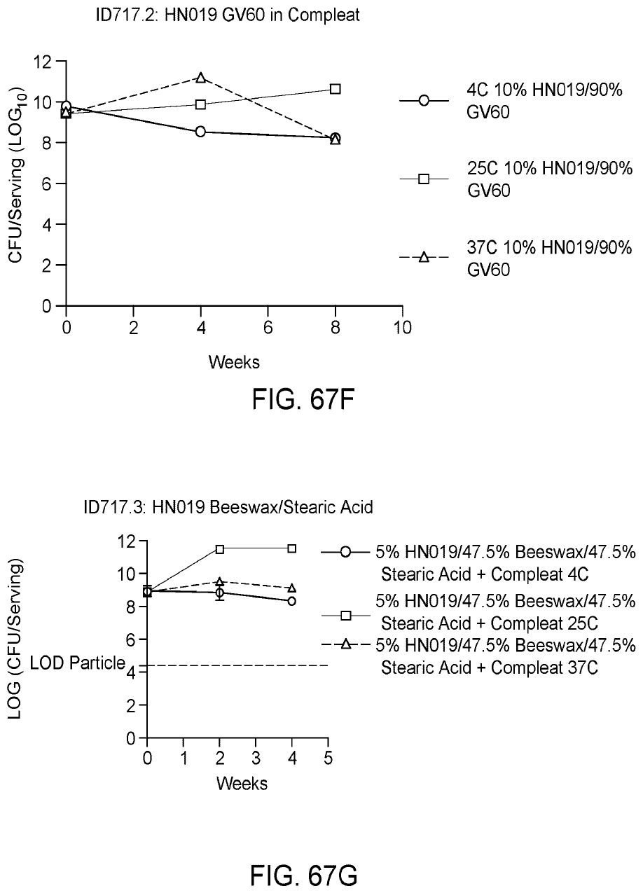

In contrast, seven formulations (Formulations 15-21 as described in Table 8, above) of particle compositions had increased HN019 probiotic viability after two weeks of storage at 25° C. (FIG. 67A-FIG. 67G) when mixed with Compleat standard 1.4 plant-based tube/enteral feed formula. For three formulations, it was demonstrated that increased viability was maintained for as long as four weeks of storage in Compleat standard 1.4 plant-based tube/enteral feed formula (FIG. 67E, FIG. 67F, FIG. 67G). It was further demonstrated that for two formulations, increased viability was maintained for as long as four weeks of storage in Compleat standard 1.4 plant-based tube/enteral feed formula (FIG. 67E, FIG. 67F).

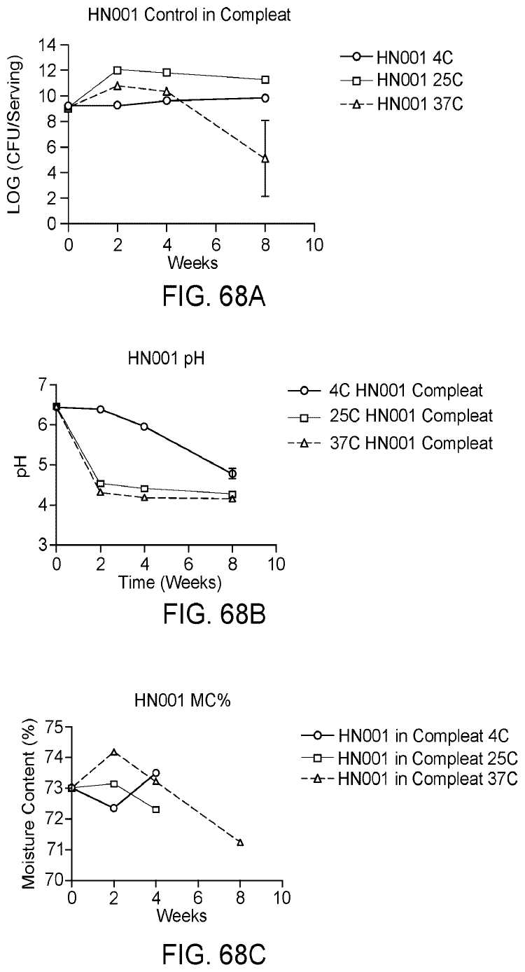

As shown in FIG. 68A, unencapsulated HN001 probiotic had decreased viability after eight weeks of storage at 37° C. when mixed with Compleat standard 1.4 plant-based tube/enteral feed formula as compared to unencapsulated HN019 probiotic stored at 4° C. or 25 C. This reduced viability was concomitant with a lowered pH of the formula (FIG. 68B) and a decrease in moisture content (FIG. 68C) as measured by a standard moisture balance.

TABLE 10
Exemplary Formulations 22-28

| | Formulation | Formulation | | | | | |
|---|---|---|---|---|---|---|---|
| | 22 (530-2) | 23 (530-3) | | | | | |
| | *double | *triple | Formulation | Formulation | Formulation | Formulation | Formulation |
| | extruded | extruded | 24 (524-1) | 25 (525-1) | 26 (558-1) | 27 (558-2) | 28 (558-3) |
| | wt, % | wt, % | wt, % | wt, % | wt, % | wt, % | wt, % |
| HN001 | 5.9% | 15.8% | 83% | 13% | 5% | 5% | 2.5% |
| GV60 | 56.28% | 65.75% | | 74.5% | | | 47.5% |
| Ethyl | | | 12% | 1.86% | | | |
| Cellulose | | | | | | | |
| PEG 3350 | | | | | | 10% | |
| Polydextrose | | | | | | | 2.5% |
| Stearic Acid | | | | | 47.5% | 42.5% | |
| Oleic Acid | | | 5% | 0.75% | | | |
| Calcium | 15.62% | 18.47% | | 10% | | | |
| carbonate | | | | | | | |
| Beeswax | | | | | 47.5% | 42.5% | 47.5% |
| Processing | Extrusion; | Extrusion; | Fluid Bed | Fluid Bed | Prilling | Prilling | Prilling |
| Method | Extrusion | Extrusion; | Coating | Coating; | | | |
| | | Extrusion | | Extrusion; | | | |

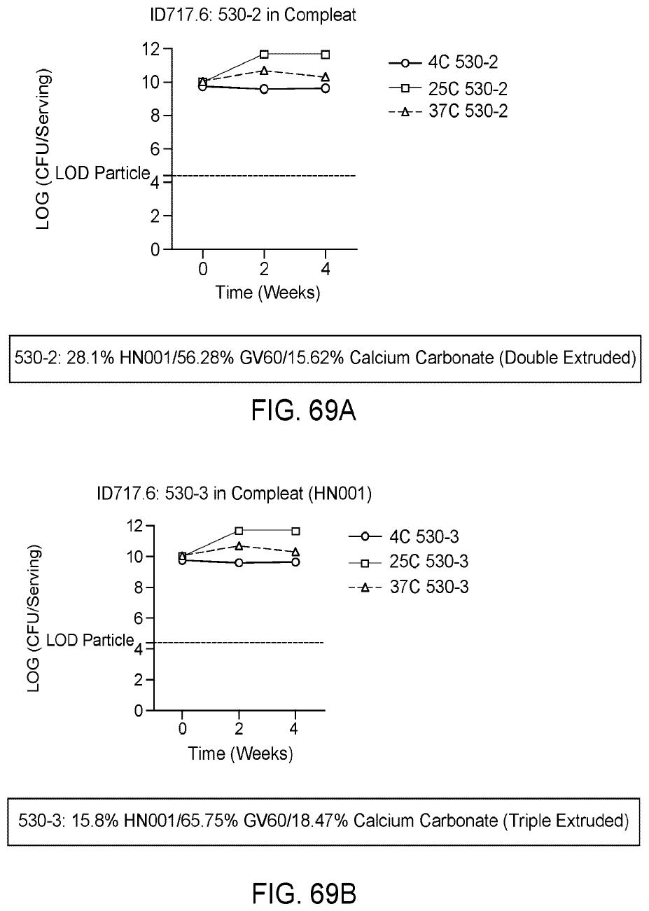

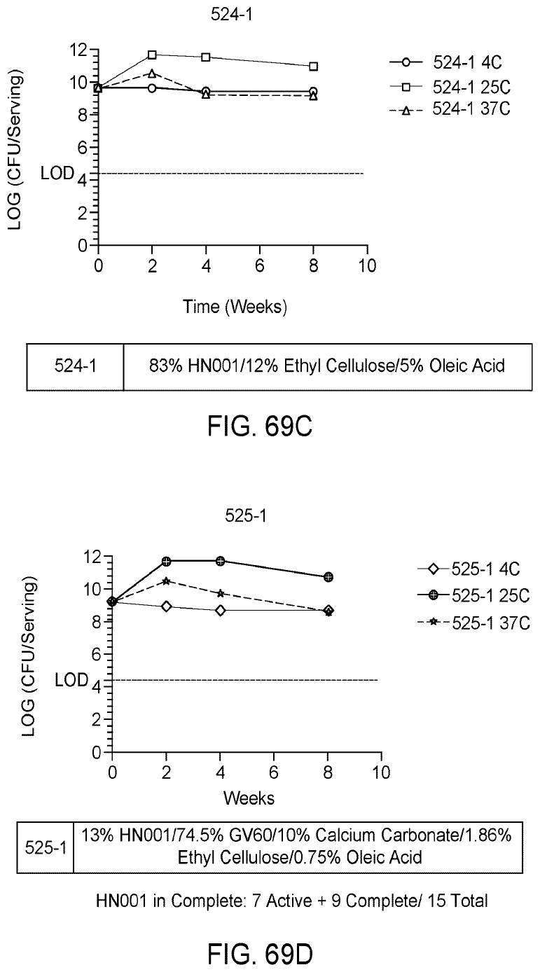

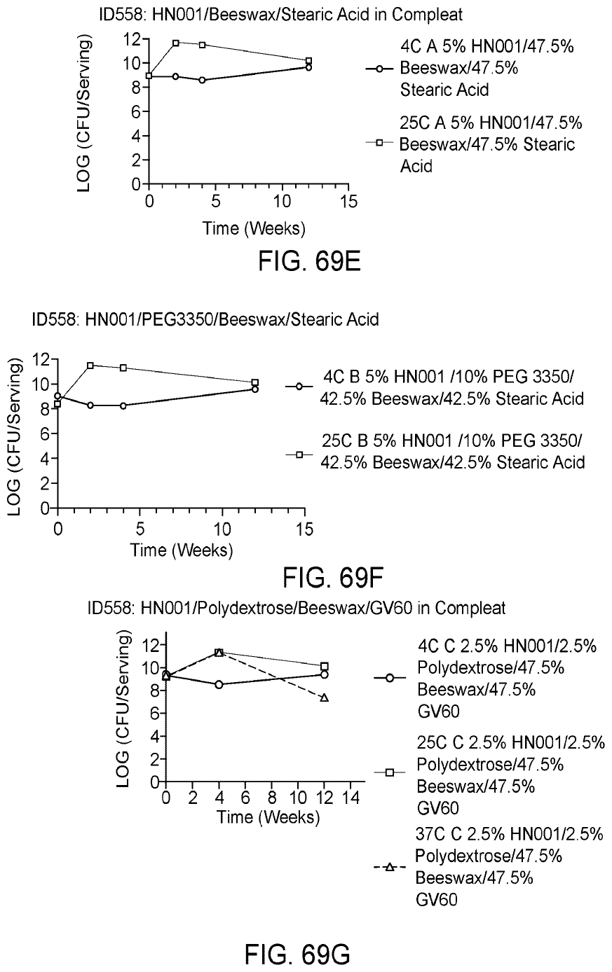

In contrast, as shown in FIG. 69C, FIG. 69D, two formulations (Formulation 24 and Formulation 25, as described in Table 10, above) of particle compositions had increased probiotic viability after eight weeks of storage at 37° C. when mixed with Compleat standard 1.4 plant-based tube/enteral feed formula.

These data indicate that exemplary particle compositions of the present disclosure can be used to stabilize probiotics in enteral-feed formula.

### Y. Example 25: Probiotic Viability of Particle Compositions in Water

The following non-limiting example describes the viability of particle compositions of the present invention when added to water and stored.

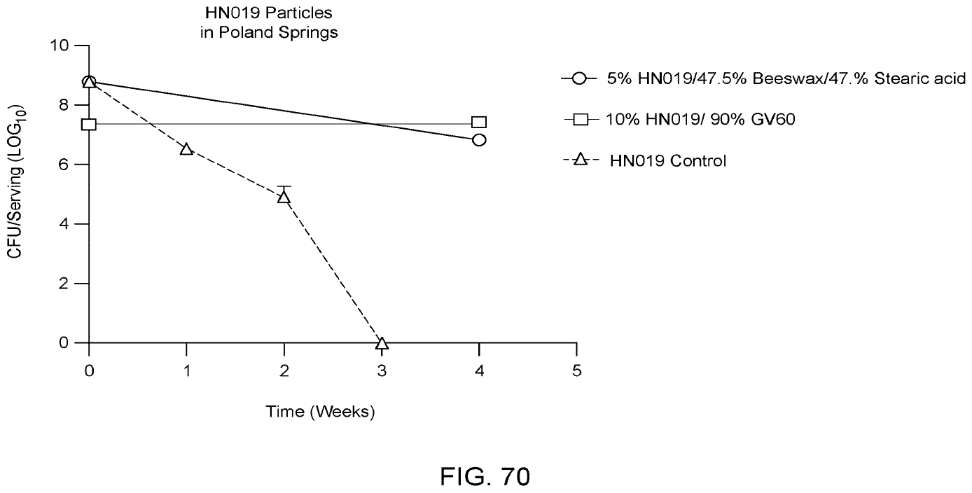

As shown in FIG. 70, particle compositions having Formulations 20 and 21 (as described in Table 10, above), had maintained HN019 probiotic viability over the course of a four-week storage period when the equivalent of 10⁹ cfu/serving (355 mL) of each was directly added to vessels containing 500 mL of Poland Springs water and mixed by inversion and shaking then stored at 25° C. In contrast, unencapsulated HN019 probiotic exhibited a complete loss of viability by 3 weeks of storage in Poland Springs water.

Without wishing to be bound by any particular theory, exposure to moisture and humidity generally lead to degradation and loss of viability and stability of probiotics. These data indicate that exemplary particle compositions of the present disclosure protect probiotics from moisture to promote their viability when stored in liquid products (e.g., beverages).

### Z. Example 26: Probiotic Viability of Particle Compositions in Milk

The following non-limiting example describes the viability of particle compositions of the present invention when added to milk and stored.

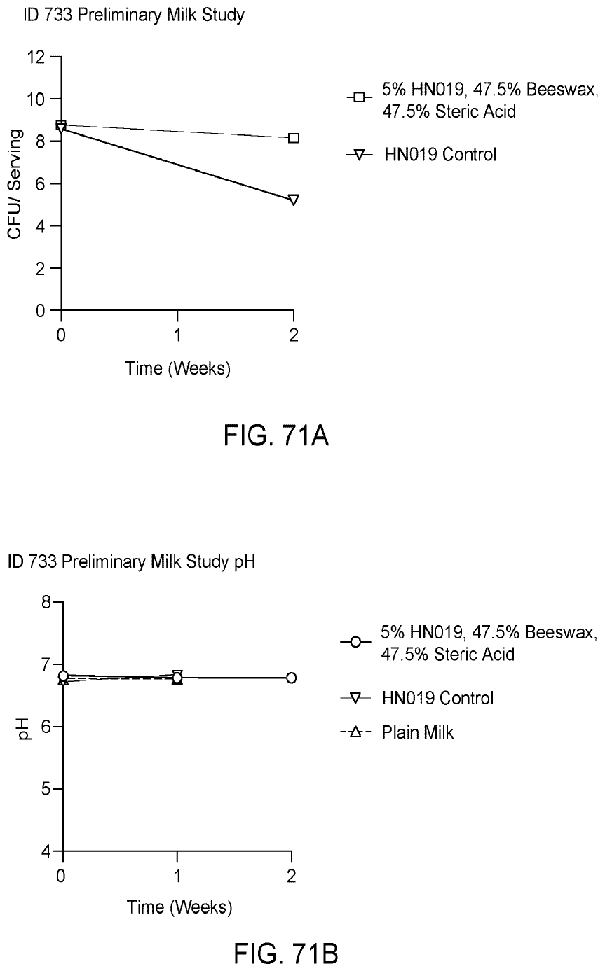

As shown in FIG. 71A, particle compositions having Formulation 21 (as described in Table 9, above) had maintained HN019 probiotic viability over the course of a two-week storage period when the equivalent of 10⁹ cfu/serving (355 mL) of each was directly added to vessels containing 500 mL of fresh milk and mixed by inversion and shaking then stored at 4° C. The particle compositions of the present invention did not affect the neutral pH of the milk (FIG. 71B).

Without wishing to be bound by any particular theory, exposure to moisture and humidity generally lead to degradation and loss of viability and stability of probiotics. These data indicate that exemplary particle compositions of the present disclosure protect probiotics from moisture to promote their viability when stored in fat-containing liquid products (e.g., milk).

### AA. Example 27: Probiotic Viability of Particle Compositions in Electrolyte Beverage

The following non-limiting example describes the viability of particle compositions of the present invention when added to an electrolyte beverage and stored.

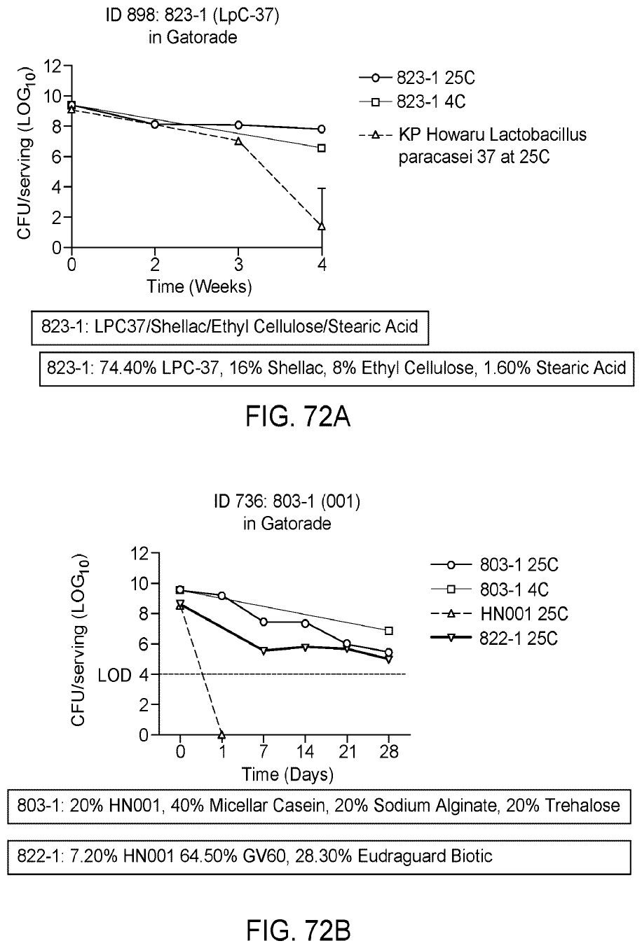

As shown in FIG. 72A, particle compositions having Formulation 14 (as described in Table 8, above), had maintained LPC37 probiotic viability over the course of a four-week storage period when approximately 2.82×10⁶ cfu bacteria/mL (Gatorade) was added to vessels containing Gatorade, mixed by inverting and shaking, and stored at 4° C. or 25° C. In contrast, unencapsulated KP Howaru Lactobacillus paracasei 37 probiotic exhibited a significant loss in viability after four-weeks of storage in Gatorade at 25° C.

TABLE 11
Exemplary Formulations 29 and 10

| | Formulation 29 (803-1) | Formulation 10 (822-1) |
|---|---|---|
| | wt, % | wt, % |
| HN001 | 20% | 7.2% |
| GV60 | | 64.50% |
| Micellar Casein | 40% | |
| Sodium Alginate | 20% | |
| Trehalose | 20% | |
| Eudraguard Biotic | | 28.30% |

As shown in FIG. 72B, particle compositions having Formulations 29 and 10 (as described in Table 11, above), had maintained HN001 probiotic viability over the course of a 28 day storage period when approximately 2.82×10⁶ cfu bacteria/mL (Gatorade) was added to vessels containing Gatorade, mixed by inverting and shaking, and stored at 4° C. or 25° C. In contrast, unencapsulated HN001 probiotic exhibited a significant loss in viability after 1 day of storage in Gatorade at 25 C.

Without wishing to be bound by any particular theory, exposure to moisture and humidity generally lead to degradation and loss of viability and stability of probiotics. These data indicate that exemplary particle compositions of the present disclosure protect probiotics from moisture to promote their viability when stored in electrolyte beverages (e.g., Gatorade).

### BB. Example 28: Probiotic Viability of Particle Compositions after High Temperature Treatment

The following non-limiting example describes the viability of particle compositions of the present invention when added to water and boiled for 30 minutes.

TABLE 12
Exemplary Formulations 30 and 31

| | Formulation 30 | Formulation 31 |
|---|---|---|
| | wt, % | wt, % |
| HN001 | 35% | 9% |
| Hydrogenated Palm Oil | 65% | 81.5% |
| (ADM) | | |
| Ethyl cellulose | | 6.79% |
| Oleic Acid | | 2.71% |
| Processing Method | Prilling; Fluid Bed | Prilling; Fluid Bed |
| | Coating | Coating |

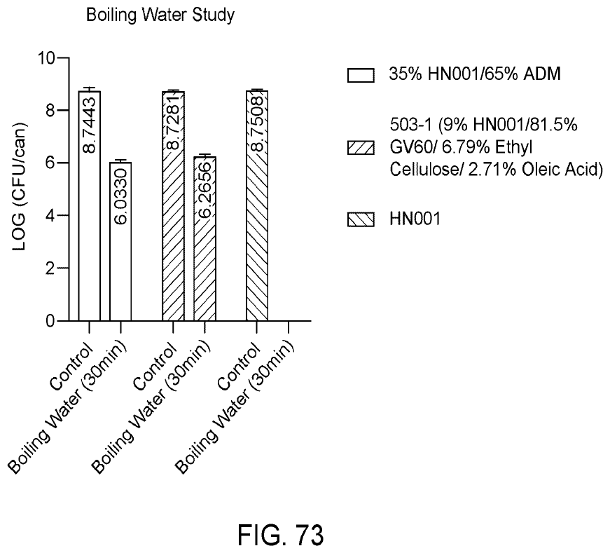

As shown in FIG. 73, no viable HN001 probiotic was detectable following 30 minutes of exposure to 100° C. water. In contrast, particle compositions having Formulations 30 and 31, exhibited only an approximate 2.5 log loss of viability after 30 minutes of exposure to 100° C. water.

Without wishing to be bound by any particular theory, exposure to high temperatures generally lead to degradation and loss of viability and stability of probiotics. These data indicate that exemplary particle compositions of the present disclosure protect probiotics from high temperature liquids to promote their viability when stored in liquid products.

### CC. Example 29: Probiotic Viability of Particle Compositions in Yogurt

The following non-limiting example describes the long-term viability of particle compositions of the present invention when added to yogurt.

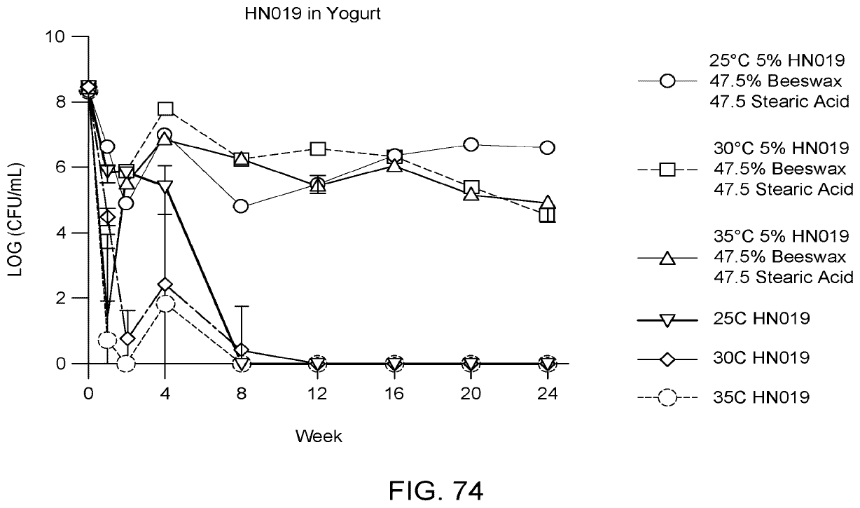

As shown in FIG. 74, particle compositions having Formulation 21 (as described in Table 9, above) had maintained HN019 probiotic viability over a 24-week storage period when 1×10⁸ cfu/mL (yogurt) was added to a vessel containing 10 mL of yogurt, vortexed for approximately 30 seconds, and held at 25° C., 30° C., or 35° C. In contrast, no viable HN019 probiotic was detectable after about 8 weeks in yogurt held at 25° C., 30° C., or 35° C.

Without wishing to be bound by any particular theory, exposure to moisture and humidity generally lead to degradation and loss of viability and stability of probiotics. These data indicate that exemplary particle compositions of the present disclosure protect probiotics from high moisture high viscosity liquid products (e.g. yogurt).

### DD. Example 30: Probiotic Viability of Particle Compositions in Simulated Gastric Fluid

The following non-limiting example describes the viability of particle compositions of the present invention (having higher probiotic loading) when added to low pH simulated gastric fluid.

TABLE 13
Exemplary Formulation 32

| | Formulation 32 |
|---|---|
| | wt, % |
| HN001 | 35% |
| Paraffin | 65% |
| Processing Method | Prilling |

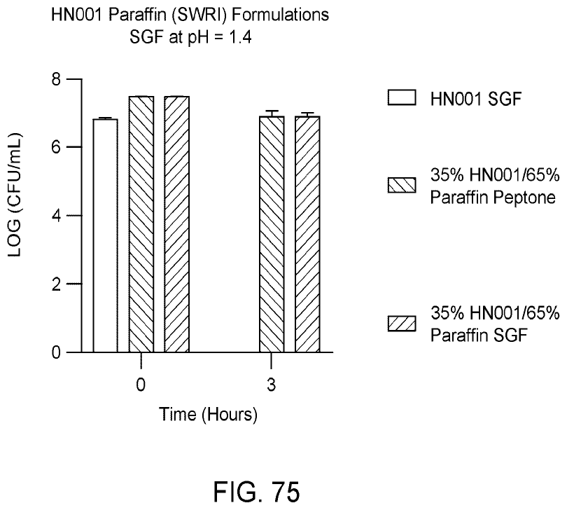

As shown in FIG. 75, particle compositions having Formulation 32 (as described in Table 13, above) had maintained HN001 probiotic viability after three hours in peptone or simulated gastric fluid (“SGF”; pH 1.4). In contrast, no viable unencapsulated HN001 probiotic was detectable after three hours in SGF.

Without wishing to be bound by any particular theory, exposure to extreme pHs generally lead to degradation and loss of viability and stability of probiotics. These data indicate that exemplary particle compositions of the present disclosure protect probiotics from low pH liquid environments (e.g. gastric fluid) and suggest that they can be used to prolong the bioavailability of probiotics when ingested by a subject, as compared to probiotics that are not encapsulated as described herein.

### EE. Example 31: Probiotic Viability of Particle Compositions in Carbonated Liquid

The following non-limiting example describes the viability of particle compositions of the present invention when added to a carbonated liquid.

TABLE 14
Exemplary Formulations 33 and 30

| | Formulation 33 | Formulation 30 |
|---|---|---|
| | wt, % | wt, % |
| HN001 | 35% | 35% |
| Hydrogenated Palm Oil (Dritex) | 65% | |
| Hydrogenated Palm Oil (ADM) | | 65% |
| Processing Method | Prilling | Prilling |

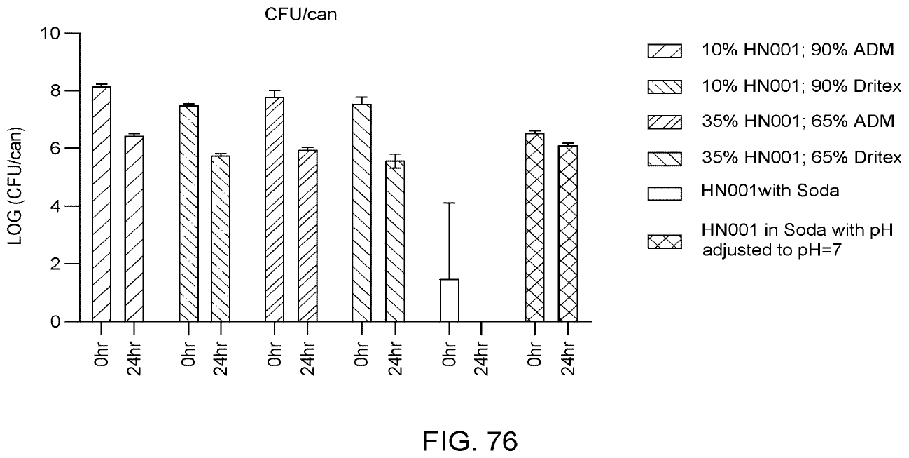

Particle compositions having Formulation 33 or Formulation 30 (as described in Table 14, above) were added to vessels containing 355 mL of Pepsi soda to achieve the equivalent of 10⁹ cfu/355 mL. As shown in FIG. 76, particle compositions having Formulation 33 or Formulation 30 had only about a two log decrease in HN001 probiotic viability after 24 hours in carbonated Pepsi soda. In contrast, unencapsulated HN001 probiotic had an almost six log reduction in viability immediately upon addition to Pepsi and no detectable viability after 24 hours. However, adjusting the Pepsi to pH 7.0 maintained HN001 probiotic viability.

These results suggest that particle compositions of the present disclosure are capable of protecting pH sensitive probiotics in carbonated, low pH liquid environments.

### FE. Example 32: Exemplary Formulations and Methods for Preparing Same

The following non-limiting example presents exemplary formulations and preparation methods in accordance with the present disclosure.

TABLE 15
Exemplary Formulations

| | | Expected | | | |
|---|---|---|---|---|---|
| | | Loading, | Component 1 | Component 2 | Component 3 |
| Formulation | Probiotic | CFU/g | (wt %) | (wt %) | (wt %) |
| Formulation A | HN001 | | GV60 | | |
| | (10 wt %) | | (90 wt %) | | |
| Formulation B | HN001 | 1.18E+10 | Bees Wax | GV60 | PEG 3350 |
| | (5 wt %) | | (42.5 wt %) | (42.5 wt %) | (10 wt %) |
| Formulation C | HN001 | | Bees Wax | Stearic | PEG 3350 |
| | (5 wt %) | | (42.5 wt %) | Acid | (10 wt %) |
| | | | | (42.5 wt %) | |
| Formulation D | HN001 | 1.18E+10 | Bees Wax | Stearic Acid | |
| | (5 wt %) | | (47.5 wt %) | (47.5 wt %) | |
| Formulation E | HN001 | 5.90E+09 | Bees Wax | GV60 | Polydextrose |
| | (2.5 wt %) | | (47.5 wt %) | (47.5 wt %) | (2.5 wt %) |
| Formulation F | HN019 | | GV60 | | |
| | (10 wt %) | | (90 wt %) | | |
| Formulation G | HN019 | | Bees Wax | Stearic Acid | |
| | (5 wt %) | | (47.5 wt %) | (47.5 wt %) | |
| Formulation H | HN019 | 5.13E+10 | GV60 | Calcium | Ethy Cellulose |
| | (8.55 wt %) | | (68.4 wt %) | Carbonate | (Colorcon) |
| | | | | (8.55 wt %) | (14.5 wt %) |
| Formulation I | HN019 | 5.00E+11 | Ethyl Cellulose | | |
| | (83.33 wt %) | | (16.67 wt %) | | |
| Formulation J | HN001 | 2.81E+11 | GV60 | Calcium | |
| | (46.8 wt %) | | (56.28 wt %) | Carbonate | |
| | | | | (15.62 wt %) | |
| Formulation K | HN001 | 1.58E+11 | GV60 | Calcium | |
| | (26.3 wt %) | | (65.74788 wt %) | Carbonate | |
| | | | | (7.95 wt %) | |
| Formulation L | HN001 | 2.19E+10 | GV60 | Ethyl Cellulose | Oleic Acid |
| | (9.3 wt %) | | (83.7 wt %) | (4.98 wt %) | (2 wt %) |
| Formulation M | HN001 | 3.21E+11 | Ethyl Cellulose | Oleic Acid | |
| | (83 wt %) | | (12.1 wt %) | (4.9 wt %) | |
| Fomulation N | HN001 | 5.03E+10 | Ethyl Cellulose | Oleic Acid | GV60 |
| | (13 wt %) | | (1.864 wt %) | (0.78 wt %) | (74.46 wt %) |
| Formulation O | HN001 | 2.19E+10 | GV60 | Ethyl Cellulose | Oleic Acid |
| | (9 wt %) | | (81.5 wt %) | (6.785 wt %) | (2.714 wt %) |
| Formulation P | HN019 | 3.54E+10 | GV60 | Shellac | Ethyl Cellulose |
| | (5.9 wt %) | | (53.3 wt %) | (25.3 wt %) | (12.7 wt %) |
| Formulation Q | HN019 | 5.01E+10 | GV60 | Eudraguard | |
| | (8.35 wt %) | | (75.16 wt %) | Biotic | |
| | | | | (16.49 wt %) | |
| Formulation R | HN019 | 1.21E+10 | GV60 | Shellac | Ethyl Cellulose |
| | (2.01 wt %) | | (67.29 wt %) | (19.14 wt %) | (9.57 wt %) |
| Formulation S | HN019 | 8.40E+09 | GV60 | Eudraguard | |
| | (2.1 wt %) | | (69.3 wt %) | Biotic | |
| | | | | (28.6 wt %) | |
| Formulation T | HN001 | 1.20E+10 | GV60 | Shellac | Ethyl Cellulose |
| | (2 wt %) | | (67.4 wt %) | (19.1 wt %) | (9.55 wt %) |
| Formulation U | HN001 | 3.54E+10 | GV60 | Shellac | Ethyl Cellulose |
| | (5.9 wt %) | | (53.5 wt %) | (25.3 wt %) | (12.6 wt %) |
| Formulation V | LPC-37 | 9.20E+09 | GV60 | Eudraguard | |
| | (2.3 wt %) | | (74.8 wt %) | Biotic | |
| | | | | (22.8 wt %) | |
| Formulation W | LPC-37 | 2.98E+11 | Eudraguard | | |
| | (3 wt %) | | Biotic | | |
| | | | (97 wt %) | | |

| | | | | Coating, if |
|---|---|---|---|---|
| | Component 4 | Component 5 | Component 6 | present |
| Formulation | (wt %) | (wt %) | (wt %) | (wt %) |
| Formulation A | | | | |
| Formulation B | | | | |
| Formulation C | | | | |
| Formulation D | | | | |
| Formulation E | | | | |
| Formulation F | | | | |
| Formulation G | | | | |
| Formulation H | | | | Ethyl Cellulose |
| | | | | (14.5 wt %) |
| Formulation I | | | | Ethyl Cellulose |
| | | | | (16.67 wt %) |
| Formulation J | | | | |
| Formulation K | | | | |
| Formulation L | | | | Ethyl Cellulose/ |
| | | | | Oleic Acid |
| | | | | (6.97 wt %) |
| Formulation M | | | | |
| Fomulation N | Calcium | | | |
| | Carbonate | | | |
| | (10 wt %) | | | |
| Formulation O | | | | Ethyl Cellulose/ |
| | | | | Oleic Acid |
| | | | | (9.5 wt %) |
| Formulation P | Stearic Acid | | | Shellac/EC/ |
| | (2.5 wt %) | | | Stearic Acid |
| | | | | (40.7 wt %) |
| Formulation Q | | | | Eudraguard |
| | | | | Biotic |
| | | | | (16.49 wt %) |
| Formulation R | Stearic Acid | | | Shellac/EC/ |
| | (1.91 wt %) | | | Stearic Acid |
| | | | | (30.6 wt %) |
| Formulation S | | | | Eudraguard |
| | | | | Biotic |
| | | | | (28.6 wt %) |
| Formulation T | Stearic Acid | | | Shellac/EC/ |
| | (1.91 wt %) | | | Stearic Acid |
| | | | | (30.56 wt %) |
| Formulation U | Stearic Acid | | | Shellac/EC/ |
| | (2.5 wt %) | | | Stearic Acid |
| | | | | (40.5 wt %) |
| Formulation V | | | | Eudraguard |
| | | | | Biotic |
| | | | | (22.8 wt %) |
| Formulation W | | | | Shellac/EC/ |
| | | | | Stearic Acid |
| | | | | (25.6 wt %) |

TABLE 16
Exemplary Preparation Methods

| | |
|---|---|
| Formulation | Step(s) |

| | | | |
|---|---|---|---|
| Formulation A | Prilling | | |
| Formulation B | Prilling | | |
| Formulation C | Prilling | | |
| Formulation D | Prilling | | |
| Formulation E | Prilling | | |
| Formulation F | Prilling | | |
| Formulation G | Prilling | | |
| Formulation H | Extrusion | Fluid Bed | |
| Formulation I | Fluid Bed | | |
| Formulation J | Extrusion | Extrusion | |
| Formulation K | Extrusion | Extrusion | Extrusion |
| Formulation L | Prilling | Fluid Bed | |
| Formulation M | Fluid Bed | | |
| Formulation N | Fluid Bed | Extrusion | |
| Formulation O | Extrusion | | |
| Formulation P | Prilling | Fluid Bed | |
| Formulation Q | Prilling | Fluid Bed | |
| Formulation R | Prilling | Extrusion | Fluid Bed |
| Formulation S | Prilling | Extrusion | Fluid Bed |
| Formulation T | Prilling | Extrusion | Fluid Bed |
| Formulation U | Prilling | Fluid Bed | |
| Formulation V | Extrusion | Fluid Bed | |
| Formulation W | Fluid Bed | | |

### GG. Example 33: Exemplary Formulations and Methods for Preparing Same

The following non-limiting example presents exemplary formulations which may be included in an electrolyte beverage (e.g., Gatorade) and their preparation methods in accordance with the present disclosure.

TABLE 16
Exemplary Formulations for Inclusion in Electrolyte Beverages

| | | Expected | | | | |
|---|---|---|---|---|---|---|
| | | Loading, | Component 1 | Component 2 | Component 3 | Component 4 |
| Formulation | Probiotic | CFU/g | (wt %) | (wt %) | (wt %) | (wt %) |
| Formulation X | HN001 | 1.80E+10 | GV60 | Precirol | | |
| | (3 wt %) | | (27 wt %) | ATO 5 | | |
| | | | | (70 wt %) | | |
| Formulation Y | HN001 | 1.80E+10 | GV60 | | | |
| | (3 wt %) | | (97 wt %) | | | |
| Formulation Z | HN001 | 1.20E+10 | GV60 | Shellac | Ethyl | Stearic |
| | (2 wt %) | | (67.4 wt %) | (19.1 wt %) | Cellulose | Acid |
| | | | | | (9.55 wt %) | (1.91 wt %) |
| Formulation AA | HN001 | 3.54E+10 | GV60 | Shellac | Ethyl | Stearic |
| | (5.9 wt %) | | (53.5 wt %) | (25.3 wt %) | Cellulose | Acid |
| | | | | | (12.6 wt %) | (2.5 wt %) |
| Formulation AB | HN019 | 3.54E+10 | GV60 | Shellac | Ethyl | Stearic |
| | (5.9 wt %) | | (53.3 wt %) | (25.3 wt %) | Cellulose | Acid |
| | | | | | (12.7 wt %) | (2.5 wt %) |
| Formulation AC | HN019 | 5.01E+10 | GV60 | Eudraguard | | |
| | (8.35 wt %) | | (75.16 wt %) | Biotic | | |
| | | | | (16.49 wt %) | | |
| Formulation AD | HN019 | 1.21E+10 | GV60 | Shellac | Ethyl | Stearic |
| | (2.01 wt %) | | (67.29 wt %) | (19.14 wt %) | Cellulose | Acid |
| | | | | | (9.57 wt %) | (1.91 wt %) |
| Formulation AE | HN019 | 8.40E+09 | GV60 | Eudraguard | | |
| | (2.1 wt %) | | (69.3 wt %) | Biotic | | |
| | | | | (28.6 wt %) | | |
| Formulation AF | HN019 | 2.31E+10 | Bees Wax | Stearic | Eudraguard | |
| | (3.85 wt %) | | (36.7 wt %) | Acid | Biotic | |
| | | | | (36.7 wt %) | (22.8 wt %) | |
| Formulation AG | HN019 | 1.60E+10 | Bees Wax | Stearic | Shellac | Ethyl |
| | (4 wt %) | | (37.6 wt %) | Acid | (13.1 wt %) | Cellulose |
| | | | | (37.6 wt %) | | (6.5 wt %) |
| Formulation AH | HN001 | 2.88E+10 | GV60 | Eudraguard | | |
| | (7.2 wt %) | | (64.5 wt %) | Biotic | | |
| | | | | (28.3 wt %) | | |
| Formulation AI | LPC-37 | 2.98E+11 | Shellac | Ethyl | | |
| | (74.4 wt %) | | (16 wt %) | Cellulose | Stearic | |
| | | | | (8 wt %) | Acid | |
| | | | | | (1.6 wt %) | |
| Formulation AJ | HN001 | 2.98E+11 | Eudraguard | | | |
| | (3 wt %) | | Biotic | | | |
| | | | (97 wt %) | | | |
| Formulation AK | LPC-37 | 6.40E+09 | Eudraguard | Trehalose | | |
| | (1.6 wt %) | | Biotic | (26.6 wt %) | | |
| | | | (as | | | |
| | | | Recieved) | | | |
| | | | (71.8 wt %) | | | |
| Formulation AL | LPC-37 | 9.20E+09 | GV60 | Eudraguard | | |
| | (2.3 wt %) | | (74.8 wt %) | Biotic | | |
| | | | | (22.8 wt %) | | |
| Formulation AM | LPC-37 | 2.00E+10 | Eudraguard | Trehalose | | |
| | (5 wt %) | | Biotic | (8.6 wt %) | | |
| | | | (as | | | |
| | | | Recieved) | | | |
| | | | (86.3 wt %) | | | |
| Formulation AN | LPC-37 | 2.00E+10 | Eudraguard | | | |
| | (5 wt %) | | Biotic | | | |
| | | | (as | | | |
| | | | Recieved) | | | |
| | | | (95 wt %) | | | |
| Formulation AO | LPC-37 | 8.40E+09 | GV60 | Shellac | Ethyl | Stearic |
| | (2.1 wt %) | | (67.4 wt %) | (19.1 wt %) | Cellulose | Acid |
| | | | | | (9.5 wt %) | (1.9 wt %) |
| Formulation AP | LPC-37 | 8.00E+10 | Eudraguard | | | |
| | (20 wt %) | | Biotic | | | |
| | | | (as | | | |
| | | | Recieved) | | | |
| | | | (80 wt %) | | | |
| Formulation AQ | HN019 | 0.00E+00 | GV60 | Calcium | Ethyl | |
| | (8.5 wt %) | | (68.2 wt %) | Carbonate | Cellulose | |
| | | | | (8.5 wt %) | (14.7 wt %) | |
| Formulation AR | LPC-37 | 0.00E+00 | EG Biotic | | | |
| | (5 wt %) | | (95 wt %) | | | |
| Formulation AS | LPC-37 | 0.00E+00 | Vikram | | | |
| | (5 wt %) | | FS 30D | | | |
| | | | (95 wt %) | | | |
| Formulation AT | HN001 | 1.04E+11 | Micellar | Sodium | Trehalose | |
| | (20 wt %) | | Casein | Alginate | (20 wt %) | |
| | | | (40 wt %) | (20 wt %) | | |
| Formulation AU | HN001 | 1.20E+10 | Citric Acid | Beeswax | β- | γ- |
| | (2 wt %) | | Esters of | (10 wt %) | Sitosterol | Oryzanol |
| | | | Mono- and Di- | | (24 wt %) | (16 wt %) |
| | | | glycerides | | | |
| | | | (30 wt %) | | | |
| Formulation AV | HN001 | 1.20E+10 | Citric Acid | Beeswax | β- | γ- |
| | (2 wt %) | | Esters of | (10 wt %) | Sitosterol | Oryzanol |
| | | | Mono- and Di- | | (24 wt %) | (16 wt %) |
| | | | glycerides | | | |
| | | | (30 wt %) | | | |
| Formulation AW | HN001 | 6.00E+09 | 27 Stearine | Cholesterol | Beeswax | Sodium |
| | (1 wt %) | | (55 wt %) | (15 wt %) | (10 wt %) | Alginate |
| | | | | | | (6.6 wt %) |
| Formulation AX | HN001 | 6.00E+09 | 27 Stearine | Cholesterol | Beeswax | Calcium |
| | (1 wt %) | | (67 wt %) | (10 wt %) | (10 wt %) | Carbonate |
| | | | | | | (3.3 wt %) |
| Formulation AY | HN001 | 6.00E+09 | Carnauba | Citric Acid | Cholesterol | Calcium |
| | (1 wt %) | | Wax | Esters of | (15 wt %) | Carbonate |
| | | | (40 wt %) | Mono- and Di- | | (5 wt %) |
| | | | | glycerides | | |
| | | | | (30 wt %) | | |
| Formulation AZ | HN001 | 6.00E+09 | Carnauba | Citric Acid | β- | Calcium |
| | (1 wt %) | | Wax | Esters of | Sitosterol | Carbonate |
| | | | (40 wt %) | Mono- and Di- | (15 wt %) | (5 wt %) |
| | | | | glycerides | | |
| | | | | (30 wt %) | | |
| Formulation BA | HN001 | 6.00E+09 | Carnauba | Citric Acid | β- | Calcium |
| | (1 wt %) | | Wax | Esters of | Sitosterol | Carbonate |
| | | | (35 wt %) | Mono- and Di- | (15 wt %) | (5 wt %) |
| | | | | glycerides | | |
| | | | | (25 wt %) | | |
| Formulation BB | HN001 | 6.00E+09 | Carnauba | Citric Acid | β- | Calcium |
| | (1 wt %) | | Wax | Esters of | Sitosterol | Carbonate |
| | | | (35 wt %) | Mono- and Di- | (15 wt %) | (5 wt %) |
| | | | | glycerides | | |
| | | | | (25 wt %) | | |
| Formulation BC | HN001 | 6.00E+09 | Carnauba | Citric Acid | β- | Calcium |
| | (1 wt %) | | Wax | Esters of | Sitosterol | Carbonate |
| | | | (35 wt %) | Mono- and Di- | (15 wt %) | (5 wt %) |
| | | | | glycerides | | |
| | | | | (25 wt %) | | |
| Formulation BD | HN001 | 6.00E+09 | Carnauba | Stearic Acid | β- | GV60 |
| | (1 wt %) | | Wax | (25 wt %) | Sitosterol | (9 wt %) |
| | | | (50 wt %) | | (15 wt %) | |
| Formulation BE | HN001 | 6.00E+09 | Carnauba | Stearic Acid | β- | GV60 |
| | (1 wt %) | | Wax | (22 wt %) | Sitosterol | (9 wt %) |
| | | | (44 wt %) | | (15 wt %) | |
| Formulation BF | HN001 | 6.00E+09 | Carnauba | Stearic Acid | β- | GV60 |
| | (1 wt %) | | Wax | (22 wt %) | Sitosterol | (9 wt %) |
| | | | (44 wt %) | | (15 wt %) | |
| Formulation BG | HN001 | 6.00E+09 | Carnauba | Stearic Acid | β- | GV60 |
| | (1 wt %) | | Wax | (22 wt %) | Sitosterol | (9 wt %) |
| | | | (44 wt %) | | (15 wt %) | |
| Formulation BH | HN001 | 1.95E+11 | Sodium | Succinic Acid | Trehalose | Calcium |
| | (32.5 wt %) | | Alginate | (16.25 wt %) | (16.25 wt %) | Hydrogen |
| | | | (32.5 wt %) | | | Phosphate |
| | | | | | | (2.4 wt %) |
| Formulation BI | HN001 | 1.50E+11 | Sodium | Micellar | Trehalose | |
| | (25 wt %) | | Alginate | Casein | (12.5 wt %) | Sodium |
| | | | (25 wt %) | (25 wt %) | | Phosphate |
| | | | | | | Dibasic |
| | | | | | | (12.5 wt %) |
| Formulation BK | LPC-37 | 1.95E+11 | Sodium | Succinic | Trehalose | Calcium |
| | (32.5 wt %) | | Alginate | Acid | (16.25 wt %) | Hydrogen |
| | | | (32.5 wt %) | (16.25 wt %) | | Phosphate |
| | | | | | | (2.4 wt %) |
| Formulation BL | LPC-37 | 2.90E+09 | Sodium | Succinic | Trehalose | Calcium |
| | (2.9 wt %) | | Alginate | Acid | (1.45 wt %) | Hydrogen |
| | | | (2.9 wt %) | (1.45 wt %) | | Phosphate |
| | | | | | | (0.21 wt %) |

| | | | | | Coating, |
|---|---|---|---|---|---|
| | Component 5 | Component 6 | Component 7 | Component 8 | if present |
| Formulation | (wt %) | (wt %) | (wt %) | (wt %) | (wt %) |
| Formulation X | | | | | |
| Formulation Y | | | | | |
| Formulation Z | | | | | Shellac/EC/ |
| | | | | | Stearic Acid |
| | | | | | (30.56 wt %) |
| Formulation AA | | | | | Shellac/EC/ |
| | | | | | Stearic Acid |
| | | | | | (40.5 wt %) |
| Formulation AB | | | | | Shellac/EC/ |
| | | | | | Stearic Acid |
| | | | | | (40.7 wt %) |
| Formulation AC | | | | | Eudraguard |
| | | | | | Biotic |
| | | | | | (16.49 wt %) |
| Formulation AD | | | | | Shellac/EC/ |
| | | | | | Stearic Acid |
| | | | | | (30.6 wt %) |
| Formulation AE | | | | | Eudraguard |
| | | | | | Biotic |
| | | | | | (28.6 wt %) |
| Formulation AF | | | | | Eudraguard |
| | | | | | Biotic |
| | | | | | (22.8 wt %) |
| Formulation AG | Stearic | | | | Shellac/EC/ |
| | Acid | | | | Stearic Acid |
| | (1.3 wt %) | | | | (20.9 wt %) |
| Formulation AH | | | | | Eudraguard |
| | | | | | Biotic |
| | | | | | (28.3 wt %) |
| Formulation AI | | | | | Shellac/EC/ |
| | | | | | Stearic Acid |
| | | | | | (25.6 wt %) |
| Formulation AJ | | | | | Eudraguard |
| | | | | | Biotic |
| | | | | | (97 wt %) |
| Formulation AK | | | | | Eudraguard |
| | | | | | Biotic |
| | | | | | (71.8 wt %) |
| Formulation AL | | | | | Eudraguard |
| | | | | | Biotic |
| | | | | | (22.8 wt %) |
| Formulation AM | | | | | Eudraguard |
| | | | | | Biotic |
| | | | | | (86.3 wt %) |
| Formulation AN | | | | | Eudraguard |
| | | | | | Biotic |
| | | | | | (95 wt %) |
| Formulation AO | | | | | Shellac/EC/ |
| | | | | | Stearic Acid |
| | | | | | (30.6 wt %) |
| Formulation AP | | | | | Eudraguard |
| | | | | | Biotic |
| | | | | | (80 wt %) |
| Formulation AQ | | | | | Ethyl |
| | | | | | Cellulose |
| | | | | | (14.7 wt %) |
| Formulation AR | | | | | EG Biotic |
| | | | | | (95 wt %) |
| Formulation AS | | | | | Viram FS |
| | | | | | 30D |
| | | | | | (95 wt %) |
| Formulation AT | | | | | |
| Formulation AU | GV60 | | | | |
| | (18 wt %) | | | | |
| Formulation AV | GV60 | | | | |
| | (18 wt %) | | | | |
| Formulation AW | Calcium | GV60 | | | |
| | Carbonate | (9 wt %) | | | |
| | (3.3 wt %) | | | | |
| Formulation AX | GV60 | | | | |
| | (9 wt %) | | | | |
| Formulation AY | GV60 | | | | |
| | (9 wt %) | | | | |
| Formulation AZ | GV60 | | | | |
| | (9 wt %) | | | | |
| Formulation BA | GV60 | Methyl | | | |
| | (9 wt %) | Cellulose | | | |
| | | 4000 cP | | | |
| | | (10 wt %) | | | |
| Formulation BB | GV60 | Sodium | | | |
| | (9 wt %) | Alginate | | | |
| | | (10 wt %) | | | |
| Formulation BC | GV60 | Hydroxypropyl | | | |
| | (9 wt %) | methylcellulose | | | |
| | | 2000 cP | | | |
| | | (10 wt %) | | | |
| Formulation BD | | | | | |
| Formulation BE | Sodium | | | | |
| | Alginate | | | | |
| | (10 wt %) | | | | |
| Formulation BF | Methyl | | | | |
| | Cellulose | | | | |
| | 4000 cP | | | | |
| | (10 wt %) | | | | |
| Formulation BG | Hydroxypropyl | | | | |
| | Methylcellulose | | | | |
| | 2000 cP | | | | |
| | (10 wt %) | | | | |
| Formulation BH | | | | | |
| Formulation BI | | | | | |
| Formulation BK | | | | | |
| Formulation BL | Carnauba | Stearic | Sitosterol | Calcium | Shellac/EC/ |
| | Wax | Acid | (5.7 wt %) | Carbonate | Stearic Acid |
| | (45 wt %) | (22 wt %) | | (4 wt %) | (30 wt %) |

TABLE 17
Exemplary Methods for Preparing Formulations
for Inclusion in Electrolyte Beverages

| Formulation | Step(s) | | | |
|---|---|---|---|---|
| Formulation X | Prilling | Extrusion | | |
| Formulation Y | Prilling | Extrusion | | |
| Formulation Z | Prilling | Extrusion | Fluid Bed | |
| Formulation AA | Prilling | Fluid Bed | | |
| Formulation AB | Prilling | Fluid Bed | | |
| Formulation AC | Prilling | Fluid Bed | | |
| Formulation AD | Prilling | Extrusion | Fluid Bed | |
| Formulation AE | Prilling | Extrusion | Fluid Bed | |
| Formulation AF | Prilling | Fluid Bed | | |
| Formulation AG | Prilling | Fluid Bed | | |
| Formulation AH | Prilling | Fluid Bed | | |
| Formulation AI | Fluid Bed | | | |
| Formulation AJ | Freeze Dryer | | | |
| Formulation AK | Spray Drying | | | |
| Formulation AL | Extrusion | Fluid Bed | | |
| Formulation AM | Spray Drying | | | |
| Formulation AN | Spray Drying | | | |
| Formulation AO | Extrusion | Fluid Bed | | |
| Formulation AP | Spray Drying | | | |
| Formulation AQ | Extrusion | Fluid Bed | | |
| Formulation AR | Freeze Dryer | | | |
| Formulation AS | Freeze Dryer | | | |
| Formulation AT | Spray Drying | | | |
| Formulation AU | Prilling | Extrusion | Cryo Mill | |
| Formulation AV | Prilling | Extrusion | Cryo Mill | |
| Formulation AW | Prilling | Extrusion | Cryo Mill | |
| Formulation AX | Prilling | Extrusion | Cryo Mill | |
| Formulation AY | Prilling | Extrusion | Cryo Mill | |
| Formulation AZ | Prilling | Extrusion | Cryo Mill | |
| Formulation BA | Prilling | Extrusion | Cryo Mill | |
| Formulation BB | Prilling | Extrusion | Cryo Mill | |
| Formulation BC | Prilling | Extrusion | Cryo Mill | |
| Formulation BD | Prilling | Extrusion | Cryo Mill | |
| Formulation BE | Prilling | Extrusion | Cryo Mill | |
| Formulation BF | Prilling | Extrusion | Cryo Mill | |
| Formulation BG | Prilling | Extrusion | Cryo Mill | |
| Formulation BH | Spray Drying | CLAM | | |
| Formulation BI | Spray Drying | | | |
| Formulation BK | Spray Drying | CLAM | | |
| Formulation BL | Spray Drying | CLAM | Extrusion | Cryo Mill |

### HH. Example 34: Exemplary Formulations and Methods for Preparing Same

The following non-limiting example presents exemplary formulations which may be included in yogurt and their preparation methods in accordance with the present disclosure.

TABLE 18
Exemplary Formulations for Inclusion in Yogurt

| | | Expected | | |
|---|---|---|---|---|
| | Probiotic | Loading, | Component 1 | Component 2 |
| Formulation | (wt %) | CFU/g | (wt %) | (wt %) |
| Formulation BM | HN019 | Bees Wax | Bees Wax | |
| | (20 wt %) | | (80 wt %) | |
| Formulation BN | HN019 | GV60 | GV60 | |
| | (10 wt %) | | (90 wt %) | |
| Formulation BO | HN019 | Bees Wax | Bees Wax | |
| | (10 wt %) | | (90 wt %) | |
| Formulation BP | HN019 | Bees Wax | Bees Wax | |
| | (20 wt %) | | (80 wt %) | |
| Formulation BQ | HN019 | Bees Wax | Bees Wax | Stearic Acid |
| | (20 wt %) | | (64 wt %) | (16 wt %) |
| Formulation BR | HN019 | Bees Wax | Bees Wax | Stearic Acid |
| | (5 wt %) | | (45 wt %) | (45 wt %) |
| Formulation BS | HN019 | Bees Wax | Bees Wax | Stearic Acid |
| | (10 wt %) | | (45 wt %) | (45 wt %) |
| Formulation BT | HN019 | GV60 | GV60 | PEG 3350 |
| | (10 wt %) | | (80 wt %) | (10 wt %) |
| Formulation BU | HN019 | Bees Wax | Bees Wax | |
| | (10 wt %) | | (90 wt %) | |
| Formulation BV | HN019 | Bees Wax | Bees Wax | Carnauba |
| | (10 wt %) | | (45 wt %) | Wax |
| | | | | (45 wt %) |
| Formulation BW | HN019 | Bees Wax | Bees Wax | Carnauba |
| | (5 wt %) | | (68 wt %) | Wax |
| | | | | (17 wt %) |
| Formulation BX | HN019 | Bees Wax | Bees Wax | PEG 3350 |
| | (5 wt %) | | (85 wt %) | (10 wt %) |
| Formulation BY | HN019 | GV60 | GV60 | Sorbitol |
| | (5 wt %) | | (86 wt %) | (1.8 wt %) |
| Formulation BZ | HN019 | Polydextrose | Polydextrose | |
| | (35 wt %) | | (65 wt %) | |
| Formulation CA | HN019 | Polydextrose | Polydextrose | Purity Gum |
| | (35 wt %) | | (32.5 wt %) | Starch |
| | | | | (32.5 wt %) |
| Formulation CB | HN019 | Polydextrose | Polydextrose | Inulin |
| | (20 wt %) | | (40 wt %) | (38.7 wt %) |
| Formulation CC | HN019 | Polydextrose | Polydextrose | Inulin |
| | (20 wt %) | | (60 wt %) | (18.7 wt %) |
| Formulation CD | HN019 | Polydextrose | Polydextrose | Inulin |
| | (20 wt %) | | (40 wt %) | (18.7 wt %) |
| Formulation CE | HN019 | Polydextrose | Polydextrose | Inulin |
| | (25 wt %) | | (40 wt %) | (13.7 wt %) |
| Formulation CF | HN001 | Polydextrose | Polydextrose | Inulin |
| | (25 wt %) | | (40 wt %) | (13.7 wt %) |
| Formulation CG | HN001 | GV60 | GV60 | Polydextrose |
| | (5 wt %) | | (70 wt %) | (8 wt %) |
| Formulation CH | HN001 | Bees Wax | Bees Wax | Stearic Acid |
| | (5 wt %) | | (47.5 wt %) | (47.5 wt %) |
| Formulation CI | HN001 | Bees Wax | Bees Wax | GV60 |
| | (5 wt %) | | (42.5 wt %) | (42.5 wt %) |
| Formulation CJ | HN001 | Bees Wax | Bees Wax | GV60 |
| | (2.5 wt %) | | (47.5 wt %) | (47.5 wt %) |
| Formulation CK | HN001 | Polydextrose | Polydextrose | Inulin |
| | (25 wt %) | | (33.7 wt %) | (20 wt %) |
| Formulation CL | HN001 | Polydextrose | Polydextrose | Trehalose |
| | (25 wt %) | | (60 wt %) | (14 wt %) |
| Formulation CM | HN001 | GV60 | GV60 | Calcium |
| | (25 wt %) | | (65 wt %) | Carbonate |
| | | | | (10 wt %) |
| Formulation CN | HN001 | GV60 | GV60 | Calcium |
| | (5 wt %) | | (83 wt %) | Carbonate |
| | | | | (12 wt %) |
| Formulation CO | HN001 | Beef | Beef | Isomalt |
| | (25 wt %) | Gelatin | Gelatin | (25 wt %) |
| | | | (25 wt %) | |
| Formulation CP | HN001 | Beef | Beef | Isomalt |
| | (25 wt %) | Gelatin | Gelatin | (33 wt %) |
| | | | (25 wt %) | |
| Formulation CQ | HN001 | Beef | Beef | Isomalt |
| | (5 wt %) | Gelatin | Gelatin | (5 wt %) |
| | | | (5 wt %) | |
| Formulation CR | HN001 | Amidated | Amidated | Calcium |
| | (5 wt %) | LM Pectin | LM Pectin | Lactate |
| | | | (5 wt %) | Glucanate |
| | | | | (0.5 wt %) |
| Formulation CS | HN001 | Sodium | Sodium | Calcium |
| | (5 wt %) | Alginate | Alginate | Chloride |
| | | | (10 wt %) | (0.9 wt %) |
| Formulation CT | HN001 | Sunflower | Sunflower | GV60 |
| | (10 wt %) | Lecithin | Lecithin | (70 wt %) |
| | | | (10 wt %) | |
| Formulation CU | HN001 | Sunflower | Sunflower | Sodium |
| | (10 wt %) | Lecithin | Lecithin | Alginate |
| | | | (5 wt %) | (10 wt %) |
| Formulation CV | HN001 | Sunflower | Sunflower | GV60 |
| | (5 wt %) | Lecithin | Lecithin | (75 wt %) |
| | | | (5 wt %) | |
| Formulation CW | HN001 | Sunflower | Sunflower | Sodium |
| | (5 wt %) | Lecithin | Lecithin | Alginate |
| | | | (2.5 wt %) | (5 wt %) |
| Formulation CX | HN001 | GV60 | GV60 | Calcium |
| | (10 wt %) | | (80 wt %) | Carbonate |
| | | | | (10 wt %) |
| Formulation CY | HN001 | GV60 | GV60 | Calcium |
| | (10 wt %) | | (80 wt %) | Carbonate |
| | | | | (10 wt %) |
| Formulation CZ | HN001 | GV60 | GV60 | Calcium |
| | (10 wt %) | | (80 wt %) | Carbonate |
| | | | | (10 wt %) |
| Formulation DA | HN001 | GV60 | GV60 | Calcium |
| | (10 wt %) | | (80 wt %) | Carbonate |
| | | | | (10 wt %) |
| Formulation DB | HN001 | GV60 | GV60 | |
| | (10 wt %) | | (90 wt %) | |
| Formulation DC | HN001 | GV60 | GV60 | |
| | (10 wt %) | | (90 wt %) | |
| Formulation DE | HN001 | GV60 | GV60 | Calcium |
| | (10 wt %) | | (80 wt %) | Carbonate |
| | | | | (10 wt %) |
| Formulation DF | HN001 | Methocel | Methocel | Magnesium |
| | (5 wt %) | | (20 wt %) | Stearate |
| | | | | (1 wt %) |
| Formulation DG | HN001 | Methocel | Methocel | Magnesium |
| | (5 wt %) | | (20 wt %) | Stearate |
| | | | | (1 wt %) |
| Formulation DH | HN001 | GV60 | GV60 | |
| | (10 wt %) | | (90 wt %) | |
| Formulation DI | HN001 | Ethyl | Ethyl | Oleic Acid |
| | (35 wt %) | Cellulose | Cellulose | (9 wt %) |
| | | | (21 wt %) | |
| Formulation DJ | HN001 | Ethyl | Ethyl | Oleic Acid |
| | (28.24 wt %) | Cellulose | Cellulose | (12.45 wt %) |
| | | | (31.07 wt %) | |
| Formulation DK | HN001 | Ethyl | Ethyl | Oleic Acid |
| | (5 wt %) | Cellulose | Cellulose | (1 wt %) |
| | | | (3 wt %) | |
| Formulation DL | HN001 | GV60 | GV60 | Ethyl |
| | (2.5 wt %) | | (81.15 wt %) | Cellulose |
| | | | | (2.75 wt %) |
| Formulation DM | HN001 | Shellac | Shellac | Calcium |
| | (25 wt %) | | (50 wt %) | Carbonate |
| | | | | (25 wt %) |
| Formulation DN | HN001 | Shellac | Shellac | GV60 |
| | (5 wt %) | | (10 wt %) | (70 wt %) |
| Formulation DO | HN001 | Ethyl | Ethyl | Oleic Acid |
| | (37.8 wt %) | Cellulose | Cellulose | (15 wt %) |
| | | | (37.8 wt %) | |
| Formulation DP | HN001 | Sodium | Sodium | Calcium |
| | (5 wt %) | Alginate | Alginate | Chloride |
| | | | (16.7 wt %) | (1.7 wt %) |
| Formulation DQ | HN001 | Ethyl | Ethyl | Oleic Acid |
| | (4.25 wt %) | Cellulose | Cellulose | (1.75 wt %) |
| | | | (4.25 wt %) | |
| Formulation DR | HN001 | Ethyl | Ethyl | Oleic Acid |
| | (50 wt %) | Cellulose | Cellulose | (25 wt %) |
| | | | (25 wt %) | |
| Formulation DS | HN001 | Ethyl | Ethyl | Oleic Acid |
| | (10 wt %) | Cellulose | Cellulose | (15 wt %) |
| | | | (45 wt %) | |
| Formulation DT | HN001 | Ethyl | Ethyl | Oleic Acid |
| | (10 wt %) | Cellulose | Cellulose | (15 wt %) |
| | | | (45 wt %) | |
| Formulation DU | HN001 | Ethyl | Ethyl | Oleic Acid |
| | (10 wt %) | Cellulose | Cellulose | (15 wt %) |
| | | | (45 wt %) | |
| Formulation DV | HN001 | GV60 | GV60 | Ethyl |
| | (5 wt %) | | (9.3 wt %) | Cellulose |
| | | | | (57.4 wt %) |
| Formulation DW | HN001 | GV60 | GV60 | Ethyl |
| | (10 wt %) | | (40 wt %) | Cellulose |
| | | | | (33 wt %) |
| Formulation DX | HN001 | GV60 | GV60 | Ethyl |
| | (33.3 wt %) | | (61.9 wt %) | Cellulose |
| | | | | (3.4 wt %) |
| Formulation DY | HN001 | GV60 | GV60 | Ethyl |
| | (9.3 wt %) | | (83.7 wt %) | Cellulose |
| | | | | (4.98 wt %) |
| Formulation DZ | HN001 | GV60 | GV60 | Calcium |
| | (5 wt %) | | (85 wt %) | Carbonate |
| | | | | (10 wt %) |
| Formulation EA | HN001 | GV60 | GV60 | Calcium |
| | (4.3 wt %) | | (72.6 wt %) | Carbonate |
| | | | | (8.54 wt %) |
| Formulation EB | HN001 | Ethyl | Ethyl | Oleic Acid |
| | (86.5 wt %) | Cellulose | Cellulose | (3.8571 wt %) |
| | | | (9.6428 wt %) | |
| Formulation EC | HN001 | GV60 | GV60 | Ethyl |
| | (9 wt %) | | (81.5 wt %) | Cellulose |
| | | | | (6.785 wt %) |
| Formulation ED | HN001 | Ethyl | Ethyl | Oleic Acid |
| | (5 wt %) | Cellulose | Cellulose | (8.3 wt %) |
| | | | (50 wt %) | |
| Formulation EF | HN001 | GV60 | GV60 | Calcium |
| | (10 wt %) | | (80 wt %) | Carbonate |
| | | | | (10 wt %) |
| Formulation EG | HN001 | Ethyl | Ethyl | Oleic Acid |
| | (83 wt %) | Cellulose | Cellulose | (4.85724 wt %) |
| | | | (12.1 wt %) | |
| Formulation EH | HN001 | Ethyl | Ethyl | Olcic Acid |
| | (13 wt %) | Cellulose | Cellulose | (0.78 wt %) |
| | | | (1.8 wt %) | |
| Formulation EI | HN001 | GV60 | GV60 | Calcium |
| | (50 wt %) | | (40 wt %) | Carbonate |
| | | | | (10 wt %) |
| Formulation EJ | HN001 | GV60 | GV60 | Calcium |
| | (28.0898876404494 wt %) | | (56.28 wt %) | Carbonate |
| | | | | (15.62 wt %) |
| Formulation EK | HN001 | GV60 | GV60 | Calcium |
| | (15.7808357530615 wt %) | | (65.74788 wt %) | Carbonate |
| | | | | (18.4712842469385 wt %) |
| Formulation EL | HN001 | GV60 | GV60 | Calcium |
| | (8.86563806351769 wt %) | | (70.4363307933333 wt %) | Carbonate |
| | | | | (20.698031143149 wt %) |
| Formulation EM | HN001 | GV60 | GV60 | Calcium |
| | (4.98069554130207 wt %) | | (73.3784057955889 wt %) | Carbonate |
| | | | | (21.640898663109 wt %) |
| Formulation EN | HN001 | Blue | Blue | Calcium |
| | (10 wt %) | GV60 | GV60 | Carbonate |
| | | | (80 wt %) | (10 wt %) |
| Formulation EO | HN001 | Ethyl | Ethyl | Oleic Acid |
| | (11 wt %) | Cellulose | Cellulose | (5.1 wt %) |
| | | | (12.4 wt %) | |
| Formulation EP | HN001 | Ethyl | Ethyl | Oleic Acid |
| | (5 wt %) | Cellulose | Cellulose | (1.8 wt %) |
| | | | (4.4 wt %) | |
| Formulation EQ | HN019 | GV60 | GV60 | Calcium |
| | (10 wt %) | | (80 wt %) | Carbonate |
| | | | | (10 wt %) |
| Formulation ER | HN019 | GV60 | GV60 | |
| | (10 wt %) | | (90 wt %) | |
| Formulation ES | HN019 | GV60 | GV60 | Ethyl |
| | (9.4 wt %) | | (84.6 wt %) | Cellulose |
| | | | | (Colorcon) |
| | | | | (6 wt %) |
| Formulation ET | HN019 | GV60 | GV60 | Calcium |
| | (8.55 wt %) | | (68.4 wt %) | Carbonate |
| | | | | (8.55 wt %) |
| Formulation EU | HN019 | Ethyl | Ethyl | |
| | (83.33 wt %) | Cellulose | Cellulose | |
| | | | (16.67 wt %) | |
| Formulation EV | HN019 | Bees Wax | Bees Wax | Carnauba |
| | (4.13 wt %) | | (39.24 wt %) | Wax |
| | | | | (39.24 wt %) |
| Formulation EW | HN019 | Bees Wax | Bees Wax | Stearic Acid |
| | (4.3 wt %) | | (40.7 wt %) | (40.7 wt %) |
| Formulation EX | HN001 | Bees Wax | Bees Wax | Stearic Acid |
| | (13 wt %) | | (43.5 wt %) | (43.5 wt %) |
| Formulation EY | HN001 | Bees Wax | Bees Wax | Carnauba |
| | (13 wt %) | | (43.5 wt %) | Wax |
| | | | | (43.5 wt %) |

| | | | | | Coating, |
|---|---|---|---|---|---|
| | Component 3 | Component 4 | Component 5 | Component 6 | if present |
| Formulation | (wt %) | (wt %) | (wt %) | (wt %) | (wt %) |
| Formulation BM | | | | | |
| Formulation BN | | | | | |
| Formulation BO | | | | | |
| Formulation BP | | | | | |
| Formulation BQ | | | | | |
| Formulation BR | Microcrystalline | | | | |
| | Cellulose | | | | |
| | (5 wt %) | | | | |
| Formulation BS | | | | | |
| Formulation BT | | | | | |
| Formulation BU | | | | | |
| Formulation BV | | | | | |
| Formulation BW | PEG 3350 | | | | |
| | (10 wt %) | | | | |
| Formulation BX | | | | | |
| Formulation BY | Sucrose | Maltitol | | | |
| | (3.6 wt %) | (3.6 wt %) | | | |
| Formulation BZ | | | | | |
| Formulation CA | | | | | |
| Formulation CB | Magnesium | | | | |
| | Stearate | | | | |
| | (1.3 wt %) | | | | |
| Formulation CC | Magnesium | | | | |
| | Stearate | | | | |
| | (1.3 wt %) | | | | |
| Formulation CD | Sorbitol | Magnesium | | | |
| | (20 wt %) | Stearate | | | |
| | | (1.3 wt %) | | | |
| Formulation CE | Sorbitol | Magnesium | | | |
| | (20 wt %) | Stearate | | | |
| | | (1.3 wt %) | | | |
| Formulation CF | Sorbitol | Magnesium | | | |
| | (20 wt %) | Stearate | | | |
| | | (1.3 wt %) | | | |
| Formulation CG | Inulin | Sorbitol | Magnesium | Carbonate | |
| | (2.7 wt %) | (12 wt %) | Stearate | Calcium | |
| | | | (0.3 wt %) | (10 wt %) | |
| Formulation CH | | | | | |
| Formulation CI | PEG 3350 | | | | |
| | (10 wt %) | | | | |
| Formulation CJ | Polydextrose | | | | |
| | (2.5 wt %) | | | | |
| Formulation CK | Sorbitol | Tylose | Magnesium | | |
| | (13.3 wt %) | (6.7 wt %) | Stearate | | |
| | | | (1.3 wt %) | | |
| Formulation CL | Magnesium | | | | |
| | Stearate | | | | |
| | (1 wt %) | | | | |
| Formulation CM | | | | | |
| Formulation CN | | | | | |
| Formulation CO | Polydextrose | | | | |
| | (25 wt %) | | | | |
| Formulation CP | Polydextrose | | | | |
| | (17 wt %) | | | | |
| Formulation CQ | Polydextrose | GV60 | Calcium | | |
| | (5 wt %) | (70 wt %) | Carbonate | | |
| | | | (10 wt %) | | |
| Formulation CR | Isomalt | Polydextrose | GV60 | Calcium | |
| | (4.5 wt %) | (5 wt %) | (70 wt %) | Carbonate | |
| | | | | (10 wt %) | |
| Formulation CS | Isomalt | GV60 | Calcium | | |
| | (4.1 wt %) | (70 wt %) | Carbonate | | |
| | | | (10 wt %) | | |
| Formulation CT | Calcium | | | | |
| | Carbonate | | | | |
| | (10 wt %) | | | | |
| Formulation CU | Calcium | GV60 | Calcium | | |
| | Chloride | (64 wt %) | Carbonate | | |
| | (1 wt %) | | (10 wt %) | | |
| Formulation CV | Calcium | | | | |
| | Carbonate | | | | |
| | (15 wt %) | | | | |
| Formulation CW | Calcium | GV60 | Calcium | | |
| | Chloride | (72 wt %) | Carbonate | | |
| | (0.5 wt %) | | (15 wt %) | | |
| Formulation CX | | | | | |
| Formulation CY | | | | | Shellac |
| Formulation CZ | | | | | Ethyl |
| | | | | | Cellulose |
| Formulation DA | | | | | Shellac |
| Formulation DB | | | | | Shellac |
| Formulation DC | | | | | Shellac |
| Formulation DE | | | | | Shellac |
| Formulation DF | GV60 | | | | |
| | (74 wt %) | | | | |
| Formulation DG | GV60 | | | | Ethyl |
| | (74 wt %) | | | | Cellulose/ |
| | | | | | Oleic Acid |
| Formulation DH | | | | | Ethyl |
| | | | | | Cellulose/ |
| | | | | | Oleic Acid |
| Formulation DI | Calcium | | | | Ethyl |
| | Carbonate | | | | Cellulose/ |
| | (35 wt %) | | | | Oleic Acid |
| | | | | | (21 wt %) |
| Formulation DJ | Calcium | | | | Ethyl |
| | Carbonate | | | | Cellulose/ |
| | (28.24 wt %) | | | | Oleic Acid |
| | | | | | (31.1 wt %) |
| Formulation DK | Calcium | GV60 | | | Ethyl |
| | Carbonate | (76 wt %) | | | Cellulose/ |
| | (15 wt %) | | | | Oleic Acid |
| | | | | | (4 wt %) |
| Formulation DL | Oliec Acid | Calcium | | | |
| | (1.1 wt %) | Carbonate | | | |
| | | (12.5 wt %) | | | |
| Formulation DM | | | | | Shellac |
| | | | | | (50 wt %) |
| Formulation DN | Calcium | | | | |
| | Carbonate | | | | |
| | (15 wt %) | | | | |
| Formulation DO | Calcium | | | | |
| | Carbonate | | | | |
| | (9.4 wt %) | | | | |
| Formulation DP | Magnesium | GV60 | | | |
| | Stearate | (75.6 wt %) | | | |
| | (1 wt %) | | | | |
| Formulation DQ | GV60 | Calcium | | | |
| | (78.75 wt %) | Carbonate | | | |
| | | (11 wt %) | | | |
| Formulation DR | | | | | |
| Formulation DS | Stearic Acid | | | | |
| | (30 wt %) | | | | |
| Formulation DT | Stearic Acid | | | | |
| | (30 wt %) | | | | |
| Formulation DU | Stearic Acid | | | | |
| | (30 wt %) | | | | |
| Formulation DV | Oleic Acid | | | | |
| | (28.3 wt %) | | | | |
| Formulation DW | Oleic Acid | | | | |
| | (17 wt %) | | | | |
| Formulation DX | Oleic Acid | | | | Ethyl |
| | (1.36 wt %) | | | | Cellulose/ |
| | | | | | Oleic Acid |
| | | | | | (4.76 wt %) |
| Formulation DY | Olcic Acid | | | | Ethyl |
| | (2 wt %) | | | | Cellulose/ |
| | | | | | Oleic Acid |
| | | | | | (6.97 wt %) |
| Formulation DZ | | | | | |
| Formulation EA | Ethyl | Oleic Acid | | | Ethyl |
| | Cellulose | (4.171366 wt %) | | | Cellulose/ |
| | (10.35706 wt %) | | | | Oleic Acid |
| | | | | | (14.6 wt %) |
| Formulation EB | | | | | Ethyl |
| | | | | | Cellulose/ |
| | | | | | Oleic Acid |
| | | | | | (13.5 wt %) |
| Formulation EC | Oleic Acid | | | | Ethyl |
| | (2.714 wt %) | | | | Cellulose/ |
| | | | | | Oleic Acid |
| | | | | | (9.5 wt %) |
| Formulation ED | MCC | Stearic | | | |
| | (18.3 wt %) | Acid - 90% | | | |
| | | (18.3 wt %) | | | |
| Formulation EF | | | | | |
| Formulation EG | | | | | |
| Formulation EH | GV60 | Calcium | | | |
| | (74.46 wt %) | Carbonate | | | |
| | | (10 wt %) | | | |
| Formulation EI | | | | | |
| Formulation EJ | | | | | |
| Formulation EK | | | | | |
| Formulation EL | | | | | |
| Formulation EM | | | | | |
| Formulation EN | | | | | |
| Formulation EO | GV60 | Calcium | | | Ethyl |
| | (63.1 wt %) | Carbonate | | | Cellulose/ |
| | | (8.5 wt %) | | | Oleic Acid |
| | | | | | (17.5 wt %) |
| Formulation EP | GV60 | Calcium | | | Ethyl |
| | (50 wt %) | Carbonate | | | Cellulose/ |
| | | (38.8 wt %) | | | Oleic Acid |
| | | | | | (0 wt %) |
| Formulation EQ | | | | | |
| Formulation ER | | | | | |
| Formulation ES | | | | | Ethyl |
| | | | | | Cellulose |
| | | | | | (6 wt %) |
| Formulation ET | Ethy | | | | Ethyl |
| | Cellulose | | | | Cellulose |
| | (Colorcon) | | | | (14.5 wt %) |
| | (14.5 wt %) | | | | |
| Formulation EU | | | | | Ethyl |
| | | | | | Cellulose |
| | | | | | (16.67 wt %) |
| Formulation EV | Ethy | | | | Ethyl |
| | Cellulose | | | | Cellulose |
| | (Colorcon) | | | | (17.39 wt %) |
| | (17.39 wt %) | | | | |
| Formulation EW | Ethy | | | | Ethyl |
| | Cellulose | | | | Cellulose |
| | (Colorcon) | | | | (14.3 wt %) |
| | (14.3 wt %) | | | | |
| Formulation EX | | | | | |
| Formulation EY | | | | | |

TABLE 19
Exemplary Methods for Preparing Formulations for Inclusion in Yogurt

| Formulation | Step(s) | | | | |
|---|---|---|---|---|---|
| Formulation BM | Prilling | | | | |
| Formulation BN | Prilling | | | | |
| Formulation BO | Prilling | | | | |
| Formulation BP | Prilling | | | | |
| Formulation BQ | Prilling | | | | |
| Formulation BR | Prilling | | | | |
| Formulation BS | Prilling | | | | |
| Formulation BT | Prilling | | | | |
| Formulation BU | Prilling | | | | |
| Formulation BV | Prilling | | | | |
| Formulation BW | Prilling | | | | |
| Formulation BX | Prilling | | | | |
| Formulation BY | Prilling | | | | |
| Formulation BZ | Extrusion | | | | |
| Formulation CA | Extrusion | | | | |
| Formulation CB | Extrusion | | | | |
| Formulation CC | Extrusion | | | | |
| Formulation CD | Extrusion | | | | |
| Formulation CE | Extrusion | | | | |
| Formulation CF | Extrusion | | | | |
| Formulation CG | Extrusion | Extrusion | | | |
| Formulation CH | Prilling | | | | |
| Formulation CI | Prilling | | | | |
| Formulation CJ | Prilling | | | | |
| Formulation CK | Extrusion | | | | |
| Formulation CL | Extrusion | | | | |
| Formulation CM | Extrusion | | | | |
| Formulation CN | Extrusion | | | | |
| Formulation CO | Extrusion | | | | |
| Formulation CP | Extrusion | | | | |
| Formulation CQ | Extrusion | | | | |
| Formulation CR | Extrusion | | | | |
| Formulation CS | Extrusion | | | | |
| Formulation CT | Extrusion | | | | |
| Formulation CU | Extrusion | | | | |
| Formulation CV | Extrusion | | | | |
| Formulation CW | Extrusion | Extrusion | | | |
| Formulation CX | Extrusion | | | | |
| Formulation CY | Extrusion | Dip Coat | | | |
| Formulation CZ | Extrusion | Dip Coat | | | |
| Formulation DA | Extrusion | Dip Coat | | | |
| Formulation DB | Extrusion | Dip Coat | | | |
| Formulation DC | Prilling | Pan Coat | | | |
| Formulation DE | Extrusion | Dip Coat | | | |
| Formulation DF | Extrusion | | | | |
| Formulation DG | Extrusion | Dip Coat | | | |
| Formulation DH | Prilling | Pan Coat | | | |
| Formulation DI | Extrusion | | | | |
| Formulation DJ | Extrusion | | | | |
| Formulation DK | Vacuum Dry/Coat | Extrusion | | | |
| Formulation DL | Extrusion | | | | |
| Formulation DM | Extrusion | | | | |
| Formulation DN | Extrusion | | | | |
| Formulation DO | Vacuum Dry/Coat | | | | |
| Formulation DP | Extrusion | | | | |
| Formulation DQ | Extrusion | | | | |
| Formulation DR | Extrusion | | | | |
| Formulation DS | Extrusion | | | | |
| Formulation DT | Extrusion | | | | |
| Formulation DU | Extrusion | | | | |
| Formulation DV | Prilling | Extrusion | | | |
| Formulation DW | Extrusion | | | | |
| Formulation DX | Prilling | Fuild Bed | | | |
| Formulation DY | Prilling | Fluid Bed | | | |
| Formulation DZ | Extrusion | | | | |
| Formulation EA | Extrusion | | | | |
| Formulation EB | Fluid Bed | | | | |
| Formulation EC | Extrusion | | | | |
| Formulation ED | Extrusion | | | | |
| Formulation EF | Extrusion | | | | |
| Formulation EG | Fluid Bed | | | | |
| Formulation EH | Fluid Bed | Extrusion | | | |
| Formulation EI | Extrusion | | | | |
| Formulation EJ | Extrusion | Extrusion | | | |
| Formulation EK | Extrusion | Extrusion | Extrusion | | |
| Formulation EL | Extrusion | Extrusion | Extrusion | Extrusion | |
| Formulation EM | Extrusion | Extrusion | Extrusion | Extrusion | Extrusion |
| Formulation EN | Extrusion | | | | |
| Formulation EO | Fluid Bed | Extrusion | Fluid Bed | | |
| Formulation EP | Extrusion | Fluid Bed | Extrusion | | |
| Formulation EQ | Extrusion | | | | |
| Formulation ER | Extrusion | | | | |
| Formulation ES | Extrusion | Fluid Bed | | | |
| Formulation ET | Extrusion | Fluid Bed | | | |
| Formulation EU | Fluid Bed | | | | |
| Formulation EV | Prilling | Fluid Bed | | | |
| Formulation EW | Prilling | Fluid Bed | | | |
| Formulation EX | Extrusion | | | | |
| Formulation EY | Extrusion | | | | |

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

What is claimed is:

1. A probiotic composition comprising one or more microbes and one or more barrier materials wherein the barrier materials comprise, on a dry weight basis:
about 1% to about 99% (w/w) lipid, the lipid comprising at least one of a wax, a plant oil, and a fatty acid; and
about 0% to about 98% (w/w) of:
a carbohydrate;
a protein;
a polymer;
or combinations thereof,
wherein the microbes comprise probiotic bacteria.

2. A particle comprising the composition of claim 1, wherein the particle comprises a diameter in a range from about 10 μm to about 300 μm.

3. The particle of claim 176, wherein the particle comprises a diameter in a range from about 100 μm to about 250 μm.

4. The particle comprising the composition of claim 1, wherein the particle comprises a diameter in a range from about 10 m to about 100 m.

5. The particle comprising the composition of claim 1, wherein the particle comprises a diameter in a range from about 10 μm to about 50 μm.

6. The particle comprising the composition of claim 1, wherein the particle comprises a diameter in a range from about 10 μm to about 25 μm.

7. The composition of claim 1, wherein the probiotic bacteria comprises at least one of Bacillus coagulans, Bacillus licheniformis, Bacillus subtilis, Bifidobacterium angulatum, Bifidobacterium animalis, Bifidobacterium bifidum, Bifidobacterium breve, Bifidobacterium infantis, Bifidobacterium Bifidobacterium longum, Enterococcus faecium, Enterococcus faecalis, Lactobacillus acidophilus, Lactobacillus amylovorus, Lactobacillus alimentarius, Lactobacillus bulgaricus, Lactobacillus casei subsp. casei, Lactobacillus casei Sinrota, Lactobacillus curvatus, Lactobacillus delbrueckii subsp lactis, Lactobacillus fermentum, Lactobacillus farciminis, Lactobacillus gasseri, Lactobacillus helveticus, Lactobacillus johnsonii, Lactobacillus lacti, Lactobacillus paracasei, Lactobacillus pentosaceus, Lactobacillus plantarum, Lactobacillus reuteri, Lactobacillus rhamnosus (Lactobacillus GG), Lactobacillus sake, Lactobacillus salivarius, Lactococcus lactis, Micrococcus varians, Pediococcus acidilactici Pediococcus pentosaceus, Pediococcus acidilactici, Pediococcus halophilus, Streptococcus faecalis, Streptococcus thermophilus, Staphylococcus camosus, and Staphylococcus xylosus.

8. The composition of claim 7, wherein the carbohydrate comprises amylose, amylopectin, cellulose, methyl cellulose, ethyl cellulose, hydroxypropyl cellulose, hydroxypropyl methyl cellulose, hydroxypropyl ethyl cellulose, cellulose triacetate, cellulose acetate succinate, cellulose acetate butyrate, cellulose acetate phthalate, hydroxypropyl methylcellulose acetate succinate, sodium alginate, potassium alginate, calcium alginate, magnesium alginate, zinc alginate, sodium pectinate, potassium pectinate, calcium pectinate, zinc pectinate, sodium hyaluronate, potassium hyaluronate, calcium hyaluronate, magnesium hyaluronate, zinc hyaluronate, or a combination thereof.

9. The composition of claim 7, wherein the protein comprises whey protein, β-lactoglobulin, α-lactalbumin, casein, bovine serum albumin, ovalbumin, zein, hordein, gliadin, secalin, kafirin, avenin, or a combination thereof.

10. The composition of claim 1, wherein loss of log(CFU) of the probiotic bacteria in the probiotic composition is less than about 2, less than about 1, or less than about 0.5, when mixed and stored in a food and/or beverage product having a pH less than about 5.0 for at least 1 week.

11. The composition of claim 1, wherein loss of log(CFU) of the probiotic bacteria in the probiotic composition is less than about 2, less than about 1, or less than about 0.5, when mixed and stored in a food and/or beverage product having a pH less than about 5.0 for at least 2 weeks.

12. The composition of claim 1, wherein loss of log(CFU) of the probiotic bacteria in the probiotic composition is less than about 2, less than about 1, or less than about 0.5, when mixed and stored in a food and/or beverage product having a pH less than about 5.0 for at least 4 weeks.

13. The composition of claim 1, wherein loss of log(CFU) of the probiotic bacteria in the probiotic composition is less than about 8, less than about 7, less than bout 6, less than about 5, or less than about 4, when mixed and in a food and/or beverage product and exposed to a temperature about 100° C. for 30 minutes.

14. The composition of claim 1, wherein loss of log(CFU) of the probiotic bacteria in the probiotic composition is less than about 2, less than about 1, or less than about 0.5, when mixed and stored in a food and/or beverage product having a fat content of about 1% to about 5% for at least 1 week.

15. The composition of claim 1, wherein loss of log(CFU) of the probiotic bacteria in the probiotic composition is less than about 2, less than about 1, or less than about 0.5, when mixed and stored in a food and/or beverage product having a fat content of about 1% to about 5% for at least 2 weeks.

16. The composition of claim 1, wherein loss of log(CFU) of the probiotic bacteria in the probiotic composition is less than about 2, less than about 1, or less than about 0.5, when mixed and stored in a food and/or beverage product having a fat content of about 1% to about 5% for at least 4 weeks.

17. The composition of claim 1, wherein loss of log(CFU) of the probiotic bacteria in the probiotic composition is less than about 2, less than about 1, or less than about 0.5, when mixed and stored in a liquid food and/or beverage product at about 25° C. for at least 1 week.

18. The composition of claim 1, wherein loss of log(CFU) of the probiotic bacteria in the probiotic composition is less than about 2, less than about 1, or less than about 0.5, when mixed and stored in a food and/or beverage product at about 25° C. for at least 2 weeks.

19. The composition of claim 1, wherein loss of log(CFU) of the probiotic bacteria in the probiotic composition is less than about 2, less than about 1, or less than about 0.5, when mixed and stored in a food and/or beverage product at about 25° C. for at least 4 weeks.

19. The composition of claim 1, wherein the probiotic composition does not significantly alter the pH of a food and/or beverage product when mixed and stored in the food and/or beverage product at about 25° C. for at least 1 week.

20. The composition of claim 1, wherein the probiotic composition does not significantly alter the pH of a food and/or beverage product when mixed and stored in the food and/or beverage product at about 25° C. for at least 2 weeks.

21. The composition of claim 1, wherein the probiotic composition does not significantly alter the pH of a food and/or beverage product when mixed and stored in the food and/or beverage product at about 25° C. for at least 4 weeks.

22. The composition of claim 1, wherein the particle preparation comprises a water activity from about 0.1 to about 0.3.

23. The composition of claim 1, wherein the particle preparation comprises a dispersity from about 0.1 to about 0.4.

24. The composition of claim 1, further comprising an excipient component.

25. The composition of claim 24, wherein the excipient component comprises at least one of calcium carbonate, an anti-caking component, an anti-agglomerating component, an anti-clumping component, an anti-aggregating component, a surfactant component, a plasticizing component, an acid scavenger, an oxygen scavenger, a moisture scavenger, a water scavenger, a desiccant.

26. The composition of claim 7, wherein the probiotic bacteria comprises at least one of Lacticaseibacillus rhamnosus (HN001), Bifidobacterium lactis (HN019), Bifidobacterium lactis (BI-07), and Lactobacillus acidophilus.

27. The composition of claim 26, wherein the probiotic bacteria comprises at least one of Lacticaseibacillus rhamnosus (HN001) and Bifidobacterium lactis (HN019).

28. The composition of claim 1, wherein the lipid comprises at least one of paraffin wax, hydrogenated palm oil, and palmitic acid.

29. The composition of claim 26, comprising about 5% (w/w) of the probiotic bacteria and about 95% (w/w) of the lipid, wherein the lipid comprises at least one of paraffin wax, hydrogenated palm oil, and palmitic acid.

30. The composition of claim 26, comprising about 35% (w/w) of the probiotic bacteria and about 65% (w/w) of the lipid, wherein the lipid comprises at least one of paraffin wax, hydrogenated palm oil, and palmitic acid.

31. The composition of claim 26, comprising from about 5% to about 35% (w/w) of the probiotic bacteria, and from about 65% to about 95% (w/w) of the lipid, wherein the lipid comprises at least one of paraffin wax, hydrogenated palm oil, and palmitic acid.

32. The composition of claim 1, wherein the barrier material comprises:
a first inner layer comprising at least one of a hydrophilic material and a water soluble material; and
a second outer layer comprising at least one of a hydrophobic material and a fat soluble material.

33. The composition of claim 1, wherein the polymer comprises at least one of a bile-responsive polymer, a pH-responsive polymer, and a microbiome-responsive polymer.

34. The composition of claim 1, wherein the probiotic bacteria is encapsulated with at least one of a desiccant and a cryoprotectant.

35. The composition of claim 1, further comprising a carotenoid comprising at least one of alpha-lipoic acid, astaxanthin, adonixanthin, adonirubin, beta-carotene, coenzyme Q10, lutein, lycopene, zeaxanthin, and meso-zeaxanthin.

36. The composition of claim 1, wherein the probiotic bacteria comprises at least one spore forming species.

37. The composition of claim 24, wherein the excipient imparts a change to at least one of:
(i) an environment within the particle composition comprising at least one of a pH change, an oxygen concentration change, and a water concentration change; and
(ii) a local environment in which the probiotic composition resides comprising at least one of a stomach, a food matrix, and a beverage;
wherein a change to the environment within the particle composition

38. The composition of claim 1, wherein the composition comprises a particle preparation comprising the microbes, wherein a diameter of the composition is about 10% to about 30% larger than a diameter of the particle preparation.

39. A food product coated with the probiotic composition of claim 1, wherein the food product comprising at least one of a gelatin-based matrix and a pectin-based matrix.

40. The food product of claim 191, wherein the at least one gelatin-based matrix and/or the pectin-based matrix comprises a water activity in a range from 0.50 plus or minus 0.01 to 0.70 plus or minus 0.01.

41. A method of manufacturing a probiotic composition comprising microbes and a barrier material comprising the steps of:
i. milling a freeze-dried microbes solution, forming milled microbes;
ii. dispersing the milled microbes within a liquid, thereby forming a suspension;
iii. homogenizing the suspension of microbes within a liquid matrix;
iv. atomizing the homogenized liquid matrix;
v. air-cooling the atomized liquid matrix, forming cooled compositions;
vi. collecting the cooled compositions;
vii. coating the collected cooled compositions forming coated compositions; and
viii. drying the coated compositions.

42. The method of claim 41, wherein the probiotic composition is a particle preparation.

43. The method of claim 42, wherein the particle preparation is about 1-10000 μm in diameter.

44. The method of claim 42, wherein the particle preparation is about 2-5000 μm in diameter.

45. The method of claim 42, wherein the particle preparation is about 3-1000 μm in diameter.

46. The method of claim 42, wherein the particle preparation is about 10-1000 μm in diameter.

47. The method of any one of claims 42-46, wherein the dispersity of the particle preparation is <about 0.4, <about 0.3, <about 0.2, and/or <about 0.1.

48. The method of any one of claims 42-47, wherein the diameter and dispersity are measured using a Malvern Mastersizer.

49. The method of any one of claims 42-47, wherein the diameter and dispersity are measured using Scanning Electron Microscopy.

50. The method of any one of claims 42-49, wherein the particle preparation comprises a water activity of <about 0.6, <about 0.3, <about 0.2, <about 0.1.

51. The method of claim 50, wherein the water activity is measured using a TDL2 water activity meter.

52. The method of any one of claims 42-51, wherein the particle composition comprises core materials encapsulated by a shell material.

53. The method of any one of claims 41-52, wherein the microbes are bacteria.

54. The method of claim 53, wherein the bacteria are probiotics.

55. The method of claim 54, wherein the probiotics are selected from the group comprising Bacillus coagulans, Bacillus licheniformis, Bacillus subtilis, Bifidobacterium angulatum, Bifidobacterium animalis, Bifidobacterium bifidum, Bifidobacterium breve, Bifidobacterium infantis, Bifidobacterium Bifidobacterium longum, Enterococcus faecium, Enterococcus faecalis, Lactobacillus acidophilus, Lactobacillus amylovorus, Lactobacillus alimentarius, Lactobacillus bulgaricus, Lactobacillus casei subsp. casei, Lactobacillus casei Sinrota, Lactobacillus curvatus, Lactobacillus delbrueckii subsp lactis, Lactobacillus fermentum, Lactobacillus farciminis, Lactobacillus gasseri, Lactobacillus helveticus, Lactobacillus johnsonii, Lactobacillus lacti, Lactobacillus paracasei, Lactobacillus pentosaceus, Lactobacillus plantarum, Lactobacillus reuteri, Lactobacillus rhamnosus (Lactobacillus GG), Lactobacillus sake, Lactobacillus salivarius, Lactococcus lactis, Micrococcus varians, Pediococcus acidilactici Pediococcus pentosaceus, Pediococcus acidilactici, Pediococcus halophilus, Streptococcus faecalis, Streptococcus thermophilus, Staphylococcus camosus, or Staphylococcus xylosus.

56. The method of any one of claims 41-54, wherein the barrier material is a solid at 25° C.

57. The method of claim 56, wherein the barrier material is moisture resistant.

58. The method of any one of claims 56-57, wherein the barrier material melts between 30-90° C.

59. The method of any one of claims 56-57, wherein the barrier material melts between 36-70° C.

60. The method of any one of claims 56-57, wherein the barrier material is characterized to melt between 40-60° C.

61. The method of any one of claims 56-60, wherein the barrier material is a lipid.

62. The method of claim 61, wherein the lipid comprises, on a dry weight basis, between about 40% to about 99% (w/w) of the total mass of the probiotic composition.

63. The method of any one of claims 61-62, wherein the lipid is a wax.

64. The method of claim 63, wherein the wax comprises paraffin wax, montan wax, microcrystalline wax, polyethylene wax, petrolatum wax, ozokerite wax, ceresin wax, beeswax, lanolin wax, spermaceti wax, tallow wax, lac wax, chinese insect wax, ambergris wax, soy wax, camauba wax, candelilla wax, coconut wax, palm kernel wax, rice bran wax, or combinations thereof.

65. The method of any one of claims 61-62, wherein the lipid is a plant oil.

66. The method of claim 65, wherein the plant oil comprises fatty acid monoglyceride esters, fatty acid diglyceride esters, fatty acid triglyceride esters, coconut oil, cottonseed oil, palm oil, soybean oil, sunflower oil, fully hydrogenated palm oil, partially hydrogenated palm oil, fully hydrogenated sunflower oil, partially hydrogenated sunflower oil, fully hydrogenated soybean oil, partially hydrogenated soybean oil, fully hydrogenated vegetable oil, partially hydrogenated vegetable oil, fully hydrogenated cottonseed oil, partially hydrogenated cottonseed oil, or a combination thereof.

67. The method of any one of claims 61-62, wherein the lipid is a fatty acid.

68. The method of claim 63, wherein the fatty acid comprises butyric acid, pentanoic acid, hexanoic acid, heptanoic acid, caprylic acid, nonanoic acid, capric acid, lauric acid, myristic acid, palmitic acid, stearic acid, and/or arachidonic acid, or a combination thereof.

69. The method of any one of claims 41-68, wherein milling is achieved using at least one of the following methods: planetary milling, ball milling, burr milling, roller milling, media milling, impact milling, jet milling, high-pressure homogenization, cryo milling, hammer milling, conical milling, hand screening, or granulation/extrusion.

70. The method of claim 69, wherein milling is achieved using extrusion.

71. The method of any of claims 69-70, wherein a processing aid is included in the homogenization step.

72. The method of claim 71, wherein the processing aid comprises: calcium carbonate, calcium phosphate, calcium hydroxide, calcium hydroxyapatite, zinc oxide, titanium oxide, silicon oxide, or combinations thereof.

73. The method of any one of claims 41-72, wherein dispersing the milled microbes within the liquid is achieved using an overhead stirrer, manual stirring, a stir bar, high pressure homogenization, low pressure homogenization, sonication, ultrasonication, vortexing, or a combination thereof.

74. The method of any one of claims 41-73, wherein atomizing the homogenized liquid matrix is achieved using planetary milling, ball milling, burr milling, roller milling, media milling, impact milling, jet milling, high-pressure homogenization, cryo milling, hammer milling, conical milling, hand screening, or granulation/extrusion, extrusion, spray drying, fluid bed agglomeration, spray congealing, high-shear granulation, tableting, roller compaction, crosslinking, pouring, prilling, spinning disc atomization, or a combination thereof.

75. The method of any one of claims 41-74, wherein atomizing the homogenized liquid matrix is achieved using spinning disc atomization.

76. The method of claim 75, wherein spinning disc atomization is at a disc speed between about 2000 and about 6000 rpm.

77. The method of claim 75, wherein spinning disc atomization is at a disc speed between about 4000 and about 5000 rpm.

78. The method of any one of claims 75-77, wherein spinning disc atomization is at a temperature between about 50° C. and about 90° C.

79. The method of any one of claims 75-78, wherein air-cooling the atomized liquid matrix is at a temperature between about 20° C. and about 25° C.

80. The method of claims 75-79, wherein the cooled compositions are collected on a powder bed.

81. The method of claim 80, wherein the powder bed is a material that reduces particle agglomeration.

82. The method of claim 81, wherein the powder bed is at least one of: spray dried starch, spray dried lactose, magnesium stearate, zinc stearate, stearic acid, silicon dioxide, zinc oxide, titanium oxide, aluminum oxide, or combinations thereof.

83. The method of any one of claims 41-82, wherein the relative composition of microbes to barrier material is between about 0.01% and 40% (w/w).

84. The method of any one of claims 41-82, wherein the relative composition of microbes to barrier material is between about 5% and 25% (w/w).

85. The method of any one of claims 41-84, wherein coating is achieved using at least one of the following methods: spray pan coating, fluidized bed coating, dip coating, roller coating, or sputter coating.

86. The method of any one of claims 41-85, wherein the collected cooled compositions are coated with a material selected from at least one of the following: a carbohydrate, a protein, or combinations thereof.

87. The method of claim 86, wherein the carbohydrate comprises: amylose, amylopectin, cellulose, methyl cellulose, ethyl cellulose, hydroxypropyl cellulose, hydroxvpropyl methyl cellulose, hydroxypropyl ethyl cellulose, cellulose triacetate, cellulose acetate succinate, cellulose acetate butyrate, cellulose acetate phthalate, hydroxypropyl methylcellulose acetate succinate, sodium alginate, potassium alginate, calcium alginate, magnesium alginate, zinc alginate, sodium pectinate, potassium pectinate, calcium pectinate, zinc pectinate, sodium hyaluronate, potassium hyaluronate, calcium hyaluronate, magnesium hyaluronate, zinc hyaluronate, or a combination thereof.

88. The method of claim 86, wherein the protein comprises: whey protein, β-lactoglobulin, α-lactalbumin, casein, bovine serum albumin, ovalbumin, zein, hordein, gliadin, secalin, kafirin, avenin, or a combination thereof.

89. The method of any one of claims 41-88, wherein the coating material comprises, on a dry weight basis, between about 0% to about 59% (w/w) of the total mass of the probiotic composition.

90. The method of any one of claims 41-89, wherein drying of the coated compositions is achieved using at least one of the following methods: drierite, heating, vacuum, molecular sieves, sodium sulfate, magnesium sulfate, calcium carbonate, calcium chloride, or a combination thereof.

91. The method of claim 90, wherein water activity is reduced by >about 10%, >about 20%, and/or >about 30%.

92. A probiotic composition comprising microbes and one or more barrier materials, wherein the one or more barrier materials comprise, on a dry weight basis:
about 1% to about 99% (w/w) lipid; and
about 0% to about 98% (w/w) of:
a carbohydrate;
a protein;
a polymer; or
combinations thereof,

93. The composition of claim 92, wherein the composition is a particle preparation.

94. The composition of claim 93, wherein the particle composition is about 1-10000 m in diameter.

95. The composition of claim 93, wherein the particle composition is about 2-5000 μm in diameter.

96. The composition of claim 93, wherein the particle composition is about 3-1000 μm in diameter.

97. The composition of claim 93, wherein the particle composition is about 10-1000 m in diameter.

98. The composition of any one of claims 93-97, wherein dispersity of the particle composition is <about 0.4, <about 0.3, <about 0.2, <about 0.1.

99. The composition of any one of claims 93-98, wherein the diameter and dispersity are measured using a Malvern Mastersizer.

100. The composition of any one of claims 93-98, wherein the diameter and dispersity are measured using Scanning Electron Microscopy.

101. The composition of any one of claims 93-100, wherein the particle preparation comprises a water activity of <about 0.6, <about 0.3, <about 0.2, and/or <about 0.1.

102. The composition of claim 101 wherein the water activity is measured using a TDL2 water activity meter.

103. The composition of any one of claims 93-102 wherein the particle comprises core materials encapsulated by a shell material.

104. The composition of claim 103 wherein the core materials comprise, on a dry weight basis, about 40% to about 99% (w/w) of the particle composition.

105. The composition of claim 104, wherein the core materials comprise the microbes and the one or more barrier materials.

106. The composition of claim 105, wherein the microbes are a dry powder comprising a single species or a mixture of species.

107. The composition of claim 106, wherein particles of the dry powder are about 0.01-4000 μm in diameter.

108. The composition of claim 107, wherein particles of the dry powder are about 0.05-1000 μm in diameter.

109. The composition of claim 108, wherein particles of the dry powder are about 0.06-200 μm in diameter.

110. The composition of claim 109, wherein particles of the dry powder are about 1-100 μm in diameter.

111. The composition of claim 110, wherein the diameter is measured using a Malvern Mastersizer.

112. The composition of any one of claims 106-111, wherein the microbes are dispersed within the core materials

113. The composition of any one of claims 106-112, wherein a density of one or more core materials of the microbes is between about 1×10\hat{ }5 CFU/g and about 1×10\hat{ }14 CFU/g.

114. The composition of any one of claims 106-112, wherein a density of one or more core materials of the microbes is between about 1×10\hat{ }7 CFU/g and about 1×10\hat{ }13 CFU/g.

115. The composition of any one of claims 106-112, wherein a density of one or more core materials of the microbes is between about 1×10\hat{ }9 CFU/g and about 1×10\hat{ }12 CFU/g.

116. The composition of any one of claims 106-115, wherein the microbes are probiotic bacteria.

117. The composition of any one of claims 105-116, wherein the probiotic bacteria are selected from the group comprising: Bacillus coagulans, Bacillus licheniformis, Bacillus subtilis, Bifidobacterium angulatum, Bifidobacterium animalis, Bifidobacterium bifidum, Bifidobacterium breve, Bifidobacterium infantis, Bifidobacterium Bifidobacterium longum, Enterococcus faecium, Enterococcus faecalis, Lactobacillus acidophilus, Lactobacillus amylovorus, Lactobacillus alimentarius, Lactobacillus bulgaricus, Lactobacillus casei subsp. casei, Lactobacillus casei Sinrota, Lactobacillus curvatus, Lactobacillus delbrueckii subsp lactis, Lactobacillus fermentum, Lactobacillus farciminis, Lactobacillus gasseri, Lactobacillus helveticus, Lactobacillus johnsonii, Lactobacillus lacti, Lactobacillus paracasei, Lactobacillus pentosaceus, Lactobacillus plantarum, Lactobacillus reuteri, Lactobacillus rhamnosus (Lactobacillus GG), Lactobacillus sake, Lactobacillus salivarius, Lactococcus lactis, Micrococcus varians, Pediococcus acidilactici Pediococcus pentosaceus, Pediococcus acidilactici, Pediococcus halophilus, Streptococcus faecalis, Streptococcus thermophilus, Staphylococcus camosus, and Staphylococcus xylosus.

118. The composition of any one of claims 106-117, wherein >40%, >60%, and/or >80% of probiotic introduced during the manufacturing process is entrapped within the core materials.

119. The composition of any one of claims 106-117, wherein the barrier material is a solid at 25° C.

120. The composition of any one of claims 106-117, wherein the barrier material is moisture resistant.

121. The composition of any one of claims 106-117, wherein the barrier material melts between 30-90° C.

122. The composition of any one of claims 106-117, wherein the barrier material melts between 36-70° C.

123. The composition of any one of claims 106-117, wherein the barrier material melts between 40-60° C.

124. The composition of any one of claims 120-123, wherein the barrier material is a lipid.

125. The composition of claim 124, wherein the lipid is a wax.

126. The composition of claim 125, wherein the wax comprises paraffin wax, montan wax, microcrystalline wax, polyethylene wax, petrolatum wax, ozokerite wax, ceresin wax, beeswax, lanolin wax, spermaceti wax, tallow wax, lac wax, chinese insect wax, ambergris wax, soy wax, camauba wax, candelilla wax, coconut wax, palm kernel wax, rice bran wax, or a combination thereof.

127. The composition of claim 124, wherein the lipid is a plant oil.

128. The composition of claim 125, wherein the plant oil comprises fatty acid monoglyceride esters, fatty acid diglyceride esters, fatty acid triglyceride esters, coconut oil, cottonseed oil, palm oil, soybean oil, sunflower oil, fully hydrogenated palm oil, partially hydrogenated palm oil, fully hydrogenated sunflower oil, partially hydrogenated sunflower oil, fully hydrogenated soybean oil, partially hydrogenated soybean oil, fully hydrogenated vegetable oil, partially hydrogenated vegetable oil, fully hydrogenated cottonseed oil, partially hydrogenated cottonseed oil, or a combination thereof.

129. The composition of claim 124, wherein the lipid is a fatty acid.

130. The composition of claim 129, wherein the fatty acid comprises butyric acid, pentanoic acid, hexanoic acid, heptanoic acid, caprylic acid, nonanoic acid, capric acid, lauric acid, myristic acid, palmitic acid, stearic acid, and/or arachidonic acid, or a combination thereof.

131. The composition of claim 103 wherein the shell material comprises, on a dry weight basis, about 0% to about 50% (w/w) of the particle preparation.

132. The composition of claim 131, wherein the shell material comprises a carbohydrate, a protein, or a combination thereof.

133. The composition of claim 132, wherein the carbohydrate comprises: amylose, amylopectin, cellulose, methyl cellulose, ethyl cellulose, hydroxypropyl cellulose, hydroxypropyl methyl cellulose, hydroxypropyl ethyl cellulose, cellulose triacetate, cellulose acetate succinate, cellulose acetate butyrate, cellulose acetate phthalate, hydroxypropyl methylcellulose acetate succinate, sodium alginate, potassium alginate, calcium alginate, magnesium alginate, zinc alginate, sodium pectinate, potassium pectinate, calcium pectinate, zinc pectinate, sodium hyaluronate, potassium hyaluronate, calcium hyaluronate, magnesium hyaluronate, zinc hyaluronate, or a combination thereof.

134. The composition of claim 133, wherein the protein comprises: whey protein, β-lactoglobulin, α-lactalbumin, casein, bovine serum albumin, ovalbumin, zein, hordein, gliadin, secalin, kafirin, avenin, or a combination thereof.

135. The composition of any one of claims 93-134, wherein water activity of the composition following incubation for 96 hours at 75% humidity at 25° C. is <about 0.7, <about 0.5, and/or <0.3.

136. The composition of any one of claims 93-134, wherein water activity of the composition following incubation for 96 hours at 53% humidity at 25° C. is <about 0.6, <about 0.5, and/or <0.3.

137. The composition of any one of claims 93-134, wherein water activity of the composition following incubation for 96 hours at 33% humidity at 25° C. is <about 0.5, <about 0.4, and/or <0.3.

138. The composition of any one of claims 93-137, wherein moisture content of the composition following incubation for 96 hours at 75% humidity at 25° C. is <about 8%, <about 4%, and/or <about 2% (w/w).

139. The composition of any one of claims 93-137, wherein moisture content of the composition following incubation for 96 hours at 53% humidity at 25° C. is <about 8%, <about 4%, and/or <about 2% (w/w).

140. The composition of any one of claims 93-137, wherein moisture content of the composition following incubation for 96 hours at 33% humidity at 25° C. is <about 6%, <about 4%, and/or <about 1.5% (w/w).

141. The composition of any one of claims 93-140, wherein the particle preparation is effective to protect against degradation of the microbes.

142. The composition of any one of claims 93-141, wherein degradation comprises loss of log colony forming units (log(CFUs)), changes to particle morphology, changes to particle diameter, or a combination thereof.

143. The composition of any one of claims 93-142, wherein the particle preparation is effective to protect against moisture-induced degradation (e.g., presence of water, humidity, water activity, moisture content or combinations thereof), heat-induced degradation, acid-induced degradation (e.g., presence of simulated gastric fluid), degradation as a result of storage in a food and/or beverage product, or a combination thereof.

144. The composition of any one of claims 93-143, wherein the particle preparation is effective to protect against moisture-induced degradation in aqueous media at 37° C. for at least 1 hour, 2 hours, 3 hours, 6 hours, 12 hours, and/or 24 hours.

145. The composition of claim 144, wherein the loss of log(CFU) is <about 2, <about 1, and/or <about 0.5.

146. The composition of claim 144, wherein the loss of log(CFU) relative to untreated particle preparation is <about 30%, <about 20%, <about 10%.

147. The composition of claim 144, wherein the particle composition diameter is within about 30%, within about 20%, and/or within about 10% of the untreated particle diameter.

148. The composition of any one of claims 93-147, wherein the particle preparation is effective to protect against moisture-induced degradation at elevated relative humidity for at least 1 day, 2 days, 3 days, 6 days, 8 days, or 14 days at 25° C.

149. The composition of claim 148, wherein the particle preparation is effective to protect against moisture-induced degradation at about 35% relative humidity.

150. The composition of claim 92-149, wherein the particle preparation is effective to protect against moisture-induced degradation at about 50% relative humidity.

151. The composition of any one of claims 148-150, wherein the loss of log(CFU) is <about 2, <about 1, and/or <about 0.5.

152. The composition of any one of claims 148-150, wherein the loss of log(CFU) relative to untreated particle preparation is <about 30%, <about 20%, and/or <about 10%.

153. The composition of any one of claims 93-152, wherein the particle preparation is effective to protect against degradation at >about −20° C., >about 4° C., >about 25° C., and/or >about 37° C.

154. The composition of claim 153, wherein the particle preparation is effective to protect against degradation for at least 1 month, 2 months, 6 months, 1 year, and/or 3 years.

155. The composition of any one of claims 153-154, wherein the loss of log(CFU) is <about 2, <about 1, and/or <about 0.5.

156. The composition of any one of claims 153-154, wherein the loss of log(CFU) relative to untreated particle preparation is <about 30%, <about 20%, <about 10%.

157. The composition of any one of claims 153-154, wherein the particle diameter is within about 40%, within about 30%, and/or within about 20% of the untreated particle diameter.

158. The composition of any one of claims 93-157, wherein the particle preparation is effective to protect against acid-induced degradation (i.e., simulated gastric fluid) at 37° C. up to 24 hours, up to 48 hours, up to 96 hours, and/or up to 192 hours.

159. The composition of any one of claims 93-158, wherein the particle preparation is effective to protect against degradation at a pH<about 5, a pH<about 4, a pH<about 3, a pH<about 2.

160. The composition of any one of claims 93-159, wherein the particle preparation is effective to protect against simulated gastric fluid.

161. The composition of claim 160, wherein the loss of log(CFU) is <about 2, <about 1, and/or <about 0.5.

162. The composition of claim 160, wherein the loss of log(CFU) relative to untreated particle preparation is <about 30%, <about 20%, and/or <about 10%.

163. The composition of any one of claims 93-162, wherein the particle preparation is effective to protect against degradation as a result of storage in a food and/or beverage product.

164. The composition of claim 163, wherein the food product comprises a yogurt, agricultural seed, baby formula, bread, candy, capsule, cake, cereal, chip, cookie, dry powder, fertilizer, food additive, ice cream, kefir, nutrition supplement, packaged food, pet feed, pet food, protein bar, protein powder, sachet, salad dressing, smoothie, spice, sprinkle packet, or tablet.

165. The composition of claim 163, wherein the beverage product is comprised at least of: soda, sports drink, beer, kefir, coffee, juice, liquid pharmaceutical formulation, milk, tea, water, or wine.

166. The composition of any one of claims 93-165, wherein the particle preparation is effective to protect against degradation at >about −20° C., >about 4° C., >about 25° C., and/or >about 37° C.

167. The composition of any one of claims 93-166, wherein the particle preparation is effective to protect against degradation for at least 1 week, 2 weeks, 1 month, 2 months, 6 months, 1 year, and/or 3 years.

168. The composition of claim 164, wherein the particle preparation is dispersed within a milk powder.

169. The composition of claim 168, wherein the loss of log(CFU) after 4 weeks at 25° C. and about 35% relative humidity is <about 2, <about 1, and/or <about 0.5.

170. The composition of claim 169, wherein the loss of log(CFU) after 8 weeks at 25° C. and about 35% relative humidity is <about 2, <about 1, and/or <about 0.5.

171. The composition of claim 168, wherein the loss of log(CFU) after 12 weeks at 25° C. and about 35% relative humidity is <about 2, <about 1, and/or <about 0.5.

172. The composition of claim 168, wherein the loss of log(CFU) after 4 weeks at 25° C. and about 50% relative humidity is <about 3, <about 2, and/or <about 1.

173. The composition of claim 168, wherein the loss of log(CFU) after 8 weeks at 25° C. and about 50% relative humidity is <about 3, <about 2, and/or <about 1.

174. The composition of claim 168, wherein the loss of log(CFU) after 12 weeks at 25° C. and about 50% relative humidity is <about 3, <about 2, and/or <about 1.

175. The composition of claim 168, wherein the loss of log(CFU) after 4 weeks at 37° C. and about 35% relative humidity is <about 2, <about 1, and/or <about 0.5.

176. The composition of claim 168, wherein the loss of log(CFU) after 8 weeks at 37° C. and about 35% relative humidity is <about 2, <about 1, and/or <about 0.5.

177. The composition of claim 168, wherein the loss of log(CFU) after 12 weeks at 37° C. and about 35% relative humidity is <about 2, <about 1, and/or <about 0.5.

178. The composition of claim 164, wherein the particle preparation is dispersed within a yogurt.

179. The composition of claim 178, wherein the loss of log(CFU) after 2 weeks at 37° C. and about 35% relative humidity is <about 4, <about 2, and/or <about 1.

180. The composition of claim 178, wherein the loss of log(CFU) after 4 weeks at 37° C. and about 35% relative humidity is <about 4, <about 2, and/or <about 1.

181. The composition of claim 178, wherein the loss of log(CFU) after 8 weeks at 37° C. and about 35% relative humidity is <about 4, <about 2, and/or <about 1.

182. A method for enumerating microbes in probiotic compositions comprising a step of:
i. weighing 2 portions of formulated probiotic compositions;
ii. adding the first portion of formulated probiotic compositions to a warmed, stirred oil bath;
iii. sequentially adding emulsifier and amenable salt solution to the aforementioned stirring oil bath:
iv. serially diluting an aliquot of the aforementioned emulsion;
v. performing spread plate enumeration on the aforementioned dilutions;
vi. adding the second portion of formulated probiotic compositions directly to an aqueous salt solution;
vii. mixing the aforementioned aqueous suspension salt and formulated probiotic compositions;
viii. serially diluting the aforementioned aqueous solution; and
ix. performing spread plate enumeration on the aforementioned dilutions.

183. The method of claim 182, wherein the microbes are bacteria.

184. The method of claim 183, wherein the bacteria are probiotics.

185. The method of claim 184, wherein the probiotics are selected from the group comprising, Bacillus coagulans, Bacillus licheniformis, Bacillus subtilis, Bifidobacterium angulatum, Bifidobacterium animalis, Bifidobacterium bifidum, Bifidobacterium breve, Bifidobacterium infantis, Bifidobacterium Bifidobacterium longum, Enterococcus faecium, Enterococcus faecalis, Lactobacillus acidophilus, Lactobacillus amylovorus, Lactobacillus alimentarius, Lactobacillus bulgaricus, Lactobacillus casei subsp. casei, Lactobacillus casei Sinrota, Lactobacillus curvatus, Lactobacillus delbrueckii subsp lactis, Lactobacillus fermentum, Lactobacillus farciminis, Lactobacillus gasseri, Lactobacillus helveticus, Lactobacillus johnsonii, Lactobacillus lacti, Lactobacillus paracasei, Lactobacillus pentosaceus, Lactobacillus plantarum, Lactobacillus reuteri, Lactobacillus rhamnosus (Lactobacillus GG), Lactobacillus sake, Lactobacillus salivarius, Lactococcus lactis, Micrococcus varians, Pediococcus acidilactici Pediococcus pentosaceus, Pediococcus acidilactici, Pediococcus halophilus, Streptococcus faecalis, Streptococcus thermophilus, Staphylococcus camosus, or Staphylococcus xylosus.

186. The method of any one of claims 182-185, wherein the oil is at least one of vegetable oil, castor oil, avocado oil, sunflower oil, rapeseed oil, mineral oil, or palm oil.

187. The method of claim 186, wherein the mass of oil in the warmed, stirred oil bath is between about 1 to about 100 fold the measured mass of formulated probiotic compositions.

188. The method of claim 186, wherein the mass of oil in the warmed, stirred oil bath is between about 2 to about 75 fold the measured mass of formulated probiotic compositions.

189. The method of claim 186, wherein the mass of oil in the warmed, stirred oil bath is between about 5 to about 50 fold the measured mass of formulated probiotic compositions.

190. The method of any one of claims 186-189, wherein the temperature of the oil bath is between about 20° C. and about 90° C.

191. The method of any one of claims 186-189, wherein the temperature of the oil bath is between about 35° C. and about 80° C.

192. The method of any one of claims 182-191, wherein the emulsifier is characterized as having an HLB value <18.

193. The method of claim 192, wherein the emulsifier comprises at least one of Cetearyl Alcohol, Cetearyl Glucoside, Cetyl Alcohol, Emulsifying Wax, Glyceryl Stearate, PEG-40 Hydrogenated Castor Oil, Polyoxyethylene glycol sorbitan alkyl esters, Polysorbates, Propanediol, Safflower Oleosomes, and Sorbitan alkyl esters.

194. The method of any one of claims 192-193, wherein the mass of emulsifier is between about 1 to about 20 fold relative to the mass of oil in the oil bath.

195. The method of any one of claims 192-194, wherein the amenable salt solution is at least one of Peptone water, saline solution, Phosphate buffer saline solution, Dulbecco's phosphate buffer saline solution, HEPES buffer saline solution, Earl's balanced salt solution, or Hank's balanced salt solution.

196. The method of any one of claims 192-195, wherein the mass of salt solution is added such that the final concentration of emulsifier in the emulsion is between about 0.5% and about 30% (w/w).

197. The method of any one of claims 192-196, wherein the mixing rate is between about 50 and about 500 RPM.

198. The method of any one of claims 192-196, wherein the mixing rate is between about 100 and about 400 RPM.

199. The method of any one of claims 197-198, wherein the mixing time is between about 5 and about 240 minutes.

200. The method of any one of claims 197-198, wherein the mixing time is between about 30 and about 120 minutes.

201. The method of any one of claims 182-200, wherein the emulsions are diluted between about 0 and about 12 10-fold dilutions prior to spread plate enumeration.

202. The method of claim 182, wherein the liquid into which the milled microbes are dispersed comprises a surfactant comprising sodium dodecyl sulfate.

203. An extrudate comprising:
about 85% (w/w) plant oil;
about 10% (w/w) excipient component; and
about 5% (w/w) probiotic bacteria.

204. The extrudate of claim 203, wherein the plant oil comprises hydrogenated palm oil.

205. The extrudate of claim 204, wherein the excipient component comprises CaCO3.

206. The extrudate of claim 204, wherein the probiotic bacteria comprises Bifidobacterium lactis (HN019).

207. The extrudate of claim 203, wherein the excipient component comprises at least one of microcrystalline cellulose, a starch, and maltodextrin.
